(19) United States
(12) Patent Application Publication
(10) Pub. No.: US 2026/0256929 A1
(43) Pub. Date: Sep. 3, 2026

(54) PROTEASE/ENZYME CLEAVABLE LINKER-PAYLOADS AND PROTEIN CONJUGATES

(71) Applicant: Sutro Biopharma, Inc., South San Francisco, CA (US)

(72) Inventors: Krishna BAJJURI, Union City, CA (US); Andreas MADERNA, Walnut Creek, CA (US); Valery Rostislavovich Polyakov, Oakley, CA (US)

(21) Appl. No.: 18/994,106

(22) PCT Filed: Jun. 30, 2023
(86) PCT No.: PCT/US2023/026723

§ 371 Date: Jan. 14, 2025

Related U.S. Application Data
(60) Provisional application No. 63/389,666, filed on Jul. 15, 2022.

(51) Int. Cl. A61K 47/65; A61K 47/54; A61K 47/55; A61P 35/00

(52) U.S. Cl. CPC A61K 47/65; A61K 47/545; A61K 47/552; A61P 35/00

(57) ABSTRACT

Provided herein are compounds, conjugate products thereof, methods, and pharmaceutical compositions for use in treatment and diagnosis.

### CROSS-REFERENCE TO RELATED APPLICATION

This application claims the benefit of U.S. Provisional Application No. 63/389,666 filed Jul. 15, 2022, the entirety of which is incorporated herein for all purposes.

### FIELD

The present disclosure is related to linker-payload compounds and macromolecule conjugates thereof; pharmaceutical compositions comprising linker-payload compounds and/or conjugates; methods of producing linker-payload compounds and/or conjugates; and methods of using the linker-payload compounds, conjugates, and compositions for therapy, such as cellular proliferative disorders, including, but not limited to, cancer.

### BACKGROUND

Biotherapeutics provide a wealth of treatment and diagnostic potential for patients worldwide. However, many drugs based on macromolecules, such as proteins, peptides, and antibodies, present limitations on their effective use, including limitations on bioavailability, absorption, distribution, metabolism, and excretion (ADME). Some of these limitations can affect drug dosage, half-life, side effects, and toxicities. Strategies for improving the effectiveness of biotherapeutics remain needed.

### SUMMARY

In one aspect, provided herein is a compound of Formula (I):

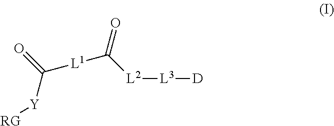

or a pharmaceutically acceptable salt and/or regioisomer thereof;
wherein
L¹ is selected from

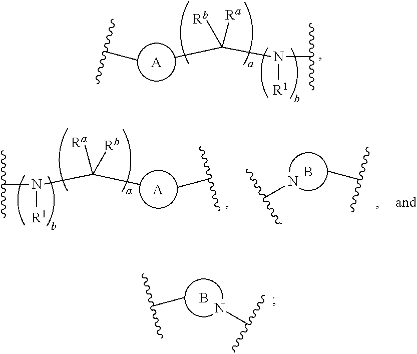

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b is an integer selected from 0 and 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RG;
c is an integer selected from 1, 2, 3, 4, 5, and 6;
RG is a reactive group;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

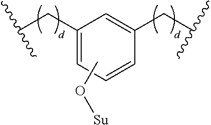

wherein Su is a hexose form of a monosaccharide;
d is an integer independently selected from 1, 2, and 3;
D is a cytotoxic payload; and
[symbol] represents attachment to the remainder of the compound.

In certain embodiments, the compound of Formula (I) is a compound of Formula (IA):

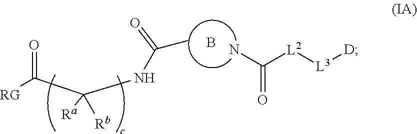

or a pharmaceutically acceptable salt and/or regioisomer thereof;
wherein integer c, RG, R^{a}, R^{b}, Ring B, L², L³, and D are as defined herein.

In certain embodiments, the compound of Formula (I) is a compound of Formula (IB):

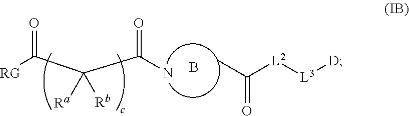

or a pharmaceutically acceptable salt and/or regioisomer thereof;
wherein integer c, RG, R^{a}, R^{b}, Ring B, L², L³, and D are as defined herein.

In certain embodiments, the compound of Formula (I) is a compound of Formula (IC):

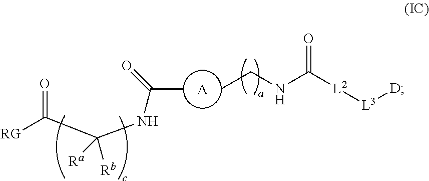

or a pharmaceutically acceptable salt and/or regioisomer thereof;
wherein integer a, integer c, RG, R^{a}, R^{b}, Ring A, L², L³, and D are as defined herein.

In certain embodiments, the compound of Formula (I) is a compound of Formula (ID):

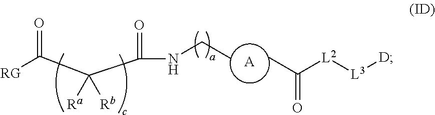

or a pharmaceutically acceptable salt and/or regioisomer thereof;
wherein integer a, integer c, RG, R^{a}, R^{b}, Ring A, L², L³, and D are as defined herein.

In certain embodiments, the compound of Formula (I) is a compound of Formula (IE):

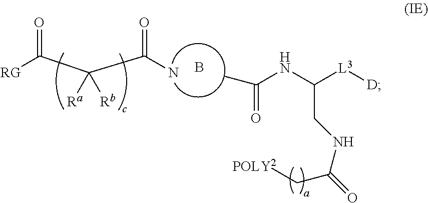

or a pharmaceutically acceptable salt and/or regioisomer thereof;
wherein integer a, integer c, RG, R^{a}, R^{b}, Ring B, L³, POLY², and D are as defined herein.

In certain embodiments, the compound of Formula (I) is a compound of Formula (IF):

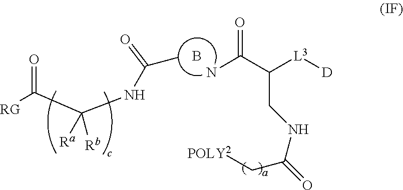

or a pharmaceutically acceptable salt and/or regioisomer thereof;
wherein integer a, integer c, RG, R^{a}, R^{b}, Ring B, L³, POLY², and D are as defined herein.

In certain embodiments, the compound of Formula (I) is a compound of Formula (IG):

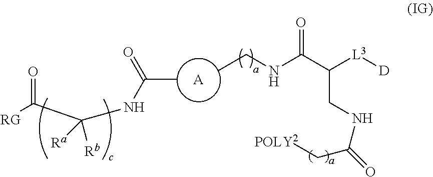

or a pharmaceutically acceptable salt and/or regioisomer thereof;
wherein integer a, integer c, RG, R^{a}, R^{b}, Ring A, L³, POLY², and D are as defined herein.

In certain embodiments, the compound of Formula (I) is a compound of Formula (IH):

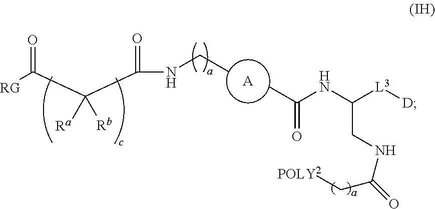

or a pharmaceutically acceptable salt and/or regioisomer thereof;
wherein integer a, integer c, RG, R^{a}, R^{b}, Ring A, L³, POLY², and D are as defined herein.

In one aspect, provided herein is a conjugate of Formula (II):

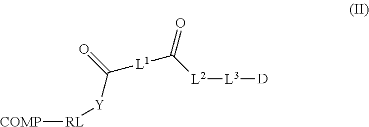

or a pharmaceutically acceptable salt and/or regioisomer thereof;
wherein
L¹ is selected from

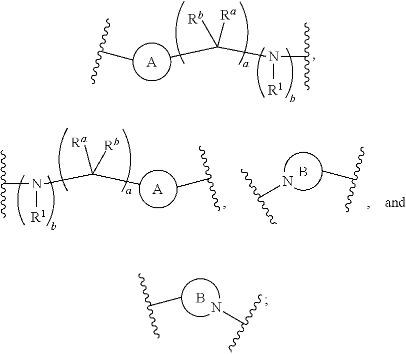

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b is an integer selected from 0 and 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RL;
c is an integer selected from 1, 2, 3, 4, 5, and 6;
RL is a reactive residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

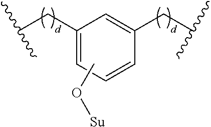

wherein Su is a hexose form of a monosaccharide;
d is an integer independently selected from 1, 2, and 3;
D is a cytotoxic payload;
COMP is a residue of a second compound; and
[symbol] represents attachment to the remainder of the compound.

In certain embodiments, the compound of Formula (II) is a compound of Formula (IIA):

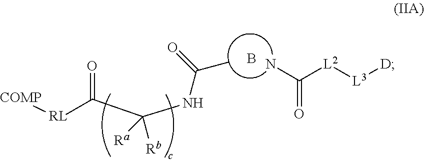

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (II) is a compound of Formula (IIB):

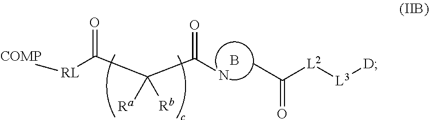

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (II) is a compound of Formula (IIC):

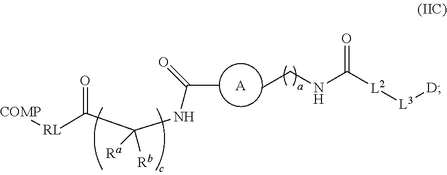

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (II) is a compound of Formula (IID):

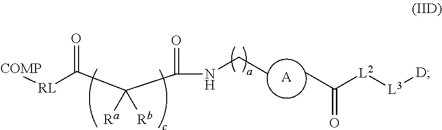

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (II) is a compound of Formula (IE):

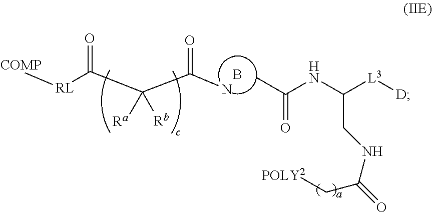

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (II) is a compound of Formula (IIF):

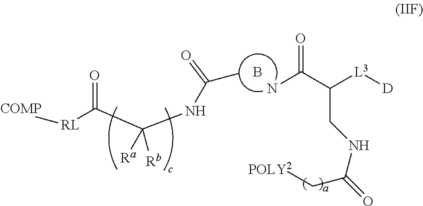

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (II) is a compound of Formula (IIG):

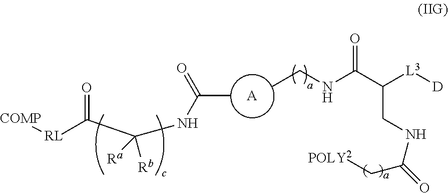

or a pharmaceutically acceptable salt and/or regioisomer thereof;
wherein integer a, integer c, RG, R^{a}, R^{b}, Ring A, L³, POLY², and D are as defined herein.

In certain embodiments, the compound of Formula (II) is a compound of Formula (IIH):

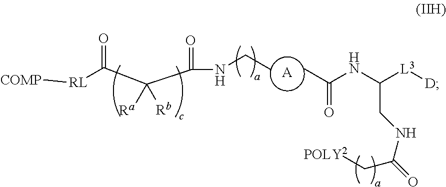

or a pharmaceutically acceptable salt and/or regioisomer thereof;
wherein integer a, integer c, RL, R^{a}, R^{b}, Ring A, L³, POLY², D, and COMP are as defined herein.

In one aspect, provided herein is a conjugate of Formula (III):

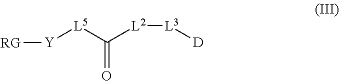

or a pharmaceutically acceptable salt and/or regioisomer thereof;
wherein
L⁵ is a linker comprising an unnatural amino acid; and
RG, Y, L², L³, and D are as defined herein.

Non-limiting unnatural amino acids include sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone.

In certain embodiments, the compound of Formula (III) is a compound of Formula (IIIA):

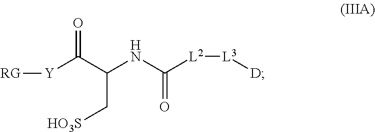

or a pharmaceutically acceptable salt and/or regioisomer thereof;
wherein RG, Y, L², L³, and D are as defined herein.

In certain embodiments, the compound of Formula (III) is a compound of Formula (IIIB):

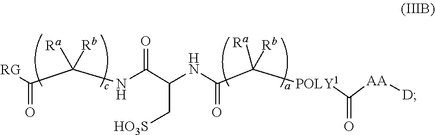

or a pharmaceutically acceptable salt and/or regioisomer thereof;
wherein integer a, integer c, RG, R^{a}, R^{b}, POLY¹, AA, and D are as defined herein.

In certain embodiments, the compound of Formula (IIIB) is a compound of the formula:

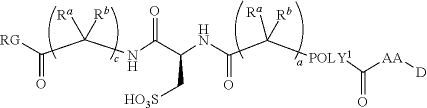

or a pharmaceutically acceptable salt and/or regioisomer thereof;
wherein integer a, integer c, RG, R^{a}, R^{b}, POLY¹, AA, and D are as defined herein.

In one aspect, provided herein is a conjugate of Formula (IV):

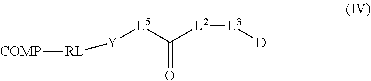

or a pharmaceutically acceptable salt and/or regioisomer thereof;
wherein
L⁵ is a linker comprising an unnatural amino acid; and
RL, COMP, Y, L², L³, and D are as defined herein.

In certain embodiments, the compound of Formula (IV) is a compound of Formula (IVA):

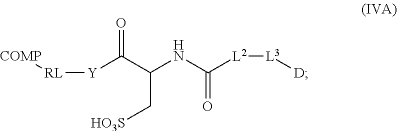

or a pharmaceutically acceptable salt and/or regioisomer thereof;
wherein RL, COMP, Y, L², L³, and D are as defined herein.

In certain embodiments, the compound of Formula (IV) is a compound of Formula (IVB):

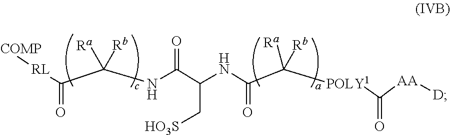

or a pharmaceutically acceptable salt and/or regioisomer thereof;
wherein integer a, integer c, RL, COMP, R^{a}, R^{b}, POLY¹, AA, and D are as defined herein.

In certain embodiments, the compound of Formula (IVB) is a compound of the formula:

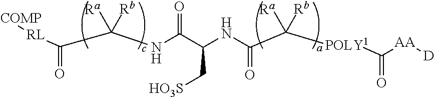

or a pharmaceutically acceptable salt and/or regioisomer thereof
wherein integer a, integer c, RL, COMP, R^{a}, R^{b}, POLY¹, AA, and D are as defined herein.

The present disclosure provides at least the following embodiments:
a) A compound of Formula (I), Formula (IA), Formula (IB), Formula (IC), Formula (ID), Formula (IE), Formula (IF), Formula (IG), Formula (IH), Formula (IIIA), or Formula (IIIB) or a pharmaceutically acceptable salt, solvate, regioisomer, and/or stereoisomer thereof;
b) A compound selected from Compound 1-Compound 25 or a pharmaceutically acceptable salt, solvate, regioisomer, and/or stereoisomer thereof;
c) A pharmaceutical composition comprising a compound of (a) or (b) and a pharmaceutically acceptable excipient, diluent, or carrier;
d) A conjugate of Formula (II), Formula (IIA), Formula (IIB), Formula (IIC), Formula (IID), Formula (IIE), Formula (IIF), Formula (IIG), Formula (IIH), Formula (IVA), or Formula (IVB) or a pharmaceutically acceptable salt, solvate, regioisomer, and/or stereoisomer thereof;
e) A compound selected from Compound 101A-Compound 125B or a pharmaceutically acceptable salt, solvate, regioisomer, and/or stereoisomer thereof;
f) A pharmaceutical composition comprising a compound of (d) or (e) and a pharmaceutically acceptable excipient, diluent, or carrier;
g) A method for the treatment of a disease or disorder in a subject in need thereof comprising administering a therapeutically effective amount of a compound of (a) or (b) or a pharmaceutical composition of (c);
h) A method for the treatment of a disease or disorder in a subject in need thereof comprising administering a therapeutically effective amount of a compound of (d) or (e) or a pharmaceutical composition of (f);
i) A method for inhibiting tubulin polymerization in a subject in need thereof comprising administering a therapeutically effective amount of a compound of (a), (b), (d), or (e) or a pharmaceutical composition of (c) or (f);
j) The method of (g) or (h) wherein the disease or disorder is abnormal cellular proliferation;
k) The method of (j) wherein the abnormal cellular proliferation is cancer;
l) The method of (k) wherein the cancer is small cell lung cancer, non-small cell lung cancer, ovarian cancer, platinum-resistant ovarian cancer, ovarian adenocarcinoma, endometrial cancer, breast cancer, breast cancer which overexpresses HER2, triple-negative breast cancer, a lymphoma, large cell lymphoma, diffuse mixed histiocytic and lymphocytic lymphoma, follicular B cell lymphoma, colon cancer, colon carcinoma, colon adenocarcinoma, colorectal adenocarcinoma, melanoma, prostate cancer, or multiple myeloma;
m) Use of a therapeutically effective amount of a compound of (a) or (b) or a pharmaceutical composition of (c) in the treatment of a disease or disorder in a subject in need thereof;
n) Use of a therapeutically effective amount of a compound of (d) or (e) or a pharmaceutical composition of (f) in the treatment of a disease or disorder in a subject in need thereof;
o) Use of a therapeutically effective amount of a compound of (a) or (b) or a pharmaceutical composition of (c) in the manufacture of a medicament in the treatment of a disease or disorder in a subject in need thereof;
p) Use of a therapeutically effective amount of a compound of (d) or (e) or a pharmaceutical composition of (f) in the manufacture of a medicament in the treatment of a disease or disorder in a subject in need thereof;
q) Use of a therapeutically effective amount of a compound of (a), (b), (d), or (e) or a pharmaceutical composition of (d) or (f) in the manufacture of a medicament for inhibiting tubulin polymerization in a subject in need thereof;
r) The use of (m)-(p) wherein the disease or disorder is abnormal cellular proliferation;
s) The use of (r) wherein the abnormal cellular proliferation is cancer;
t) The use of (s) wherein the cancer is small cell lung cancer, non-small cell lung cancer, ovarian cancer, platinum-resistant ovarian cancer, ovarian adenocarcinoma, endometrial cancer, breast cancer, breast cancer which overexpresses HER2, triple-negative breast cancer, a lymphoma, large cell lymphoma, diffuse mixed histiocytic and lymphocytic lymphoma, follicular B cell lymphoma, colon cancer, colon carcinoma, colon adenocarcinoma, colorectal adenocarcinoma, melanoma, prostate cancer, or multiple myeloma; and
u) a method for producing a conjugate of Formula (II), Formula (IIA), Formula (IIB), Formula (IIC), Formula (IID), Formula (IIE), Formula (IIF), Formula (IIG), Formula (IIH), Formula (IVA), or Formula (IVB) or a pharmaceutically acceptable salt thereof, comprising contacting a compound of Formula (I), Formula (IA), Formula (IB), Formula (IC), Formula (ID), Formula (IE), Formula (IF), Formula (IG), Formula (IH), Formula (IIIA), or Formula (IIIB) or a pharmaceutically acceptable salt thereof, with a second compound under conditions suitable for conjugating the compound of Formula I, Formula (IA), Formula (IB), Formula (IC), Formula (ID), Formula (IE), Formula (IF), Formula (IG), Formula (IH), Formula (IIIA), or Formula (IIIB) with the second compound wherein the second compound comprises an alkyne, cyclooctyne, strained alkene, tetrazine, methylcyclopropene, thiol, maleimide, carbonyl, amine, oxyamine, or azide.

### BRIEF DESCRIPTION OF THE FIGURES

FIG. 1A is a graph showing the killing activity of anti-FolRα ADCs conjugated to Compound 1, Compound 2, and Compound 3 on FolRα positive Igrov1 cells as described in Example 18.

FIG. 1B is a graph that shows anti-FolRα ADCs conjugated to Compound 1, Compound 2, and Compound 3 exhibit no killing activity on FolRα negative A549 cells as described in Example 18.

FIG. 2A is a graph showing the killing activity of anti-FolRα ADCs conjugated to Compound 6, Compound 7, Compound 8, and Compound 9 on FolRα positive Igrov1 cells as described in Example 18.

FIG. 2B is a graph that shows anti-FolRα ADCs conjugated to Compound 6, Compound 7, Compound 8, and Compound 9 exhibit no killing activity on hFolRα negative A549 cells as described in Example 18.

FIG. 3A is a graph showing the killing activity of anti-FolRα ADCs conjugated to Compound 13, Compound 14, and Compound 15 on FolRα positive Igrov1 cells as described in Example 19.

FIG. 3B is a graph that shows anti-FolRα ADCs conjugated to Compound 13, Compound 14, and Compound 15 exhibit no killing activity on FolRα negative A549 cells as described in Example 19.

### DETAILED DESCRIPTION

Described herein are compounds of Formula (I), Formula (IA), Formula (IB), Formula (IC), Formula (ID), Formula (IE), Formula (IF), Formula (IG), Formula (IH), Formula (IIA), Formula (IIB) that are useful for modulating the bioavailability and ADME of, for example, macromolecular conjugate compounds. In some instances, the compounds described herein are useful for preparing conjugates, for instance conjugates of Formula (II), Formula (IIA), Formula (IIB), Formula (IIC), Formula (IID), Formula (IIE), Formula (IIF), Formula (IIG), Formula (IIH), Formula (IVA), or Formula (IVB) for in vivo use. In certain embodiments, the compounds and conjugates feature functionality amenable to enzymatic cleavage to release a payload compound for use in vivo or elsewhere. These compounds can be varied to tune the physiochemical properties and plasma stability of the conjugates. This provides a platform for modulating the bioavailability and ADME of a macromolecule in vivo.

### Definitions

Unless otherwise defined, all terms of art, notations, and other scientific terminology used herein are intended to have the meanings commonly understood by those of skill in the art to which this disclosure pertains. In some cases, terms with commonly understood meanings are defined herein for clarity and/or ready reference. The techniques and procedures described or referenced herein are generally well understood and are commonly employed using conventional methodologies by those skilled in the art, for example, the widely utilized molecular cloning methodologies described in Green & Sambrook, Molecular Cloning: A Laboratory Manual 4^{th} ed. (2012), Cold Spring Harbor Laboratory Press, Cold Spring Harbor, NY; and Ausubel et al., Current Protocols in Molecular Biology, John Wiley & Sons. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value one standard deviation of that value. In certain embodiments, for example, logarithmic scales (e.g., pH), the term “about” indicates the designated value ±0.3, ±0.2, or ±0.1.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

The terms “alkoxy” and “alkoxyl,” as used herein, refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxyamine,” as used herein, refers to the group -alkylene-O—NH₂, wherein alkylene is as defined herein. In some embodiments, alkoxyamine groups can react with aldehydes to form oxime residues. Examples of alkoxyamine groups include —CH₂CH₂—O—NH₂, —CH₂—O—NH₂, and —O—NH₂.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆alkyl, and the like. In certain embodiments, the alkyl group is selected from the group consisting of methyl, ethyl, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with, for example, one, two, or three groups independently selected from a halogen (e.g., fluoro (F), chloro (Cl), bromo (Br), or iodo (I)), alkyl, —CN, —NO₂, amido, —C(O)—, —C(S)—, ester, carbamate, alkenyl, alkynyl, cycloalkyl, heterocycloalkyl, dialkylamino, haloalkyl, hydroxyl, amino, alkylamino, and alkoxy. In some embodiments, alkyl is unsubstituted.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

The term “alkenyl,” as used herein, refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

The term “alkenylene,” as used herein, refers to a divalent alkenyl as defined herein. Lower alkenylene is, for example, C₂-C₆-alkenylene.

The term “alkynyl,” as used herein, refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

The term “alkynylene,” as used herein, refers to a divalent alkynyl as defined herein. Lower alkynylene is, for example, C₂-C₆-alkynylene.

The term “amino,” as used herein, refers to —NH₂.

The term “alkylamino,” as used herein, and unless otherwise specified, refers to the group —NHR″ where R″ is, for example, C₁₋₁₀alkyl, C₂₋₁₀ alkenyl, C₂₋₁₀ alkynyl, C₃₋₁₂ carbocycle, 3- to 12-membered heterocycle, C₁₋₁₀ haloalkyl, and the like as defined herein. In certain embodiments, alkylamino is C₁₋₆alkylamino.

The term “dialkylamino,” as used herein, and unless otherwise specified, refers to the group —NR″R″ where each R″ is independently C₁₋₁₀alkyl, as defined herein. In certain embodiments, dialkylamino is, for example, di-C₁₋₆alkylamino, C₂₋₁₀ alkenyl, C₂₋₁₀ alkynyl, C₃₋₁₂ carbocycle, 3- to 12-membered heterocycle, C₁₋₁₀ haloalkyl, and the like.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (see, e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to a —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

The terms “carboxyl” or “carboxy,” as used herein, refer to —C(O)OH or —COOH.

The terms “cycloalkyl” or “carbocycle” as used herein, unless otherwise specified, refer to a saturated, unsaturated, or aromatic ring in which all atoms of the ring are carbon. In certain embodiments, the “cycloalkyl” or “carbocycle” group may be saturated, and/or bridged, and/or non-bridged, and/or a fused bicyclic group, and/or a spirocyclic bicyclic group. In certain embodiments, the “cycloalkyl” or “carbocycle” group includes three to ten carbon atoms (i.e., C₃ to C₁₀ cycloalkyl). In some embodiments, the “cycloalkyl” or “carbocycle” has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the “cycloalkyl” or “carbocycle” group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. Exemplary “cycloalkyl” or “carbocycles” include cyclopentyl, cyclohexyl, cyclohexenyl, adamantyl, phenyl, indanyl, and naphthyl. “Cycloalkyl” or “carbocycle” includes 3- to 10-membered monocyclic rings, 6- to 12-membered bicyclic rings, and 6- to 12-membered bridged rings. Each ring of a bicyclic cycloalkyl or carbocycle may be selected from saturated, unsaturated, and aromatic rings. A bicyclic cycloalkyl or carbocycle includes any combination of saturated, unsaturated and aromatic bicyclic rings, as valence permits. A bicyclic cycloalkyl or carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Non-limiting examples of bridged bicyclic cycloalkyl or carbocycle groups include, but are not limited to, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, and 2-oxabicyclo[2.2.2]octyl. Non-limiting examples of spirocyclic cycloalkyl or carbocycle groups include, but are not limited to, spiro[3.3]heptyl, spiro[3.4]octyl, spiro[3.5]nonyl, spiro[3.6]decyl, spiro[4.4]nonyl, spiro[4.5]decyl, spiro[5.5]undecyl, spiro[5.6]dodecyl, and spiro[5.7]tridecyl.

The term “bicyclic ring system” includes 6-12 (e.g., 8-12 or 9-, 10-, or 11-) membered structures that form two rings, wherein the two rings have at least one atom in common (e.g., two atoms in common). Bicyclic rings can be fused, bridged, or spirocyclic. Bicyclic ring systems include bicycloaliphatics (e.g., bicycloalkyl or bicycloalkenyl), bicycloheteroaliphatics, bicyclic aryls, and bicyclic heteroaryls.

The term “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The term “spiro bicyclic ring system” refers to a bicyclic heterocyclicalipahtic ring system or bicyclic cycloaliphatic ring system in which 2 or 3 rings are linked together by one common atom. Spiro compounds depicted with overlapping rings indicate that the rings can bond at any vertex. For instance, in the spiro group

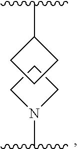

the two rings can bond at any of the three available vertex atoms in either ring.

The term “cycloalkylene,” as used herein refers to a divalent cycloalkyl group, as defined herein. In certain embodiments, the cycloalkylene group is cyclopropylene

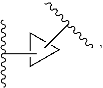

cyclobutylene

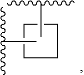

cyclopentylene

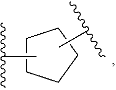

cyclohexylene

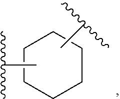

cycloheptylene

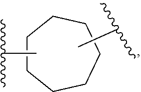

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR or —COOR where R is alkyl, as defined herein.

The term “fluorene” as used herein refers to

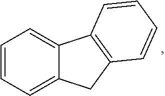 [SMILES: c1ccc2c(c1)Cc1ccccc1-2]

wherein any one or more carbons bearing one or more hydrogens can be substituted with a chemical functional group as described herein.

The term “haloalkyl” refers to an alkyl group, as defined herein, substituted with one or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycloalkyl” or “heterocycle” refers to a saturated, unsaturated or aromatic ring comprising one or more heteroatoms. Exemplary heteroatoms include N, O, Si, P, B, and S atoms where the nitrogen or sulfur atoms may be optionally oxidized, and the nitrogen atoms may be optionally quaternized and the remaining ring atoms of the non-aromatic ring are carbon atoms. A “heterocycloalkyl” or “heterocycle” includes 3- to 10-membered monocyclic rings, 6- to 12-membered bicyclic rings, and 6- to 12-membered bridged rings. In certain embodiments, “heterocycloalkyl” or “heterocycle” is a monovalent, monocyclic, or multicyclic fully-saturated ring system. In certain embodiments, the “heterocycloalkyl” or “heterocycle” group may be unsaturated, and/or bridged, and/or non-bridged, and/or a fused bicyclic group, and/or a spirocyclic bicyclic group. A bicyclic “heterocycloalkyl” or “heterocycle” includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. In certain embodiments, the “heterocycloalkyl” or “heterocycle” group has from three to twenty, from three to fifteen, from three to ten, from three to eight, from four to seven, from four to eleven, or from five to six ring atoms. The “heterocycloalkyl” or “heterocycle” may be attached to a core structure at any heteroatom or carbon atom which results in the creation of a stable compound. In certain embodiments, the “heterocycloalkyl” or “heterocycle” is a monocyclic, bicyclic, tricyclic, or tetracyclic ring system, which may include a fused or bridged or spirocyclic ring system and in which the nitrogen or sulfur atoms may be optionally oxidized, and/or the nitrogen atoms may be optionally quaternized. In some embodiments, “heterocycloalkyl” or “heterocycle” radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. Non-limiting examples of bridged heterocycloalkyl or heterocycle groups include, but are not limited to, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. Non-limiting examples of spirocyclic heterocycloalkyl or heterocycle groups include, but are not limited to, 2,8-diazaspiro[4.5]decyl; 2,7-diazaspiro[3.5]nonyl; 3,9-diazaspiro[5.5]undecyl; 3-azaspiro[5.5]undecyl; 2-oxa-6-azaspiro[3.4]octyl; 2-oxa-9-azaspiro[5.5]undecyl; 3-oxa-9-azaspiro[5.5]undecyl; 7-azaspiro[3.5]nonyl; 2-azaspiro[3.5]nonyl; 7-oxaspiro[3.5]nonyl; and, 2-oxaspiro[3.5]nonyl.

In certain embodiments, “heterocycloalkyl” or “heterocycle” may also be optionally substituted as described herein. In certain embodiments, “heterocycloalkyl” or “heterocycle” is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heterocycloalkyl or “heterocycle” group may comprise one, two, three, or four heteroatoms. Those of skill in the art will recognize that a 4-membered “heterocycloalkyl” or “heterocycle” may generally comprise one or two heteroatoms, a 5- or 6-membered “heterocycloalkyl” or “heterocycle” may generally comprise one, two, or three heteroatoms, and a 7- to 10-membered heterocycloalkyl or “heterocycle” may generally comprise one, two, three, or four heteroatoms.

“Heterocycloalkylene” refers to a divalent heterocycloalkyl as defined herein.

The term “heteroaryl” refers to a monovalent, monocyclic aromatic group and/or multicyclic aromatic group, wherein at least one aromatic ring contains one or more heteroatoms independently selected from oxygen, sulfur, and nitrogen within the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

The term “protecting group,” as used herein, and unless otherwise specified, refers to a group that is added to an oxygen, nitrogen, or phosphorus atom to prevent further reaction at the (protected) oxygen, nitrogen, or phosphorus, or for other purposes. A wide variety of oxygen and nitrogen protecting groups are known to those skilled in the art of organic synthesis (see, e.g., Greene, et al., Protective Groups in Organic Synthesis, John Wiley and Sons, Fourth Edition, 2006, which is incorporated herein by reference in its entirety).

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, and muconic acids, and the like; or (2) salts formed when an acidic proton present in the parent compound either (a) is replaced by a metal ion, for example, an alkali metal ion, an alkaline earth ion, or an aluminum ion, or alkali metal or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, omithine, choline, N,N′-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxvethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85% or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods and compounds provided herein, the compounds are substantially free of enantiomers (i.e., the compounds are not a racemic or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers, or diastereomers.

“Solvate” refers to a compound provided herein, or a salt thereof, that further includes a stoichiometric or non-stoichiometric amount of solvent bound by non-covalent intermolecular forces. Where the solvent is water, the solvate is a hydrate.

The term “substituted” refers to moieties having substituents replacing a hydrogen on one or more carbons or substitutable heteroatoms, e.g., an NH or NH₂ of a compound. It will be understood that“substitution” or “substituted with” includes the implicit proviso that such substitution is in accordance with permitted valence of the substituted atom and the substituent, and that the substitution results in a stable compound. i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term“substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b1}—OR^{a1}, —R^{b1}—OC(O)—R^{a1}, —R^{b1}—OC(O)—OR^{a1}, —R^{b1}—OC(O)—N(R^{a1})₂, —R^{b1}—N(R^{a})₂, —R^{b1}—C(O)R^{a1}, —R^{b1}—C(O)OR^{a1}, —R^{b1}—C(O)N(R)₂, —R^{b1}—O—R^{c1}—C(O)N(R^{a1})₂, —R^{b1}—N(R^{a1})C(O)OR^{a1}, —R^{b1}—N(R^{a})C(O)R^{a1}, —R^{b1}—N(R^{a1})S(O)ₜR^{a1} (where t is 1 or 2), —R^{b1}—S(O)ₜR^{a1} (where t is 1 or 2), —R^{b1}—S(O)ₜOR^{a1} (where t is 1 or 2), and —R^{b1}—S(O)ₜN(R^{a1})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b1}—OR^{a1}, —R^{b1}—OC(O)—R^{a1}, —R^{b1}—OC(O)—OR^{a1}, —R^{b1}—OC(O)—N(R^{a1})₂, —R^{b1}—N(R^{a1})₂, —R^{b1}—C(O)R^{a1}, —R^{b1}C(O)OR^{a1}, —R^{b1}—C(O)N(R)₂, —R^{b1}—O—R^{c1}—C(O)N(R^{a1})₂, —R^{b1}—N(R^{a1})C(O)OR^{a1}, —R^{b1}—N(R^{a1})C(O)R^{a1}, —R^{b1}—N(R^{a1})S(O)ₜR^{a1} (where t is 1 or 2), —R^{b1}—S(O)ₜR^{a1} (where t is 1 or 2), —R^{b1}—S(O)ₜOR^{a1} (where t is 1 or 2) and —R^{b1}—S(O)ₜN(R^{a1})₂ (where t is 1 or 2); wherein each R^{a1} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a1}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b1}—OR^{a1}, —R^{b1}—OC(O)—R^{a1}, —R^{b1}—OC(O)—OR^{a1}, —R^{b1}—OC(O)—N(R^{a1})₂, —R^{b1}—N(R^{a1})₂, —R^{b1}—C(O)R^{a1}, —R^{b1}—C(O)OR^{a1}, —R^{b1}—C(O)N(R^{a1})₂, —R^{b1}—O—R^{c1}—C(O)N(R^{a1})₂, —R^{b1}—N(R^{a1})C(O)OR^{a1}, —R^{b1}—N(R^{a1})C(O)R^{a1}, —R^{b1}—N(R^{a})S(O)ₜR^{a1} (where t is 1 or 2), —R^{b1}—S(O)Ral (where t is 1 or 2), —R^{b1}—S(O)ₜOR^{a1} (where t is 1 or 2) and —R^{b1}—S(O)tN(R^{a1})2 (where t is 1 or 2); and wherein each R^{b1} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as“unsubstituted,” references to chemical moieties herein are understood to include substituted variants. For example, reference to a “heteroaryl” group or moiety implicitly includes both substituted and unsubstituted variants, unless specified otherwise.

The term “amino acid” or “amino acid residue” refers to a D- or L-natural or non-naturally occurring amino acid. Representative amino acids include, but are not limited to, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan, or tyrosine, among others. “Unnatural amino acids” are non-proteinogenic amino acids that either occur naturally or are chemically synthesized. Non-limiting examples of unnatural amino acids include sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise deuterium (D) at one or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise carbon-13 (¹³C) at an amount other than the natural isotopic composition.

The term “macromolecule” or “macromolecular moiety” refers to a protein, peptide, antibody, nucleic acid, carbohydrate, or other large molecule composed of polymerized monomers. They include peptides of two or more residues, or ten or more residues. In certain embodiments, a macromolecule is at least 1000 Da in mass. In certain embodiments, a macromolecule has at least 1000 atoms. In certain embodiments, a macromolecule can be modified. For instance, a protein, peptide, or antibody can be modified with one or more carbohydrates and/or small molecule therapeutic compounds.

The term “immunoglobulin” refers to a class of structurally related proteins generally comprising two pairs of polypeptide chains: one pair of light (L) chains, and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H} or VH) and a heavy chain constant region (C_{H} or CH). The heavy chain constant region typically comprises three domains, abbreviated C_{H}1 (or CH1), C_{H}2 (or CH2), and C_{H}3 (or CH3). Each light chain typically comprises a light chain variable region (V_{L} or VL) and a light chain constant region. The light chain constant region typically comprises one domain, abbreviated C_{L} or CL.

The term “antibody” is used herein in its broadest sense. An antibody includes intact antibodies (e.g., intact immunoglobulins), and antibody fragments (e.g., antigen binding fragments or antigen-binding fragments of antibodies). Antibodies comprise at least one antigen-binding domain. One example of an antigen-binding domain is an antigen binding domain formed by a V_{H}-V_{L} dimer.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V), and the less common pyrrolysine and selenocysteine. Natural amino acids also include citrulline. Naturally encoded amino acids include post-translational variants of the twenty-two naturally occurring amino acids such as prenylated amino acids, isoprenylated amino acids, myristoylated amino acids, palmitoylated amino acids, N-linked glycosylated amino acids, O-linked glycosylated amino acids, phosphorylated amino acids, and acylated amino acids. The term “amino acid” also includes non-natural (or unnatural) or synthetic α-, β-, γ-, or δ-amino acids, and includes, but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. In certain embodiments, the amino acid is in the D-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl, or β-histidinyl. Unnatural amino acids are not proteinogenic amino acids, or post-translationally modified variants thereof. In particular, the term unnatural amino acid refers to an amino acid that is not one of the twenty common amino acids or pyrrolysine or selenocysteine, or post-translationally modified variants thereof.

The term “conjugate” refers to a compound or drug moiety described herein linked to one or more macromolecular moieties. The macromolecular moiety is as defined herein or is any macromolecule deemed suitable to the person of skill in the art. The compound or drug moiety can be any compound or drug moiety described herein. The compound or drug moiety can be directly linked to the macromolecular moiety via a covalent bond, or the compound or drug moiety can be linked to the macromolecular moiety indirectly via a linker. Typically, the linker is covalently bonded to the macromolecular moiety and also covalently bonded to the compound or drug moiety.

“pAMF,” “pAMF residue,” or “pAMF mutation” refers to a variant phenylalanine residue (i.e., para-azidomethyl-L-phenylalanine) added or substituted into a polypeptide.

The term “linker” refers to a molecular moiety that is capable of forming at least two covalent bonds. Typically, a linker is capable of forming at least one covalent bond to a macromolecular moiety and at least another covalent bond to a compound or drug moiety. In certain embodiments, a linker can form more than one covalent bond to a macromolecular moiety. In certain embodiments, a linker can form more than one covalent bond to a compound or drug moiety or can form covalent bonds to more than one compound or drug moiety. After a linker forms a bond to a macromolecular moiety, or a compound or drug moiety, or both, the remaining structure (i.e. the residue of the linker (“linker residue”) after one or more covalent bonds are formed) may still be referred to as a “linker” herein. The term “linker precursor” refers to a linker having one or more reactive groups capable of forming a covalent bond with a macromolecule, or compound or drug moiety, or both. A person of ordinary skill in the art, given the context of how the term linker is used, would understand whether “linker” means linker precursor with one reactive group, a linker precursor with more than one reactive groups, a linker residue which is covalently bonded to the macromolecule, a linker residue which is covalently bonded to a compound or drug moiety, and/or a linker residue which is covalently bonded to the macromolecule and is covalently bonded to a compound or drug moiety. In some embodiments, the linker is a cleavable linker. For example, a cleavable linker can be one that is released by a bio-labile or enzymatic function, which may or may not be engineered. In some embodiments, the linker is a non-cleavable linker. For example, a non-cleavable linker can be one that is released upon degradation of the macromolecular moiety.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, and mouse) and a primate (e.g., a monkey, such as a cynomolgous monkey, a chimpanzee, and a human), and in certain embodiments, a human. In certain embodiments, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound or conjugate provided herein. In certain embodiments, a therapeutic agent is an agent which is known to be useful for, or has been or is currently being used for the treatment or prevention of a disorder or one or more symptoms thereof.

“Therapeutically effective amount” refers to an amount of a compound or composition that, when administered to a subject for treating a condition, is sufficient to effect such treatment for the condition. A “therapeutically effective amount” can vary depending on, inter alia, the compound, the disease or disorder and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or retarding the progression of the disease or disorder or of one or more symptoms of the disease or disorder, or reducing the severity of the disease or disorder or of one or more symptoms of the disease or disorder.

As used herein, the term “inhibits growth” (e.g., referring to cells, such as tumor cells) is intended to include any measurable decrease in cell growth (e.g., tumor cell growth) when contacted with a compound, drug moiety, or conjugate herein, as compared to the growth of the same cells not in contact with the compound, drug moiety, or conjugate herein. In some embodiments, growth may be inhibited by at least about 10%, 20%, 30%, 40%, 50%, 60%, 70%, 80%, 90%, 99%, or 100%. The decrease in cell growth can occur via a variety of mechanisms, including but not limited to, conjugate, compound, or drug moiety internalization, apoptosis, necrosis, and/or effector function-mediated activity.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound, drug moiety, or conjugate provided herein. In certain other embodiments, the term “prophylactic agent” does not refer a compound, drug moiety, or conjugate provided herein. For example, a prophylactic agent is an agent which is known to be useful for, or has been or is currently being used to prevent or impede the onset, development, progression, and/or severity of a disorder.

As used herein, the phrase “prophylactically effective amount” refers to the amount of a therapy (e.g., prophylactic agent) which is sufficient to result in the prevention or reduction of the development, recurrence, or onset of one or more symptoms associated with a disorder or to enhance or improve the prophylactic effect(s) of another therapy (e.g., another prophylactic agent).

In some chemical structures illustrated herein, certain substituents chemical groups, and atoms are depicted with a curvy/wavy/wiggly line

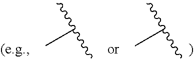

that intersects a bond or bonds to indicate the atom through which the substituents, chemical groups, and atoms are bonded. For example, in some structures, such as but not limited to,

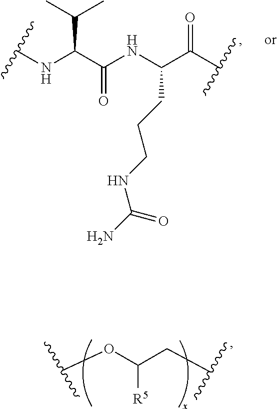

this curvy/wavy/wiggly line indicates the atoms in the backbone of a conjugate, compound, or drug moiety structure to which the illustrated chemical entity is bonded. In some structures, such as but not limited to

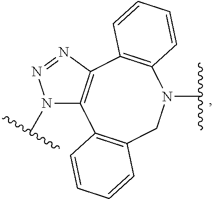 [SMILES: *N1Cc2ccccc2-c2c(nnn2*)-c2ccccc21]

this curvy/wavy/wiggly line indicates the atoms in the macromolecule as well as the atoms in the backbone of a conjugate, compound, or drug moiety structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

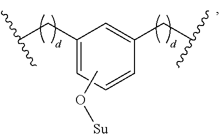

wherein the positions of substituent O-Su are described generically, i.e., not directly attached to any vertex of the bond line structure, i.e., specific ring carbon atom, includes the following, non-limiting examples of groups in which the substituent O-Su is bonded to a specific ring carbon atom:

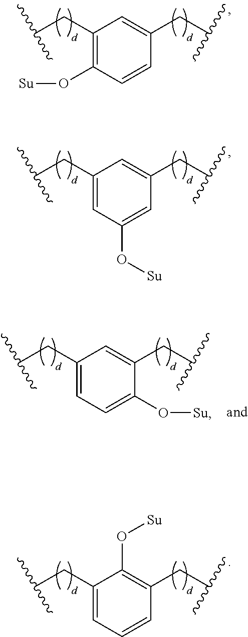

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### Compounds of Formula (I) and (III) and Conjugates of Formula (II) and (IV)

In one aspect, the compound is a compound of Formula (I):

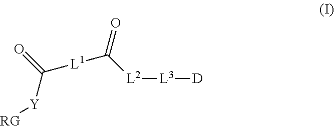

or a pharmaceutically acceptable salt and/or regioisomer thereof;
wherein
L¹ is selected from

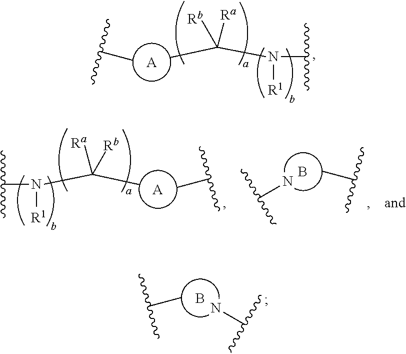

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b is an integer selected from 0 and 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RG;
c is an integer selected from 1, 2, 3, 4, 5, and 6;
RG is a reactive group;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

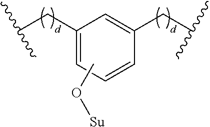

wherein Su is a hexose form of a monosaccharide;
d is an integer independently selected from 1, 2, and 3;
D is a cytotoxic payload; and
[symbol] represents attachment to the remainder of the compound.

In certain embodiments, the compound of Formula (I) is a compound of Formula (IA):

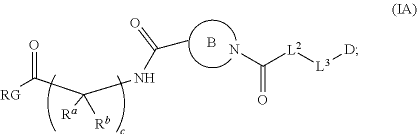

or a pharmaceutically acceptable salt and/or regioisomer thereof;
wherein integer c, RG, R^{a}, R^{b}, Ring B, L², L³, and D are as defined herein.

In certain embodiments, the compound of Formula (IA) is selected from the following:

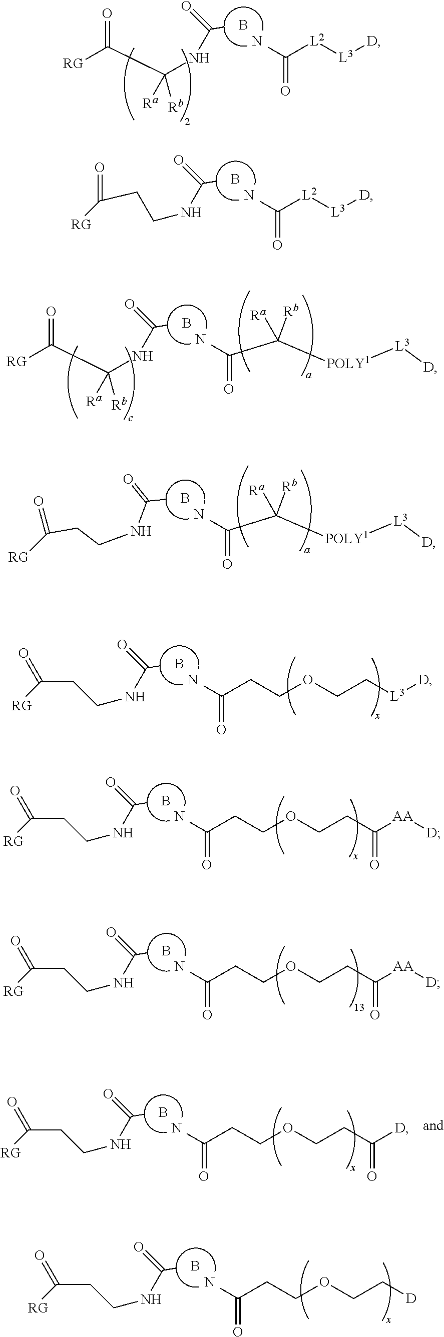

or a pharmaceutically acceptable salt and/or regioisomer thereof.

In certain embodiments, the compound of Formula (I) is a compound of Formula (IB):

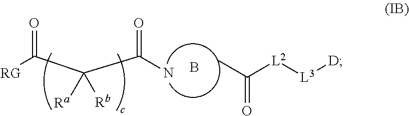

or a pharmaceutically acceptable salt and/or regioisomer thereof;
wherein integer c, RG, R^{a}, R^{b}, Ring B, L², L³, and D are as defined herein.

In certain embodiments, the compound of Formula (IB) is selected from the following:

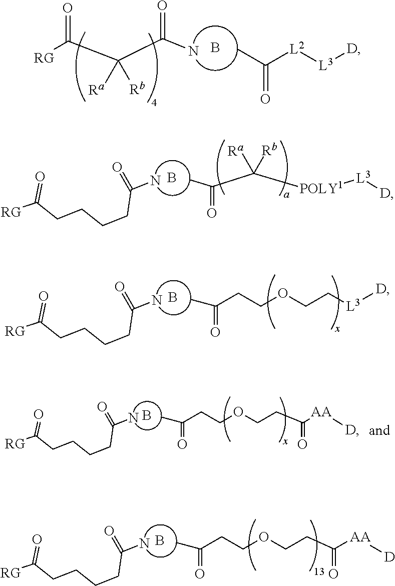

or a pharmaceutically acceptable salt and/or regioisomer thereof.

In certain embodiments, the compound of Formula (I) is a compound of Formula (IC):

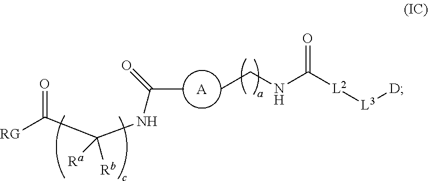

or a pharmaceutically acceptable salt and/or regioisomer thereof;
wherein integer a, integer c, RG, R^{a}, R^{b}, Ring A, L², L³, and D are as defined herein.

In certain embodiments, the compound of Formula (IC) is selected from the following:

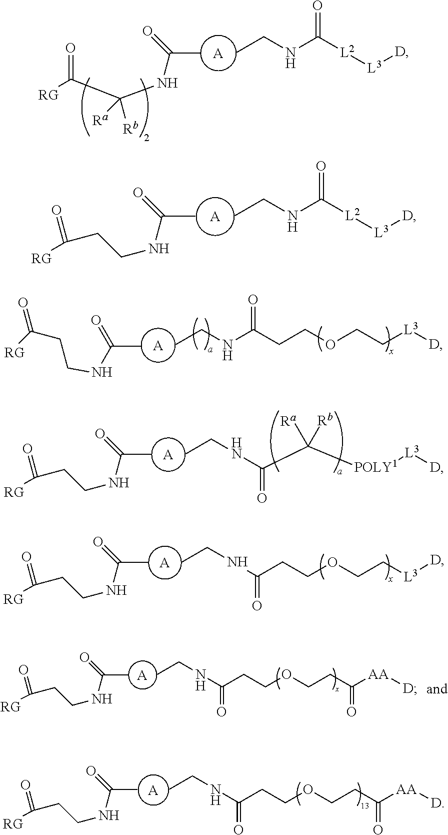

or a pharmaceutically acceptable salt d/or regioisomer thereof.

In certain embodiments, the compound of Formula (I) is a compound of Formula (ID):

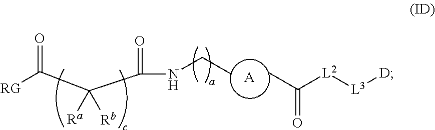

or a pharmaceutically acceptable salt and/or regioisomer thereof;
wherein integer a, integer c, RG, R^{a}, R^{b}, Ring A, L², L³, and D are as defined herein.

In certain embodiments, the compound of Formula (ID) is selected from the following:

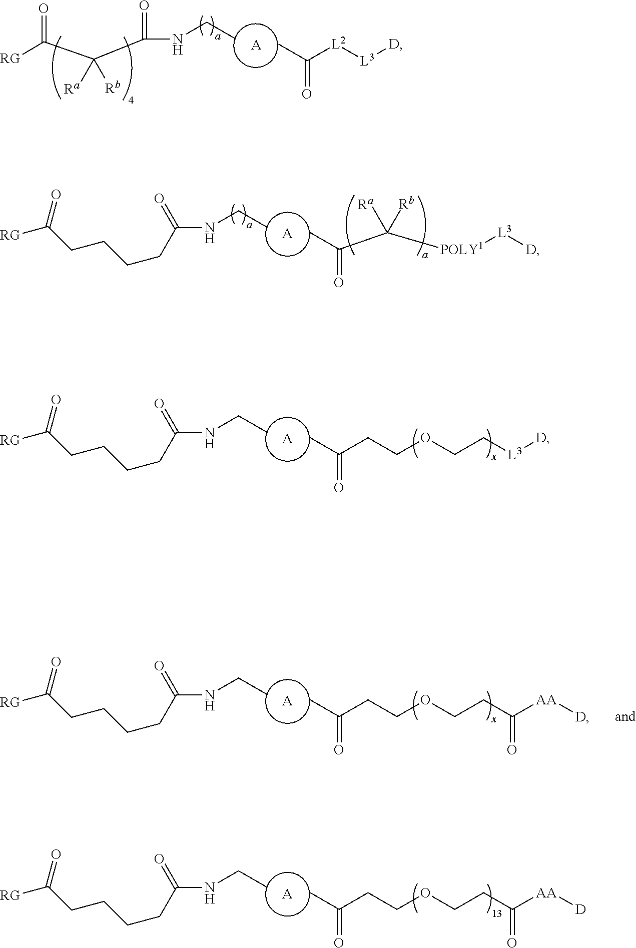

or a pharmaceutically acceptable salt and/or regioisomer thereof.

In certain embodiments, the compound of Formula (I) is a compound of Formula (IE):

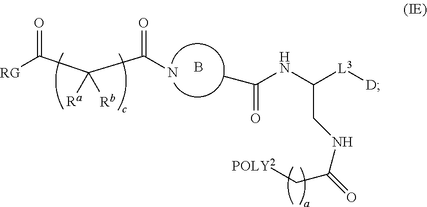

or a pharmaceutically acceptable salt and/or regioisomer thereof,
wherein integer a, integer c, RG, R^{a}, R^{b}, Ring B, L³, POLY², and D are as defined herein.

In certain embodiments, the compound of Formula (IE) is selected from the following:

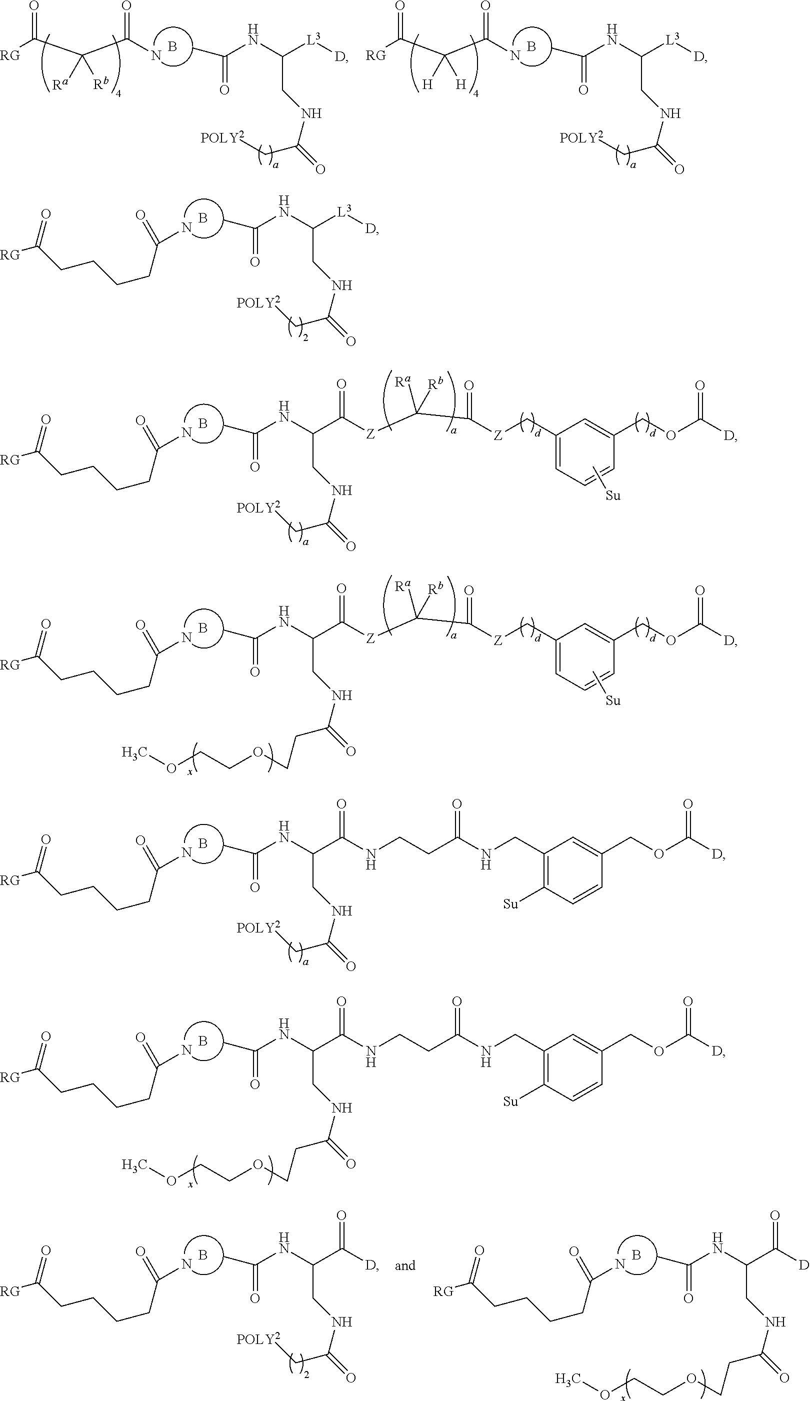

or a pharmaceutically acceptable salt and/or regioisomer thereof.

In certain embodiments, the compound of Formula (I) is a compound of Formula (IF):

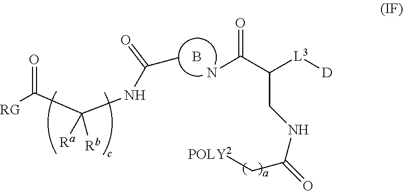

or a pharmaceutically acceptable salt and/or regioisomer thereof;
wherein integer a, integer c, RG, R^{a}, R^{b}, Ring B, L³, POLY², and D are as defined herein.

In certain embodiments, the compound of Formula (IF) is selected from the following:

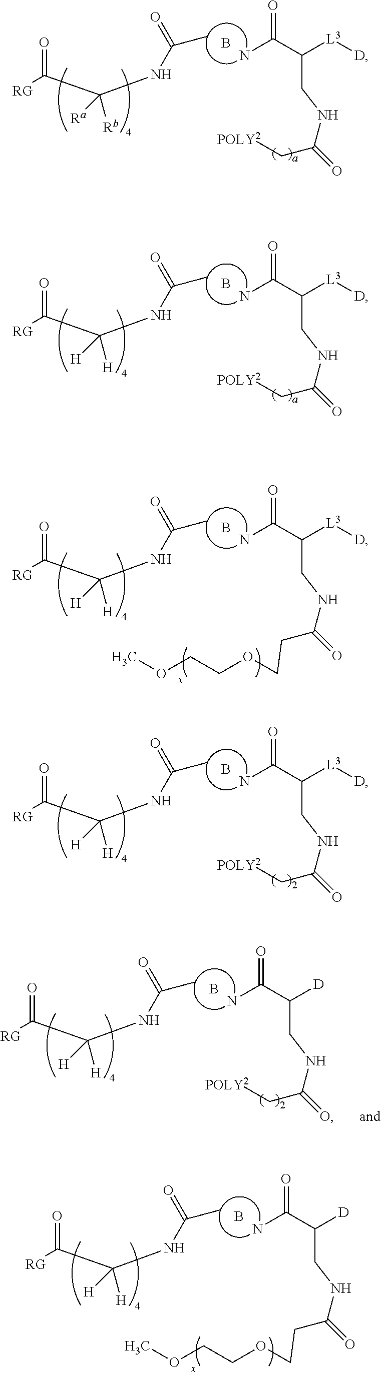

or a pharmaceutically acceptable salt and/or regioisomer thereof.

In certain embodiments, the compound of Formula (I) is a compound of Formula (IG):

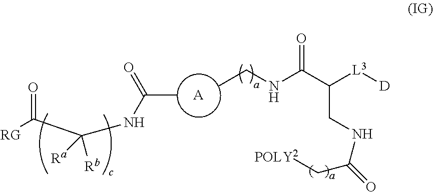

or a pharmaceutically acceptable salt and/or regioisomer thereof;
wherein integer a, integer c, RG, R^{a}, R^{b}, Ring A, L³, POLY², and D are as defined herein.

In certain embodiments, the compound of Formula (IG) is selected from the following:

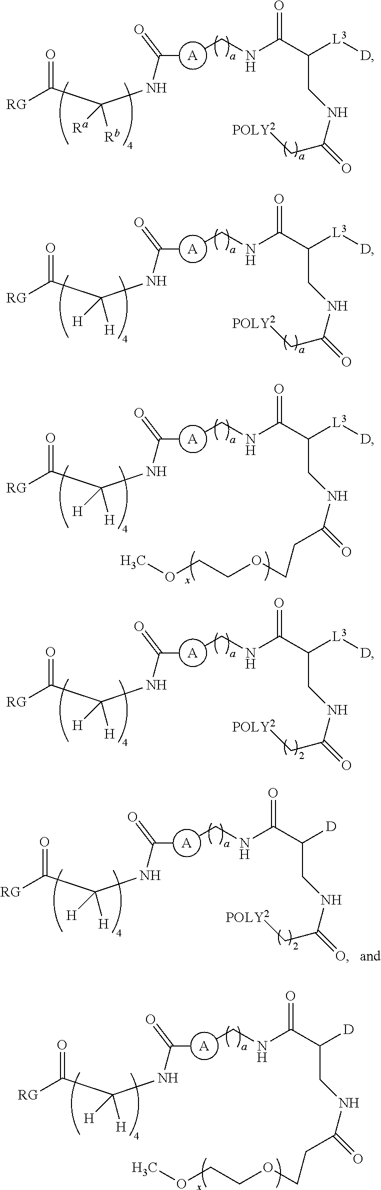

or a pharmaceutically acceptable salt and/or regioisomer thereof.

In certain embodiments, the compound of Formula (I) is a compound of Formula (IH):

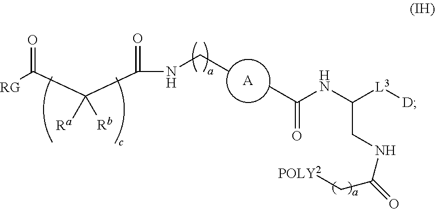

or a pharmaceutically acceptable salt and/or regioisomer thereof;
wherein integer a, integer c, RG, R^{a}, R^{b}, Ring A, L³, POLY², and D are as defined herein.

In certain embodiments, the compound of Formula (IH) is selected from the following:

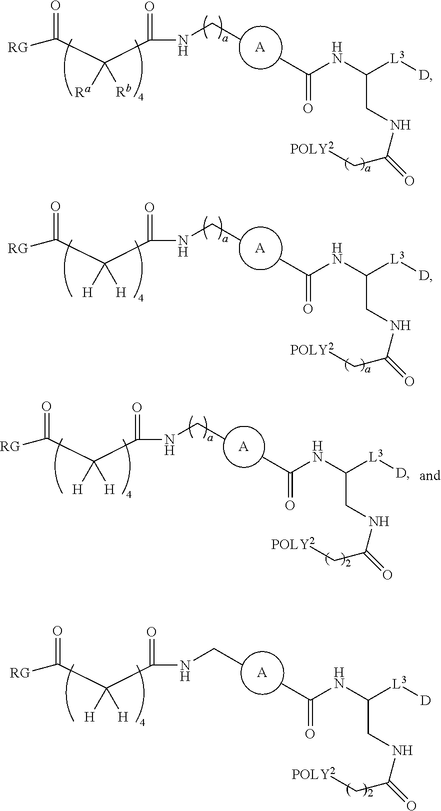

or a pharmaceutically acceptable salt and/or regioisomer thereof.

In one aspect, provided herein is a conjugate of Formula (II):

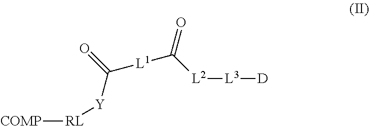

or a pharmaceutically acceptable salt and/or regioisomer thereof;
wherein
L¹ is selected from

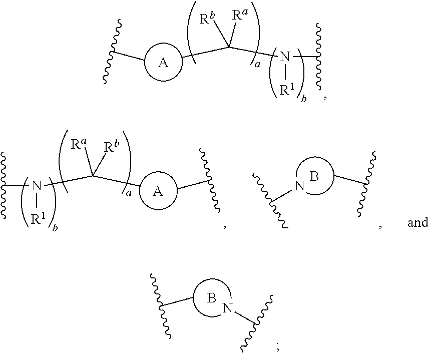

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RL;
c is an integer selected from 1, 2, 3, 4, 5, and 6;
RL is a reactive residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

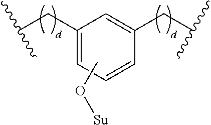

wherein Su is a hexose form of a monosaccharide;
d is an integer independently selected from 1, 2, and 3;
D is a cytotoxic payload;
COMP is a residue of a second compound; and
[symbol] represents attachment to the remainder of the compound.

In certain embodiments, the compound of Formula (II) is a compound of Formula (IIA):

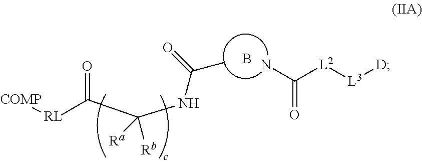

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IA) is selected from the following:

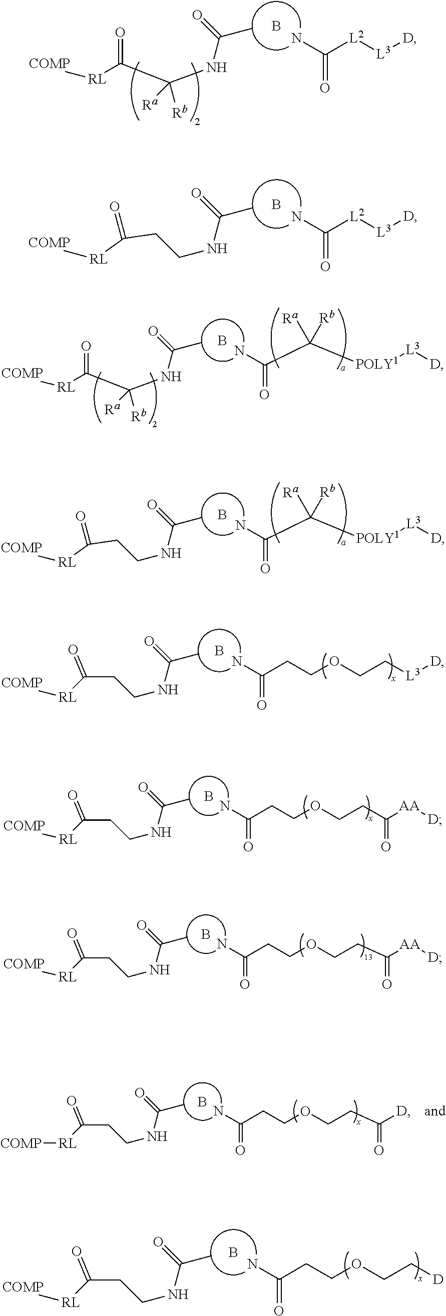

or a pharmaceutically acceptable salt and/or regioisomer thereof.

In certain embodiments, the compound of Formula (II) is a compound of Formula (IIB):

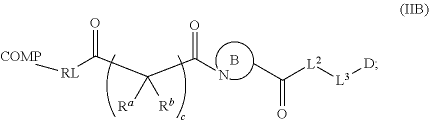

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IIB) is selected from the following:

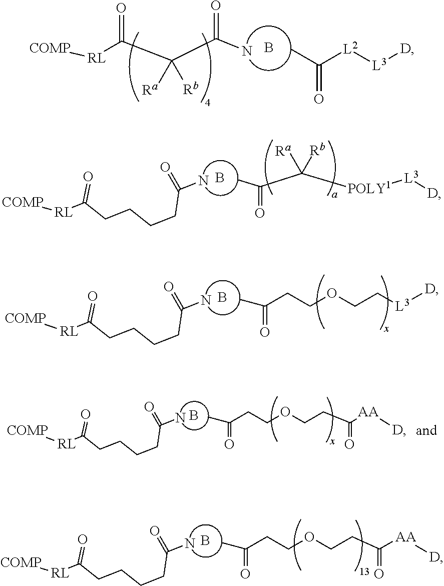

or a pharmaceutically acceptable salt and/or regioisomer thereof.

In certain embodiments, the compound of Formula (II) is a compound of Formula (IIC):

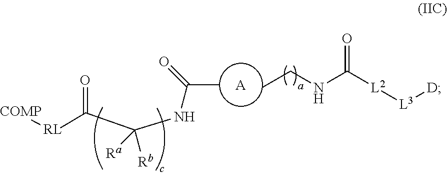

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IIC) is selected from the following:

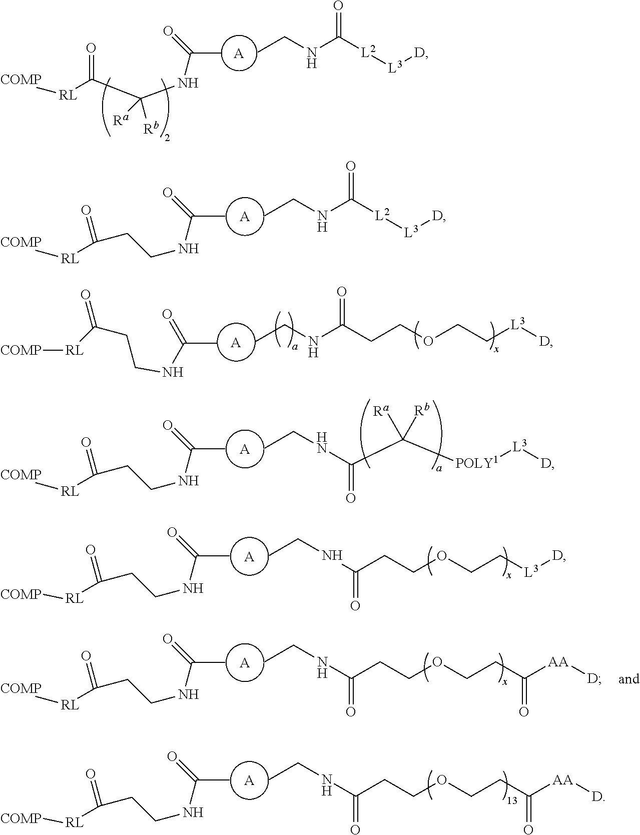

or a pharmaceutically acceptable salt and/or regioisomer thereof.

In certain embodiments, the compound of Formula (II) is a compound of Formula (IID):

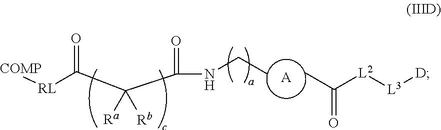

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IID) is selected from the following:

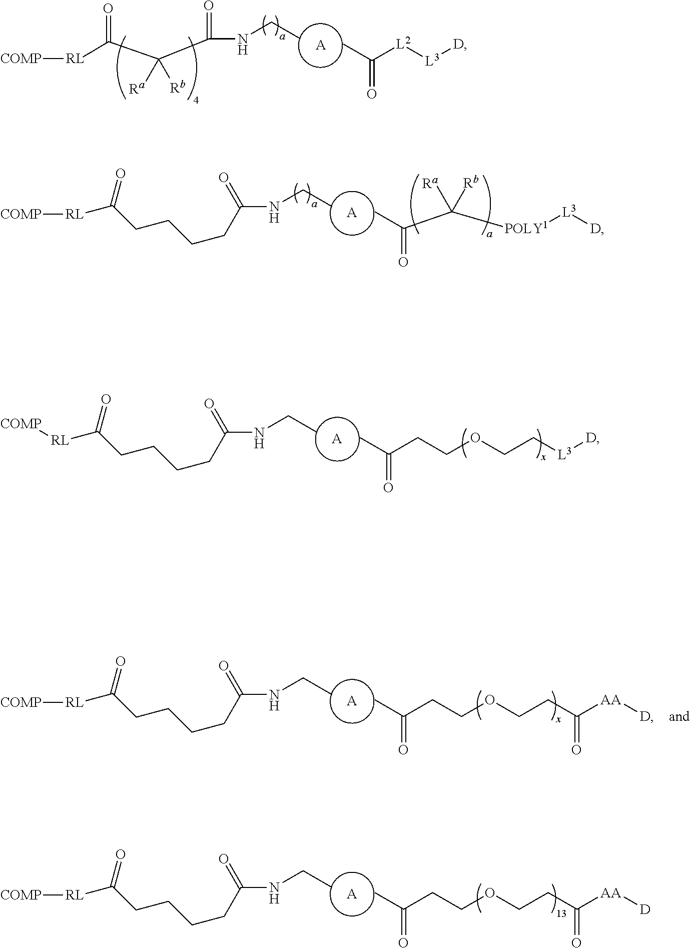

or a pharmaceutically acceptable salt and/or regioisomer thereof.

In certain embodiments, the compound of Formula (II) is a compound of Formula (IIE):

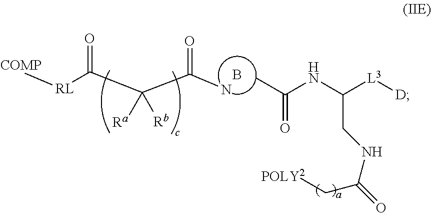

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIE) is selected from the following:

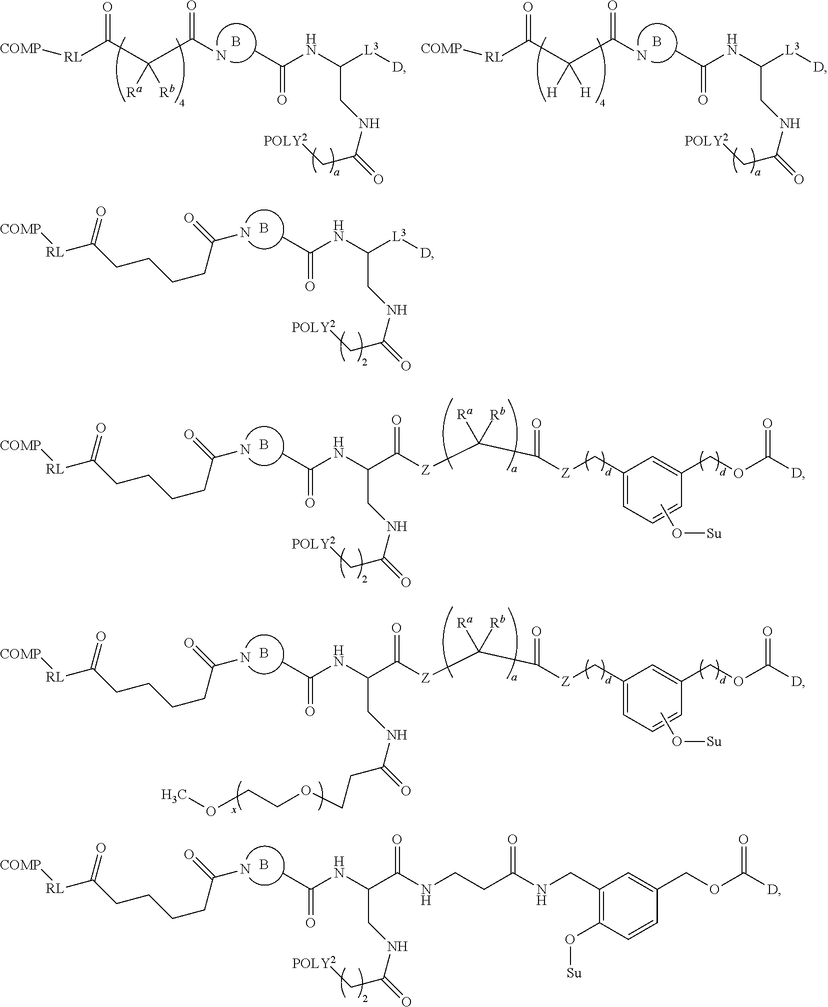

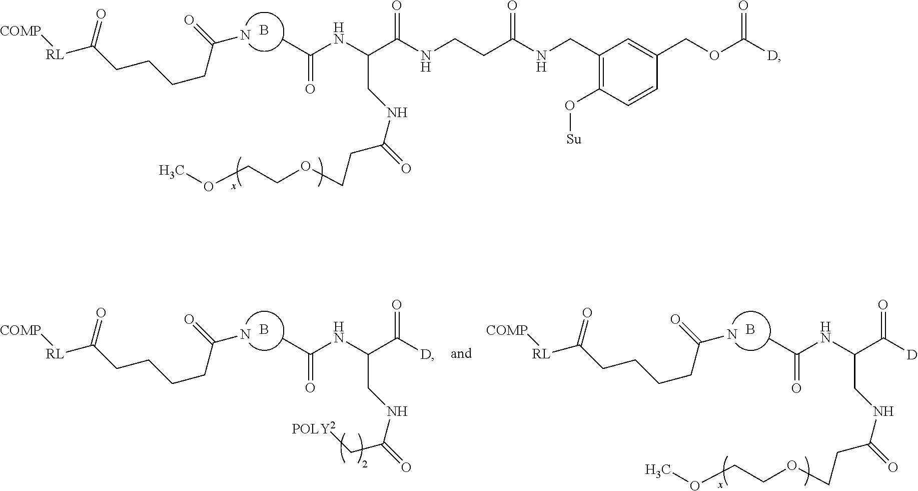

or a pharmaceutically acceptable salt and/or regioisomer thereof.

In certain embodiments, the compound of Formula (II) is a compound of Formula (IIF):

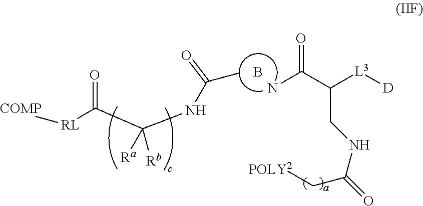

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIF) is selected from the following:

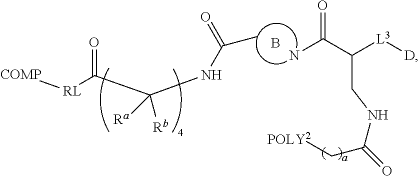

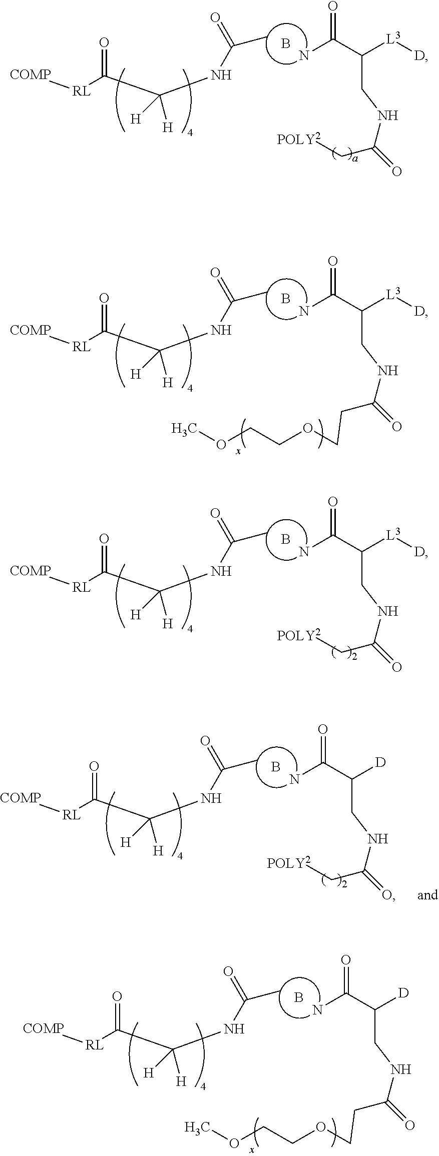

or a pharmaceutically acceptable salt and/or regioisomer thereof.

In certain embodiments, the compound of Formula (II) is a compound of Formula (IIG):

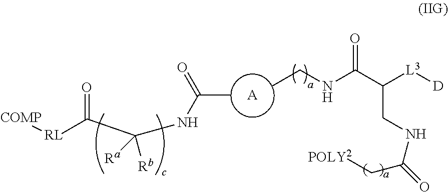

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIG) is selected from the following:

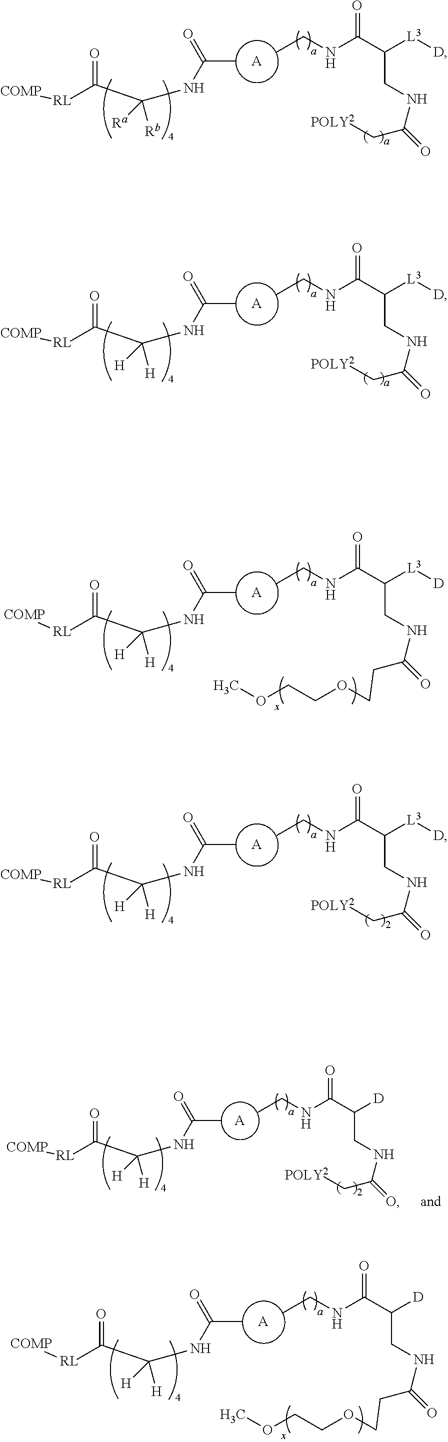

or a pharmaceutically acceptable salt and/or regioisomer thereof.

In certain embodiments, the compound of Formula (II) is a compound of Formula (IIH):

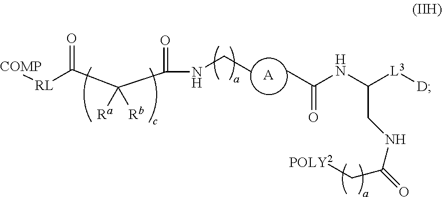

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIH) is selected from the following:

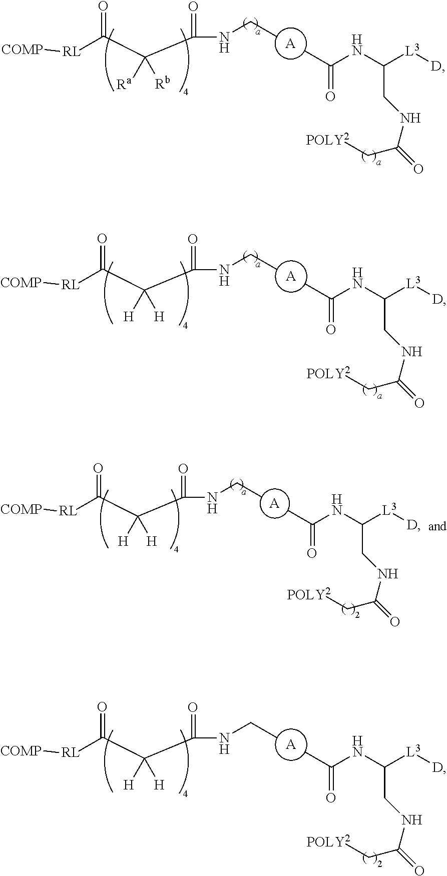

or a pharmaceutically acceptable salt and/or regioisomer thereof.

In one aspect, provided herein is a conjugate of Formula (III):

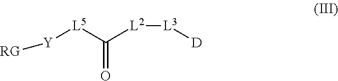

or a pharmaceutically acceptable salt and/or regioisomer thereof;
wherein
L⁵ is a linker comprising an unnatural amino acid; and
RG, Y, L², L³, and D are as defined herein.

Non-limiting unnatural amino acids include sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone.

In certain embodiments, the compound of Formula (III) is a compound of Formula (IIIA):

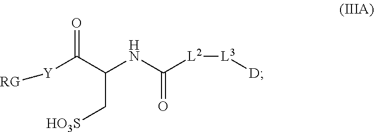

or a pharmaceutically acceptable salt and/or regioisomer thereof;
wherein RG, Y, L², L³, and D are as defined herein.

In certain embodiments, the compound of Formula (III) is a compound of Formula (IIIB):

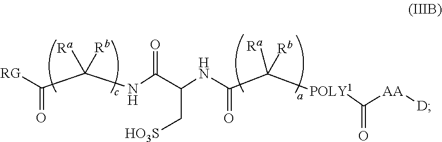

or a pharmaceutically acceptable salt and/or regioisomer thereof;
wherein integer a, integer c, RG, R^{a}, R^{b}, POLY¹, AA, and D are as defined herein.

In certain embodiments, the compound of Formula (IIIB) is a compound of the formula:

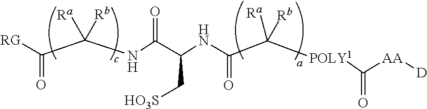

or a pharmaceutically acceptable salt and/or regioisomer thereof.

In one aspect, provided herein is a conjugate of Formula (IV):

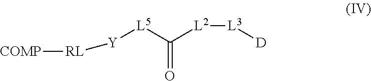

or a pharmaceutically acceptable salt and/or regioisomer thereof;
wherein
L⁵ is a linker comprising an unnatural amino acid; and
RL, COMP, Y, L², L³, and D are as defined herein.

In certain embodiments, the compound of Formula (IV) is a compound of Formula (IVA):

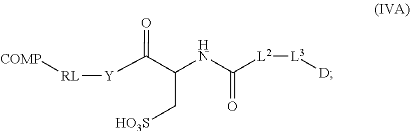

or a pharmaceutically acceptable salt and/or regioisomer thereof;
wherein RL, COMP, Y, L², L³, and D are as defined herein.

In certain embodiments, the compound of Formula (IV) is a compound of Formula (IVB):

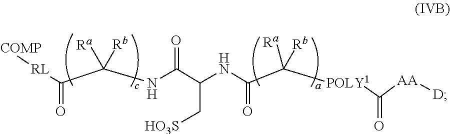

or a pharmaceutically acceptable salt and/or regioisomer thereof;
wherein integer a, integer, c, RL, COMP, R^{a}, R^{b}, POLY¹, AA, and D are as defined herein.

In certain embodiments, the compound of Formula (IVB) is a compound of the formula:

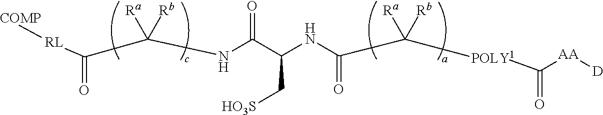

or a pharmaceutically acceptable salt and/or regioisomer thereof.

In certain embodiments of Formula (I)-(IIH), including any of the foregoing, L¹ is

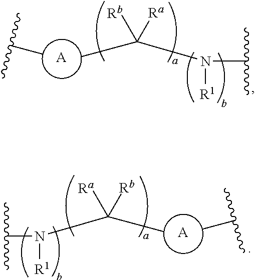

In certain embodiments of Formula (I)-(IIH), including any of the foregoing, L¹ is

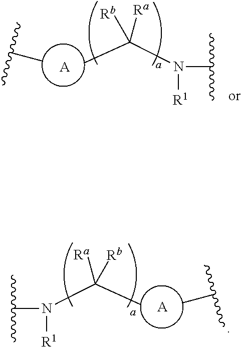

In certain embodiments of Formula (I)-(IIH), including any of the foregoing, L¹ is

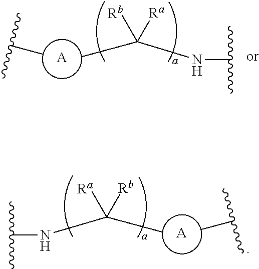

In certain embodiments of Formula (I)-(IIH), including any of the foregoing, L¹ is

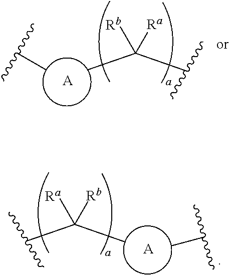

In certain embodiments of Formula (I)-(IIH), including any of the foregoing, L¹ is

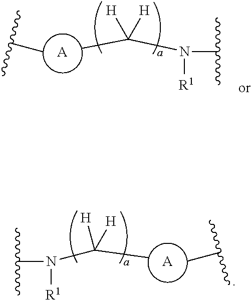

In certain embodiments of Formula (I)-(IIH), including any of the foregoing, L¹ is

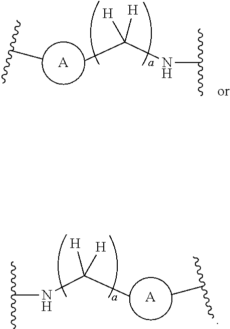

In certain embodiments of Formula (I)-(IIH), including any of the foregoing, L¹ is

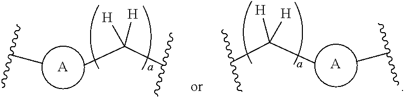

In certain embodiments of Formula (I)-(IIH), including any of the foregoing, L¹ is

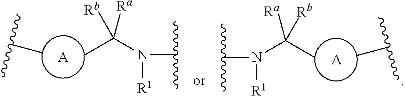

In certain embodiments of Formula (I)-(IIH), including any of the foregoing, L¹ is

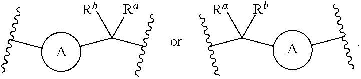

In certain embodiments of Formula (I)-(IIH), including any of the foregoing, L¹ is

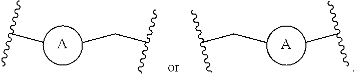

In certain embodiments of Formula (I)-(IIH), including any of the foregoing, L¹ is

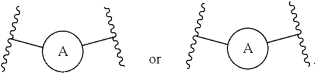

In certain embodiments of Formula (I)-(IIH), including any of the foregoing, L¹ is

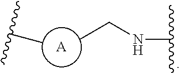

In certain embodiments of Formula (I)-(IIH), including any of the foregoing, L¹ is

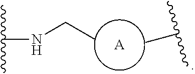

In certain embodiments of Formula (I)-(IIH), including any of the foregoing, L¹ is

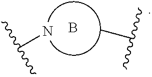

In certain embodiments of Formula (I)-(IIH), including any of the foregoing, L¹ is

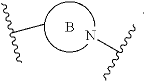

In certain embodiments of Formula (I)-(IIH), including any of the foregoing, Ring B of L¹ is a 5- to 12-membered N-linked bridged, fused, or spirocyclic bicyclic heterocycle containing 1, 2, or 3 heteroatoms independently selected from N, O, and S including the N to which the ring is attached. In certain embodiments of Formula (I)-(IIH), including any of the foregoing, Ring B of L¹ is a 5- to 12-membered N-linked spirocyclic bicyclic heterocycle containing 1, 2, or 3 heteroatoms independently selected from N, O, and S including the N to which the ring is attached.

In certain embodiments of Formula (I)-(IIH), including any of the foregoing, Ring B of L¹ is an optionally substituted 5- to 12-membered N-linked bridged, fused, or spirocyclic bicyclic heterocycle containing 1, 2, or 3 heteroatoms independently selected from N, O, and S including the N to which the ring is attached, wherein the heterocycle of Ring B is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (I)-(IIH), including any of the foregoing, Ring B of L¹ is an optionally substituted 5- to 12-membered N-linked spirocyclic bicyclic heterocycle containing 1, 2, or 3 heteroatoms independently selected from N, O, and S including the N to which the ring is attached, wherein the heterocycle of Ring B is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH. —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl.

In certain embodiments of Formula (I)-(IIH), including any of the foregoing, Ring B of L¹ is selected from

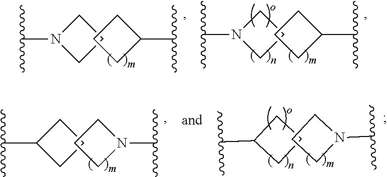

wherein m is an integer selected from 1, 2, 3, 4, and 5; and
each of n and o is an integer independently selected from 1, 2, and 3.

In certain embodiments of Formula (I)-(IIH), including any of the foregoing, Ring B of L¹ is

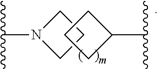

In certain embodiments of Formula (I)-(IIH),

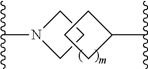

is selected from

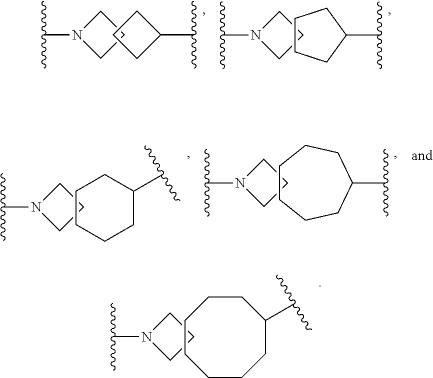

In certain embodiments of Formula (I)-(IIH),

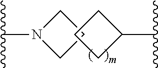

is selected from

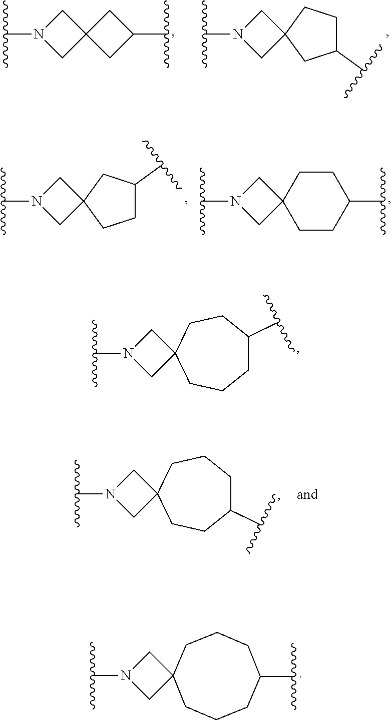

In certain embodiments of Formula (I)-(IIH), Ring B of L¹ is

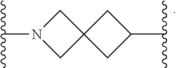 [SMILES: *C1CC2(C1)CN(*)C2]

In certain embodiments, Ring B of L¹ is

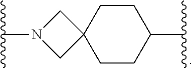 [SMILES: *C1CCC2(CC1)CN(*)C2]

In certain embodiments of Formula (I)-(IIH), including any of the foregoing, Ring B of L¹ is

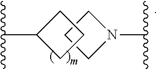

In certain embodiments of Formula (I)-(IIH),

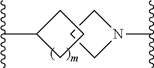

is selected from

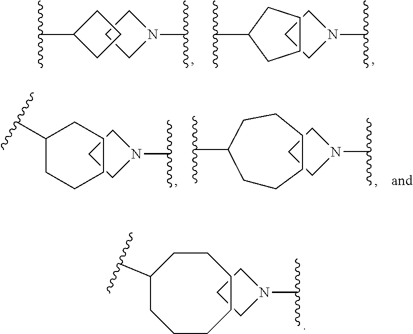

In certain embodiments of Formula (I)-(IIH),

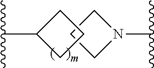

is selected from

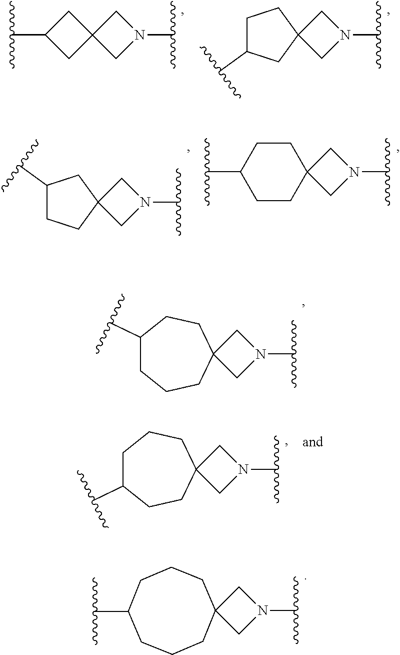

In certain embodiments of Formula (I)-(IIH), Ring B of L¹ is

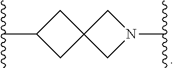 [SMILES: *C1CC2(C1)CN(*)C2]

In certain embodiments of Formula (I)-(IIH), Ring B of L¹ is

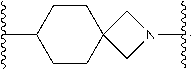 [SMILES: *C1CCC2(CC1)CN(*)C2]

In certain embodiments of Formula (I)-(IIH), including any of the foregoing, Ring B of L¹ is

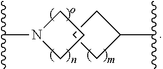

In certain embodiments of Formula (I)-(IIH),

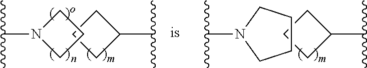

wherein m is 1, 2, or 3. In certain embodiments of Formula (I)-(IIH),

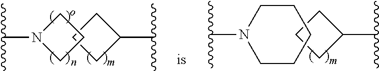

wherein m is 1, 2, or 3. In certain embodiments of Formula (I)-(IIH),

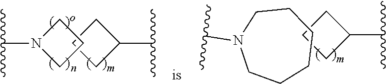

wherein m is 1, 2, or 3. In certain embodiments of Formula (I)-(IIH),

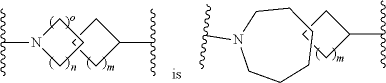

wherein m is 1, 2, or 3.

In certain embodiments of Formula (I)-(IIH),

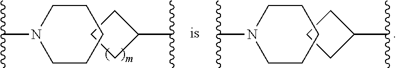

In certain embodiments of Formula (I)-(IIH),

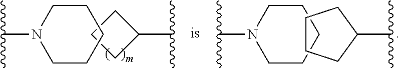

In certain embodiments of Formula (I)-(IIH),

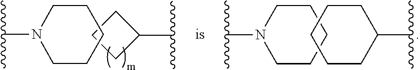

In certain embodiments of Formula (I)(IIH),

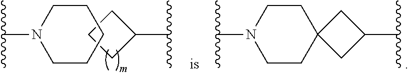

In certain embodiments of Formula (I)-(IIH),

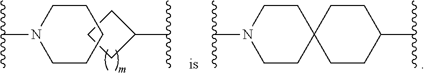

In certain embodiments of Formula (I)-(IIH), including any of the foregoing, Ring B of L¹ is

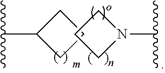

In certain embodiments of Formula (I)-(IIH),

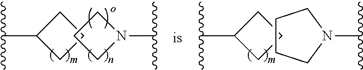

wherein m is 1, 2, or 3. In certain embodiments of Formula (I)-(IIH),

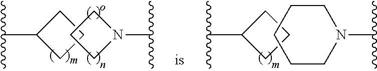

wherein m is 1, 2, or 3. In certain embodiments of Formula (I)-(IIH),

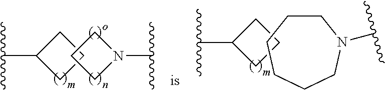

wherein m is 1, 2, or 3. In certain embodiments of Formula (I)-(IIH),

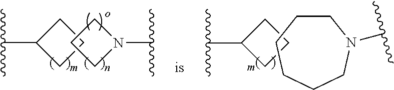

wherein m is 1, 2, or 3.

In certain embodiments of Formula (I)-(IIH),

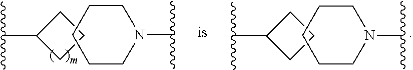

In certain embodiments of Formula (I)-(IIH,

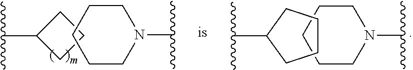

In certain embodiments of Formula (I)-(IIH),

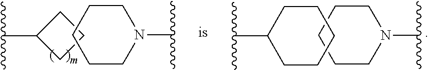

In certain embodiments,

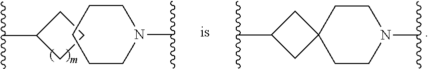

In certain embodiments of Formula (I)-(IIH),

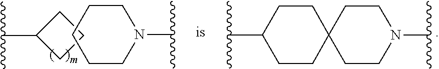

In certain embodiments of Formula (I)-(IIH), including any of the foregoing, Ring B of L¹ is

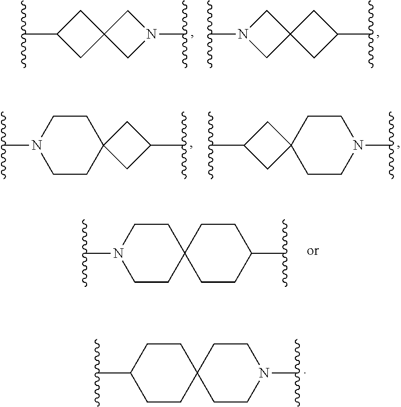

In certain embodiments of Formula (I)-(IIH), including any of the foregoing, Ring B of L¹ is selected from

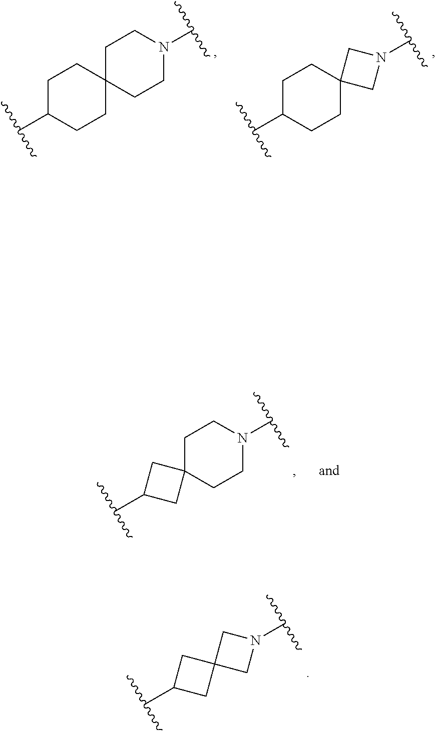 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

In certain embodiments of Formula (I)-(IIH), including any of the foregoing, Ring B of L¹ is selected from

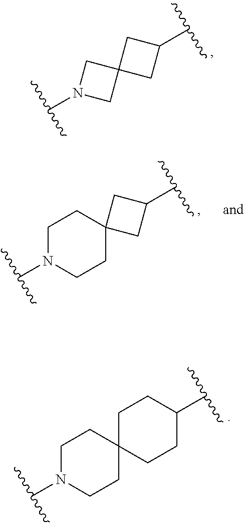 [SMILES: *C1CC2(C1)CN(*)C2 ; *C1CC2(CCN(*)CC2)C1 ; *C1CCC2(CC1)CCN(*)CC2]

In certain embodiments of Formula (I)-(IIH), including any of the foregoing, L¹ is selected from

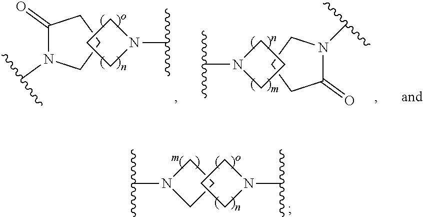

wherein m is an integer selected from 1, 2, 3, 4, and 5; and
each of n and o is an integer independently selected from 1, 2, and 3.

In certain embodiments of Formula (I)-(IIH), including any of the foregoing, L¹ is selected from

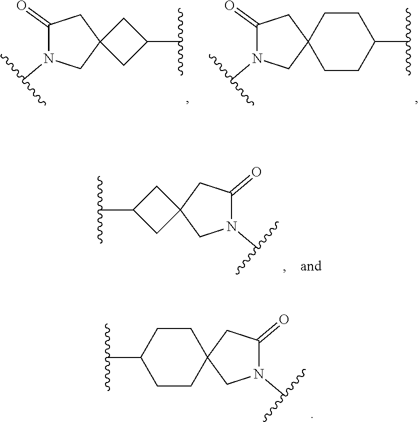 [SMILES: *C1CC2(CC(=O)N(*)C2)C1 ; *C1CCC2(CC1)CC(=O)N(*)C2 ; *C1CC2(CC(=O)N(*)C2)C1 ; *C1CCC2(CC1)CC(=O)N(*)C2]

In certain embodiments of Formula (I)-(IIH), including any of the foregoing, Ring B of L is selected from

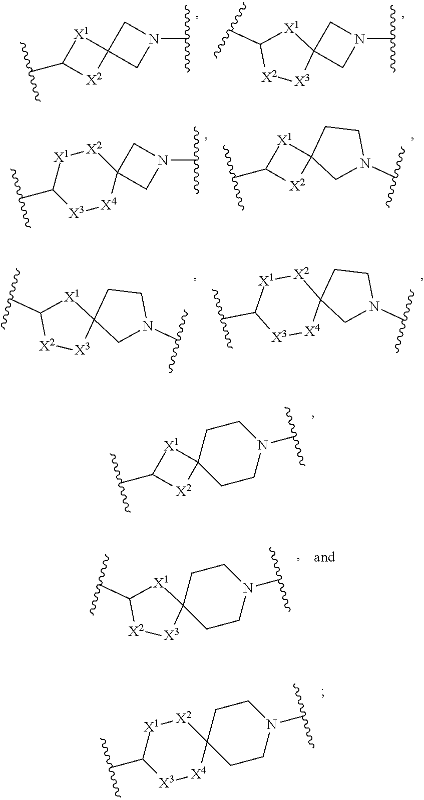

wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—; and
R⁴ is independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group. In some embodiments, R⁴ is independently selected from hydrogen, C₁₋₆ alkyl, C₂₋₆alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (I)-(IIH), including any of the foregoing, Ring B of L¹ is selected from

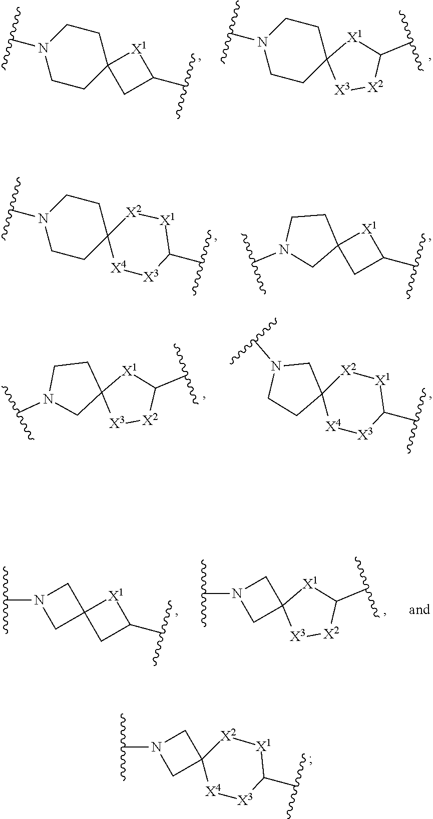

wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—; and
R⁴ is independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group. In some embodiments, R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (I)-(IIH), including any of the foregoing, Ring A of L¹ is a bridged, fused, or spirocyclic bicyclic carbocycle. In certain embodiments, including any of the foregoing, Ring A of L¹ is a C₄₋₁₂ bridged, fused, or spirocyclic bicyclic carbocycle. In certain embodiments of Formula (I)-(IIH), including any of the foregoing, Ring A of L¹ is a C₄₋₁₂ bridged bicyclic carbocycle. In certain embodiments of Formula (I)-(IIH), including any of the foregoing, Ring A of L¹ is a C₄₋₈ bridged bicyclic carbocycle.

In certain embodiments of Formula (I)-(IIH), including any of the foregoing, Ring A of L¹ is an optionally substituted bridged, fused, or spirocyclic bicyclic carbocycle, wherein the carbocycle of Ring A is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, Ring A of L¹ is an optionally substituted C₄₋₁₂ bridged, fused, or spirocyclic bicyclic carbocycle. In certain embodiments of Formula (I)-(IIH), including any of the foregoing, Ring A of L¹ is an optionally substituted C₄₋₁₂ bridged bicyclic carbocycle. In certain embodiments of Formula (I)-(IIH), including any of the foregoing, Ring A of L¹ is an optionally substituted C₄₋₈ bridged bicyclic carbocycle.

In certain embodiments of Formula (I)-(IIH), including any of the foregoing, Ring A of L¹ is selected from

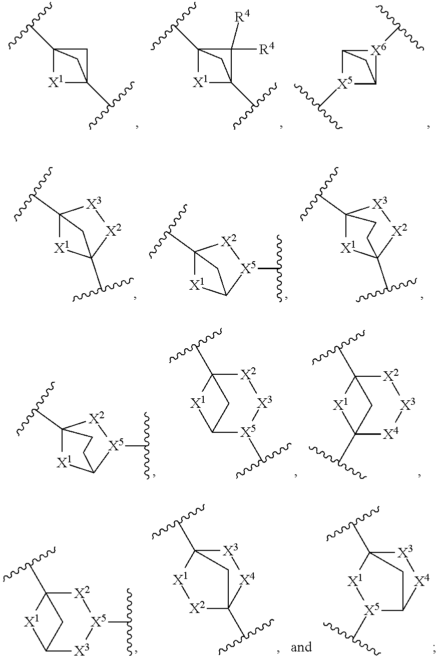

wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—;
X⁵ is CR⁴ or N; and
R⁴ is independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group. In some embodiments, R⁴ is independently selected from hydrogen, C₁₋₆ alkyl, C₂₋₆ alkenyl, C₂₋₆ alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (I)-(IIH), including any of the foregoing, Ring A of L¹ is selected from

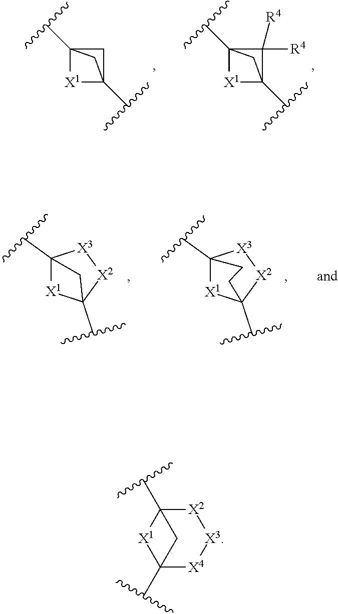

In certain embodiments of Formula (I)-(IIH), including any of the foregoing, Ring A of L¹ is selected from

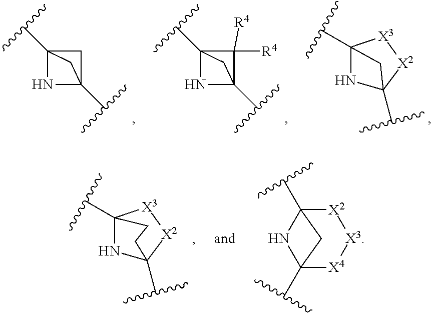

In certain embodiments of Formula (I)-(IIH), including an of the foregoing, Ring A of L¹ is selected from

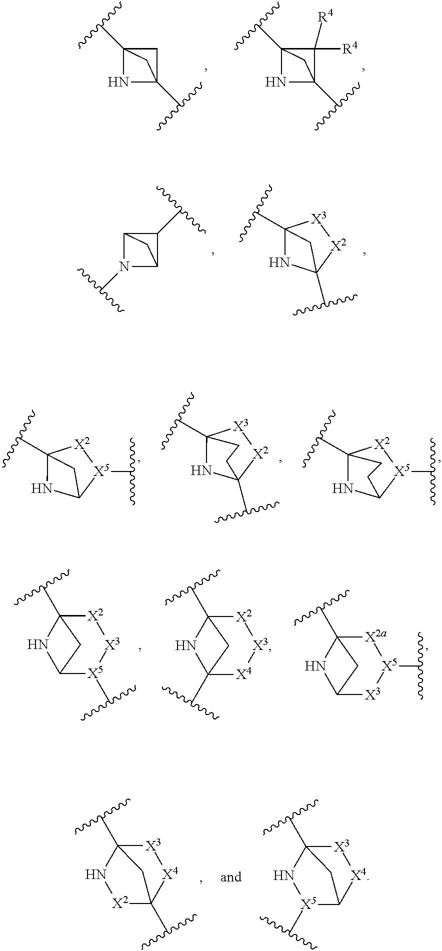

In certain embodiments of Formula (I)-(IIH), including any of the foregoing, Ring A of L¹ is selected from

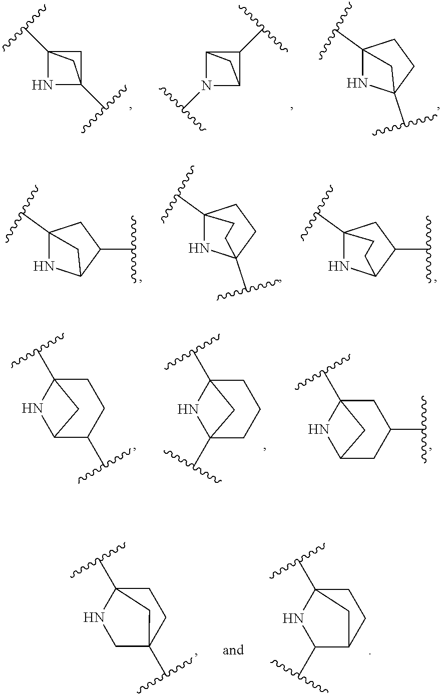

In certain embodiments of Formula (I)-(IIH), including any of the foregoing, Ring A of L¹ is

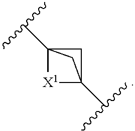

In certain embodiments of Formula (I)-(IIH), including any of the foregoing, Ring A of L¹ is

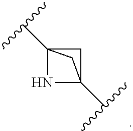 [SMILES: *C12CC(*)(C1)N2]

In certain embodiments of Formula (I)-(IIH), including any of the foregoing, X¹, X², X³, and/or X⁴ is —C(R⁴)₂—. In certain embodiments of Formula (I)-(IIH), including any of the foregoing, X¹ and X² are —C(R⁴)₂—. In certain embodiments of Formula (I)-(IIH), including any of the foregoing, X¹, X², and X³ are —C(R⁴)₂—. In certain embodiments of Formula (I)-(IIH), including any of the foregoing, X¹, X², X³, and X⁴ are —C(R⁴)₂—. In certain embodiments of Formula (I)-(IIH), including any of the foregoing, X¹ is —NH—. In certain embodiments of Formula (I)-(IIH), including any of the foregoing, X² is NH—. In certain embodiments of Formula (I)-(IIH), including any of the foregoing, X³ is —NH—. In certain embodiments of Formula (I)-(IIH), including any of the foregoing, X⁴ is NH—. In certain embodiments, including any of the foregoing, X¹ is —O—. In certain embodiments of Formula (I)-(IIH), including any of the foregoing, X² is —O—. In certain embodiments of Formula (I)-(IIH), including any of the foregoing, X³ is —O—. In certain embodiments of Formula (I)-(IIH), including any of the foregoing, X⁴ is —O—.

In certain embodiments of Formula (I)-(IIH), including any of the foregoing, L¹ is

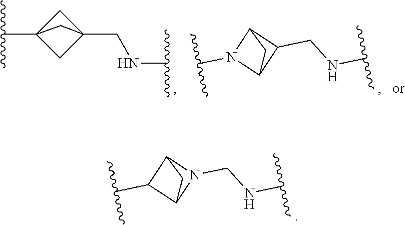

In certain embodiments, L¹ is

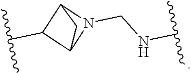 [SMILES: *NCN1C2CC1C2*]

In certain embodiments, including any of the foregoing, a is 0. In certain embodiments, including any of the foregoing, a is 1. In certain embodiments, including any of the foregoing, a is 2. In certain embodiments, including any of the foregoing, a is 3. In certain embodiments, including any of the foregoing, a is 4. In certain embodiments, including any of the foregoing, a is 5. In certain embodiments, including any of the foregoing, a is 6.

In certain embodiments of Formula (I)-(IIH), b is 0. In certain embodiments of Formula (I)-(IIH), b is 1.

In certain embodiments of Formula (I)-(IIH), b is 0 and a is 0. In certain embodiments of Formula (I)-(IIH), b is 0 and a is 1. In certain embodiments of Formula (I)-(IIH), b is 0 and a is 2. In certain embodiments of Formula (I)-(IIH), b is 0 and a is 3. In certain embodiments of Formula (I)-(IIH), b is 0 and a is 4. In certain embodiments of Formula (I)-(IIH), b is 0 and a is 5. In certain embodiments of Formula (I)-(IIH), b is 0 and a is 6. In certain embodiments of Formula (I)-(IIH), b is 1 and a is 1. In certain embodiments of Formula (I)-(IIH), b is 1 and a is 2. In certain embodiments of Formula (I)-(IIH), b is 1 and a is 3. In certain embodiments of Formula (I)-(IIH), b is 1 and a is 4. In certain embodiments of Formula (I)-(IIH), b is 1 and a is 5. In certain embodiments of Formula (I)-(IIH), b is 1 and a is 6.

In certain embodiments of Formula (I)-(IIH), including any of the foregoing, R¹ is hydrogen. In certain embodiments of Formula (I)-(IIH), including any of the foregoing, R¹ is unsubstituted alkyl. In certain embodiments of Formula (I)-(IIH), including any of the foregoing, R¹ is methyl. In certain embodiments of Formula (I)-(IIH), including any of the foregoing, R¹ is alkyl optionally substituted with one or more substituent selected from cycloalkyl, halogen, alkoxy, —CN, —NO₂, and —OH.

In certain embodiments of Formula (III)—(IVB), L⁵ is a linker that comprises at least one amino acid selected from sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone. In certain embodiments of Formula (III)—(IVB), L⁵ is a linker that comprises

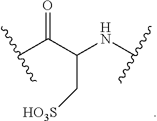 [SMILES: *NC(CS(=O)(=O)O)C(*)=O]

In certain embodiments of Formula (III)—(IVB), L⁵ is a linker that comprises

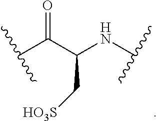 [SMILES: *N[C@@H](CS(=O)(=O)O)C(*)=O]

In certain embodiments of Formula (III)—(IVB), L⁵ is

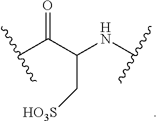 [SMILES: *NC(CS(=O)(=O)O)C(*)=O]

In certain embodiments of Formula (III)—(IVB), L⁵ is

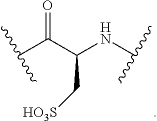 [SMILES: *N[C@@H](CS(=O)(=O)O)C(*)=O]

In certain embodiments, including any of the foregoing, R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, alkenyl, alkynyl, cycloalkyl, halogen, alkoxy, —CN, —NO₂, —OH, —NH₂, —C(O)NH₂, —C(O)OH, aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, halogen, alkoxy, —CN, —NO₂, —OH, —NH₂, —C(O)NH₂, and —C(O)OH. In certain embodiments, including any of the foregoing, R^{a} and R^{b} are both hydrogen.

In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 1; and b is 1. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 2; and b is 1. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 3; and b is 1.

In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and b is 0. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 2, and b is 0. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3, and b is 0.

In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 1; and b is 1. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 2; and b is 1. In certain embodiments of Formula (I)-(IIH), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 3, and b is 1.

In certain embodiments, including any of the foregoing of Formula (I)-(IIH), R^{a} and R^{b} are both hydrogen; R¹ is hydrogen; a is 1, and b is 1. In certain embodiments of Formula (I)-(IIH), including any of the foregoing, R^{a} and R^{b} are both hydrogen; R¹ is hydrogen; a is 2, and b is 1. In certain embodiments of Formula (I)-(IIH), including any of the foregoing, R^{a} and R^{b} are both hydrogen; R¹ is hydrogen; a is 3, and b is 1. In certain embodiments of Formula (I)-(IIH), including any of the foregoing, R^{a} and R^{b} are both hydrogen; R¹ is hydrogen; a is 4, and b is 1. In certain embodiments of Formula (I)-(IIH), including any of the foregoing, R^{a} and R^{b} are both hydrogen; R¹ is hydrogen; a is 5, and b is 1. In certain embodiments of Formula (I)-(IIH), including any of the foregoing, R^{a} and R^{b} are both hydrogen; R¹ is hydrogen; a is 6, and b is 1.

In certain embodiments of Formula (I)-(IIH), including any of the foregoing, R^{a} and R^{b} are both hydrogen; a is 1; and b is 0. In certain embodiments of Formula (I)-(IIH), including any of the foregoing, R^{a} and R^{b} are both hydrogen; a is 2; and b is 0. In certain embodiments of Formula (I)-(IIH), including any of the foregoing, R^{a} and R^{b} are both hydrogen; a is 3; and b is 0. In certain embodiments of Formula (I)-(IIH), including any of the foregoing, R^{a} and R^{b} are both hydrogen; a is 4; and b is 0. In certain embodiments of Formula (I)-(IIH), including any of the foregoing, R^{a} and R^{b} are both hydrogen; a is 5; and b is 0. In certain embodiments of Formula (I)-(IIH), including any of the foregoing, R^{a} and R^{b} are both hydrogen; a is 6; and b is 0.

In certain embodiments of Formula (I)-(IIH), including any of the foregoing, R^{a} and R^{b} are both hydrogen; R¹ is methyl; a is 1; and b is 0. In certain embodiments of Formula (I)-(IIH), including any of the foregoing, R^{a} and R^{b} are both hydrogen; R¹ is methyl; a is 2; and b is 0. In certain embodiments of Formula (I)-(IIH), including any of the foregoing, R^{a} and R^{b} are both hydrogen; R¹ is methyl; a is 3; and b is 0. In certain embodiments of Formula (I)-(IIH), including any of the foregoing, R^{a} and R^{b} are both hydrogen; R¹ is methyl; a is 4; and b is 0. In certain embodiments of Formula (I)-(IIH), including any of the foregoing, R^{a} and R^{b} are both hydrogen; R¹ is methyl; a is 5; and b is 0. In certain embodiments of Formula (I)-(IIH), including any of the foregoing, R^{a} and R^{b} are both hydrogen; R¹ is methyl; a is 6; and b is 0.

In certain embodiments of Formula (III)—(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and c is 1. In certain embodiments of Formula (III)—(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 2; and c is 1. In certain embodiments of Formula (III)—(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3; and c is 1.

In certain embodiments, including any of the foregoing, Y is *—C(O)—(CR^{a}R^{b})_{c}—NH— wherein * represents where Y is bound to RG in Formula (I)—(IH) and (III)—(IIIB) and RL in Formula (II)-(IIH) and (IV)—(IVB). In certain embodiments, including any of the foregoing, Y is *—C(O)—(CH₂)_{c}—NH—. In certain embodiments, including any of the foregoing, Y is *—C(O)—(CH₂)—NH—. In certain embodiments, including any of the foregoing, Y is *—C(O)—(CH₂)₂—NH—. In certain embodiments, including any of the foregoing, Y is *—C(O)—(CH₂)₃—NH—. In certain embodiments, including any of the foregoing, Y is *—C(O)—(CH₂)₄—NH—. In certain embodiments, including any of the foregoing, Y is *—C(O)—(CH₂)₅—NH—. In certain embodiments, including any of the foregoing, Y is *—C(O)—(CH₂)₆—NH—.

In certain embodiments, including any of the foregoing, Y is *—C(O)—(CR^{a}R^{b})—NH—. In certain embodiments, including any of the foregoing, Y is *—C(O)—(CR^{a}R^{b})₂—NH—. In certain embodiments, including any of the foregoing, Y is *—C(O)—(CR^{a}R^{b})₃—NH—. In certain embodiments, including any of the foregoing, Y is *—C(O)—(CR^{a}R^{b})₄—NH—. In certain embodiments, including any of the foregoing, Y is *—C(O)—(CR^{a}R^{b})₅—NH—. In certain embodiments, including any of the foregoing, Y is *—C(O)—(CR^{a}R^{b})₆—NH—.

In certain embodiments, including any of the foregoing, Y is *—C(O)—(CR^{a}R^{b})_{c}—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, Y is *—C(O)—(CR^{a}R^{b})₂—NH—, *—C(O)—(CR^{a}R^{b})₃—NH—, or *—C(O)(CR^{a}R^{b})₄—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl.

In certain embodiments, including any of the foregoing, Y is *—C(O)—(CR^{a}R^{b})_{c}— wherein * represents where Y is bound to RG in Formula (I)—(IH) and (III)—(IIIB) and RL in Formula (II)-(IIH) and (IV)—(IVB). In certain embodiments, including any of the foregoing, Y is *—C(O)—(CH₂)_{c}—. In certain embodiments, including any of the foregoing, Y is *—C(O)—(CH₂)—. In certain embodiments, including any of the foregoing, Y is *—C(O)—(CH₂)₂—. In certain embodiments, including any of the foregoing, Y is *—C(O)—(CH₂)₃—. In certain embodiments, including any of the foregoing, Y is *—C(O)—(CH₂)₄—. In certain embodiments, including any of the foregoing, Y is *—C(O)—(CH₂)₅—. In certain embodiments, including any of the foregoing, Y is *—C(O)—(CH₂)₆—.

In certain embodiments, including any of the foregoing, Y is *—C(O)—(CR^{a}R^{b})—. In certain embodiments, including any of the foregoing, Y is *—C(O)—(CR^{a}R^{b})₂—. In certain embodiments, including any of the foregoing, Y is *—C(O)—(CR^{a}R^{b})₃—. In certain embodiments, including any of the foregoing, Y is *—C(O)—(CR^{a}R^{b})₄—. In certain embodiments, including any of the foregoing, Y is *—C(O)—(CR^{a}R^{b})₅—. In certain embodiments, including any of the foregoing, Y is *—C(O)—(CR^{a}R^{b})₆—.

In certain embodiments, including any of the foregoing, Y is *—C(O)—(CR^{a}R^{b})_{c}— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, Y is *—C(O)H(CR^{a}R^{b})₂—, *—C(O)—(CR^{a}R^{b})₃—, or *—C(O)—(CR^{a}R^{b})₄— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl.

In certain embodiments, including any of the foregoing, Y is *—C(O)—(CH₂)₂—NH— or *—C(O)—(CH₂)₄—.

In certain embodiments, including any of the foregoing, L² is absent. In certain embodiments, including any of the foregoing, L² is a linker comprising a hydrophilic polymer residue.

In certain embodiments, including any of the foregoing, L² is —(CR^{a}R^{b})ₐ-POLY¹-. In certain embodiments, including any of the foregoing, L² is —CH₂—POLY¹-. In certain embodiments, including any of the foregoing, L² is —(CH₂)₂-POLY¹-. In certain embodiments, including any of the foregoing, L² is —(CH₂)₃-POLY¹-. In certain embodiments, including any of the foregoing, L² is —(CH₂)₄-POLY¹-. In certain embodiments, including any of the foregoing, L² is —(CH₂)₅-POLY¹-. In certain embodiments, including any of the foregoing, L² is —(CH₂)₆-POLY¹-. In certain embodiments, including any of the foregoing, L² is —CR^{a}R^{b}-POLY¹-. In certain embodiments, including any of the foregoing, L² is —(CR^{a}R^{b})₂-POLY¹-. In certain embodiments, including any of the foregoing, L² is —(CR^{a}R^{b})₃-POLY¹-. In certain embodiments, including any of the foregoing, L² is —(CR^{a}R^{b})₄-POLY¹-.

In certain embodiments, including any of the foregoing, L² is -POLY¹-.

In certain embodiments, including any of the foregoing, L² is —(CR^{a}R^{b})ₐ-POLY¹-(CR^{a}R^{b})ₐ—. In certain embodiments, including any of the foregoing, L² is —(CR^{a}R^{b})ₐ-POLY¹-(CR^{a}R^{b})ₐ— wherein a is independently selected from 0, 1, 2, 3, 4, 5, or 6. In certain embodiments, including any of the foregoing, L² is —(CH₂)ₐ-POLY¹-(CH₂)ₐ— wherein a is independently selected from 0, 1, 2, 3, 4, 5, or 6. In certain embodiments, including any of the foregoing, L² is —(CH₂)ₐ-POLY¹-(CR^{a}R^{b})ₐ— wherein a is selected from 1, 2, 3, 4, 5, or 6. In certain embodiments, including any of the foregoing, L² is —(CR^{a}R^{b})ₐ-POLY¹-(CH₂)ₐ— wherein a is independently selected from 0, 1, 2, 3, 4, 5, or 6.

In certain embodiments, including any of the foregoing, POLY¹ is a diavalent residue of a nonpeptidic, hydrophilic polymer. In certain embodiments, POLY¹ is a diavalent residue of polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof. In certain embodiments, including any of the foregoing, POLY¹ is a divalent residue of polyethylene glycol (PEG), poly(propylene glycol) (PPG), or a copolymer of ethylene glycol and propylene glycol.

In certain embodiments, including any of the foregoing, POLY¹ is a diavalent residue of polyethylene glycol (PEG). In certain embodiments, including any of the foregoing, POLY¹ is a diavalent residue of poly(propylene glycol) (PPG). In certain embodiments, including any of the foregoing, POLY¹ is a diavalent residue of copolymers of ethylene glycol and propylene glycol. In certain embodiments, including any of the foregoing, POLY¹ is a diavalent residue of poly(oxyethylated polyol). In certain embodiments, including any of the foregoing, POLY¹ is a diavalent residue of poly(olefinic alcohol). In certain embodiments, including any of the foregoing, POLY¹ is a diavalent residue of poly(vinylpyrrolidone). In certain embodiments, including any of the foregoing, POLY¹ is a diavalent residue of poly(hydroxyalkylmethacrylamide). In certain embodiments, including any of the foregoing, POLY¹ is a diavalent residue of poly(hydroxyalkylmethacrylate). In certain embodiments, including any of the foregoing, POLY¹ is a diavalent residue of poly(saccharides). In certain embodiments, including any of the foregoing, POLY¹ is a diavalent residue of poly((x-hydroxy acid). In certain embodiments, including any of the foregoing, POLY¹ is a diavalent residue of poly(vinyl alcohol). In certain embodiments, including any of the foregoing, POLY¹ is a diavalent residue of polyphosphazene. In certain embodiments, including any of the foregoing, POLY¹ is a diavalent residue of polyoxazolines (POZ). In certain embodiments, including any of the foregoing, POLY¹ is a diavalent residue of poly(N-acryloylmorpholine). In certain embodiments, including any of the foregoing, POLY¹ is a diavalent residue of polysarcosine.

In certain embodiments, including any of the foregoing, POLY¹ is

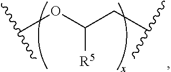

wherein R⁵ is hydrogen or methyl, x is an integer from 1 to 100, inclusive, and [symbol] resents attachment to the remainder of the compound or conjugate. In certain embodiments, including any of the foregoing, x is an integer between 1 to 25. In certain embodiments, including any of the foregoing, x is an integer between 5 to 15. In some embodiments, including any of the foregoing, x is 1. In some embodiments, including any of the foregoing, x is 2. In some embodiments, including any of the foregoing, x is 3. In some embodiments, including any of the foregoing, x is 4. In some embodiments, including any of the foregoing, x is 5. In some embodiments, including any of the foregoing, x is 6. In some embodiments, including any of the foregoing, x is 7. In some embodiments, including any of the foregoing, x is 8. In some embodiments, including any of the foregoing, x is 9. In some embodiments, including any of the foregoing, x is 10. In some embodiments, including any of the foregoing, x is 11. In some embodiments, including any of the foregoing, x is 12. In some embodiments, including any of the foregoing, x is 13. In some embodiments, including any of the foregoing, x is 14. In some embodiments, including any of the foregoing, x is 15. In some embodiments, including any of the foregoing, x is 16. In some embodiments, including any of the foregoing, x is 17. In some embodiments, including any of the foregoing, x is 18. In some embodiments, including any of the foregoing, x is 19. In some embodiments, including any of the foregoing, x is 20. In certain embodiments, including any of the foregoing, x is an integer between 25 and 50. In certain embodiments, including any of the foregoing, x is an integer between 35 and 45. In certain embodiments, including any of the foregoing, x is an integer between 50 and 75. In certain embodiments, including any of the foregoing, x is an integer between 55 and 65. In certain embodiments, including any of the foregoing, x is an integer between 75 and 100. In certain embodiments, including any of the foregoing, x is an integer between 85 and 95. In certain embodiments, including any of the foregoing, x is an integer in the range of 1 and 25, 20 and 45, 40 and 65, 60 and 85, 70 and 95, or 75 and 100.

In some embodiments, including any of the foregoing, R⁵ is hydrogen. In some embodiments, including any of the foregoing, R⁵ is methyl.

In certain embodiments, including any of the foregoing, L² is —(CR^{a}R^{b})ₐ-POLY¹- wherein POLY¹ is

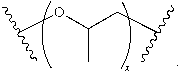

In certain embodiments, including any of the foregoing, L² is —(CR^{a}R^{b})ₐ-POLY¹- wherein POLY¹ is

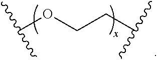

In certain embodiments, including any of the foregoing, L² is —(CH₂)ₐ-POLY¹- wherein POLY¹ is

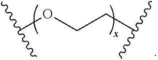

In certain embodiments, including any of the foregoing, L² is —(CH₂)₂-POLY¹- wherein POLY¹ is

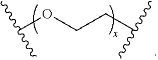

In certain embodiments, including any of the foregoing, L² is —(CR^{a}R^{b})ₐ-POLY¹- wherein POLY¹ is

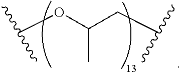

In certain embodiments, including any of the foregoing, L² is —(CR^{a}R^{b})ₐ-POLY¹- wherein POLY¹ is

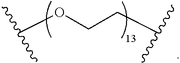

In certain embodiments, including any of the foregoing, L² is —(CH₂)₂-POLY¹- wherein POLY¹ is

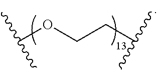

In certain embodiments, including any of the foregoing, L² is —(CH₂)₂-POLY¹- wherein POLY¹ is

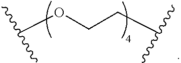

In certain embodiments, including any of the foregoing, L² is —(CH₂)₂-POLY¹-, wherein POLY¹ is

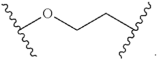 [SMILES: *CCO*]

In certain embodiments, including any of the foregoing, L² is

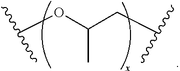

In certain embodiments, including any of the foregoing, L² is

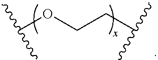

In certain embodiments, including any of the foregoing, L² is

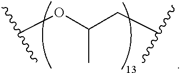

In certain embodiments, including any of the foregoing, L² is

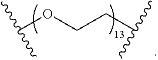

In certain embodiments, including any of the foregoing, L² is —(CR^{a}R^{b})ₐ-POLY¹-(CR^{a}R^{b})ₐ— wherein POLY¹ is

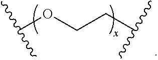

In certain embodiments, including any of the foregoing, L² is —(CR^{a}R^{b})ₐ-POLY¹-(CR^{a}R^{b})ₐ— wherein POLY¹ is

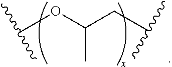

In certain embodiments, including any of the foregoing, L² is —(CR^{a}R^{b})ₐ-POLY¹-(CR^{a}R^{b})ₐ— wherein POLY¹ is

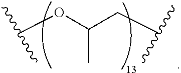

In certain embodiments, including any of the foregoing, L² is —(CR^{a}R^{b})ₐ-POLY¹-(CR^{a}R^{b})ₐ— wherein POLY¹ is

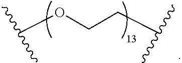

In certain embodiments, including any of the foregoing, L² is selected from

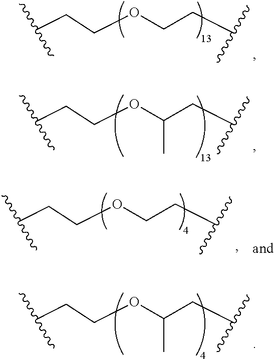

In certain embodiments, including any of the foregoing, L² is

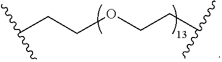

In certain embodiments, including any of the foregoing, L² is

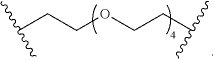

In certain embodiments, including any of the foregoing, L² is

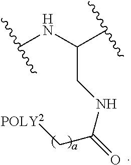

In certain embodiments, including any of the foregoing, L² is

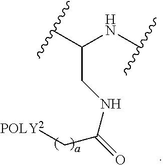

In certain embodiments, including any of the foregoing, L² is

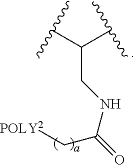

In certain embodiments, including any of the foregoing, L² is

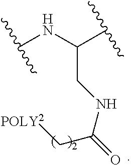

In certain embodiments, including any of the foregoing, L² is

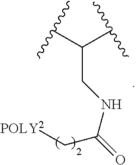

In certain embodiments, including any of the foregoing, POLY² is a residue of a nonpeptidic, hydrophilic polymer. In certain embodiments, POLY² is a residue of polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof. In certain embodiments, including any of the foregoing, POLY² is a residue of polyethylene glycol (PEG), methoxypolyethylene glycol (mPEG), poly(propylene glycol) (PPG), or a copolymer of ethylene glycol and propylene glycol. In certain embodiments, including any of the foregoing, POLY² is a residue of methoxypolyethylene glycol (mPEG).

In certain embodiments, including any of the foregoing, POLY² is a residue of polyethylene glycol (PEG). In certain embodiments, including any of the foregoing, POLY² is a residue of poly(propylene glycol) (PPG). In certain embodiments, including any of the foregoing, POLY² is a residue of copolymers of ethylene glycol and propylene glycol. In certain embodiments, including any of the foregoing, POLY² is a residue of poly(oxyethylated polyol). In certain embodiments, including any of the foregoing, POLY² is a residue of poly(olefinic alcohol). In certain embodiments, including any of the foregoing, POLY² is a residue of poly(vinylpyrrolidone). In certain embodiments, including any of the foregoing, POLY² is a residue of poly(hydroxyalkylmethacrylamide). In certain embodiments, including any of the foregoing, POLY² is a residue of poly(hydroxyalkylmethacrylate). In certain embodiments, including any of the foregoing, POLY² is a residue of poly(saccharides). In certain embodiments, including any of the foregoing, POLY² is a residue of poly((α-hydroxy acid). In certain embodiments, including any of the foregoing, POLY² is a residue of poly(vinyl alcohol). In certain embodiments, including any of the foregoing, POLY² is a residue of polyphosphazene. In certain embodiments, including any of the foregoing, POLY² is a residue of polyoxazolines (POZ). In certain embodiments, including any of the foregoing, POLY² is a residue of poly(N-acryloylmorpholine). In certain embodiments, including any of the foregoing, POLY² is a residue of polysarcosine.

In certain embodiments, including any of the foregoing, POLY² is

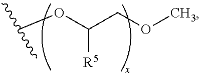

wherein R⁵ is hydrogen or methyl, x is an integer from 1 to 100, inclusive, and [symbol] represents attachment to the remainder of the compound or conjugate. In certain embodiments, including any of the foregoing, x is an integer between 1 to 25. In certain embodiments, including any of the foregoing, x is an integer between 5 to 15. In some embodiments, including any of the foregoing, x is 1. In some embodiments, including any of the foregoing, x is 2. In some embodiments, including any of the foregoing, x is 3. In some embodiments, including any of the foregoing, x is 4. In some embodiments, including any of the foregoing, x is 5. In some embodiments, including any of the foregoing, x is 6. In some embodiments, including any of the foregoing, x is 7. In some embodiments, including any of the foregoing, x is 8. In some embodiments, including any of the foregoing, x is 9. In some embodiments, including any of the foregoing, x is 10. In some embodiments, including any of the foregoing, x is 11. In some embodiments, including any of the foregoing, x is 12. In some embodiments, including any of the foregoing, x is 13. In some embodiments, including any of the foregoing, x is 14. In some embodiments, including any of the foregoing, x is 15. In some embodiments, including any of the foregoing, x is 16. In some embodiments, including any of the foregoing, x is 17. In some embodiments, including any of the foregoing, x is 18. In some embodiments, including any of the foregoing, x is 19. In some embodiments, including any of the foregoing, x is 20. In certain embodiments, including any of the foregoing, x is an integer between 25 and 50. In certain embodiments, including any of the foregoing, x is an integer between 35 and 45. In certain embodiments, including any of the foregoing, x is an integer between 50 and 75. In certain embodiments, including any of the foregoing, x is an integer between 55 and 65. In certain embodiments, including any of the foregoing, x is an integer between 75 and 100. In certain embodiments, including any of the foregoing, x is an integer between 85 and 95. In certain embodiments, including any of the foregoing, x is an integer in the range of 1 and 25, 20 and 45, 40 and 65, 60 and 85, 70 and 95, or 75 and 100.

In some embodiments, including any of the foregoing, R⁵ is hydrogen. In some embodiments, including any of the foregoing, R⁵ is methyl.

In certain embodiments, including any of the foregoing, L² is selected from the group consisting of

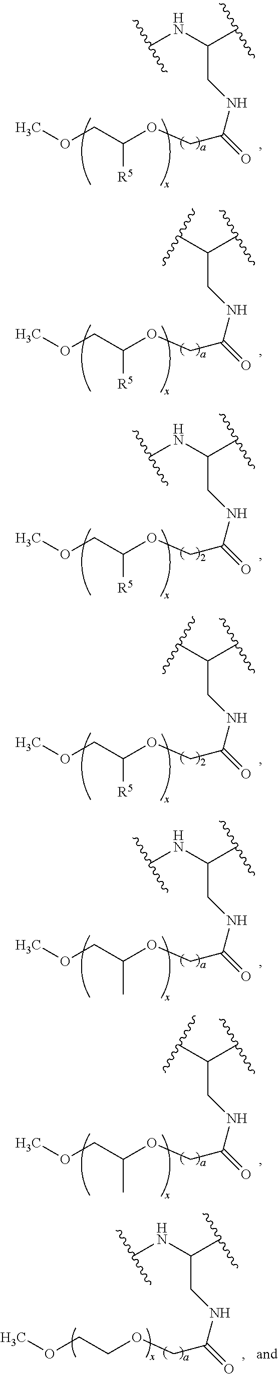

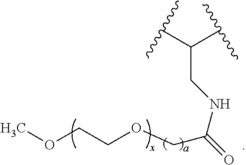

In certain embodiments, including any of the foregoing, L² is selected from the group consisting of

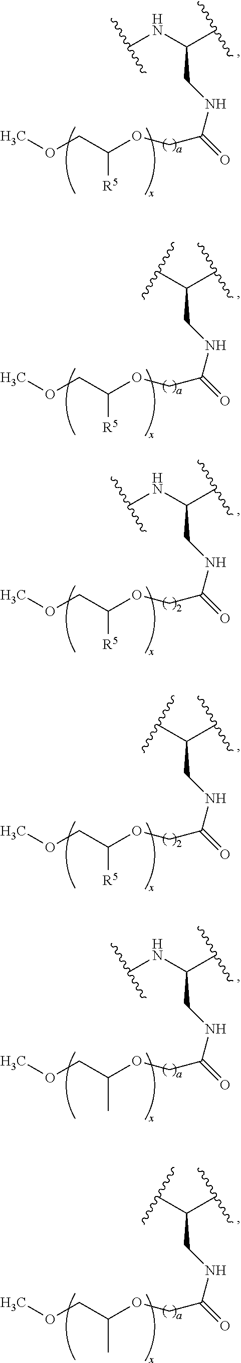

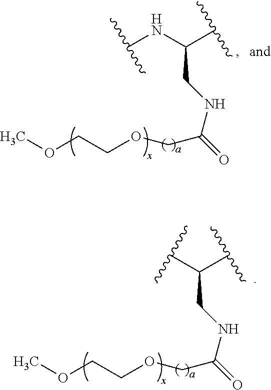

In certain embodiments, including any of the foregoing, L² is

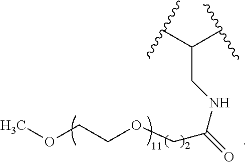

In certain embodiments, including any of the foregoing, L² is

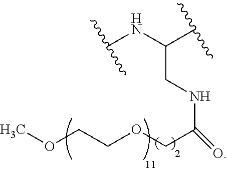

In certain embodiments, including any of the foregoing, L² is

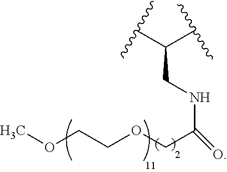

In certain embodiments, including any of the foregoing, L² is

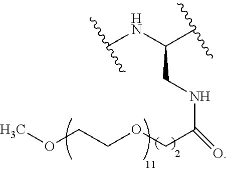

In certain embodiments, including any of the foregoing, L³ is —C(O)-AA-.

In certain embodiments, including any of the foregoing, L³ is —C(O)-AA-Z—(CR^{a}R^{b})ₐ—Z—(CR^{a}R^{b})ₐ—C(O)—. In certain embodiments, including any of the foregoing, L³ is —C(O)-AA-NR²—(CR^{a}R^{b})ₐ—NR²—(CR^{a}R^{b})ₐ—C(O)—. In certain embodiments, including any of the foregoing, L³ is —C(O)-AA-NH—(CR^{a}R^{b})ₐ—NH—(CR^{a}R^{b})ₐ—C(O)—. In certain embodiments, including any of the foregoing, L³ is —C(O)-AA-NH—(CH₂)ₐ—NH—(CH₂)ₐ—C(O)—. In certain embodiments, including any of the foregoing, L³ is —C(O)-AA-NH—(CH₂)ₐ—NH—(CH₂)ₐ—C(O)— wherein a is selected from 1, 2, and 3. In certain embodiments, including any of the foregoing, L³ is —C(O)-AA-NH—CH₂—NH—CH₂—C(O)—

In certain embodiments, including any of the foregoing, L³ is —C(O)-AA-Z—(CR^{a}R^{b})ₐ. In certain embodiments, including any of the foregoing, L³ is —C(O)-AA-NR²—(CH₂)ₐ. In certain embodiments, including any of the foregoing, L³ is —C(O)-AA-NH—(CH₂)₂.

In certain embodiments, including any of the foregoing, L³ is -AA-. In certain embodiments, including any of the foregoing, L³ is

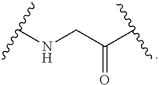 [SMILES: *NCC(*)=O]

In certain embodiments, including any of the foregoing, -AA- is an amino acid residue. In certain embodiments, including any of the foregoing, -AA- is a peptide residue. In certain embodiments, including any of the foregoing, -AA- is a dipeptide residue, a tripeptide residue, a tetrapeptide residue, or a pentapeptide residue. In certain embodiments, including any of the foregoing, -AA- comprises at least one amino acid residue selected from alanine, glycine, valine, and asparagine. In certain embodiments, including any of the foregoing, -AA- comprises at least one amino acid residue selected from alanine and glycine. In certain embodiments, including any of the foregoing, -AA- is selected from the group consisting of

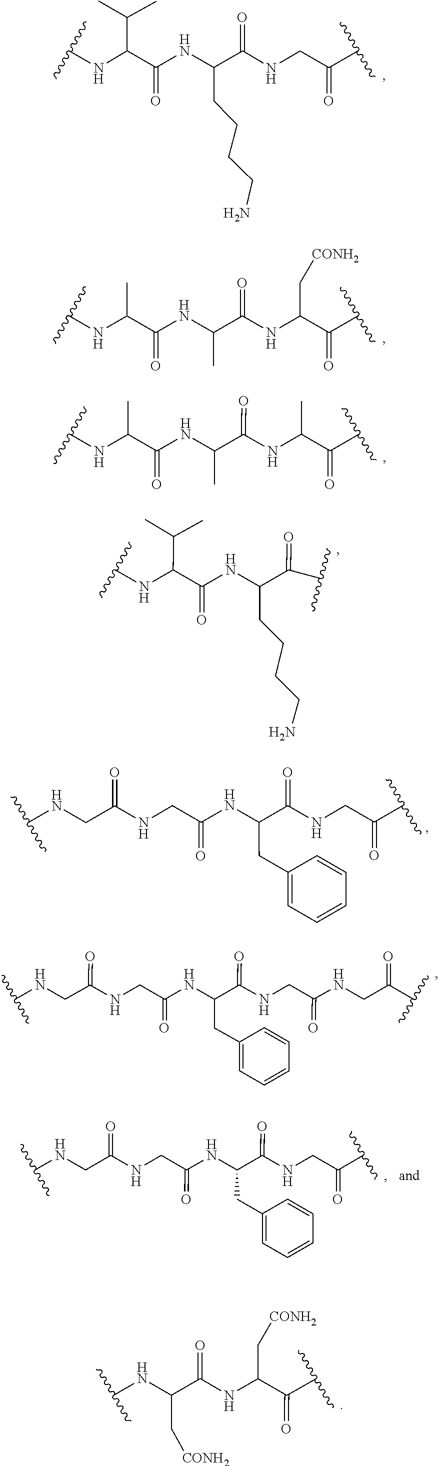

In certain embodiments, including any of the foregoing, -AA- is selected from the group consisting of

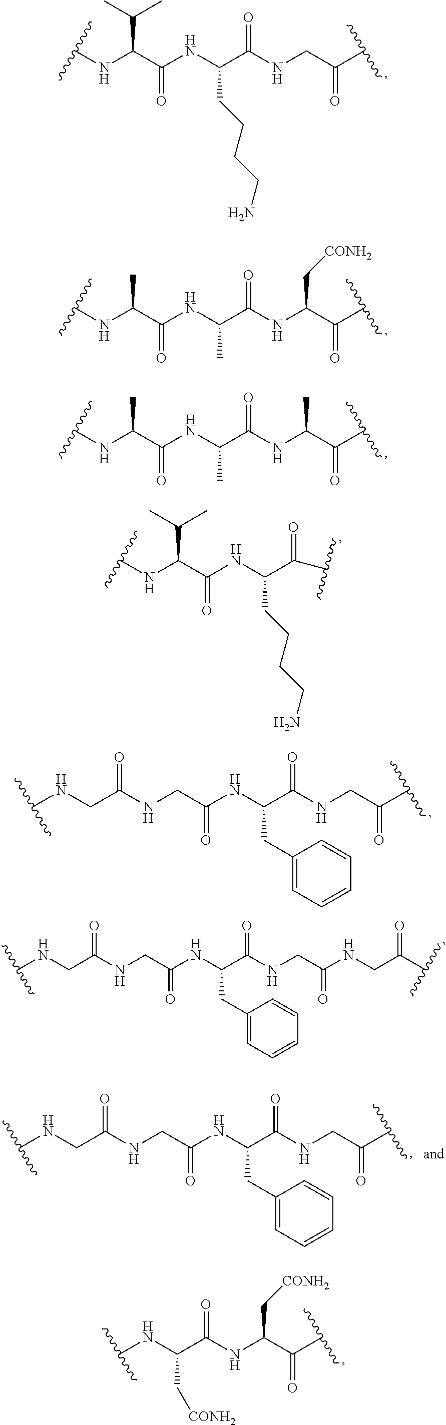

In certain embodiments, including any of the foregoing, L³ is —C(O)—.

In certain embodiments, including any of the foregoing, L³ is —C(O)—Z—(CR^{a}R^{b})ₐ—C(O)—Z-L⁴-OC(O)— wherein L⁴ is

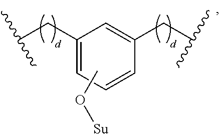

Su is a hexose form of a monosaccharide, and d is an integer independently selected from 1, 2, and 3. In certain embodiments, including any of the foregoing, L³ is —C(O)—NR²—(CR^{a}R^{b})ₐ—C(O)—NR²-L⁴-OC(O)—. In certain embodiments, including any of the foregoing, L³ is —C(O)—NR²—(CH₂)₂—C(O)—NR²-L⁴-OC(O)—. In certain embodiments, including any of the foregoing, L³ is —C(O)—NH—(CR^{a}R^{b})ₐ—C(O)—NH-L⁴-OC(O)—. In certain embodiments, including any of the foregoing, L³ is —C(O)—NH—(CH₂)₂—C(O)—NH-L⁴-OC(O)—.

In certain embodiments, including any of the foregoing, L⁴ is

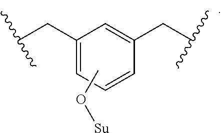

In certain embodiments, including any of the foregoing, L⁴ is

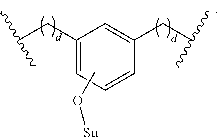

In certain embodiments, including any of the foregoing, L⁴ is

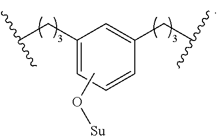

In some embodiments, including any of the foregoing, Su is a sugar moiety. In some embodiments, Su is a hexose form of a monosaccharide. Su may be a glucuronic acid or mannose residue. In certain embodiments, including any of the foregoing, Su is

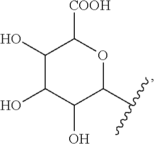

wherein [symbol] represents attachment to the remainder of the compound. In certain embodiments, including any of the foregoing, Su is

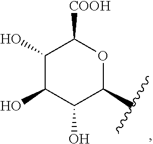

wherein [symbol] represents attachment to the remainder of the compound.

In certain embodiments, including any of the foregoing, L⁴ is

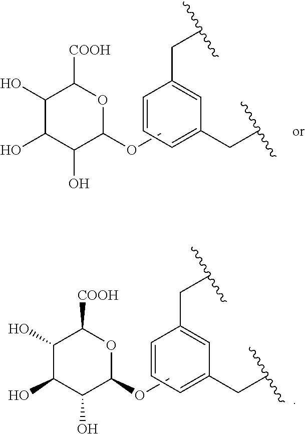

In certain embodiments, including any of the foregoing, L³ is —C(O)—NH—(CH₂)₂—C(O)—NH-L⁴-OC(O)— wherein L⁴ is

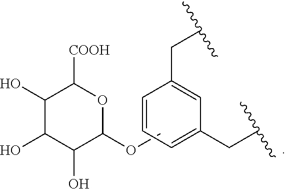

In certain embodiments, including any of the foregoing, L³ is —C(O)—NH—(CH₂)₂—C(O)—NH-L⁴-OC(O)— wherein L⁴ is

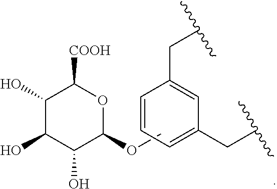

In certain embodiments, including any of the foregoing, L² is —(CR^{a}R^{b})ₐ-POLY¹- and L³ is —C(O)-AA-. In certain embodiments, including any of the foregoing, L² is —(CR^{a}R^{b})ₐ-POLY¹-; L³ is —C(O)-AA-; and, POLY¹ is

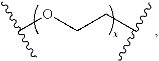

In certain embodiments, including any of the foregoing, L² is —(CH₂)ₐ-POLY¹-; L³ is —C(O)-AA-; POLY¹ is

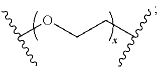

and x is an integer between 10 and 15. In certain embodiments, including any of the foregoing, L² is —(CH₂)₂-POLY¹-; L³ is —C(O)-AA-; and, POLY¹ is

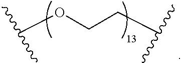

In certain embodiments, including any of the foregoing, L² is —(CH₂)₂-POLY¹-; L³ is —C(O)-AA-; POLY¹ is

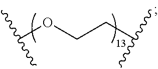

and AA is a dipeptide residue, a tripeptide residue, a tetrapeptide residue, or a pentapeptide residue.

In certain embodiments, including any of the foregoing, L² is —(CR^{a}R^{b})ₐ-POLY¹- and L³ is —C(O)-AA-Z—(CR^{a}R^{b})ₐ—Z—(CR^{a}R^{b})ₐ—C(O)—. In certain embodiments, including any of the foregoing, L² is —(CR^{a}R^{b})ₐ-POLY¹-; L³ is —C(O)-AA-Z—(CR^{a}R^{b})ₐ—Z—(CR^{a}R^{b})ₐ—C(O)—; POLY¹ is

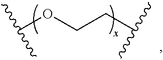

and Z is —NH—. In certain embodiments, including any of the foregoing, L² is —(CH₂)ₐ-POLY¹-; L³ is —C(O)-AA-NH—(CH₂)ₐ—NH—(CH₂)ₐ—C(O)—; POLY¹ is

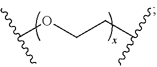

and x is an integer between 10 and 15. In certain embodiments, including any of the foregoing, L² is —(CH₂)₂-POLY¹-; L³ is —C(O)-AA-NH—CH₂—NH—CH₂—C(O)—; and, POLY¹ is

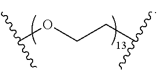

In certain embodiments, including any of the foregoing, L² is —(CH₂)₂-POLY¹-; L³ is —C(O)-AA-NH—CH₂—NH—CH₂—C(O)—; POLY¹ is

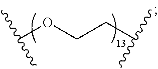

and AA is a dipeptide residue, a tripeptide residue, a tetrapeptide residue, or a pentapeptide residue.

In certain embodiments, including any of the foregoing, L² is —(CR^{a}R^{b})ₐ-POLY¹- and L³ is —C(O). In certain embodiments, including any of the foregoing, L² is —(CR^{a}R^{b})ₐ-POLY¹-; L³ is —C(O); and, POLY¹ is

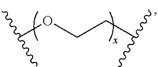

In certain embodiments, including any of the foregoing, L² is —(CH₂)ₐ-POLY¹-; L³ is —C(O); POLY¹ is

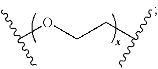

and x is an integer between 10 and 15. In certain embodiments, including any of the foregoing, L² is —(CH₂)₂-POLY¹-; L³ is —C(O); and, POLY¹ is

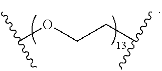

In certain embodiments, including any of the foregoing, L² is —(CH₂)₂-POLY¹-; L³ is —C(O); POLY¹ is

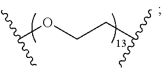

and AA is a dipeptide residue, a tripeptide residue, a tetrapeptide residue, or a pentapeptide residue.

In certain embodiments, including any of the foregoing, L² is —(CR^{a}R^{b})ₐ-POLY¹- and L³ is absent. In certain embodiments, including any of the foregoing, L² is —(CR^{a}R^{b})ₐ-POLY¹-; L³ is absent; and, POLY¹ is

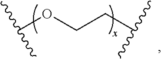

In certain embodiments, including any of the foregoing, L² is —(CH₂)ₐ-POLY¹-; L³ is absent; POLY¹ is

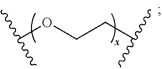

and x is an integer between 10 and 15. In certain embodiments, including any of the foregoing, L² is —(CH₂)₂-POLY¹-; L³ is absent; and, POLY¹ is

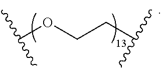

In certain embodiments, including any of the foregoing, L² is —(CH₂)₂-POLY¹-; L³ is absent; POLY¹ is

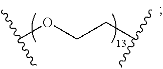

and AA is a dipeptide residue, a tripeptide residue, a tetrapeptide residue, or a pentapeptide residue.

In certain embodiments, including any of the foregoing, L² is —(CR^{a}R^{b})ₐ-POLY¹- and L³ is —C(O)-AA-Z—(CR^{a}R^{b})ₐ—. In certain embodiments, including any of the foregoing, L² is —(CR^{a}R^{b})ₐ-POLY¹-; L³ is —C(O)-AA-Z—(CR^{a}R^{b})ₐ—; and, POLY¹ is

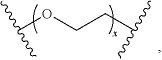

In certain embodiments, including any of the foregoing, L² is —(CH₂)ₐ-POLY¹-; —C(O)-AA-NR²—(CR^{a}R^{b})ₐ—; POLY¹ is

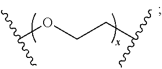

and x is an integer between 10 and 15. In certain embodiments, including any of the foregoing, L² is —(CH₂)₂-POLY¹-; L³ is —C(O)-AA-NH—(CR^{a}R^{b})ₐ—; and, POLY¹ is

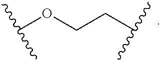 [SMILES: *CCO*]

In certain embodiments, including any of the foregoing, L² is —(CH₂)₂-POLY¹-; L³ is —C(O)-AA-NH—(CR^{a}R^{b})ₐ—; POLY¹ is

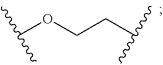 [SMILES: *CCO*]

and AA is a dipeptide residue, a tripeptide residue, a tetrapeptide residue, or a pentapeptide residue.

Non-limiting examples o f-L²-L³- include:

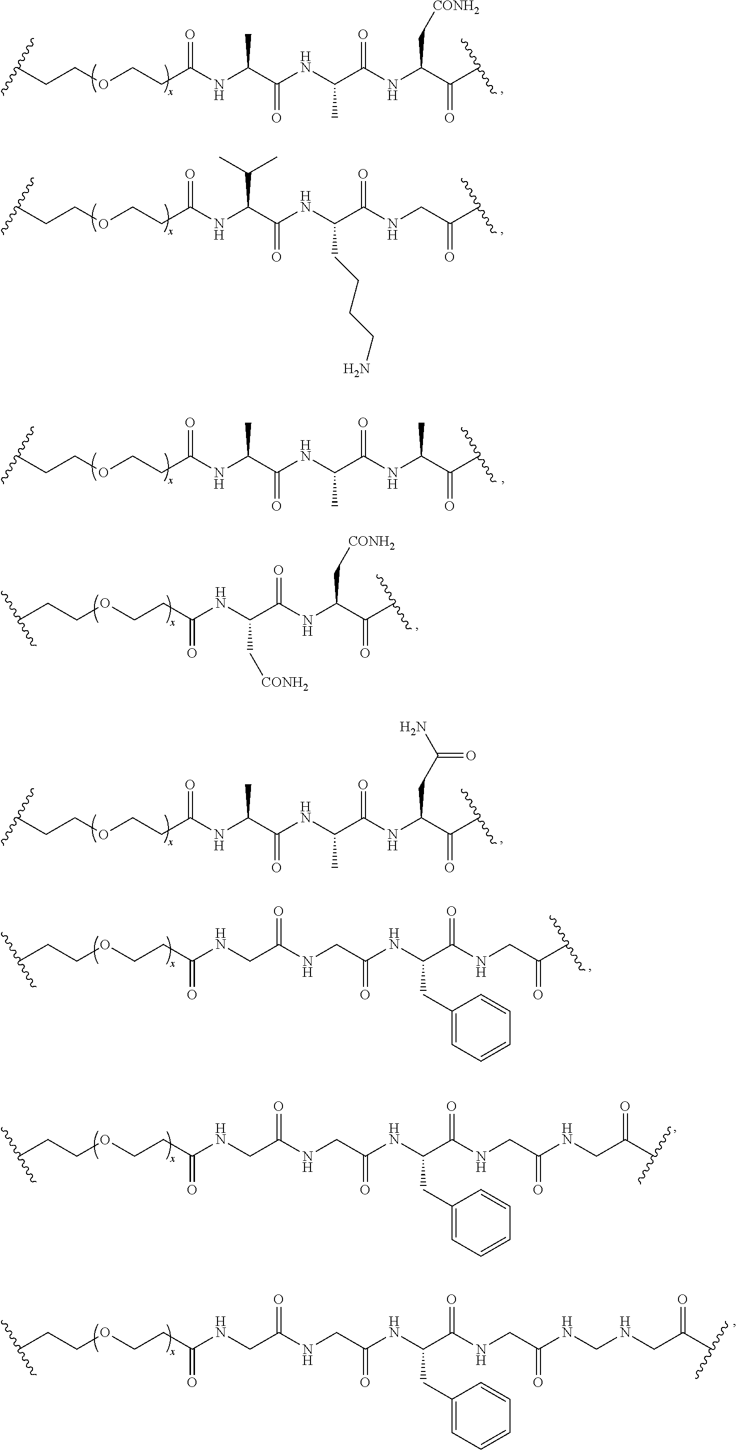

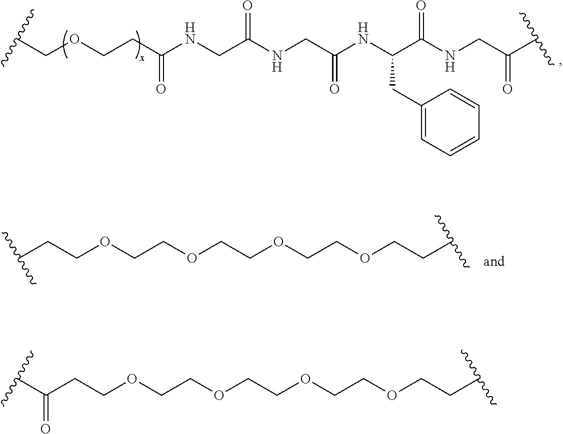

Additional non-limiting examples of -L²-L³- include:

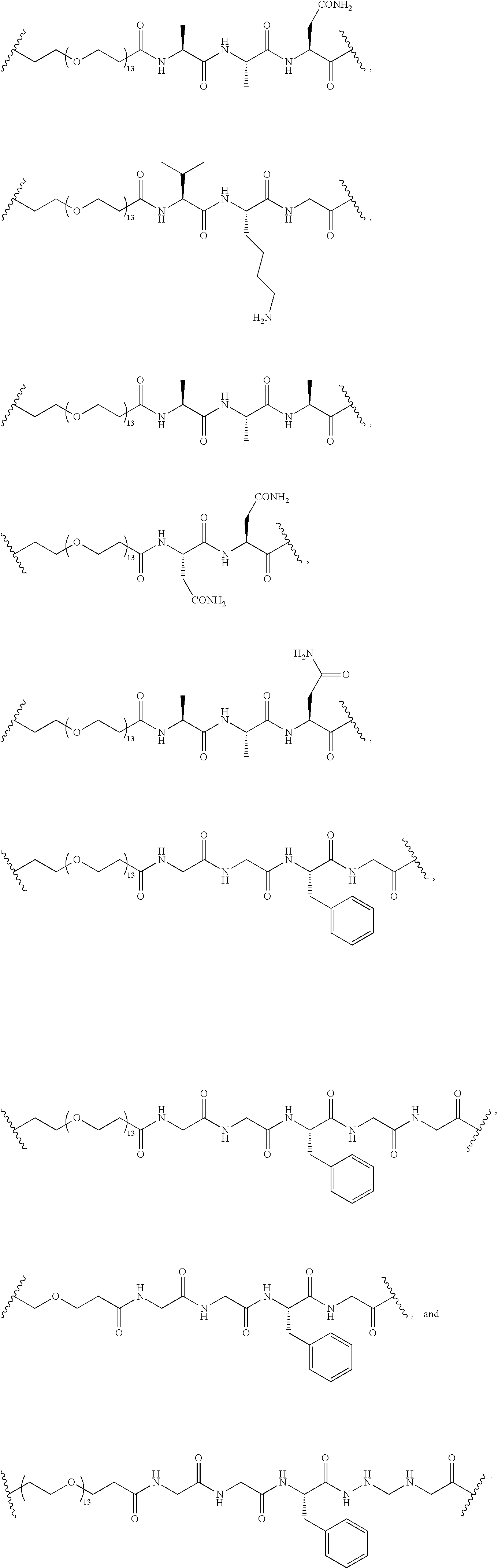

In certain embodiments, including any of the foregoing, L² is

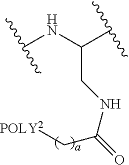

and L¹ is —C(O)—Z—(CR^{a}R^{b})ₐ—C(O)—Z-L⁴-OC(O)— wherein L⁴ is

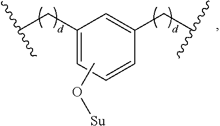

Su is a hexose form of a monosaccharide, and d is an integer independently selected from 1, 2, and 3. In certain embodiments, including any of the foregoing, L² is

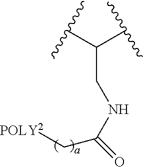

and L³ is —C(O)—Z—(CR^{a}R^{b})ₐ—C(O)—Z-L⁴-OC(O)—.

In certain embodiments, including any of the foregoing, L² is

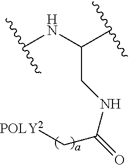

and L³ is —C(O). In certain embodiments, including any of the foregoing, L² is

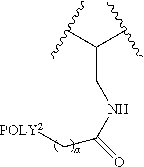

and L³ is absent.

In certain embodiments, including any of the foregoing, L² is selected from the group consisting of

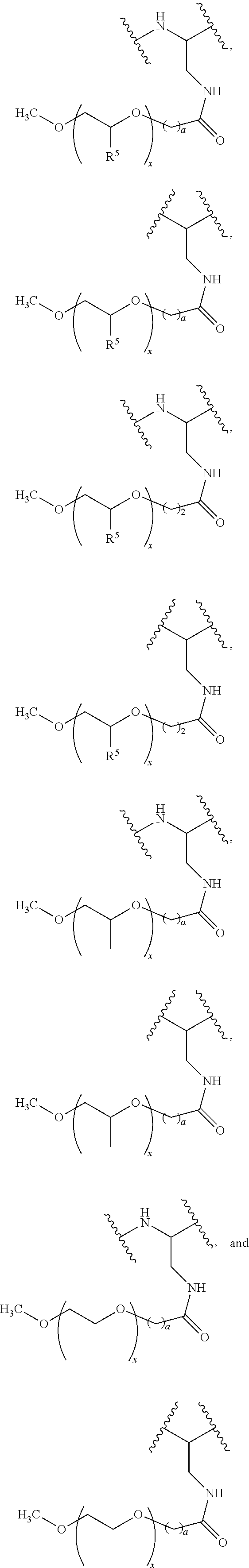

and L³ is —C(O)—NH—(CR^{a}R^{b})ₐ—C(O)—NH-L⁴-OC(O)—.

In certain embodiments, including any of the foregoing, L² is selected from the group consisting of

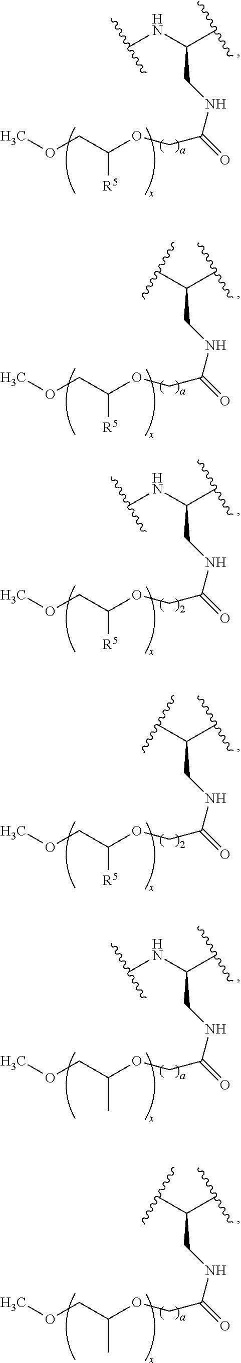

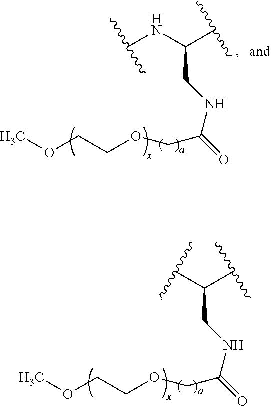

and L³ is —C(O)—NH—(CR^{a}R^{b})ₐ—C(O)—NH-L⁴-OC(O)—.

In certain embodiments, including any of the foregoing, L² is selected from the group consisting of

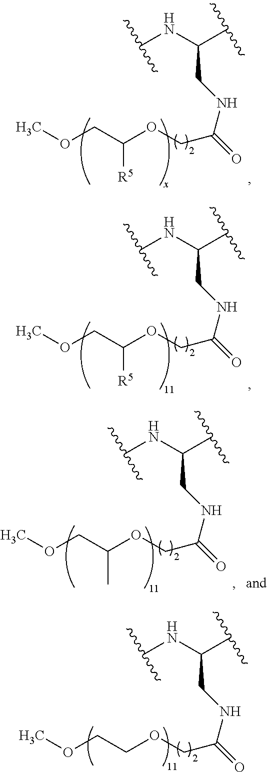

and L³ is —C(O)—NH—(CH₂)₂—C(O)—NH-L⁴-OC(O)— wherein L⁴ is

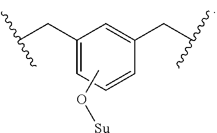

In certain embodiments, including an of the foregoing, L² is selected from the group consisting of

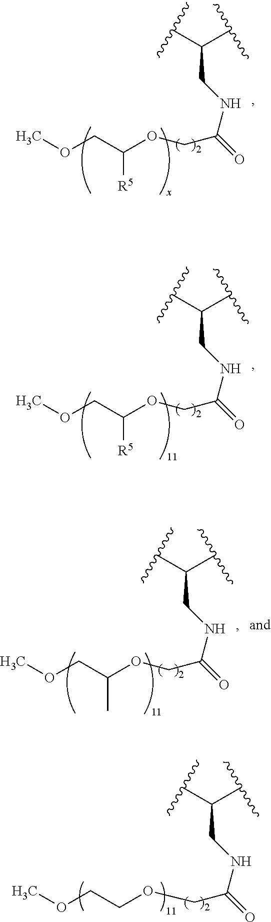

### and L³ is —C(O).

Non-limiting examples of -L²-L³- include:

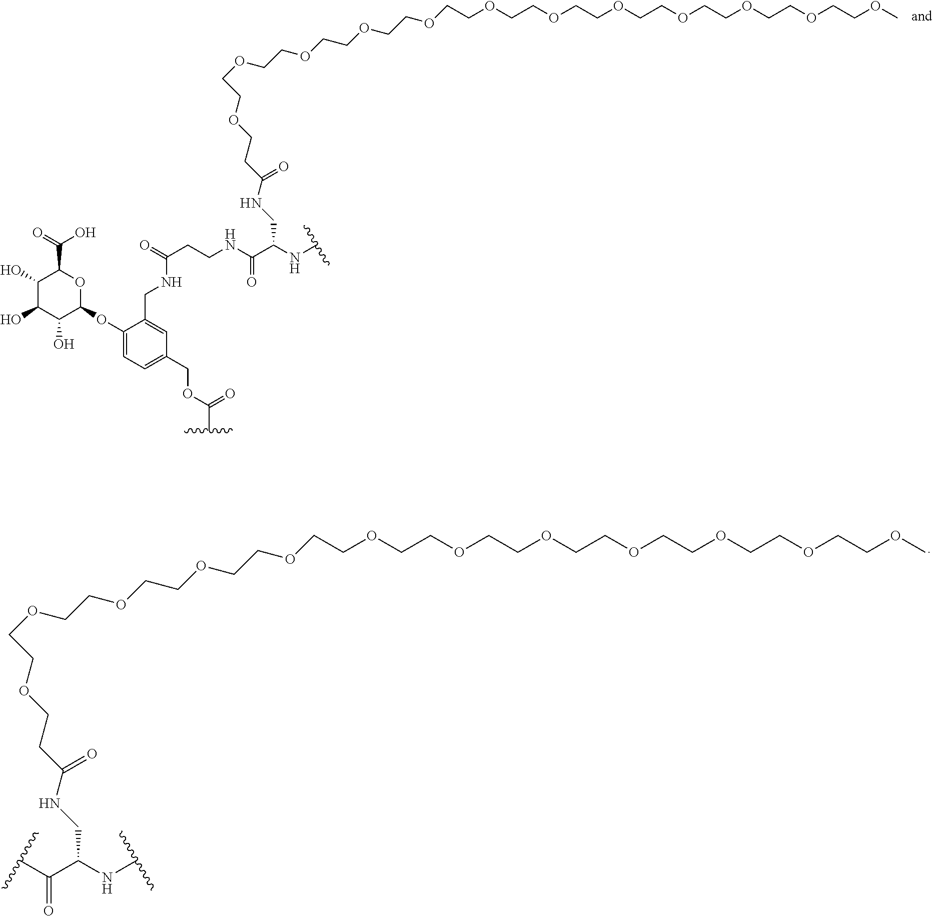 [SMILES: *N[C@@H](CNC(=O)CCOCCOCCOCCOCCOCCOCCOCCOCCOCCOCCOCCOC)C(=O)NCCC(=O)NCc1cc(COC(*)=O)ccc1O[C@@H]1O[C@H](C(=O)O)[C@@H](O)[C@H](O)[C@H]1O ; *N[C@@H](CNC(=O)CCOCCOCCOCCOCCOCCOCCOCCOCCOCCOCCOCCOC)C(*)=O]

In any embodiments of -L²-L³-, D can be selected from

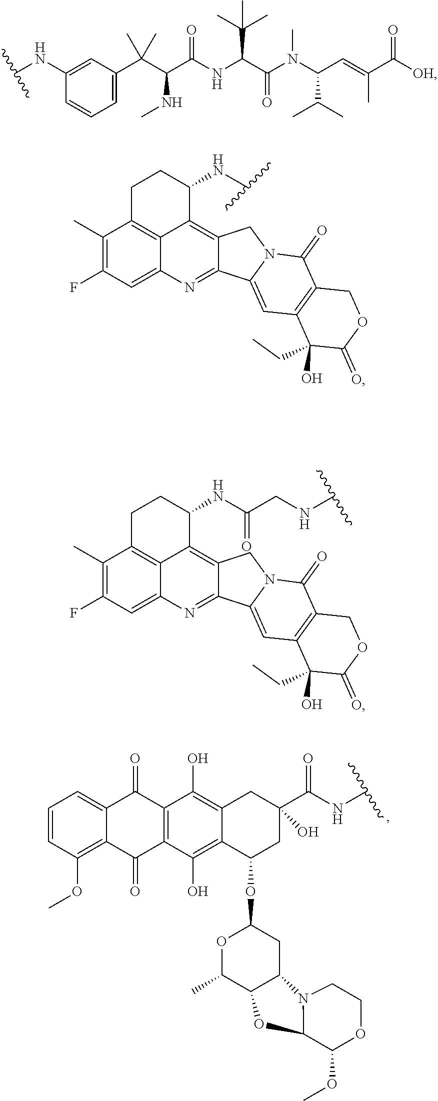 [SMILES: *Nc1cccc(C(C)(C)[C@H](NC)C(=O)N[C@H](C(=O)N(C)[C@H](/C=C(\C)C(=O)O)C(C)C)C(C)(C)C)c1 ; *N[C@H]1CCc2c(C)c(F)cc3nc4c(c1c23)Cn1c-4cc2c(c1=O)COC(=O)[C@]2(O)CC ; *NCC(=O)N[C@H]1CCc2c(C)c(F)cc3nc4c(c1c23)Cn1c-4cc2c(c1=O)COC(=O)[C@]2(O)CC ; *NC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

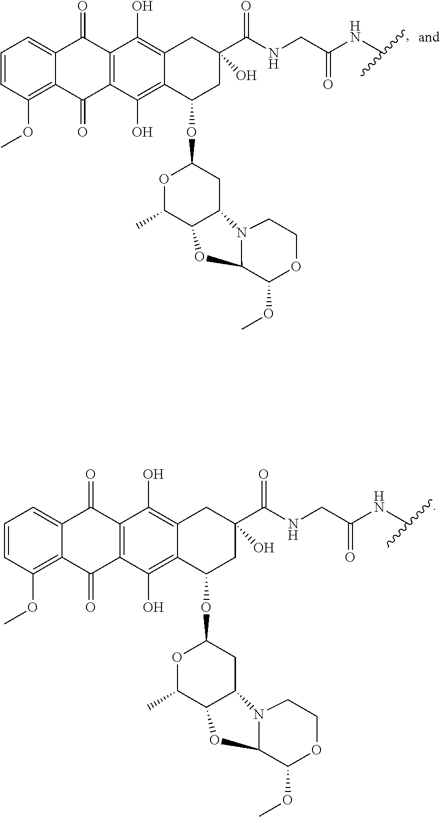 [SMILES: *NC(=O)CNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O ; *NC(=O)CNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

Non-limiting examples of -L²-L³-D include:

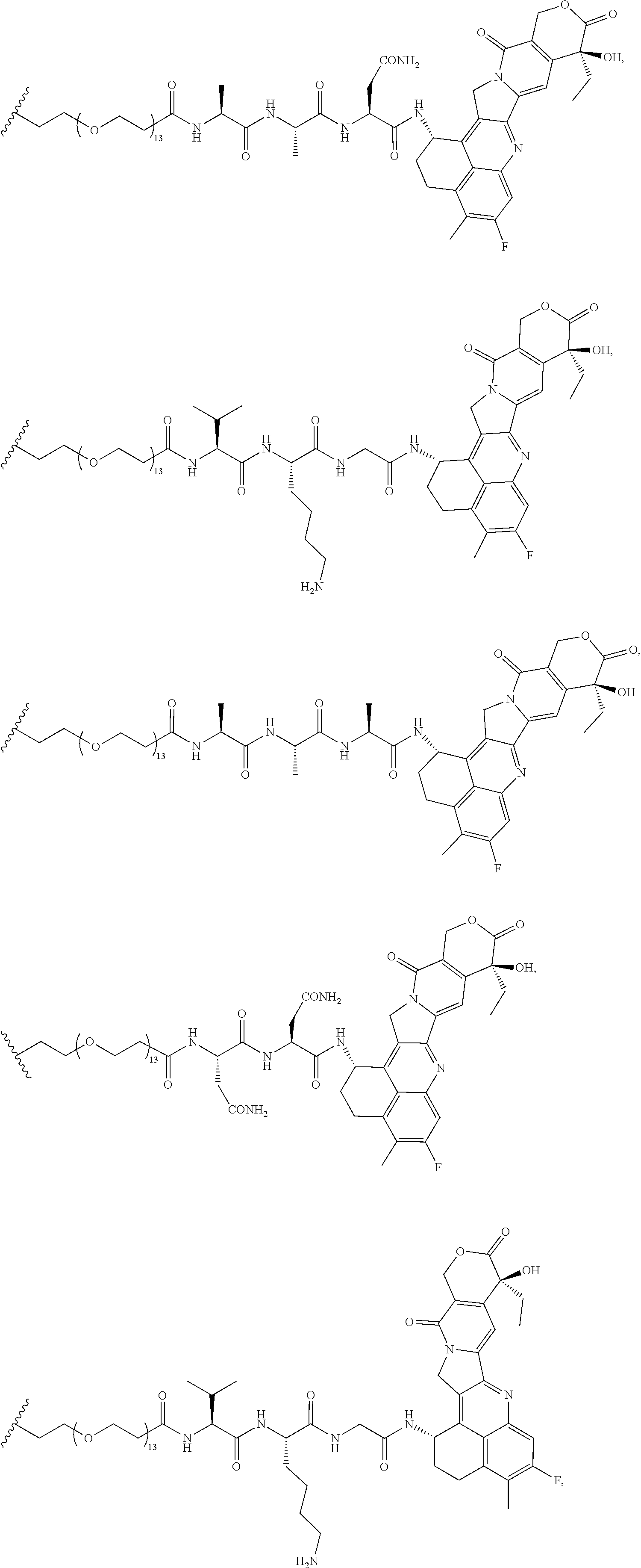

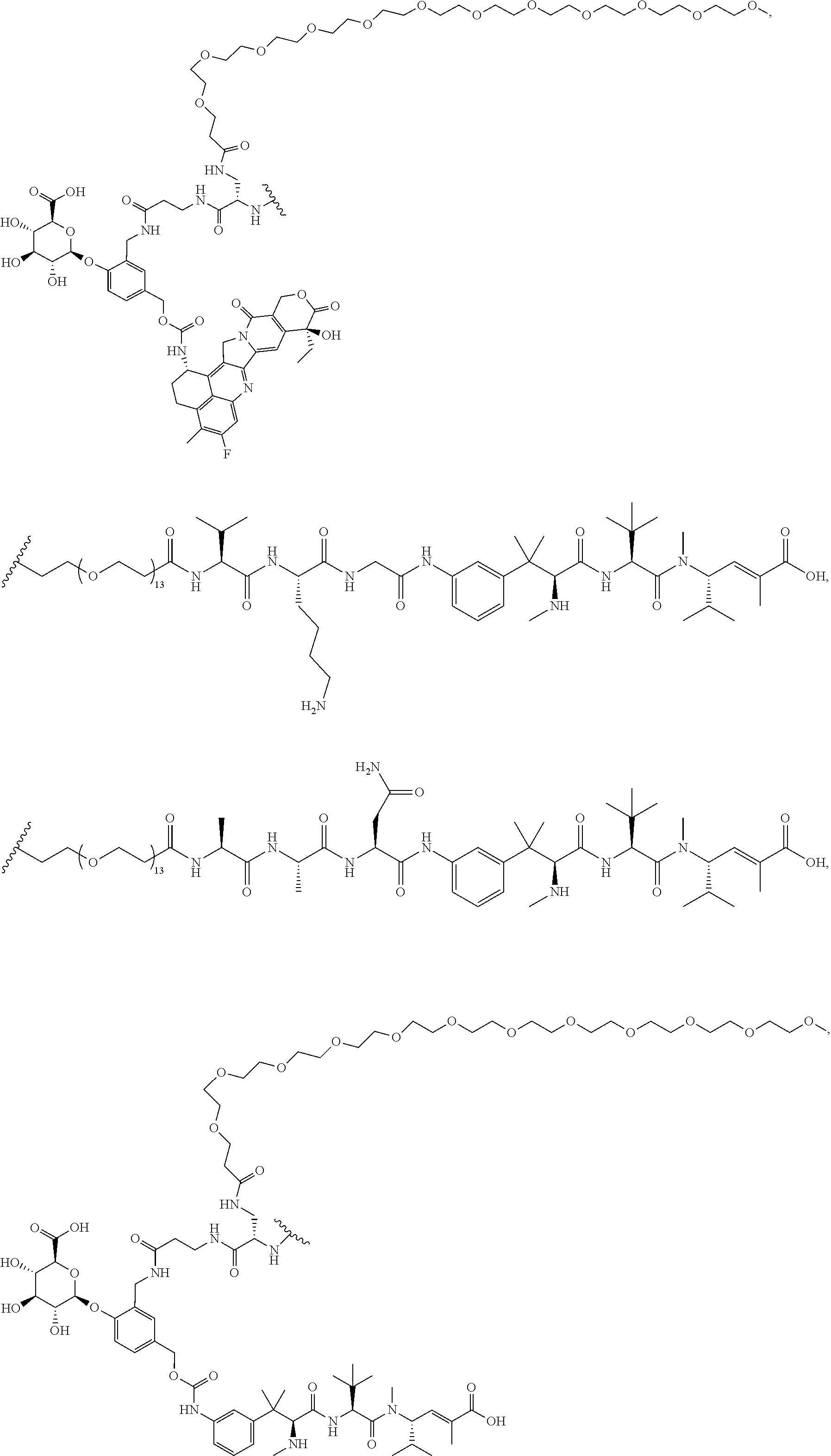

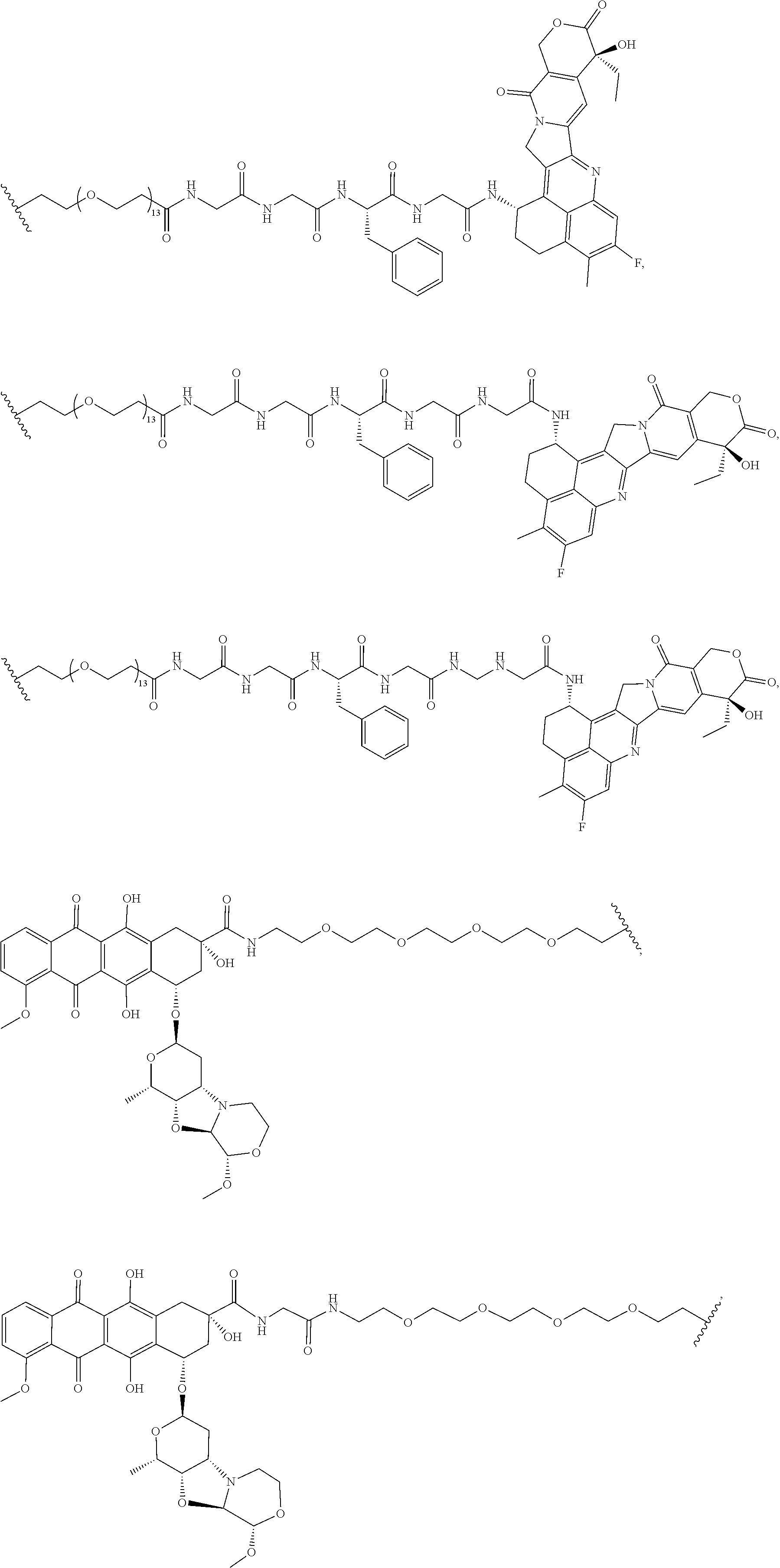

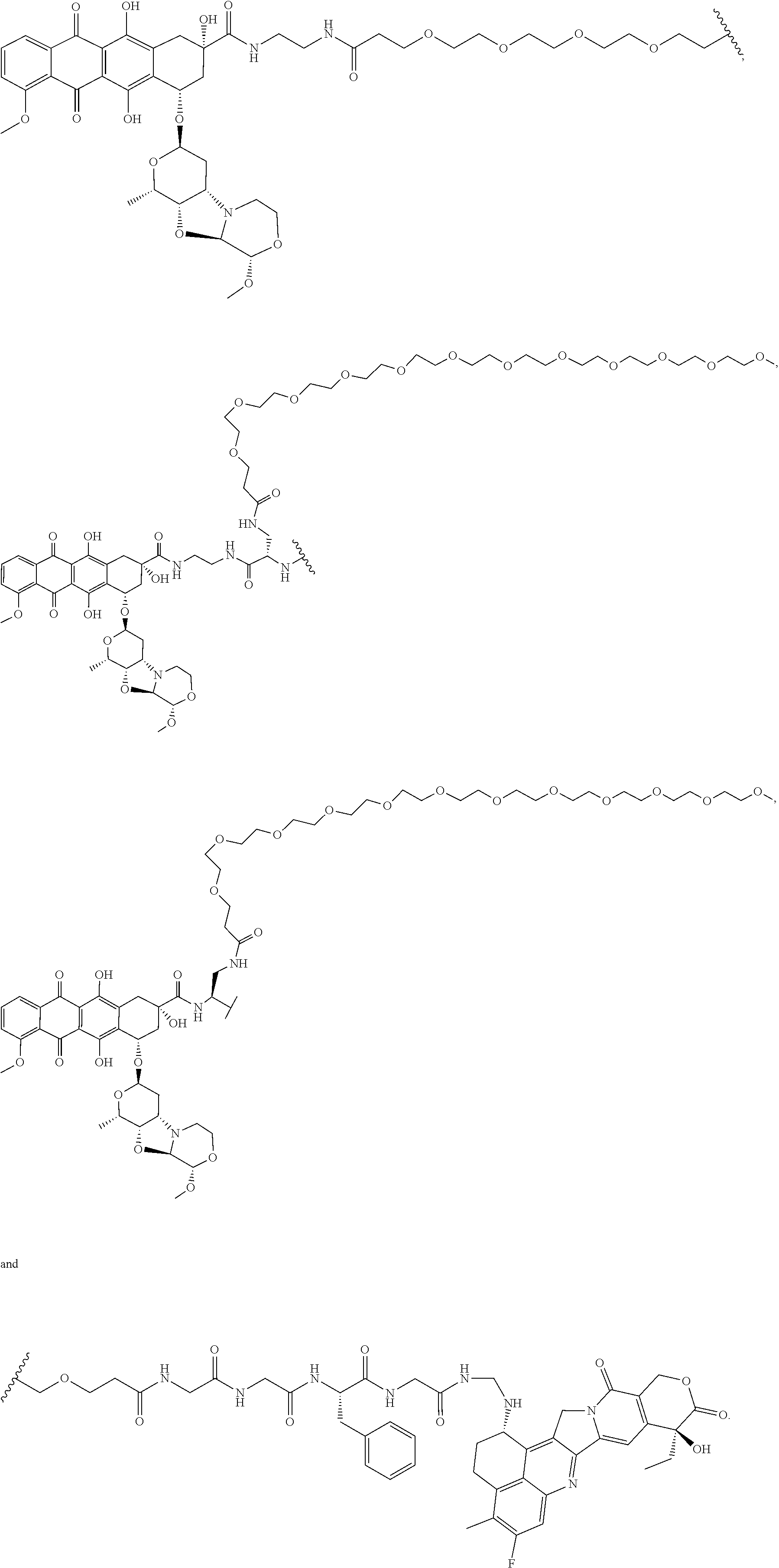

In certain embodiments of Formula (I)—(IH),

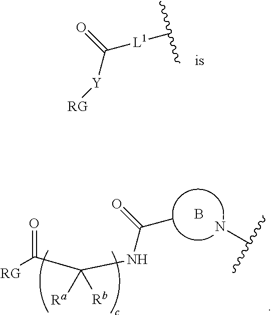

In certain embodiments of Formula (I)—(IH),

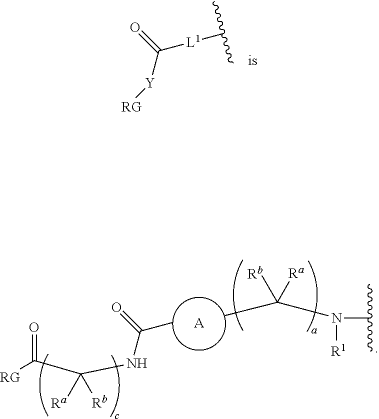

In certain embodiments of Formula (I)—(IH),

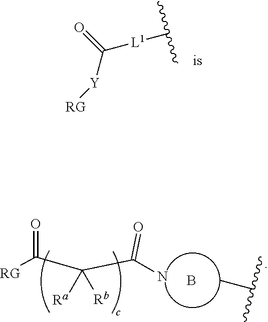

In certain embodiments of Formula (I)—(IH),

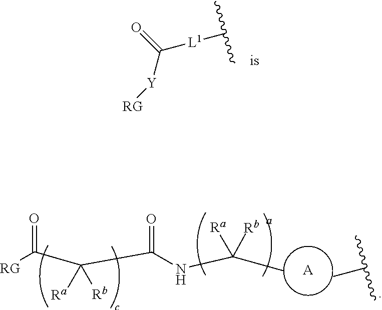

In certain embodiments of Formula (I)—(IH),

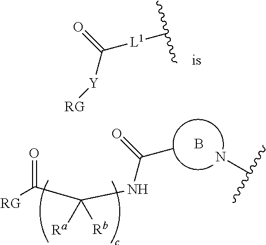

wherein R^{a} and R^{b} are hydrogen. In certain embodiments of Formula (I)—(IH),

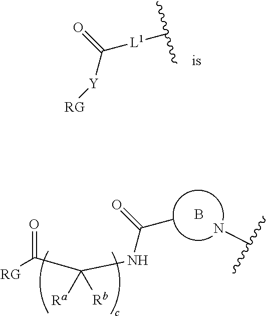

wherein R^{a} and R^{b} are hydrogen and c is 2. In certain embodiments of Formula (I)—(IH),

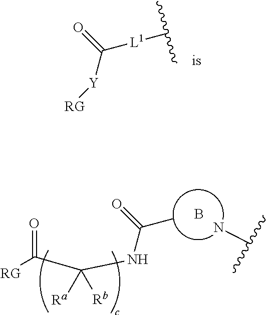

wherein R^{a} and R^{b} are hydrogen, c is 2, and RG is

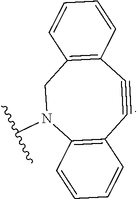 [SMILES: *N1Cc2ccccc2C#Cc2ccccc21]

In certain embodiments of Formula (I)—(IH),

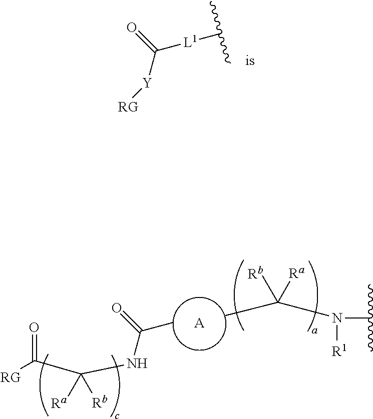

wherein R^{a}, R^{b}, and R¹ are hydrogen. In certain embodiments of Formula (I)—(IH),

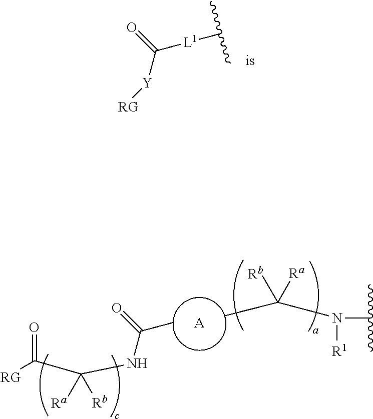

wherein R^{a}, R^{b}, and R¹ are hydrogen; c is 2; and a is 1. In certain embodiments of Formula (I)—(IH),

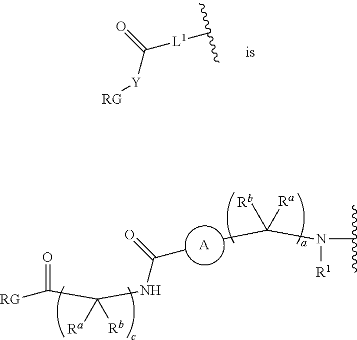

wherein R^{a}, R^{b}, and R¹ are hydrogen; c is 2; a is 1; and RG is

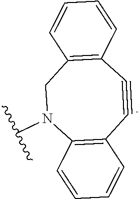 [SMILES: *N1Cc2ccccc2C#Cc2ccccc21]

In certain embodiments of Formula (I)—(IH),

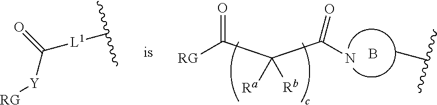

wherein R^{a} and R^{b} are hydrogen. In certain embodiments of Formula (I)—(IH),

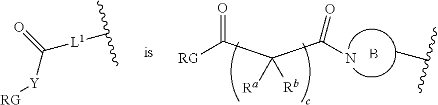

wherein R^{a} and R^{b} are hydrogen and c is 4. In certain embodiments of Formula (I)—(IH),

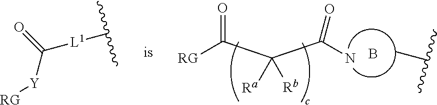

wherein R^{a} and R^{b} are hydrogen, c is 4, and RG is

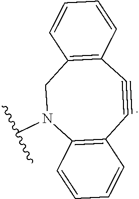 [SMILES: *N1Cc2ccccc2C#Cc2ccccc21]

In certain embodiments of Formula (I)—(IH),

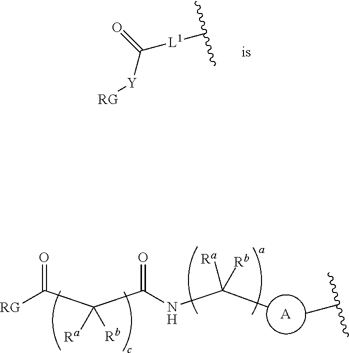

wherein R^{a} and R^{b} are hydrogen. In certain embodiments of Formula (I)—(IH),

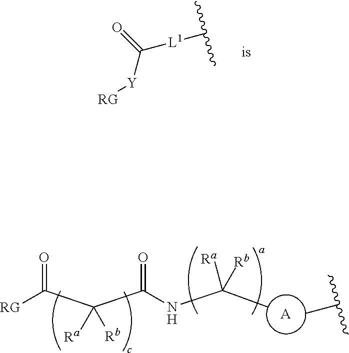

wherein R^{a} and R^{b} are hydrogen and c is 4. In certain embodiments of Formula (I)—(IH),

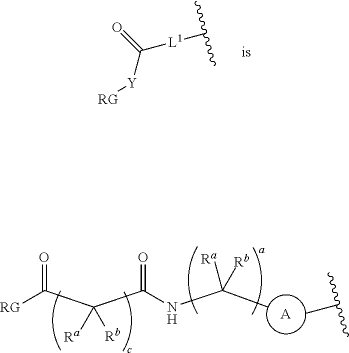

wherein R^{a} and R^{b} are hydrogen, c is 4, and RG is

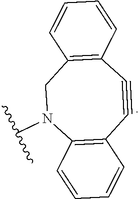 [SMILES: *N1Cc2ccccc2C#Cc2ccccc21]

Non-limiting examples of

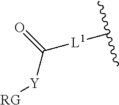

include:

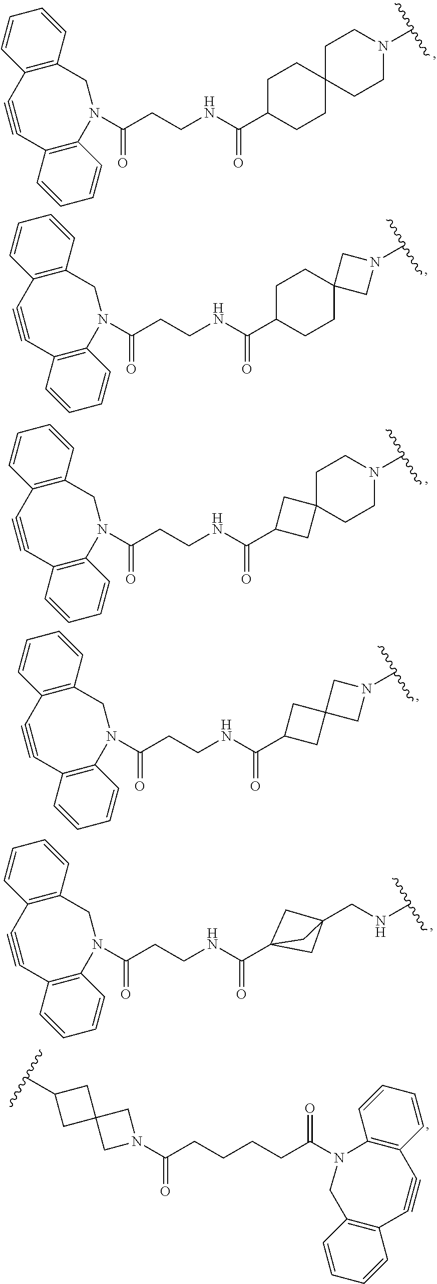

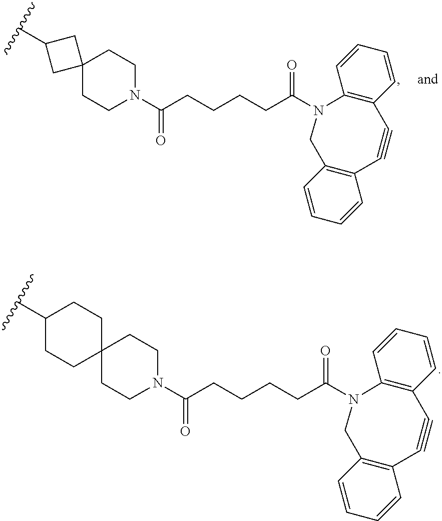 [SMILES: *C1CC2(CCN(C(=O)CCCCC(=O)N3Cc4ccccc4C#Cc4ccccc43)CC2)C1 ; *C1CCC2(CC1)CCN(C(=O)CCCCC(=O)N1Cc3ccccc3C#Cc3ccccc31)CC2]

In certain embodiments of Formula (III), (IIIA), or (IIIB),

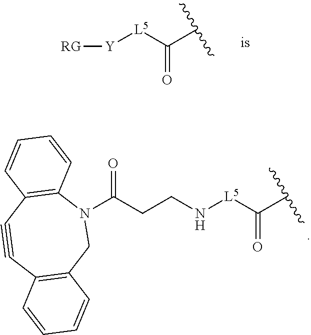

In certain embodiments Formula (III), (IIIA), or (IIIB),

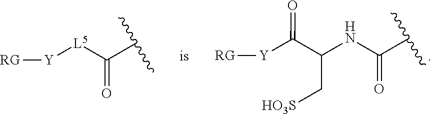

In certain embodiments Formula (III), (IIIA), or (IIIB),

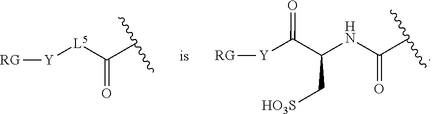

In certain embodiments Formula (III), (IIIA), or (IIIB),

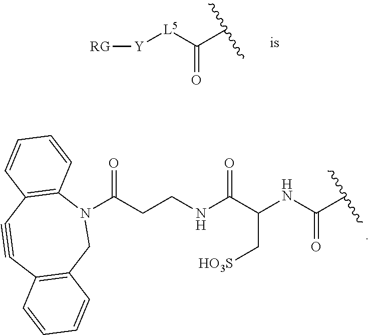

In certain embodiments Formula (III), (IIIA), or (IIIB),

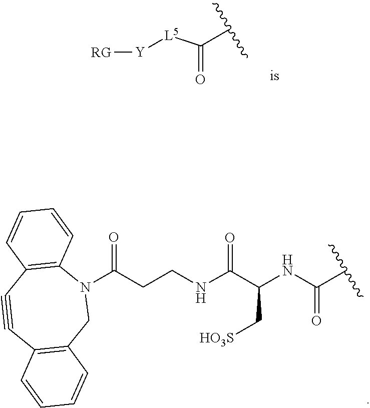

In certain embodiments of Formula (I)—(IH) or (III)—(IIIB), RG is a group comprising an alkyne, cyclooctyne, a strained alkene, a tetrazine, an amine, methylcyclopropene, a thiol, a para-acetyl-phenylalanine residue, an oxyamine, a maleimide, or an azide. In certain embodiments of Formula (I)—(IH) or (III)—(IIIB), RG comprises an alkyne. In certain embodiments of Formula (I)—(IH) or (III)—(IIIB), RG comprises a cyclooctyne. In certain embodiments of Formula (I)—(IIIB), RG comprises a strained alkene. In certain embodiments of Formula (I)—(IH) or (III)-(TIB), RG comprises a tetrazine. In certain embodiments of Formula (I)—(IH) or (III)—(IIIB), RG comprises an amine. In certain embodiments of Formula (I)—(IH) or (III)—(IIIB), RG comprises a methylcyclopropene. In certain embodiments of Formula (I)—(IH) or (III)—(IIIB), RG comprises a thiol. In certain embodiments of Formula (I)—(IH) or (III)—(IIIB), RG comprises a para-acetyl-phenylalanine residue. In certain embodiments of Formula (I)—(IH) or (III)—(IIIB), RG comprises an oxyamine. In certain embodiments of Formula (I)—(IH) or (III)—(IIIB), RG comprises a maleimide. In certain embodiments of Formula (I)—(IH) or (III)—(IIIB), RG comprises an azide. In certain embodiments of Formula (I)—(IH) or (III)—(IIIB), RG is selected from the group consisting of

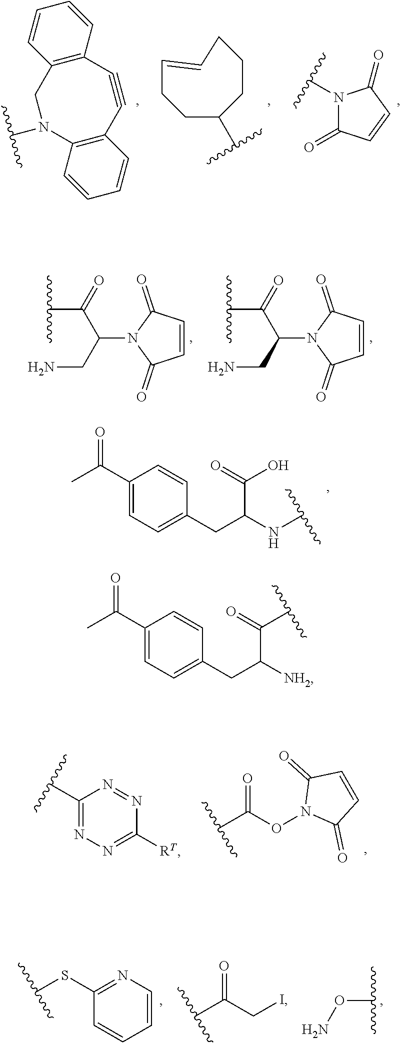

—N₃, —NH₂, methylcyclopropene, and —SH; wherein R^{T} is C₁₋₆ alkyl; and [symbol] represents attachment to the remainder of the compound. In certain embodiments of Formula (I)—(IH) or (III)—(IIIB), RG is

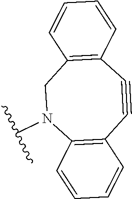 [SMILES: *N1Cc2ccccc2C#Cc2ccccc21]

and [symbol] represents attachment to the remainder of the compound. In certain embodiments of Formula (I)—(IH) or (III)—(IIIB), RG is

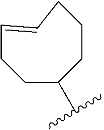 [SMILES: *C1CC/C=C/CCC1]

and [symbol] represents attachment to the remainder of the compound. In certain embodiments of Formula (I)—(IH) or (III)—(IIIB), RG is

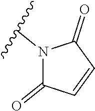 [SMILES: *N1C(=O)C=CC1=O]

and [symbol] represents attachment to the remainder of the compound. In certain embodiments of Formula (I)—(IH) or (III)—(IIIB), RG is

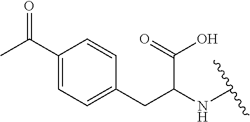 [SMILES: *NC(Cc1ccc(C(C)=O)cc1)C(=O)O]

and [symbol] represents attachment to the remainder of the compound. In certain embodiments of Formula (I)—(IH) or (III)—(IIIB), RG is

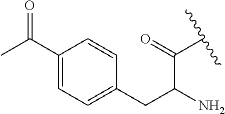 [SMILES: *C(=O)C(N)Cc1ccc(C(C)=O)cc1]

and [symbol] represents attachment to the remainder of the compound. In certain embodiments of Formula (I)—(IH) or (III)—(IIIB), RG is

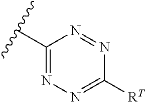

wherein R^{T} is C₁₋₆ alkyl and [symbol] represents attachment to the remainder of the compound. In certain embodiments, R^{T} is methyl, ethyl, or propyl. In certain embodiments, R^{T} is methyl. In certain embodiments, R^{T} is ethyl. In certain embodiments, R^{T} is propyl. In certain embodiments, R^{T} is butyl. In certain embodiments, R^{T} is pentyl. In certain embodiments, R^{T} is hexyl. In certain embodiments of Formula (I)—(IH) or (III)—(IIIB), RG is

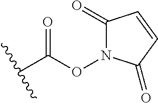 [SMILES: *C(=O)ON1C(=O)C=CC1=O]

and [symbol] represents attachment to the remainder of the compound. In certain embodiments of Formula (I)—(IH) or (III)—(IIIB), RG is

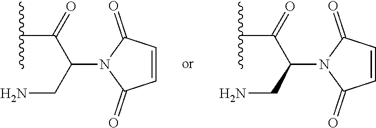 [SMILES: *C(=O)C(CN)N1C(=O)C=CC1=O ; *C(=O)[C@H](CN)N1C(=O)C=CC1=O]

and [symbol] represents attachment to the remainder of the compound. In certain embodiments of Formula (I)—(IH) or (III)—(IIIB), RG is

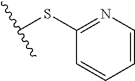 [SMILES: *Sc1ccccn1]

and [symbol] represents attachment to the remainder of the compound. In certain embodiments of Formula (I)—(IH) or (III)—(IIIB), RG is

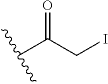 [SMILES: *C(=O)CI]

and [symbol] represents attachment to the remainder of the compound. In certain embodiments of Formula (I)—(IH) or (III)—(IIIB), RG is

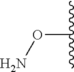 [SMILES: *ON]

and [symbol] represents attachment to the remainder of the compound. In certain embodiments of Formula (I)-(TH) or (III)—(IIIB), RG is —N₃. In certain embodiments of Formula (I)—(IH) or (III)—(IIIB), RG is —NH₂. In certain embodiments of Formula (I)—(IIIB), RG is methylcyclopropene. In certain embodiments of Formula (I)—(IH) or (III)—(IIIB), RG is —SH.

In certain embodiments of Formula (II)-(IIH),

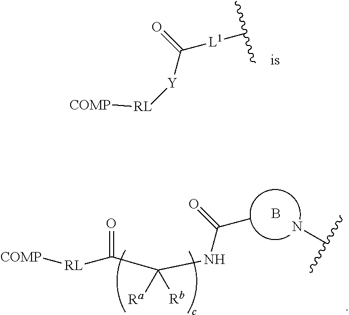

In certain embodiments of Formula (II)-(IIH),

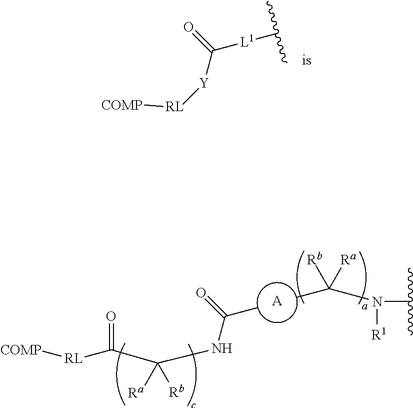

In certain embodiments of Formula (II)—(IH),

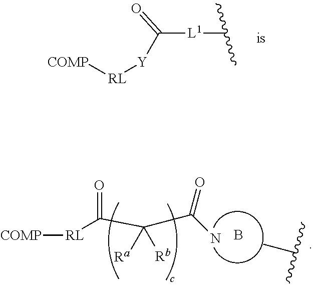

In certain embodiments of Formula (II)-(IIH),

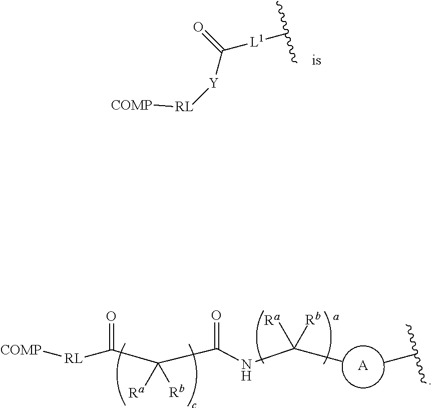

In certain embodiments of Formula (II)-(IIH),

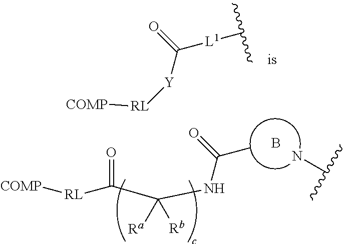

wherein R^{a} and R^{b} are hydrogen. In certain embodiments of Formula (II)-(IIH),

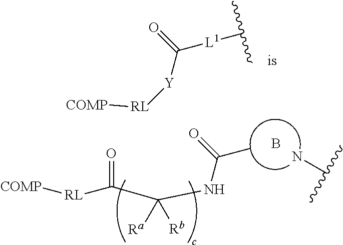

wherein R^{a} and R^{b} are hydrogen and c is 2. In certain embodiments of Formula (II)-(IIH),

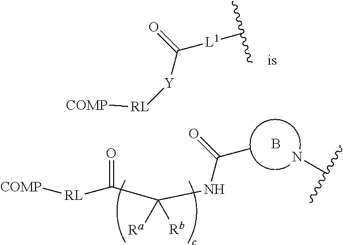

wherein R^{a} and R^{b} are hydrogen, c is 2, and RL is

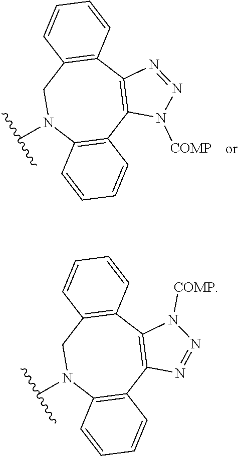

In certain embodiments of Formula (II)-(IIH),

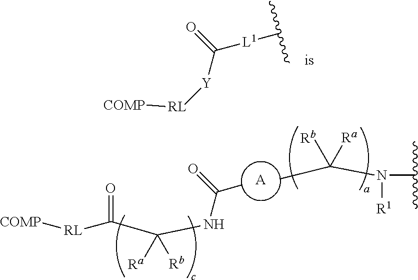

wherein R^{a}, R^{b}, and R¹ are hydrogen. In certain embodiments of Formula (II)-(IIH),

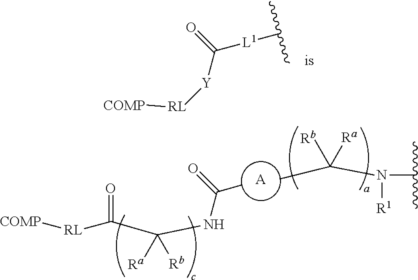

wherein R^{a}, R^{b}, and R¹ are hydrogen; c is 2; and a is 1. In certain embodiments of Formula (II)-(IIH),

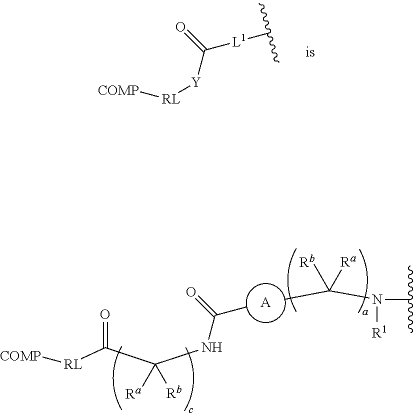

wherein R^{a}, R^{b}, and R¹ are hydrogen; c is 2; a is 1; and RL is

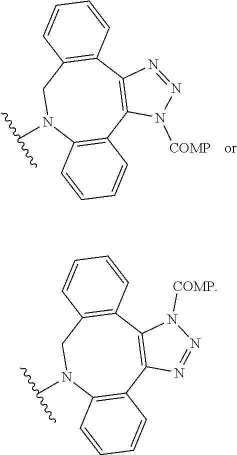

In certain embodiments of Formula (II)-(IIH),

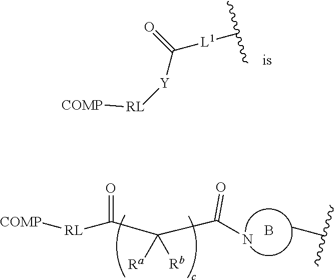

wherein R^{a} and R^{b} are hydrogen. In certain embodiments, of Formula (II)-(IIH)

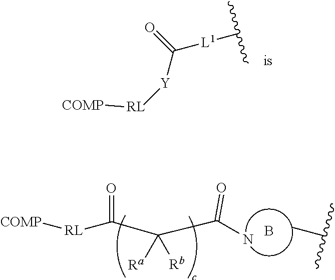

wherein R^{a} and R^{b} are hydrogen and c is 4. In certain embodiments of Formula (II)-(IIH),

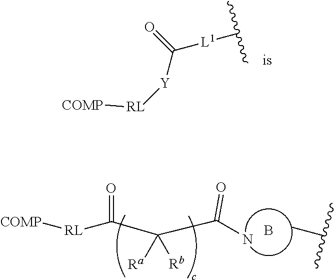

wherein R^{a} and R^{b} are hydrogen, c is 4, and RL is

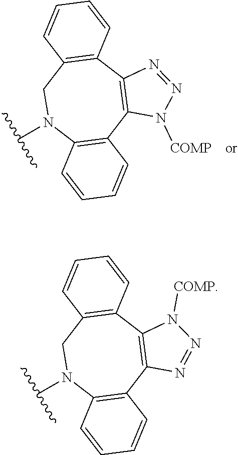

In certain embodiments of Formula (II)-(IIH),

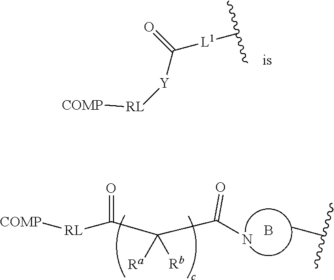

wherein R^{a} and R^{b} are hydrogen.

In certain embodiments of Formula (II)-(IIH),

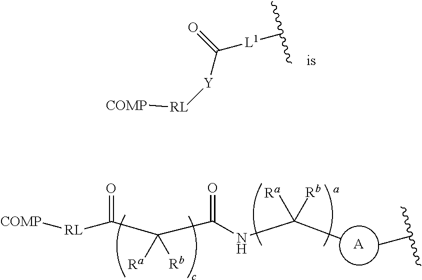

wherein R^{a} and R^{b} are hydrogen and c is 4. In certain embodiments of Formula (II)-(IIH),

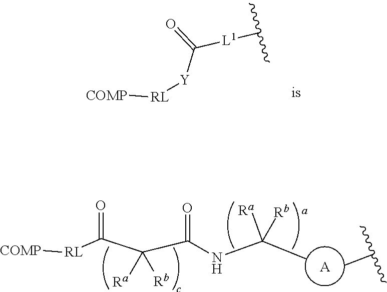

wherein R^{a} and R^{b} are hydrogen, c is 4, and RL is

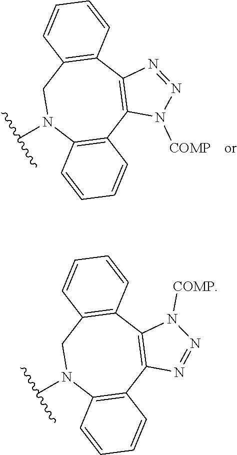

In certain embodiments of Formula (IV)—(IVB),

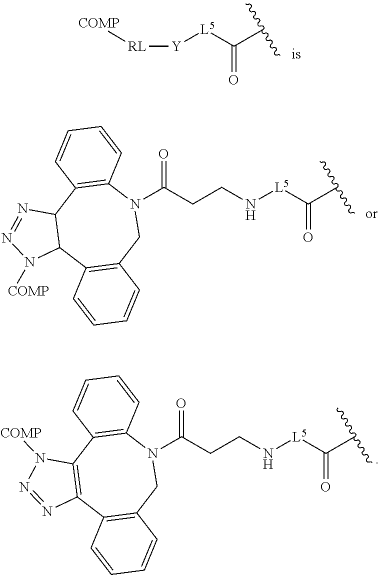

In certain embodiments of Formula (IV)—(IVB),

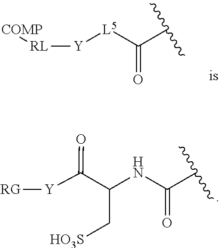

In certain embodiments of Formula (IV)—(IVB)

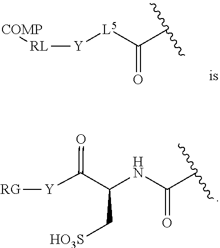

In certain embodiments of Formula (IV)—(IVB),

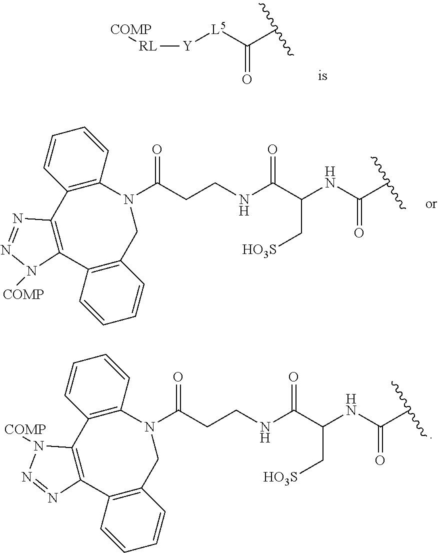

In certain embodiments of Formula (IV)—(IVB),

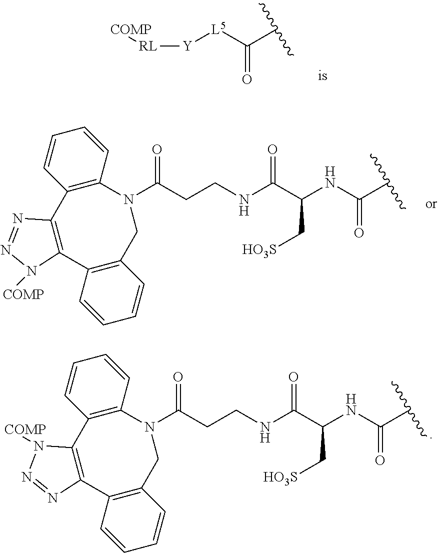

In certain embodiments of Formula (II)-(IIH), (IVA), or (IVB), RL is a group comprising a triazole, pyridazine, a thiol, or an oxime. In certain embodiments of Formula (II)-(IIH), (IV), (IVA), or (IVB), RL is a group comprising a triazole. In certain embodiments of Formula (II)-(IIH), (IV), (IVA), or (IVB), RL is a group comprising a pyridazine. In certain embodiments of Formula (II)-(IIH), (IV), (IVA), or (IVB), RL is a group comprising a thiol. In certain embodiments of Formula (II)-(IIH), (IV), (IVA), or (IVB), RL is a group comprising an oxime.

In certain embodiments of Formula (II)-(IIH), (IV), (IVA), or (IVB), RL is selected from the group consisting of

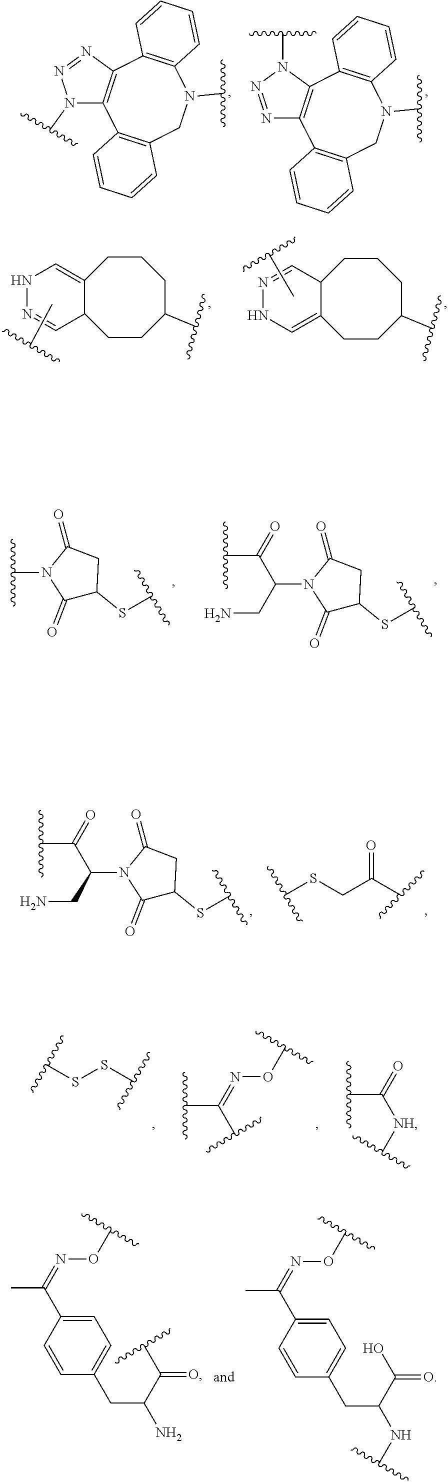

and [symbol] represents attachment to the remainder of the compound. In certain embodiments of Formula (II)-(IIH), (IV), (IVA), or (IVB), RL is

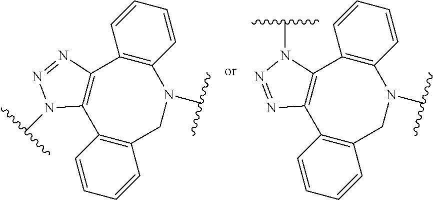 [SMILES: *N1Cc2ccccc2-c2c(nnn2*)-c2ccccc21 ; *N1Cc2ccccc2-c2nnn(*)c2-c2ccccc21]

and [symbol] represents attachment to the remainder of the compound. In certain embodiments of Formula (II)-(IIH), (IV), (IVA), or (IVB), RL is

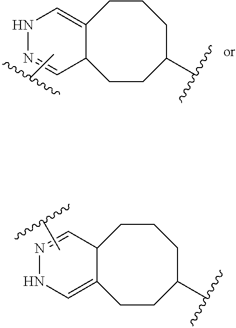

and [symbol] represents attachment to the remainder of the compound. In certain embodiments of Formula (II)-(IIH), (IV), (IVA), or (IVB), RL is

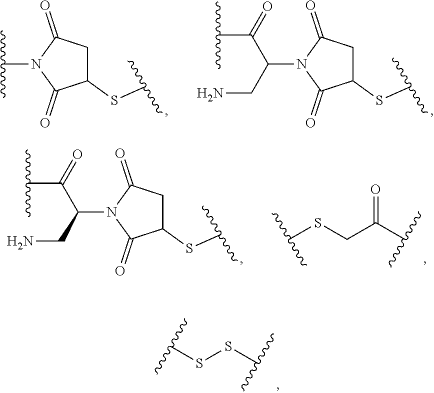

and [symbol] represents attachment to the remainder of the compound. In certain embodiments of Formula (II)-(IIH), (IV), (IVA), or (IVB), RL is

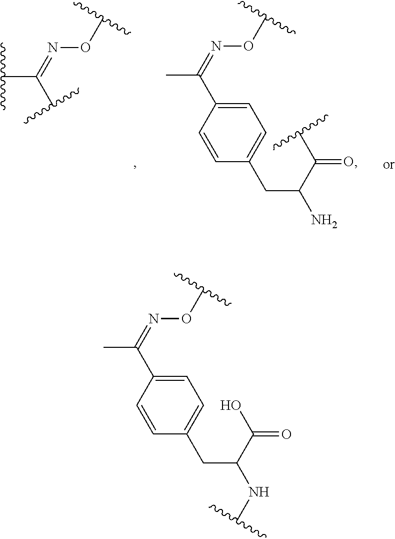 [SMILES: *ON=C(*)* ; *O/N=C(/C)c1ccc(CC(N)C(*)=O)cc1 ; *NC(Cc1ccc(/C(C)=N\O*)cc1)C(=O)O]

and [symbol] represents attachment to the remainder of the compound. In certain embodiments of Formula (II)-(IIH), (IV), (IVA), or (IVB), RL is

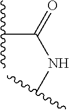 [SMILES: *NC(*)=O]

and [symbol] represents attachment to the remainder of the compound.

In certain embodiments, including any of the foregoing, D is a cytotoxic payload selected from a tubulin inhibitor, a DNA topoisomerase I inhibitor, and a DNA topoisomerase II inhibitor; or a derivative thereof. In some embodiments, including any of the foregoing, D is a tubulin inhibitor or a derivative thereof. In some embodiments, including any of the foregoing, D is a DNA topoisomerase I inhibitor or a derivative thereof. In some embodiments, including any of the foregoing, D is a DNA topoisomerase I inhibitor selected from the group consisting of irinotecan, SN-38, topotecan, exatecan; or a derivative thereof. In some embodiments, including any of the foregoing, D is irinotecan or a derivative thereof. In some embodiments, including any of the foregoing, D is SN-38 or a derivative thereof. In some embodiments, including any of the foregoing, D is topotecan or a derivative thereof. In some embodiments, including any of the foregoing, D is exatecan or a derivative thereof. In some embodiments, including any of the foregoing, D is a DNA topoisomerase II inhibitor or a derivative thereof. In some embodiments, including any of the foregoing, D is a DNA topoisomerase II inhibitor selected from the group consisting of etoposide, teniposide, and tafluposide; or a derivative thereof. In some embodiments, including any of the foregoing, D is etoposide or a derivative thereof. In some embodiments, including any of the foregoing, D is teniposide or a derivative thereof. In some embodiments, including any of the foregoing, D is tafluposide or a derivative thereof. In some embodiments, including any of the foregoing, D is a payload selected from the group consisting of hemiasterlins, camptothecins, and anthracyclines; or a derivative thereof. Anthracyclines may include PNU-159682 and EDA PNU-159682 derivatives. In some embodiments, including any of the foregoing, D is an anthracycline selected from the group consisting of daunorubicin, doxorubicin, epirubicin, idarubicin, mitoxantrone, and valrubicin; or a derivative thereof. In some embodiments, including any of the foregoing, D is daunorubicin or a derivative thereof. In some embodiments, including any of the foregoing, D is doxorubicin or a derivative thereof. In some embodiments, including any of the foregoing, D is epirubicin or a derivative thereof. In some embodiments, including any of the foregoing, D is idarubicin or a derivative thereof. In some embodiments, including any of the foregoing, D is mitoxantrone or a derivative thereof. In some embodiments, including any of the foregoing, D is valrubicin or a derivative thereof. In some embodiments, including any of the foregoing, D is hemiasterlin or a derivative thereof. In some embodiments, including any of the foregoing, D is a camptothecin or a derivative thereof. In some embodiments, including any of the foregoing, D is an anthracycline or a derivative thereof. In some embodiments, including any of the foregoing, D is PNU-159682 or a derivative thereof. In some embodiments, including any of the foregoing, D is an EDA PNU compound or a derivative thereof. In some embodiments, including any of the foregoing, D is an EDA PNU-159682 derivative. In some embodiments, including any of the foregoing, D is hemiasterlin, exatecan, PNU-159682, or an EDA PNU-159682 derivative. In some embodiments, including any of the foregoing, D is hemiasterlin or a derivative thereof. In some embodiments, including any of the foregoing, D is exatecan or a derivative thereof. In some embodiments, including any of the foregoing, D is PNU-159682 or a derivative thereof. In some embodiments, including any of the foregoing, D is an EDA PNU-159682 compound or derivative. In some embodiments, including any of the foregoing, D is not an immunostimulatory compound.

In some embodiments, including any of the foregoing, D is an alkylating agent or a derivative thereof. In some embodiments, including any of the foregoing, D is a bifunctional alkylator or a derivative thereof. In some embodiments, including any of the foregoing, D is a bifunctional alkylator selected from the group consisting of cyclophosphamide, mechlorethamine, chlorambucil, and melphalan; or a derivative thereof. In some embodiments, including any of the foregoing, D is cyclophosphamide or a derivative thereof. In some embodiments, including any of the foregoing, D is mechlorethamine or a derivative thereof. In some embodiments, including any of the foregoing, D is chlorambucil or a derivative thereof. In some embodiments, including any of the foregoing, D is melphalan or a derivative thereof. In some embodiments, including any of the foregoing, D is a monofunctional alkylator or a derivative thereof. In some embodiments, including any of the foregoing, D is a monofunctional alkylator selected from the group consisting of dacarbazine, nitrosourea, and temozolomide; or a derivative thereof. In some embodiments, including any of the foregoing, D is dacarbazine or a derivative thereof. In some embodiments, including any of the foregoing, D is nitrosourea or a derivative thereof. In some embodiments, including any of the foregoing, D is temozolomide or a derivative thereof. In some embodiments, including any of the foregoing, D is a cytoskeletal disruptor (e.g., a taxane) or a derivative thereof. In some embodiments, including any of the foregoing, D is a cytoskeletal disruptor selected from the group consisting of paclitaxel, docetaxel, abraxane, and taxotere; or a derivative thereof. In some embodiments, including any of the foregoing, D is paclitaxel or a derivative thereof. In some embodiments, including any of the foregoing, D is docetaxel or a derivative thereof. In some embodiments, including any of the foregoing, D is abraxane or a derivative thereof. In some embodiments, including any of the foregoing, D is taxotere or a derivative thereof. In some embodiments, including any of the foregoing, D is an epothilone or a derivative thereof. In some embodiments, including any of the foregoing, D is an epothilone selected from the group consisting of epothilone A, epothilone B, epothilone C, epothilone D, and ixabepilone; or a derivative thereof. In some embodiments, including any of the foregoing, D is epothilone A or a derivative thereof. In some embodiments, including any of the foregoing, D is epothilone B or a derivative thereof. In some embodiments, including any of the foregoing, D is epothilone C or a derivative thereof. In some embodiments, including any of the foregoing, D is epothilone D or a derivative thereof. In some embodiments, including any of the foregoing, D is ixabepilone or a derivative thereof. In some embodiments, including any of the foregoing, D is a histone deacetylase inhibitor or a derivative thereof. In some embodiments, including any of the foregoing, D is a histone deacetylase inhibitor selected from the group consisting of vorinostat and romidepsin; or a derivative thereof. In some embodiments, including any of the foregoing, D is vorinostat or a derivative thereof. In some embodiments, including any of the foregoing, D is romidepsin or a derivative thereof. In some embodiments, including any of the foregoing, D is a kinase inhibitor or a derivative thereof. In some embodiments, including any of the foregoing, D is a kinase inhibitor selected from the group consisting of bortezomib, erlotinib, gefitinib, imatinib, vemurafenib, and vismodegib; or a derivative thereof. In some embodiments, including any of the foregoing, D is bortezomib or a derivative thereof. In some embodiments, including any of the foregoing, D is erlotinib or a derivative thereof. In some embodiments, including any of the foregoing, D is gefitinib or a derivative thereof. In some embodiments, including any of the foregoing, D is imatinib or a derivative thereof. In some embodiments, including any of the foregoing, D is vemurafenib or a derivative thereof. In some embodiments, including any of the foregoing, D is a vismodegib or a derivative thereof. In some embodiments, including any of the foregoing, D is a nucleotide analog and/or precursor analog or a derivative thereof. In some embodiments, including any of the foregoing, D is a nucleotide analog and/or precursor analog selected from the group consisting of azacitidine, azathioprine, capecitabine, cytarabine, doxifluridine, fluorouracil, gemcitabine, hydroxyurea, mercaptopurine, methotrexate, and tioguanine (formerly thioguanine); or a derivative thereof. In some embodiments, including any of the foregoing, D is azacitidine or a derivative thereof. In some embodiments, including any of the foregoing, D is azathioprine or a derivative thereof. In some embodiments, including any of the foregoing, D is capecitabine or a derivative thereof. In some embodiments, including any of the foregoing, D is cytarabine or a derivative thereof. In some embodiments, including any of the foregoing, D is doxifluridine or a derivative thereof. In some embodiments, including any of the foregoing, D is fluorouracil or a derivative thereof. In some embodiments, including any of the foregoing, D is gemcitabine or a derivative thereof. In some embodiments, including any of the foregoing, D is hydroxyurea or a derivative thereof. In some embodiments, including any of the foregoing, D is mercaptopurine or a derivative thereof. In some embodiments, including any of the foregoing, D is methotrexate or a derivative thereof. In some embodiments, including any of the foregoing, D is tioguanine (formerly thioguanine) or a derivative thereof. In some embodiments, including any of the foregoing, D is a peptide antibiotic or a derivative thereof. In some embodiments, including any of the foregoing, D is a peptide antibiotic selected from the group consisting of bleomycin and actinomycin; or a derivative thereof. In some embodiments, including any of the foregoing, D is bleomycin or a derivative thereof. In some embodiments, including any of the foregoing, D is actinomycin or a derivative thereof. In some embodiments, including any of the foregoing, D is a platinum-based payload or a derivative thereof. In some embodiments, including any of the foregoing, D is a platinum-based payload selected from the group consisting of carboplatin, cisplatin, and oxaliplatin; or a derivative thereof. In some embodiments, including any of the foregoing, D is carboplatin or a derivative thereof. In some embodiments, including any of the foregoing, D is cisplatin or a derivative thereof. In some embodiments, including any of the foregoing, D is oxaliplatin or a derivative thereof. In some embodiments, including any of the foregoing, D is a retinoid or a derivative thereof. In some embodiments, including any of the foregoing, D is a retinoid selected from the group consisting of tretinoin, alitretinoin, and bexarotene; or a derivative thereof. In some embodiments, including any of the foregoing, D is tretinoin or a derivative thereof. In some embodiments, including any of the foregoing, D is alitretinoin or a derivative thereof. In some embodiments, including any of the foregoing, D is bexarotene or a derivative thereof. In some embodiments, including any of the foregoing, D is a vinca alkaloid or a derivative thereof. In some embodiments, including any of the foregoing, D is a vinca alkaloid or a derivative thereof selected from the group consisting of vinblastine, vincristine, vindesine, vinorelbine. In some embodiments, including any of the foregoing, D is vinblastine or a derivative thereof. In some embodiments, including any of the foregoing, D is vincristine or a derivative thereof. In some embodiments, including any of the foregoing, D is vindesine or a derivative thereof.

In some embodiments, including any of the foregoing, D is selected from

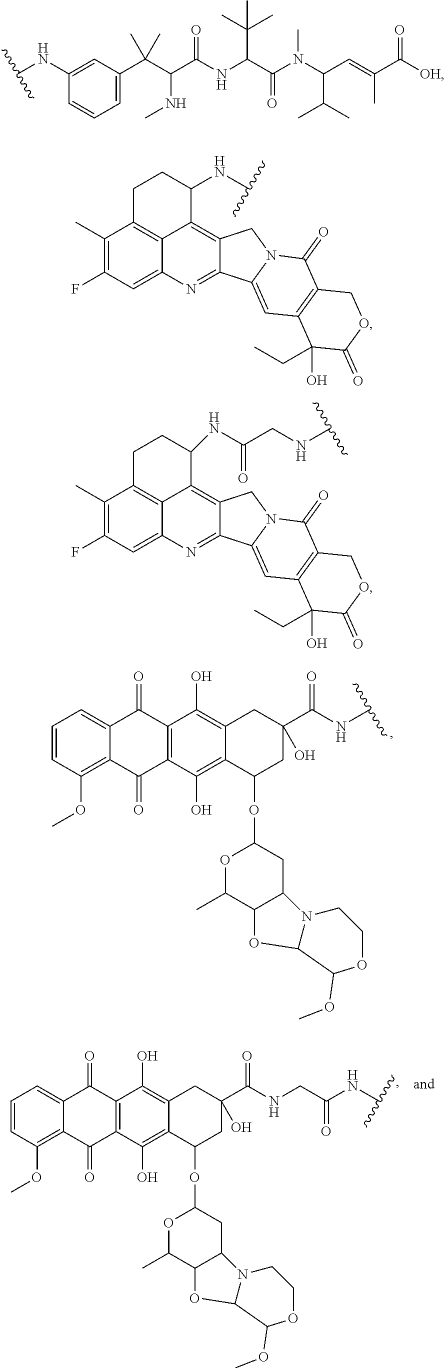

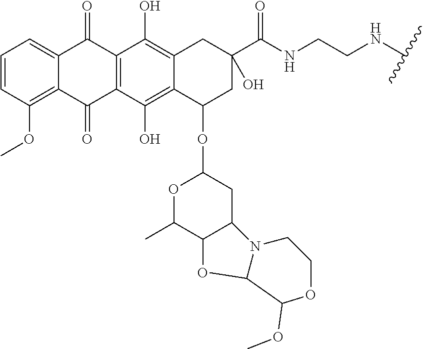 [SMILES: *NCCNC(=O)C1(O)Cc2c(O)c3c(c(O)c2C(OC2CC4C(OC5C(OC)OCCN45)C(C)O2)C1)C(=O)c1c(OC)cccc1C3=O]

or a derivative thereof.

In some embodiments, including any of the foregoing, D is selected from

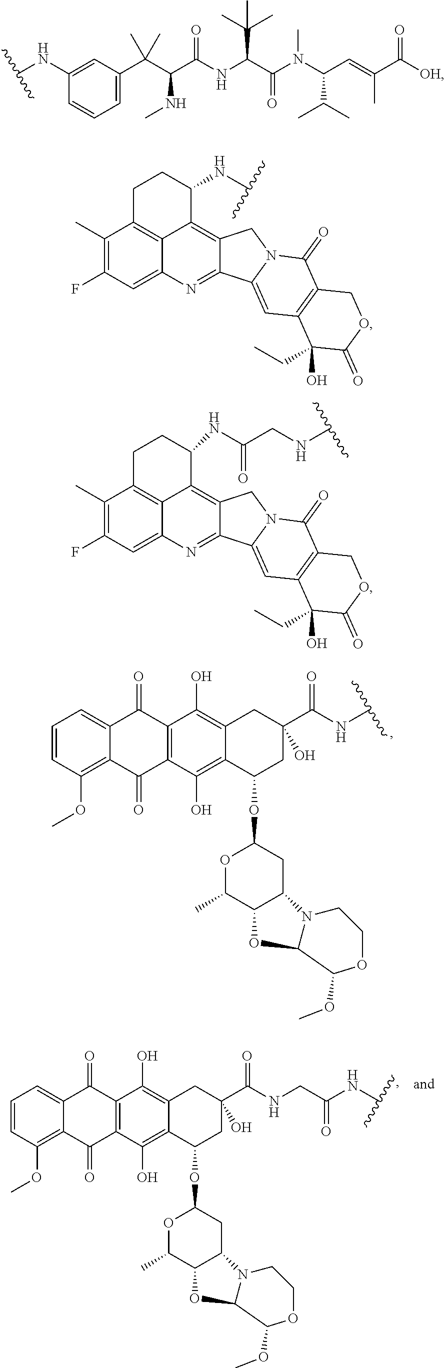

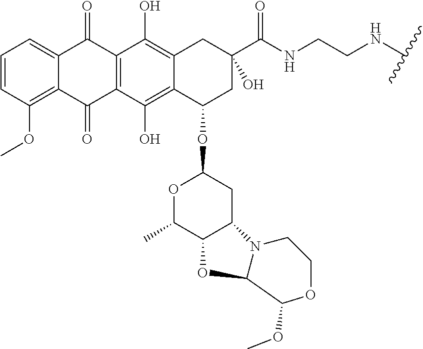 [SMILES: *NCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

or a derivative thereof.

Non-limiting examples of compounds of the present disclosure include:

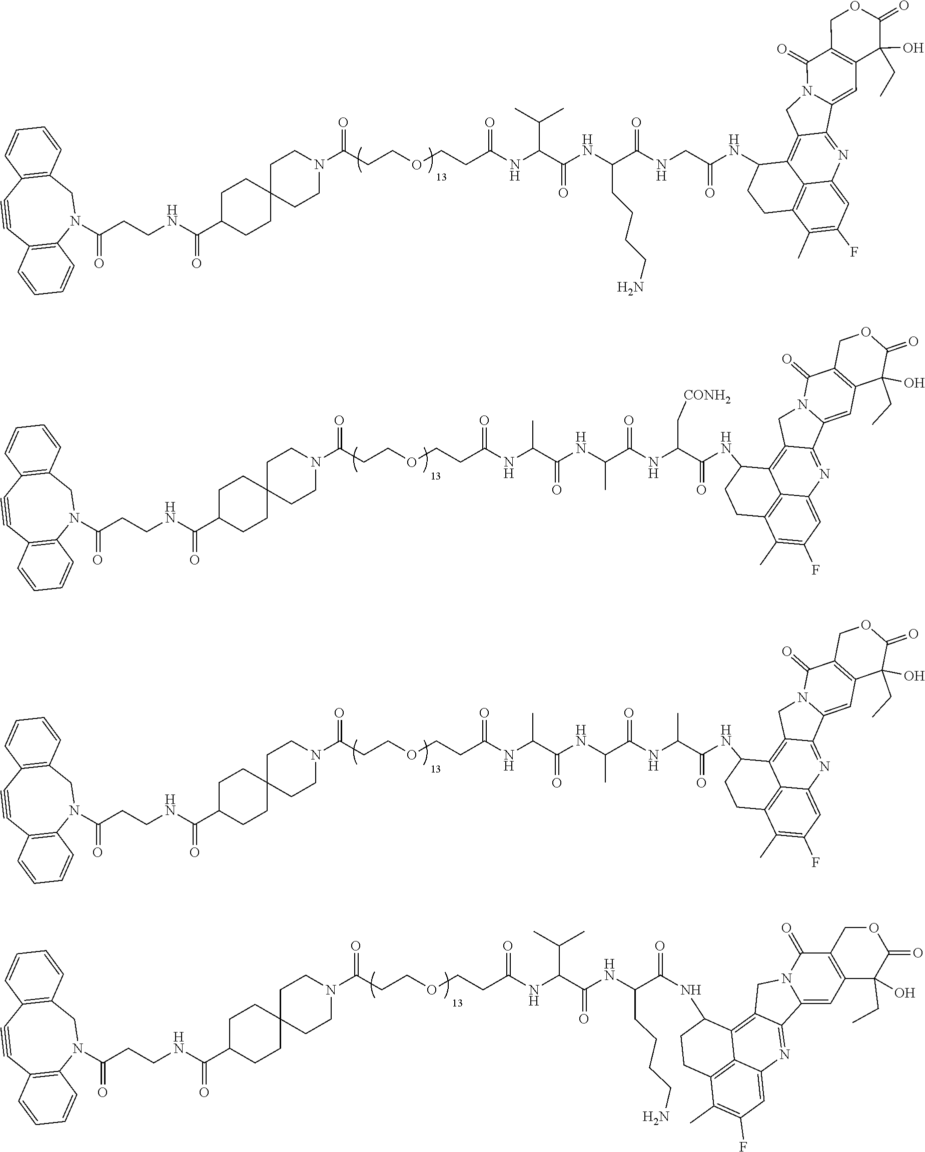

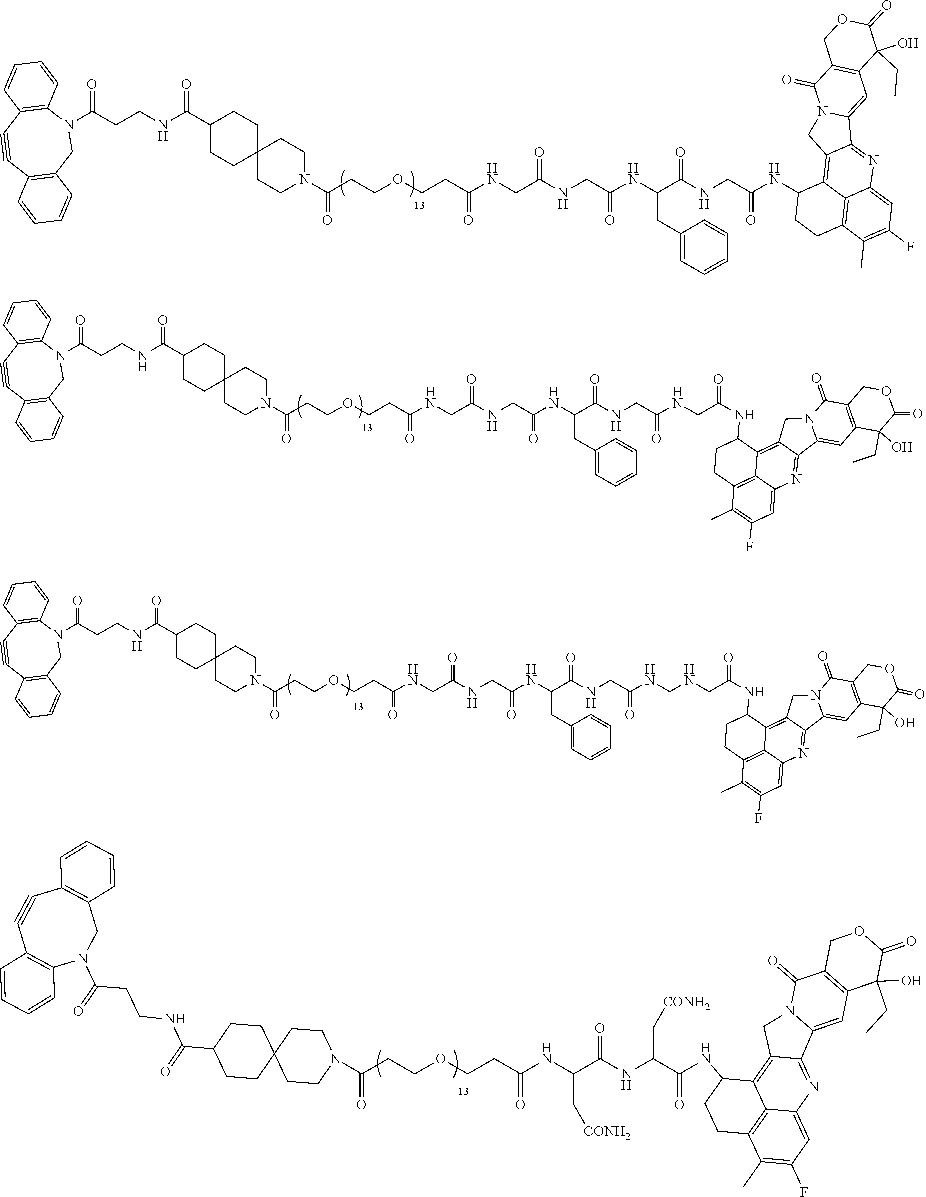

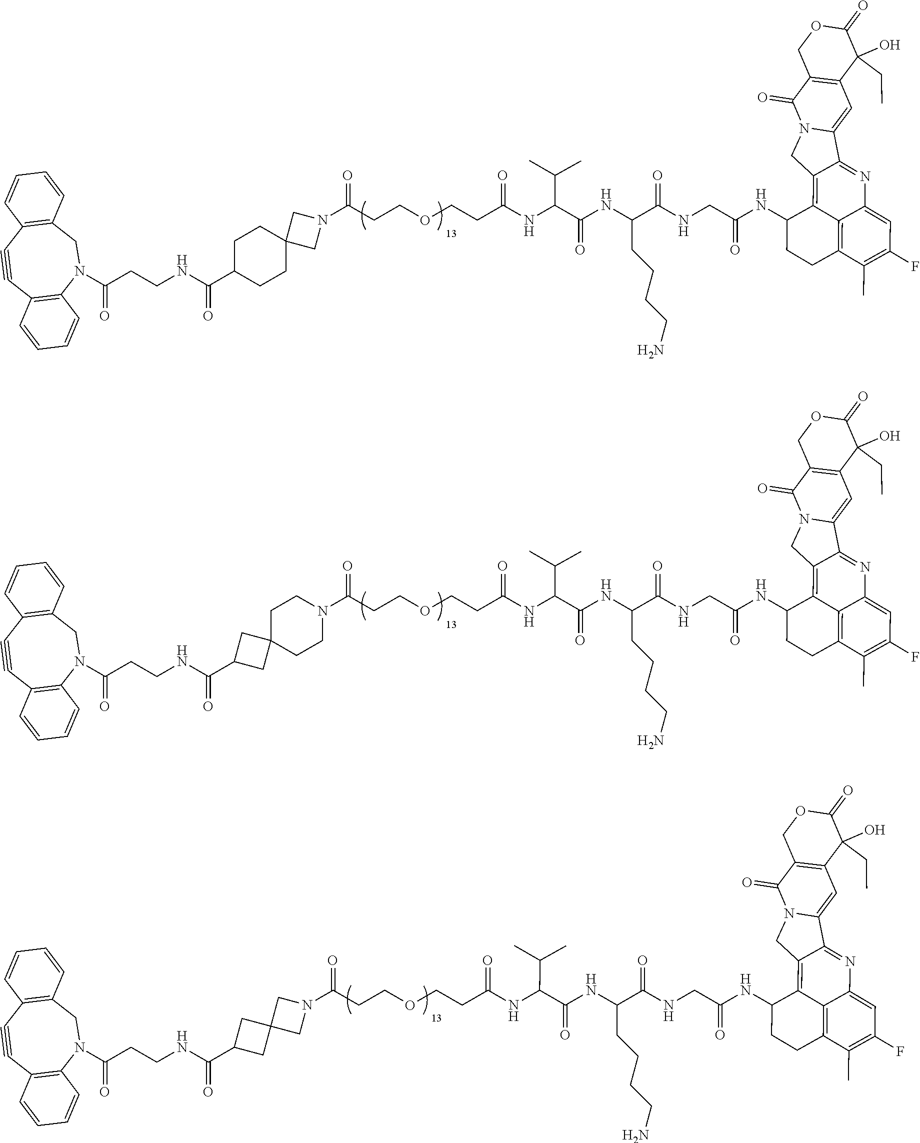

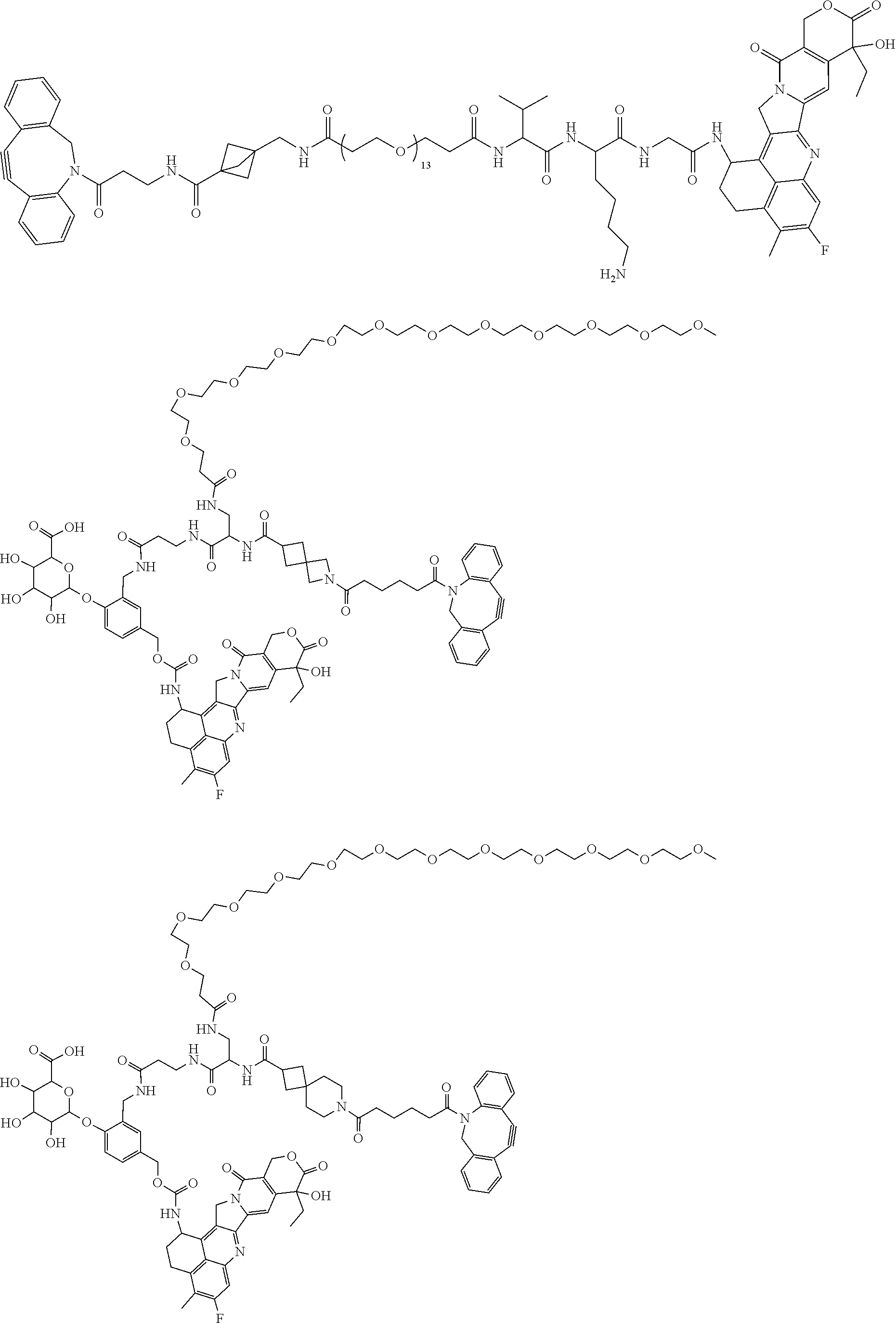

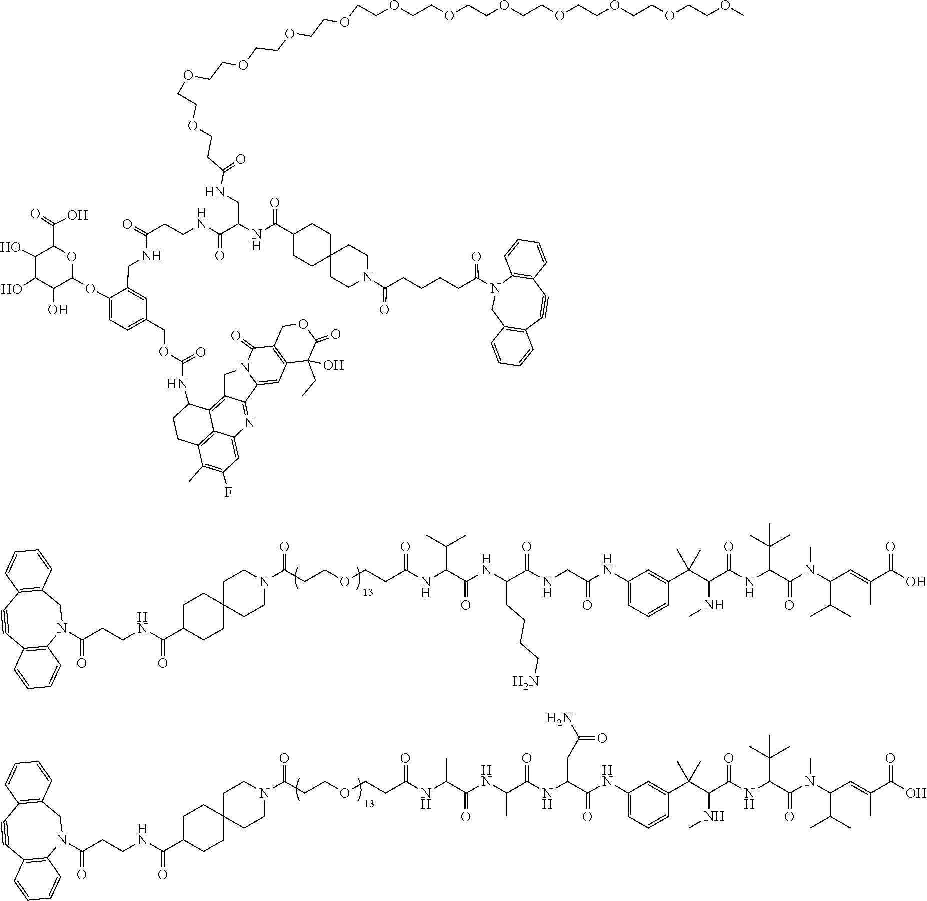

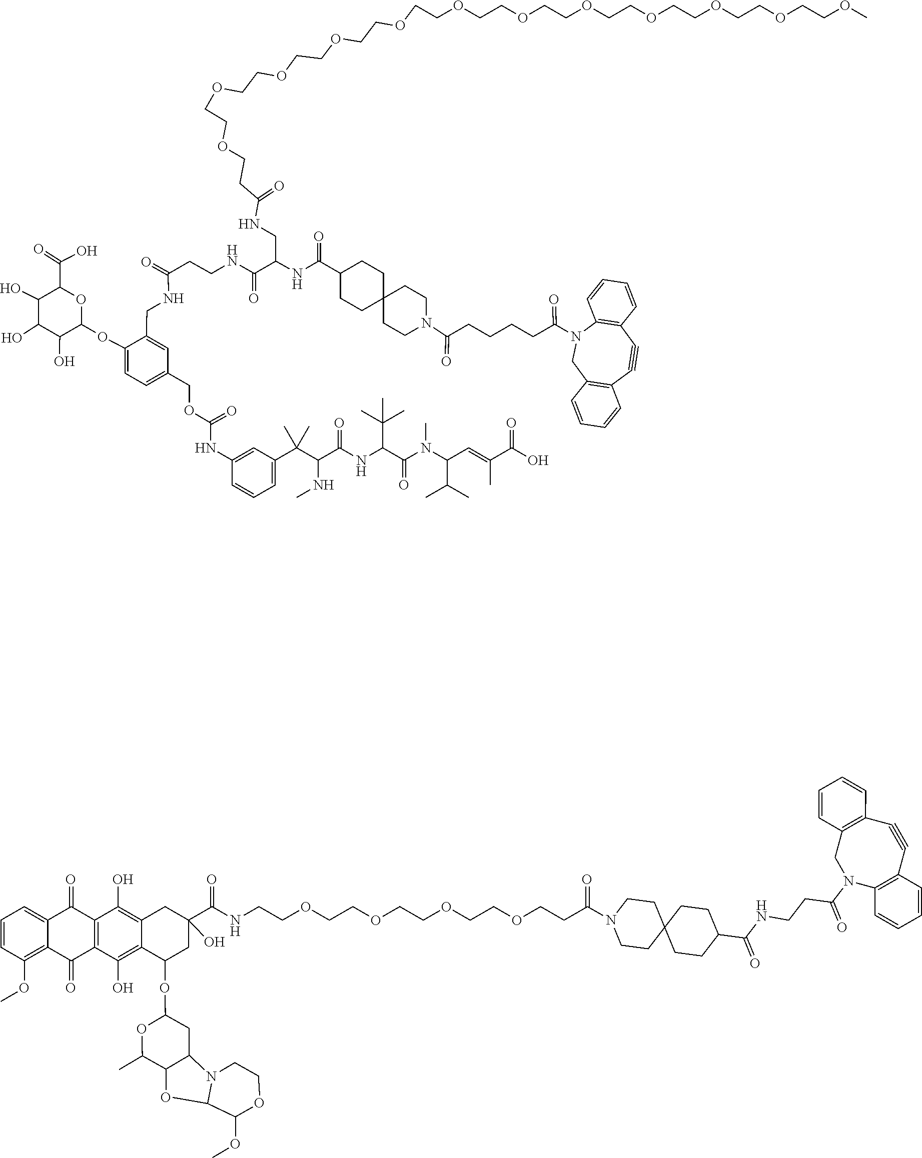

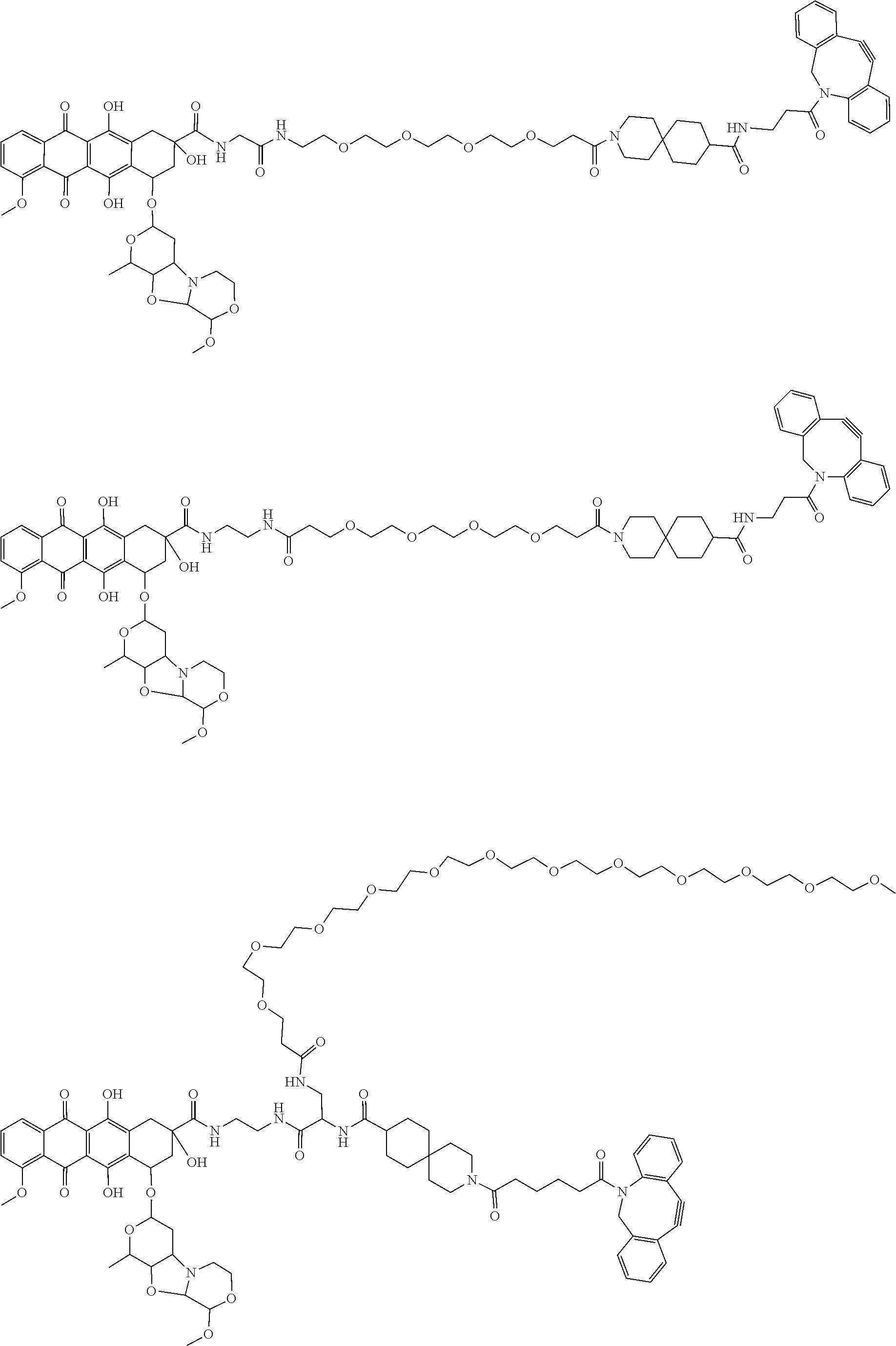

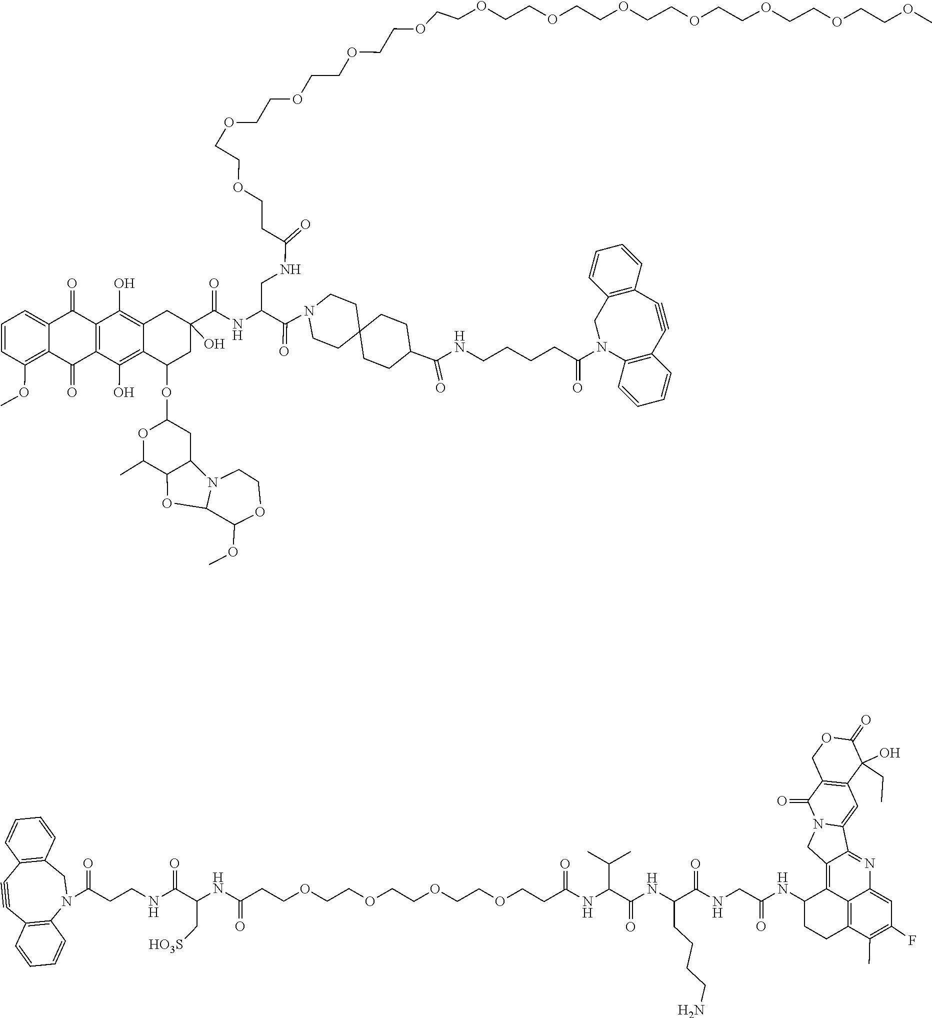

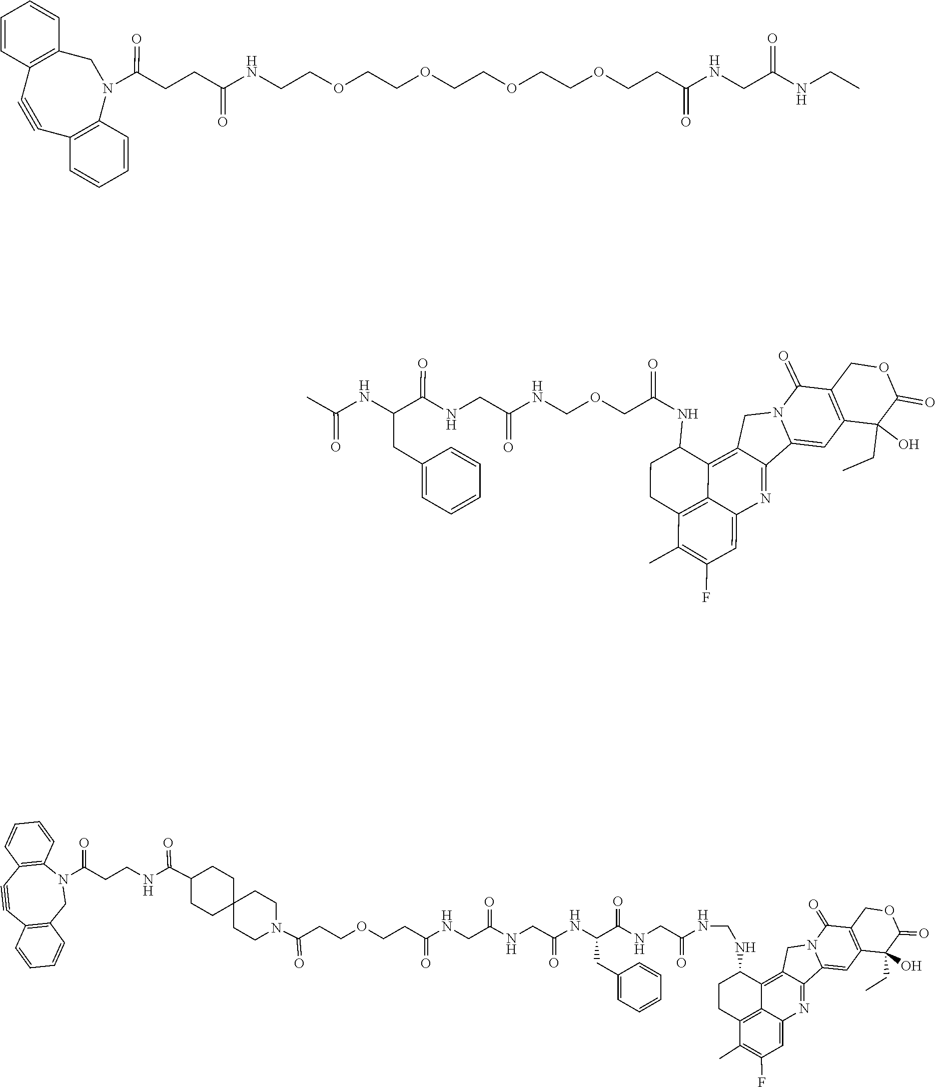

Representative compounds of the present disclosure are shown in Table A.

TABLE A

| | |
|---|---|
| 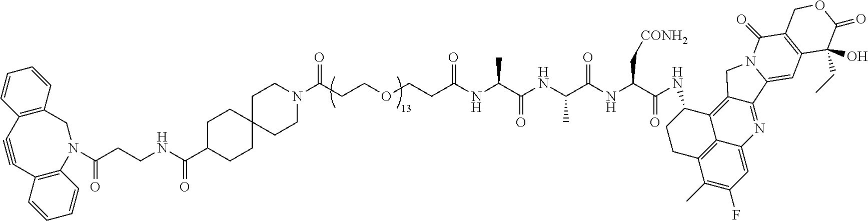 | 1 |
| 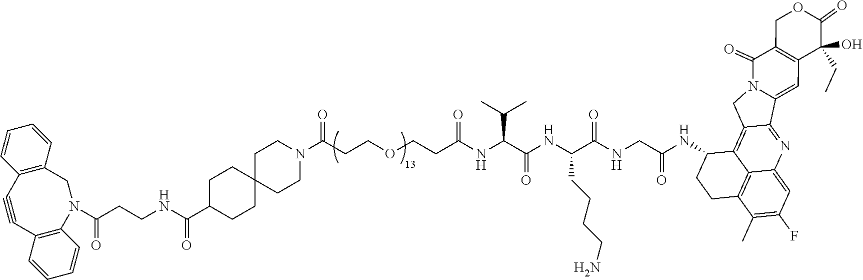 | 2 |
| 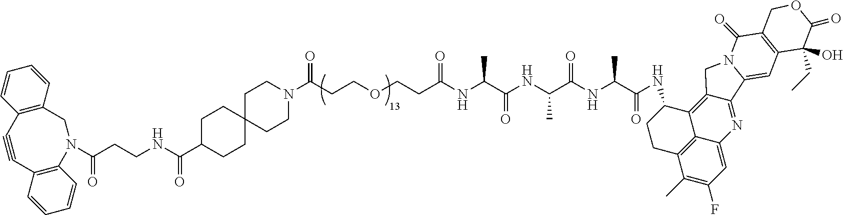 | 3 |
| 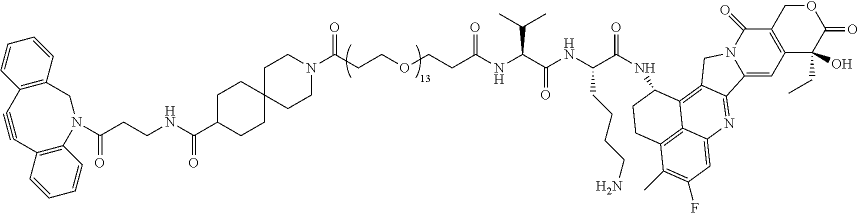 | 4 |
| 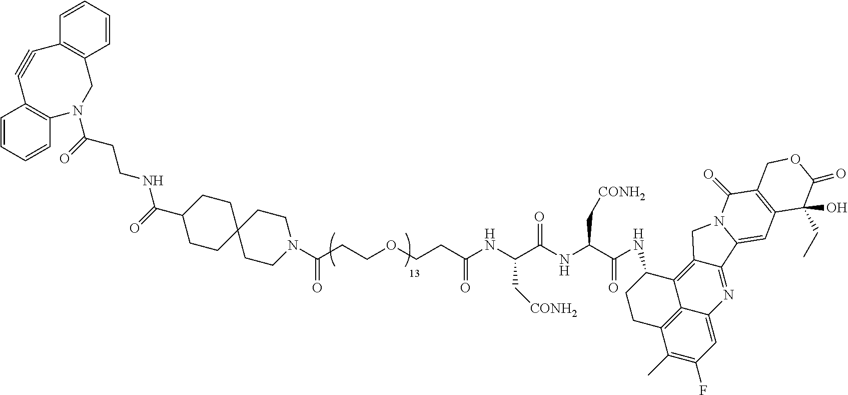 | 5 |
| 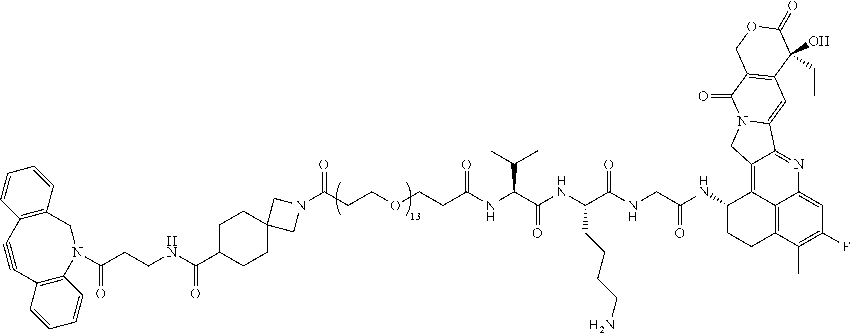 | 6 |
| 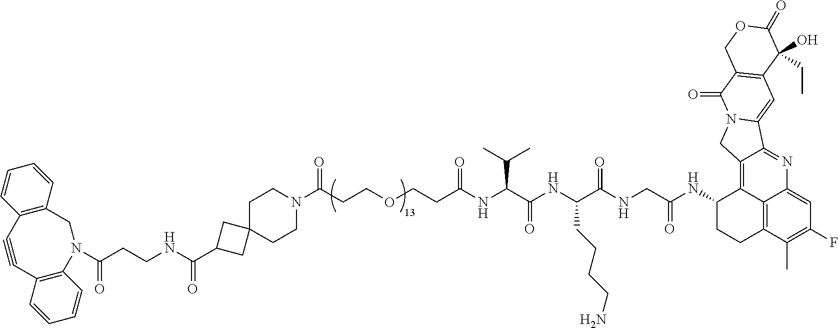 | 7 |
| 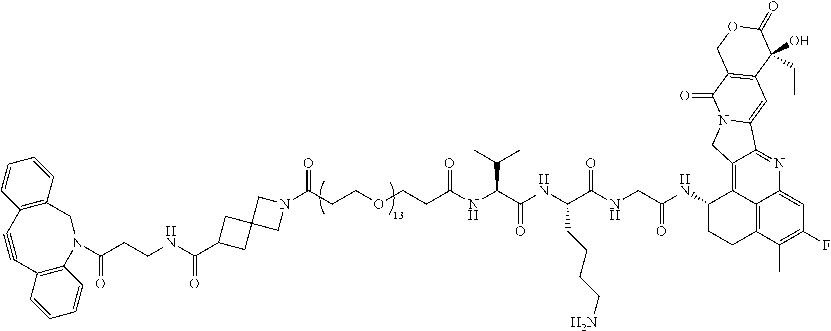 | 8 |
| 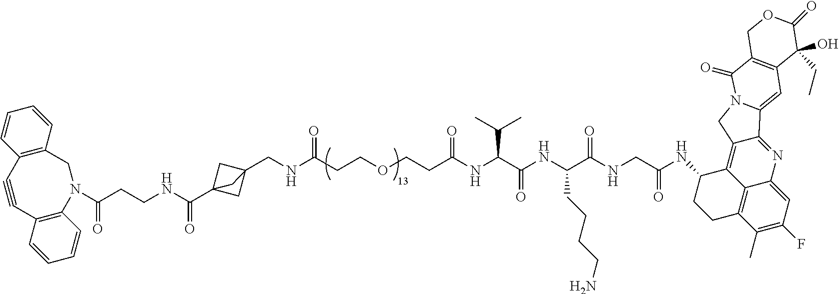 | 9 |
| 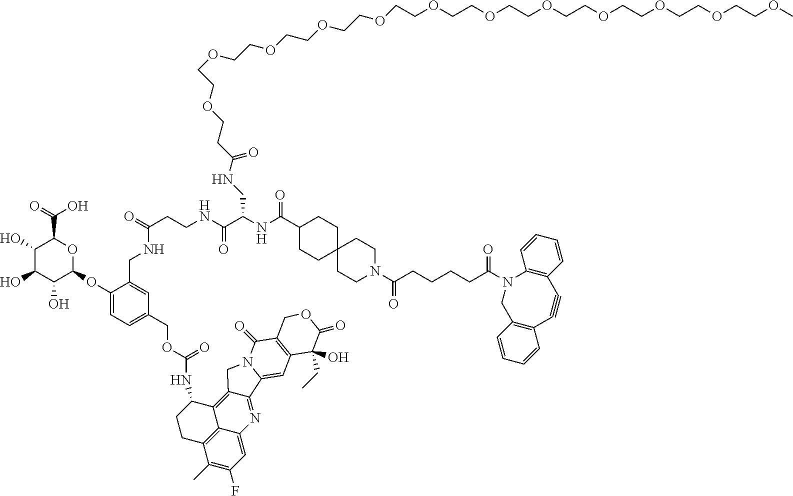 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)C2CCC4(CC2)CCN(C(=O)CCCCC(=O)N2Cc5ccccc5C#Cc5ccccc52)CC4)c1)CC3] | 10 |
| 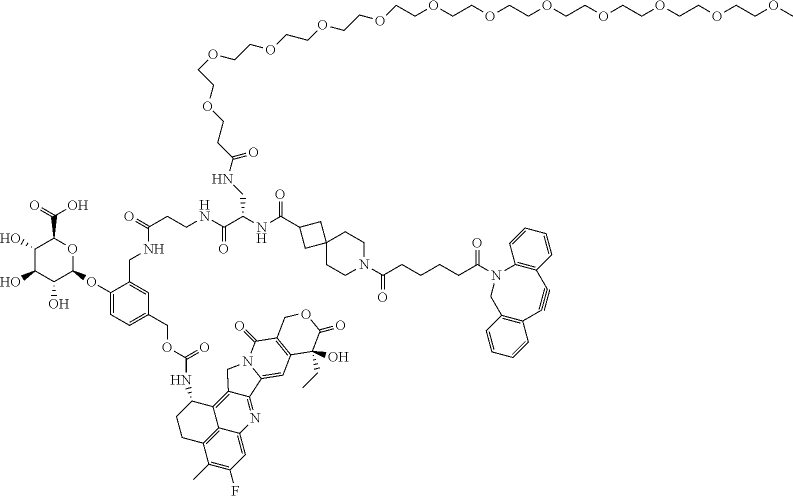 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)C2CC4(CCN(C(=O)CCCCC(=O)N5Cc6ccccc6C#Cc6ccccc65)CC4)C2)c1)CC3] | 11 |
| 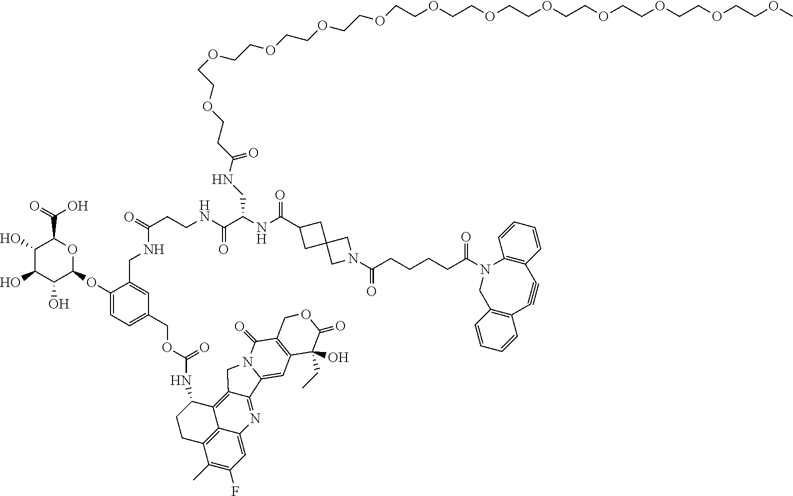 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)C2CC4(C2)CN(C(=O)CCCCC(=O)N2Cc5ccccc5C#Cc5ccccc52)C4)c1)CC3] | 12 |
| 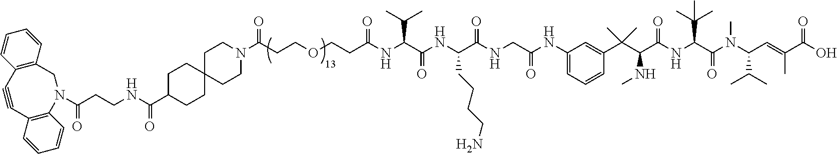 | 13 |
| 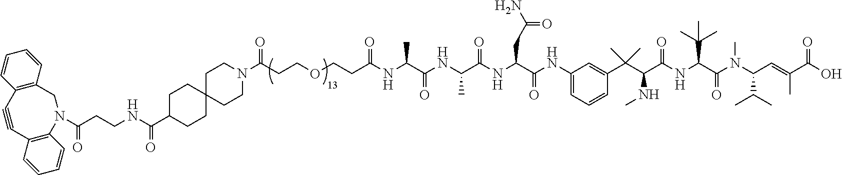 | 14 |
| 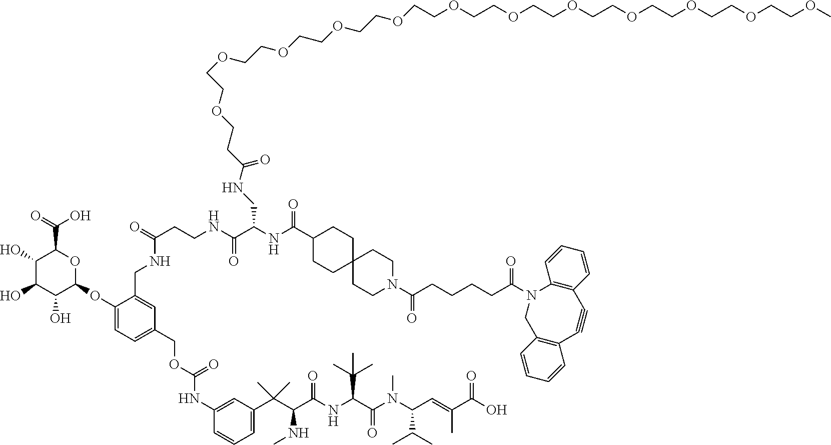 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(O[C@@H]3O[C@H](C(=O)O)[C@@H](O)[C@H](O)[C@H]3O)c(CNC(=O)CCNC(=O)[C@H](CNC(=O)CCOCCOCCOCCOCCOCCOCCOCCOCCOCCOCCOCCOC)NC(=O)C3CCC4(CC3)CCN(C(=O)CCCCC(=O)N3Cc5ccccc5C#Cc5ccccc53)CC4)c2)c1] | 15 |
| 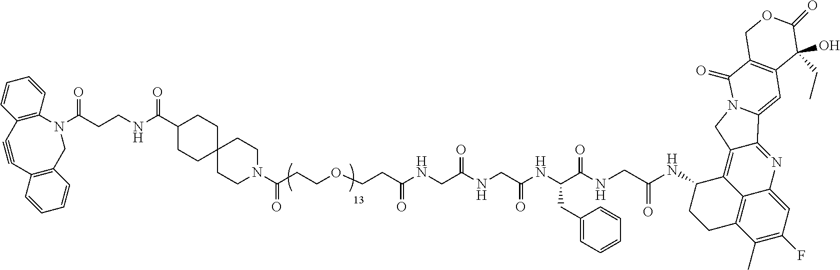 | 16 |
| 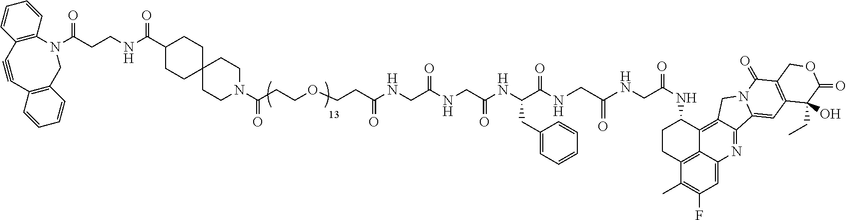 | 17 |
| 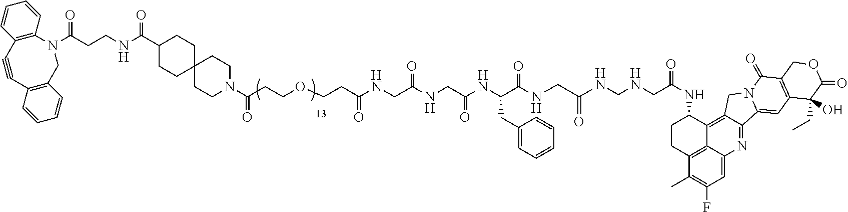 | 18 |
| 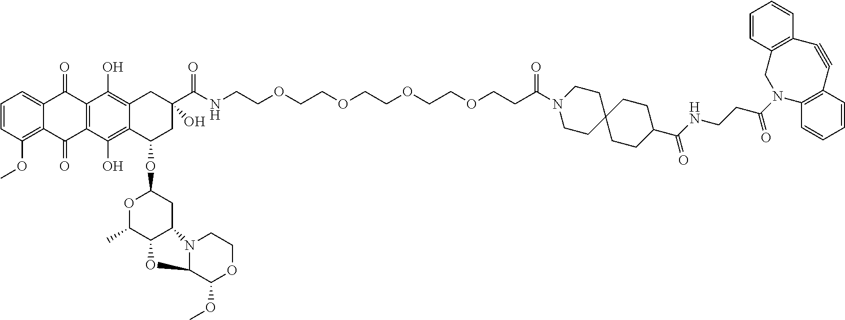 [SMILES: COc1cccc2c1C(=O)c1c(O)c3c(c(O)c1C2=O)C[C@@](O)(C(=O)NCCOCCOCCOCCOCCC(=O)N1CCC2(CCC(C(=O)NCCC(=O)N4Cc5ccccc5C#Cc5ccccc54)CC2)CC1)C[C@@H]3O[C@H]1C[C@H]2[C@H](O[C@@H]3[C@@H](OC)OCCN32)[C@H](C)O1] | 19 |
| 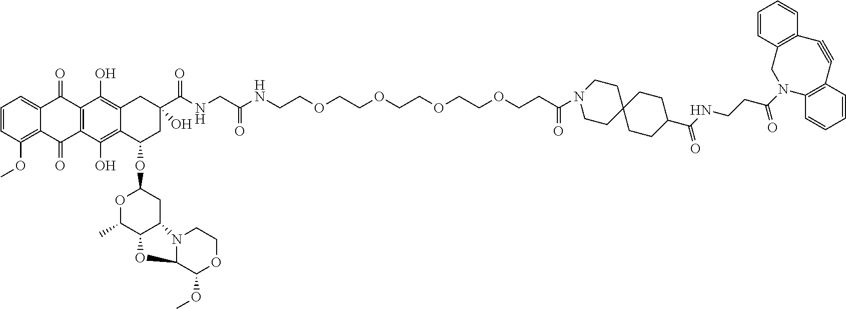 [SMILES: COc1cccc2c1C(=O)c1c(O)c3c(c(O)c1C2=O)C[C@@](O)(C(=O)NCC(=O)NCCOCCOCCOCCOCCC(=O)N1CCC2(CCC(C(=O)NCCC(=O)N4Cc5ccccc5C#Cc5ccccc54)CC2)CC1)C[C@@H]3O[C@H]1C[C@H]2[C@H](O[C@@H]3[C@@H](OC)OCCN32)[C@H](C)O1] | 20 |
| 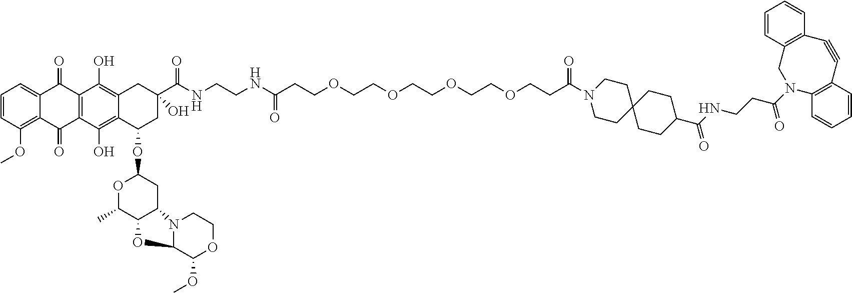 [SMILES: COc1cccc2c1C(=O)c1c(O)c3c(c(O)c1C2=O)C[C@@](O)(C(=O)NCCNC(=O)CCOCCOCCOCCOCCC(=O)N1CCC2(CCC(C(=O)NCCC(=O)N4Cc5ccccc5C#Cc5ccccc54)CC2)CC1)C[C@@H]3O[C@H]1C[C@H]2[C@H](O[C@@H]3[C@@H](OC)OCCN32)[C@H](C)O1] | 21 |
| 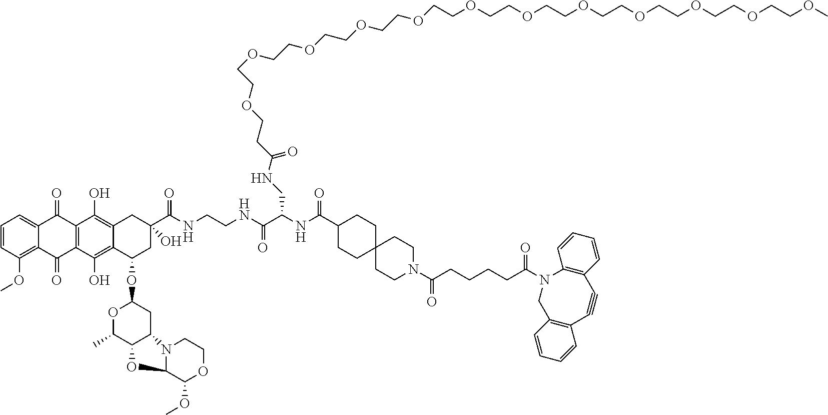 [SMILES: COCCOCCOCCOCCOCCOCCOCCOCCOCCOCCOCCOCCC(=O)NC[C@H](NC(=O)C1CCC2(CC1)CCN(C(=O)CCCCC(=O)N1Cc3ccccc3C#Cc3ccccc31)CC2)C(=O)NCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O] | 22 |
| 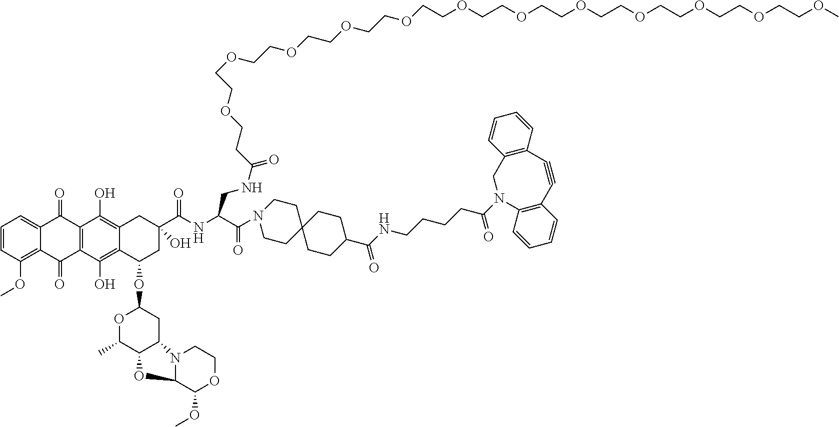 [SMILES: COCCOCCOCCOCCOCCOCCOCCOCCOCCOCCOCCOCCC(=O)NC[C@H](NC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O)C(=O)N1CCC2(CCC(C(=O)NCCCCC(=O)N3Cc4ccccc4C#Cc4ccccc43)CC2)CC1] | 23 |
| 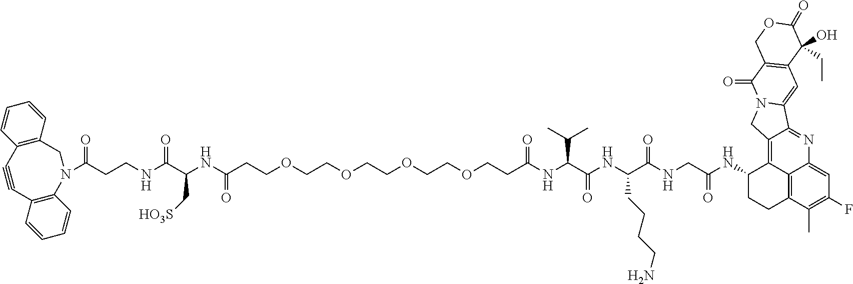 [SMILES: CC[C@@]1(O)C(=O)OCc2c1cc1n(c2=O)Cc2c-1nc1cc(F)c(C)c3c1c2[C@@H](NC(=O)CNC(=O)[C@H](CCCCN)NC(=O)[C@@H](NC(=O)CCOCCOCCOCCOCCC(=O)N[C@@H](CS(=O)(=O)O)C(=O)NCCC(=O)N1Cc2ccccc2C#Cc2ccccc21)C(C)C)CC3] | 24 |
| 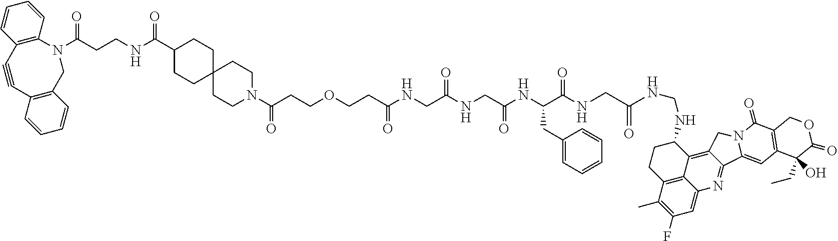 [SMILES: CC[C@@]1(O)C(=O)OCc2c1cc1n(c2=O)Cc2c-1nc1cc(F)c(C)c3c1c2[C@@H](NCNC(=O)CNC(=O)[C@H](Cc1ccccc1)NC(=O)CNC(=O)CNC(=O)CCOCCC(=O)N1CCC2(CCC(C(=O)NCCC(=O)N4Cc5ccccc5C#Cc5ccccc54)CC2)CC1)CC3] | 25 |

Non-limiting examples of conjugates of the present disclosure include:

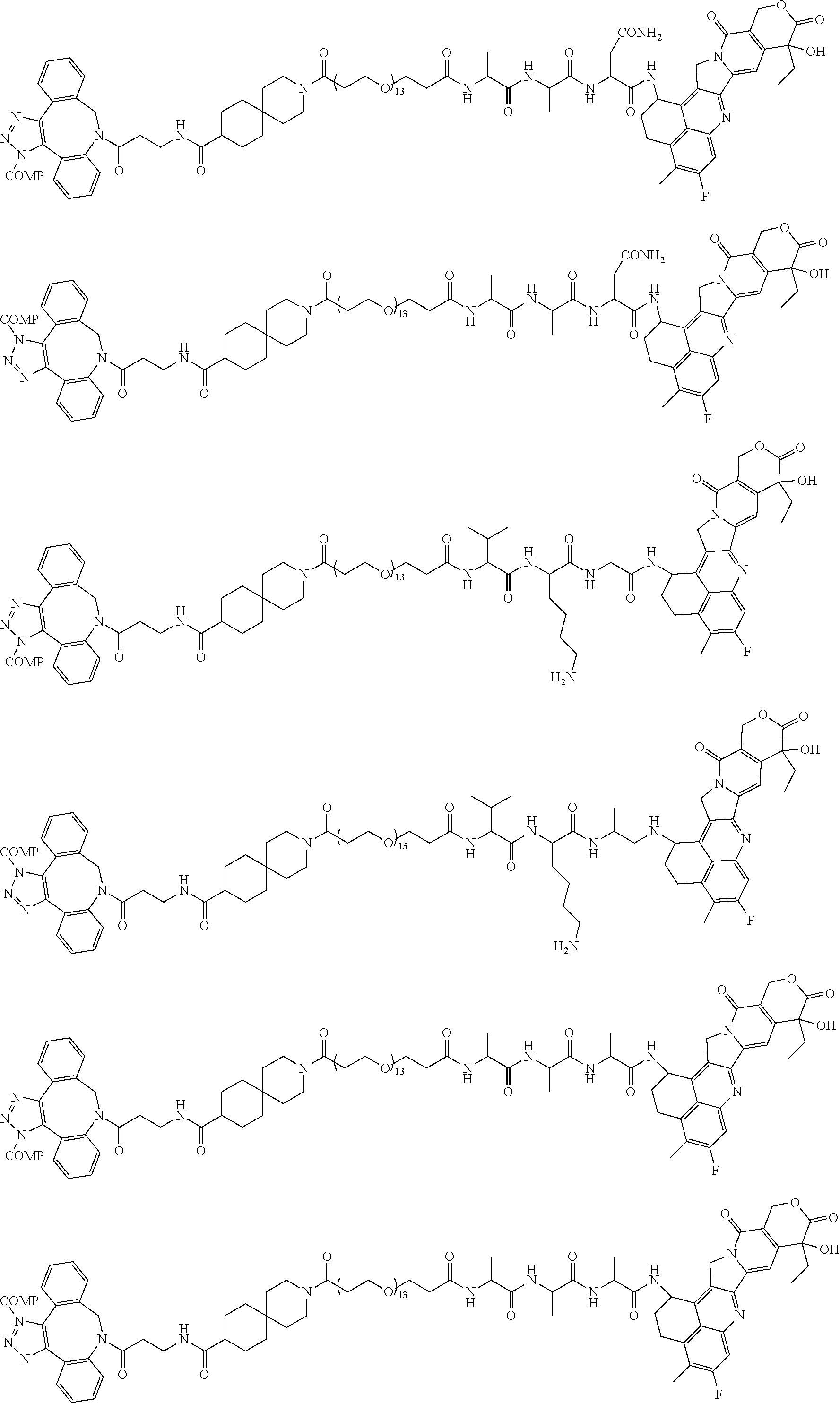

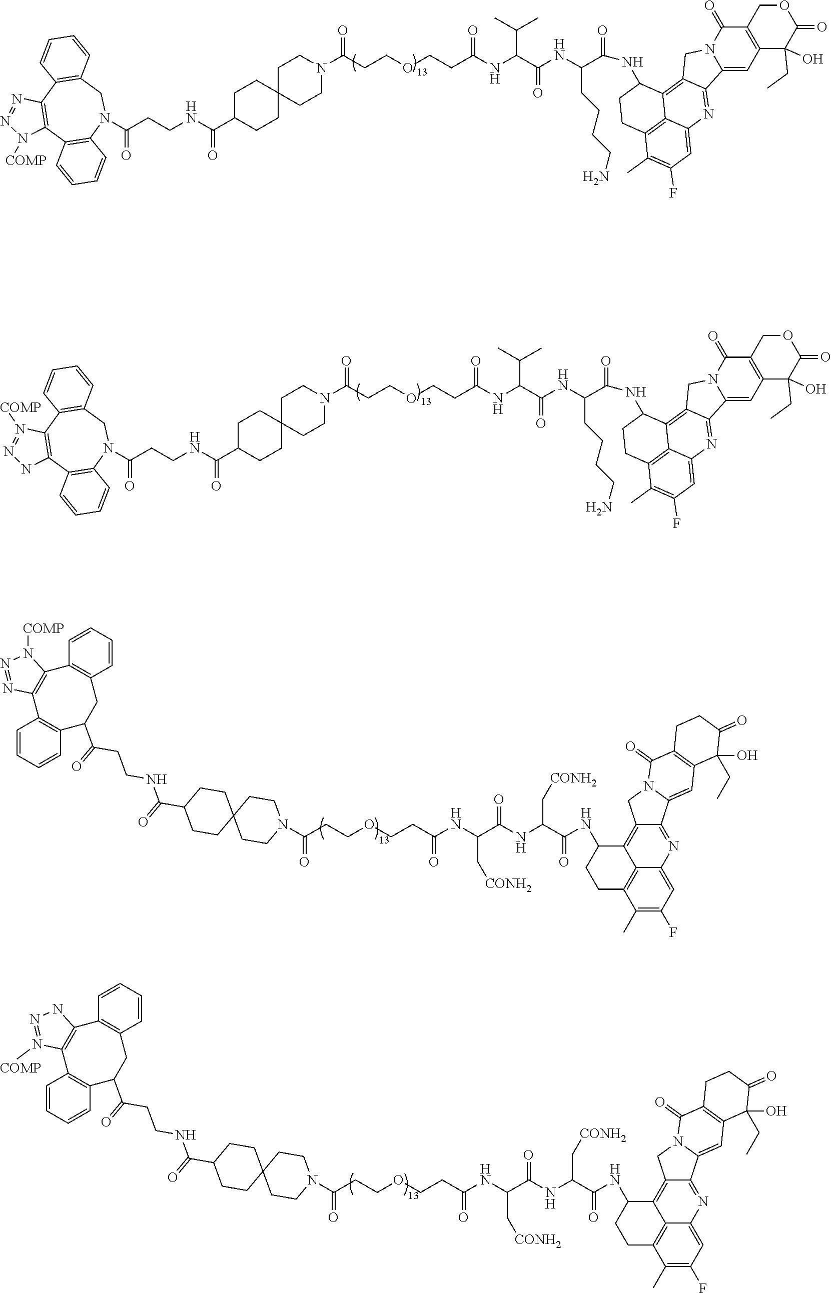

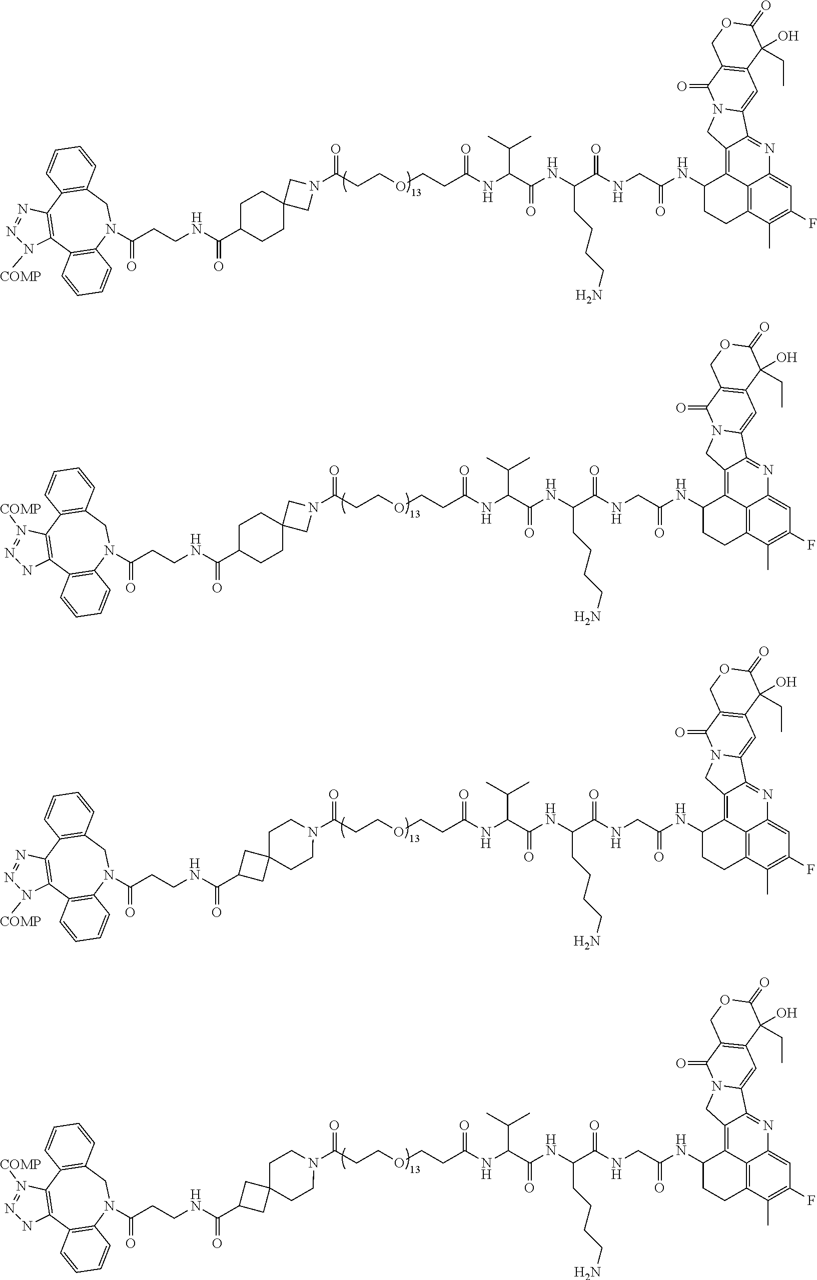

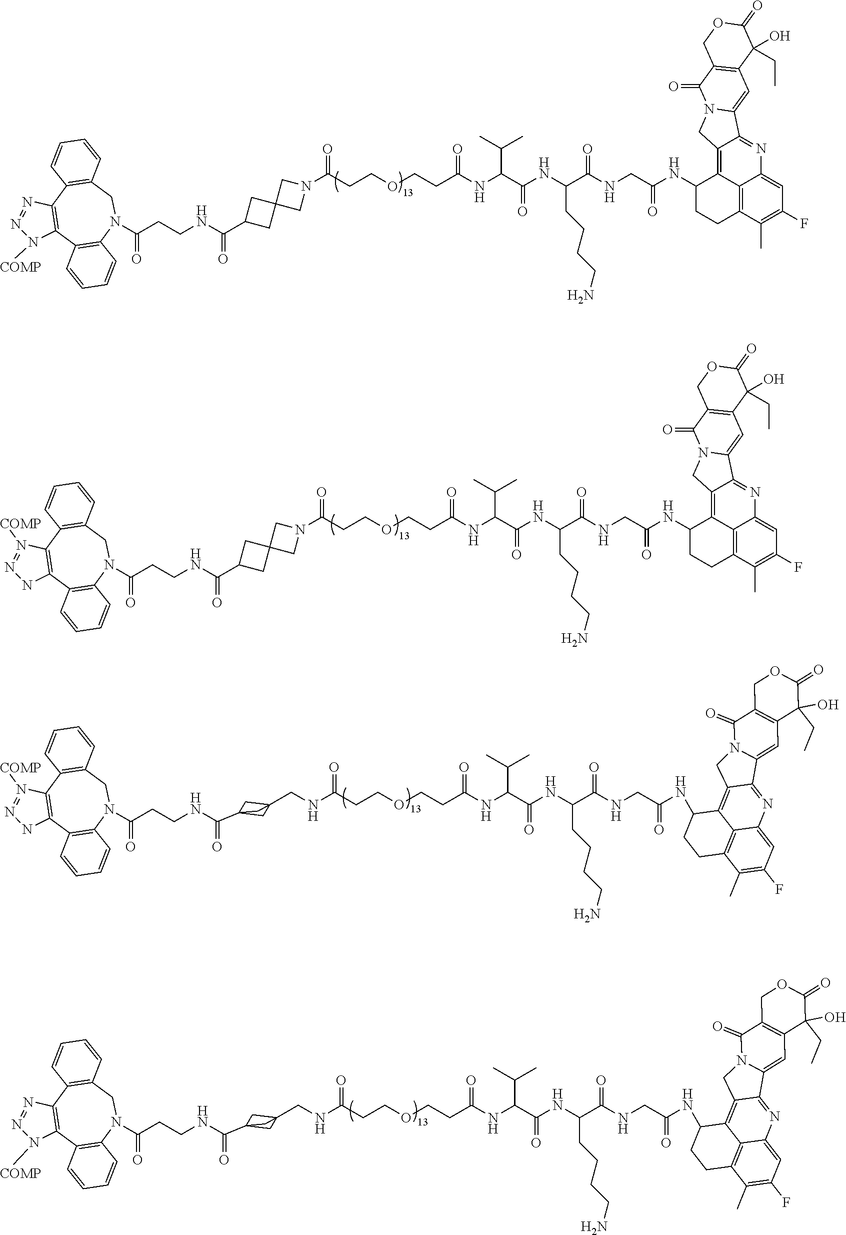

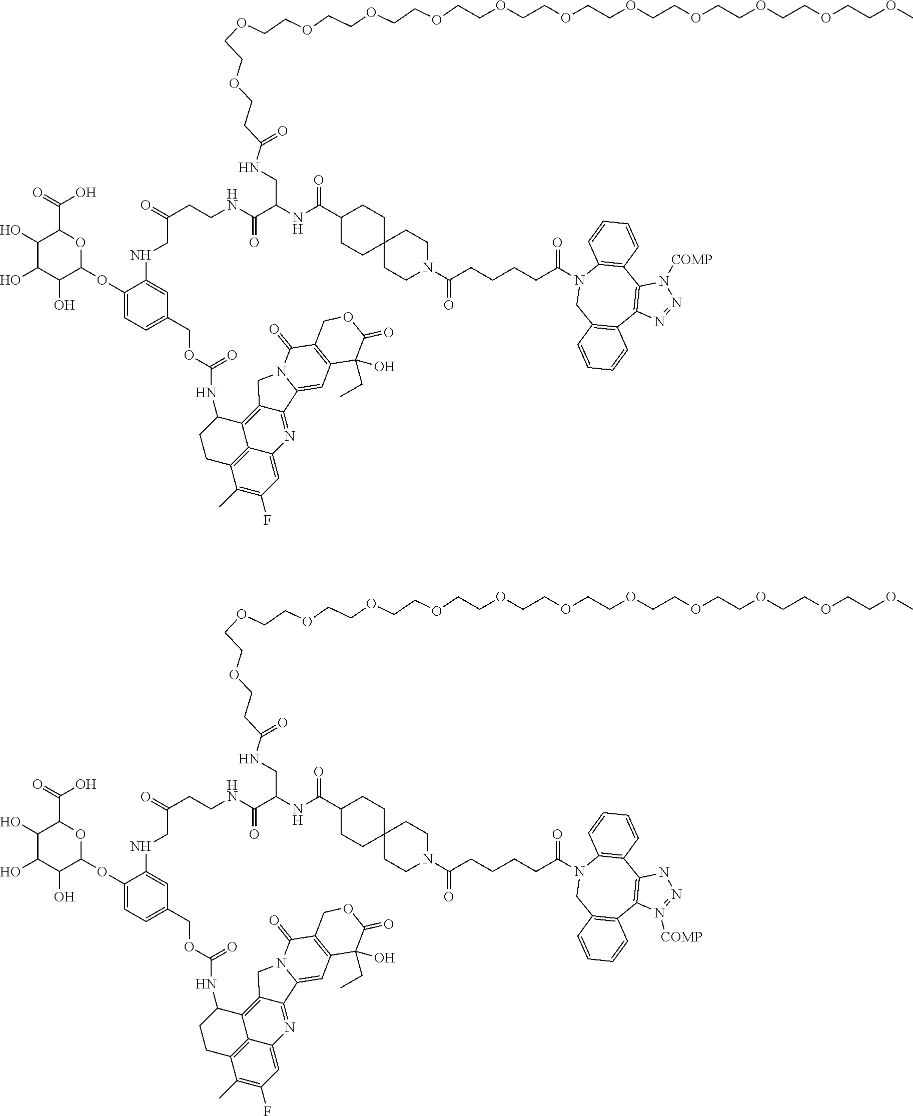

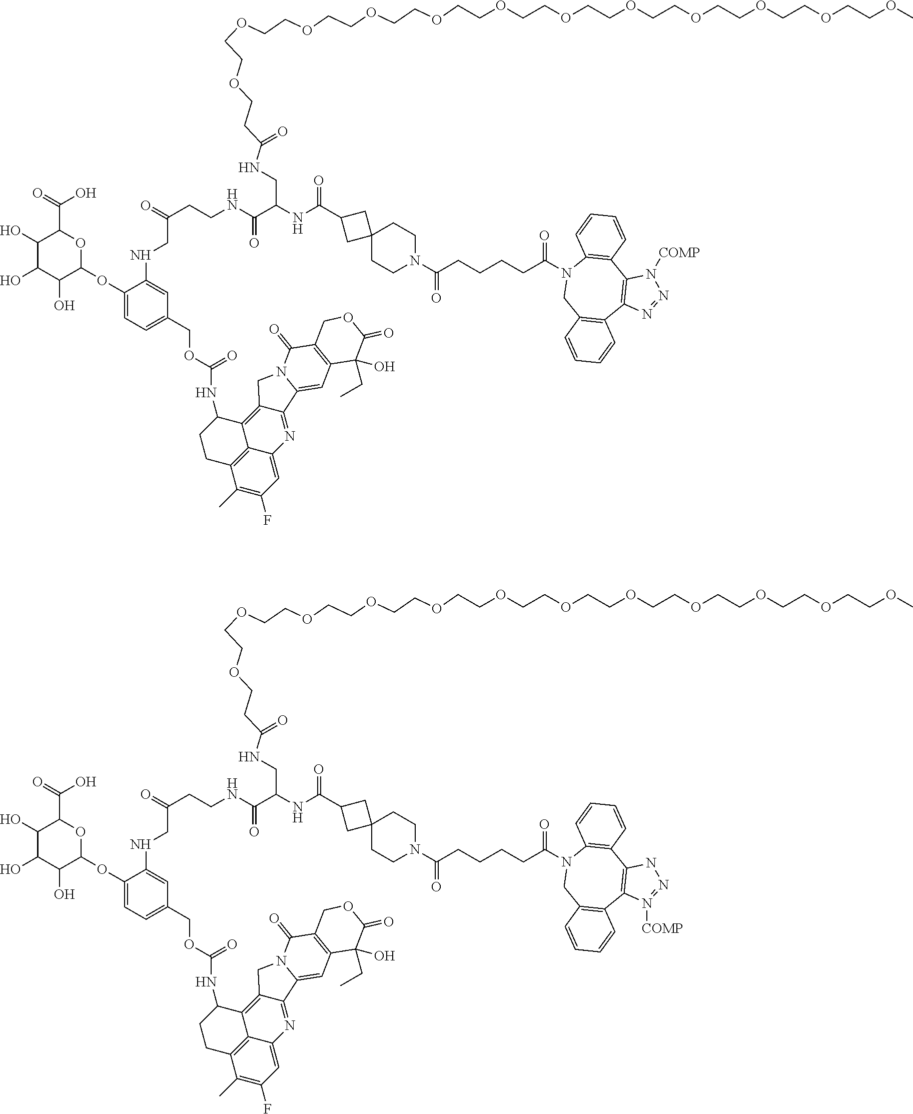

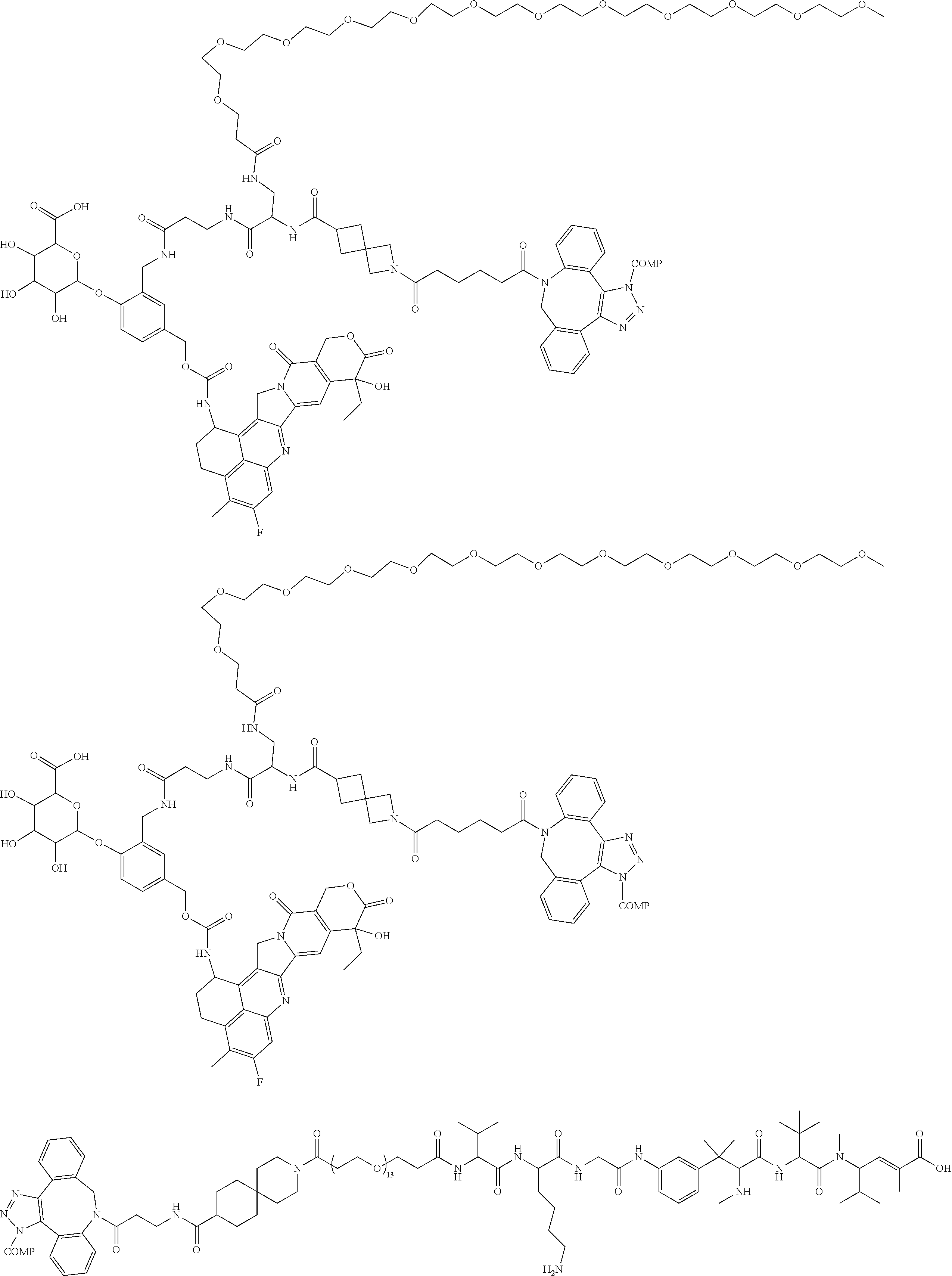

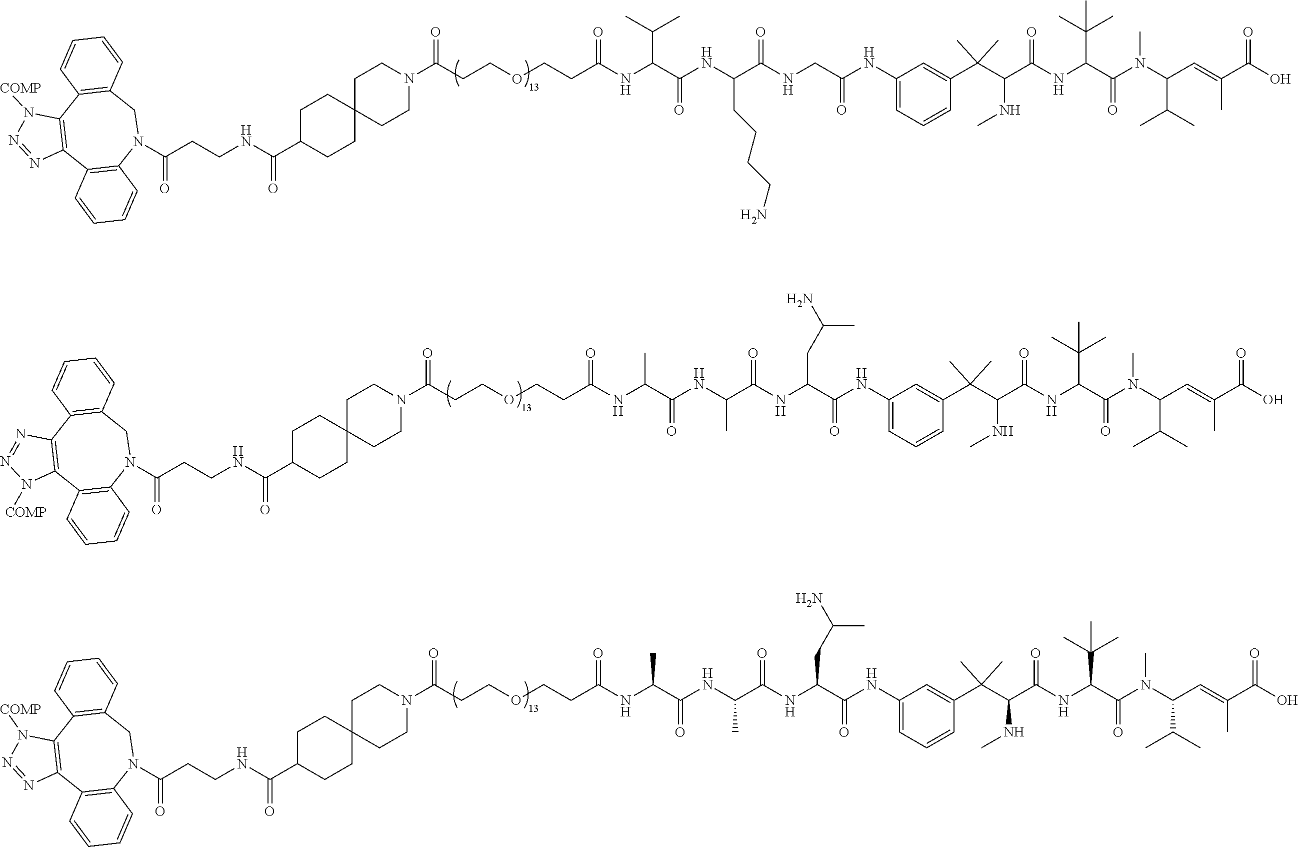

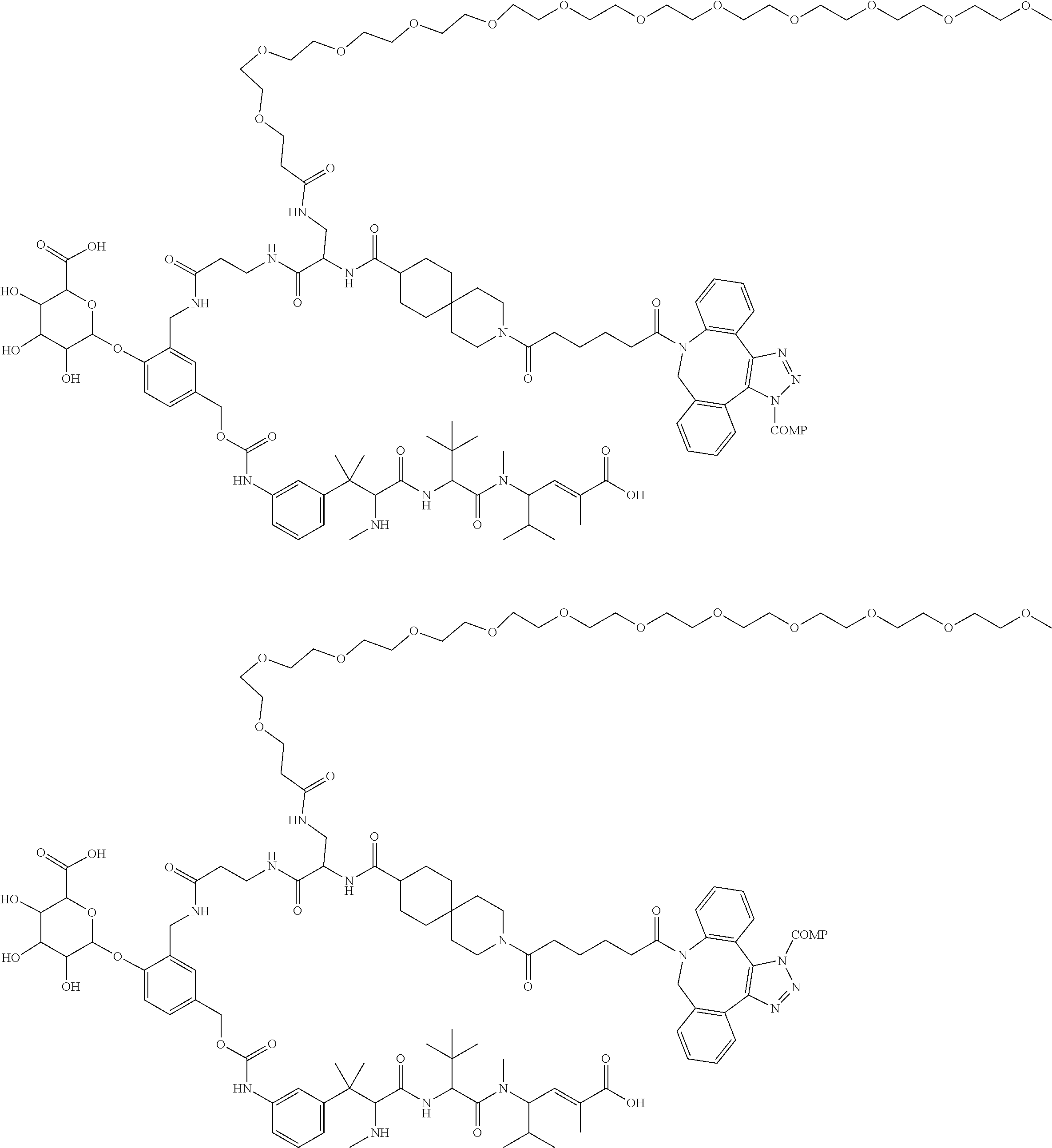

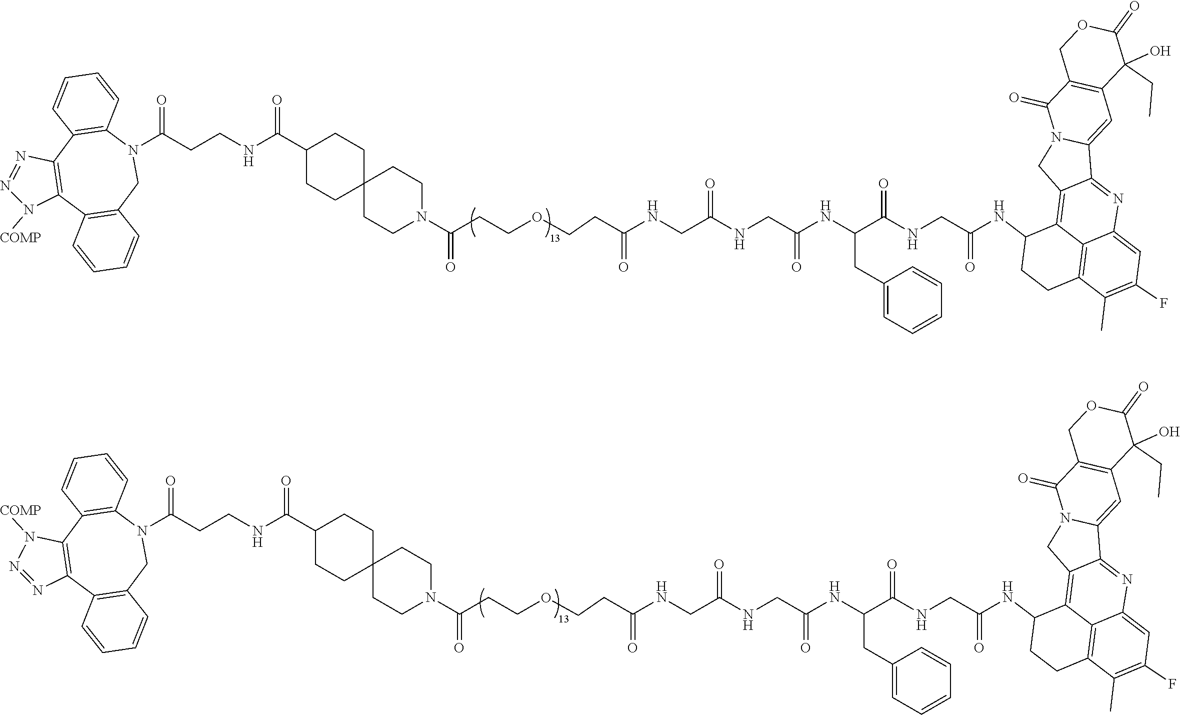

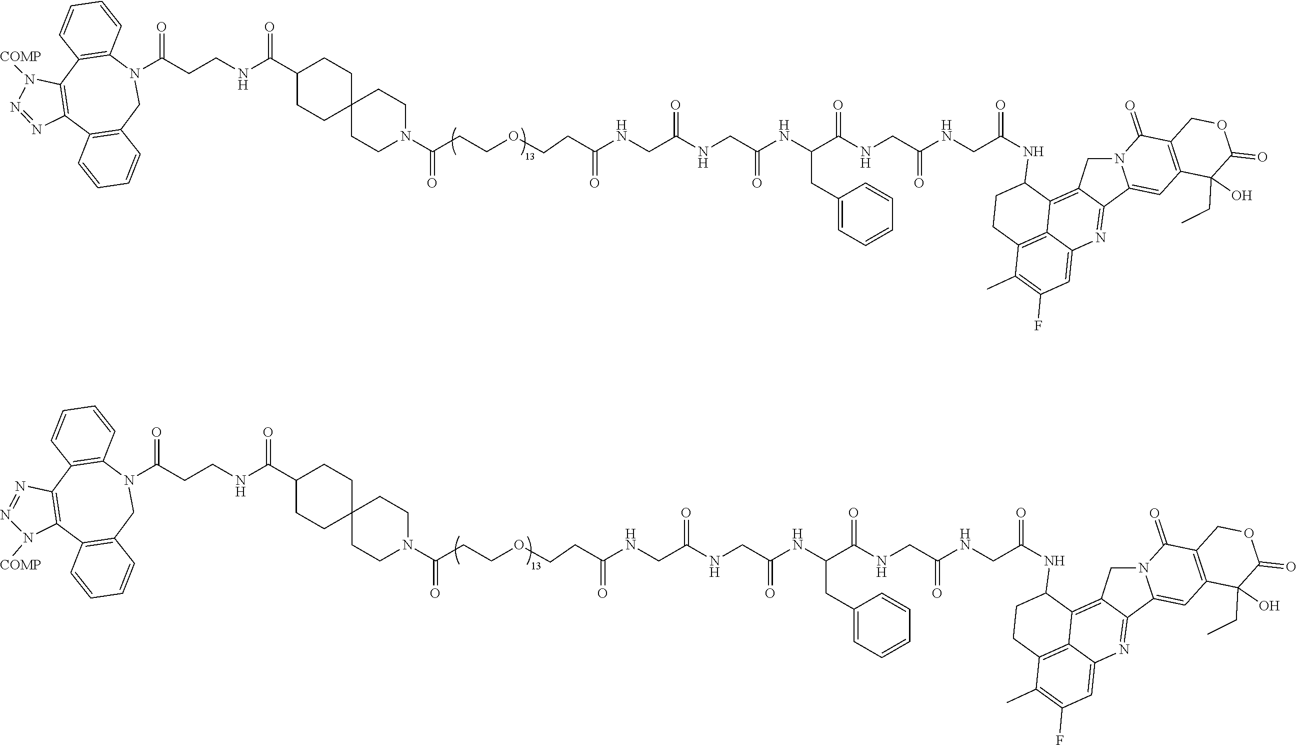

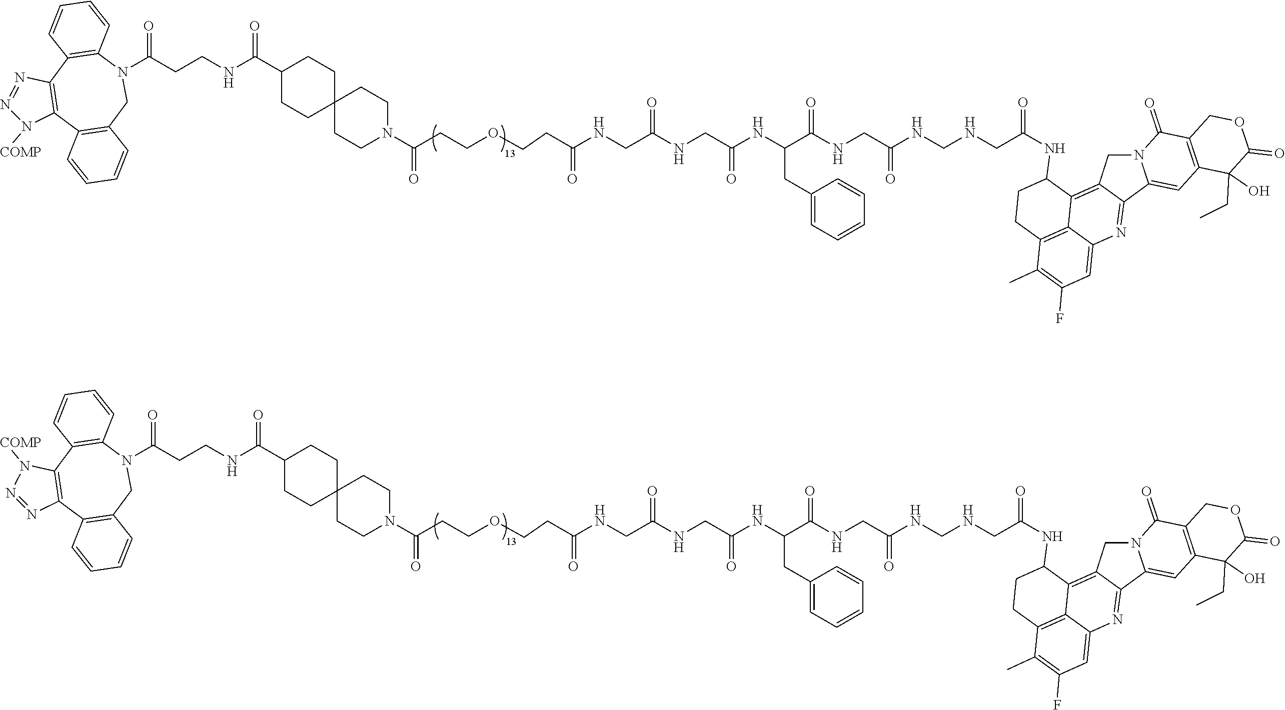

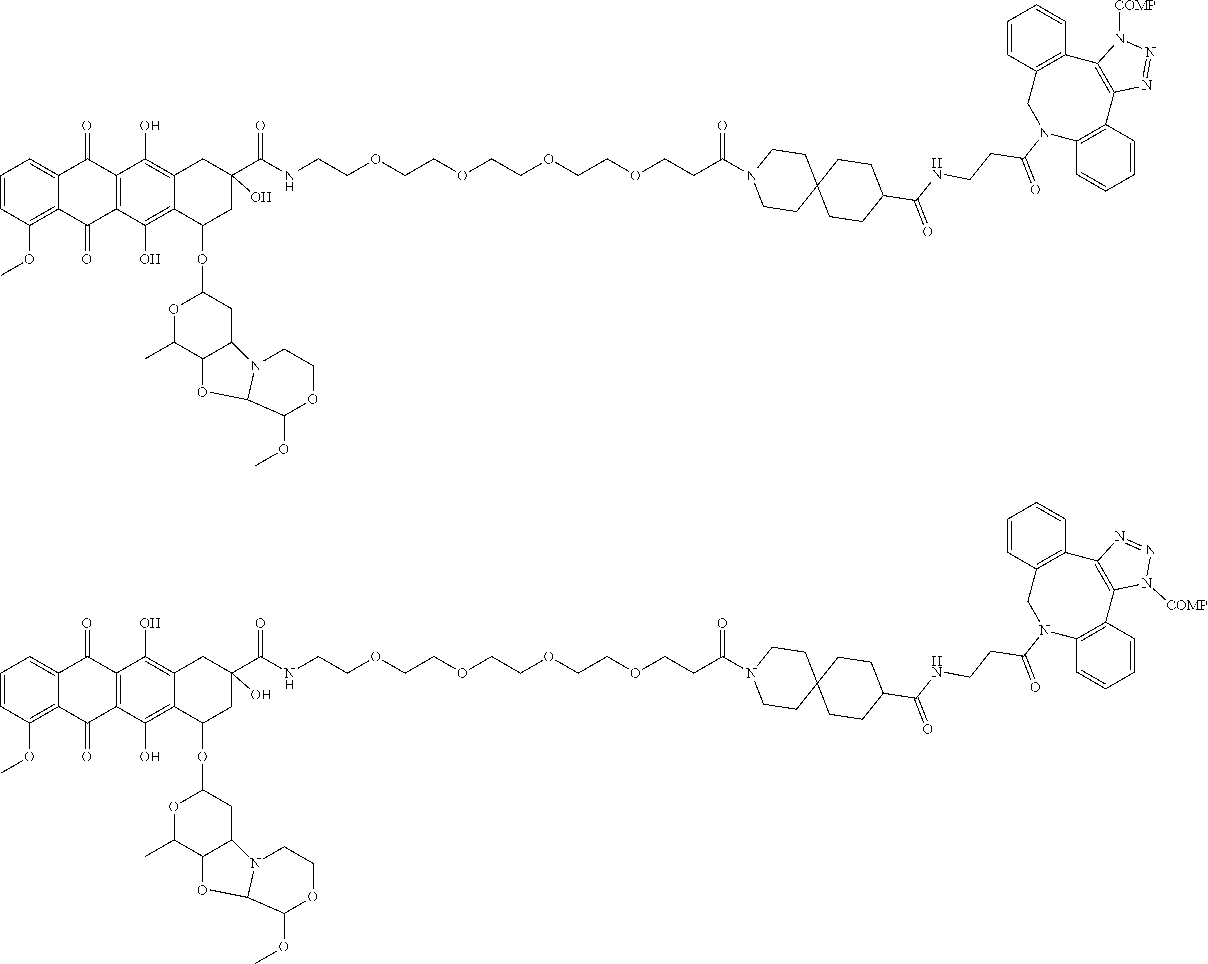

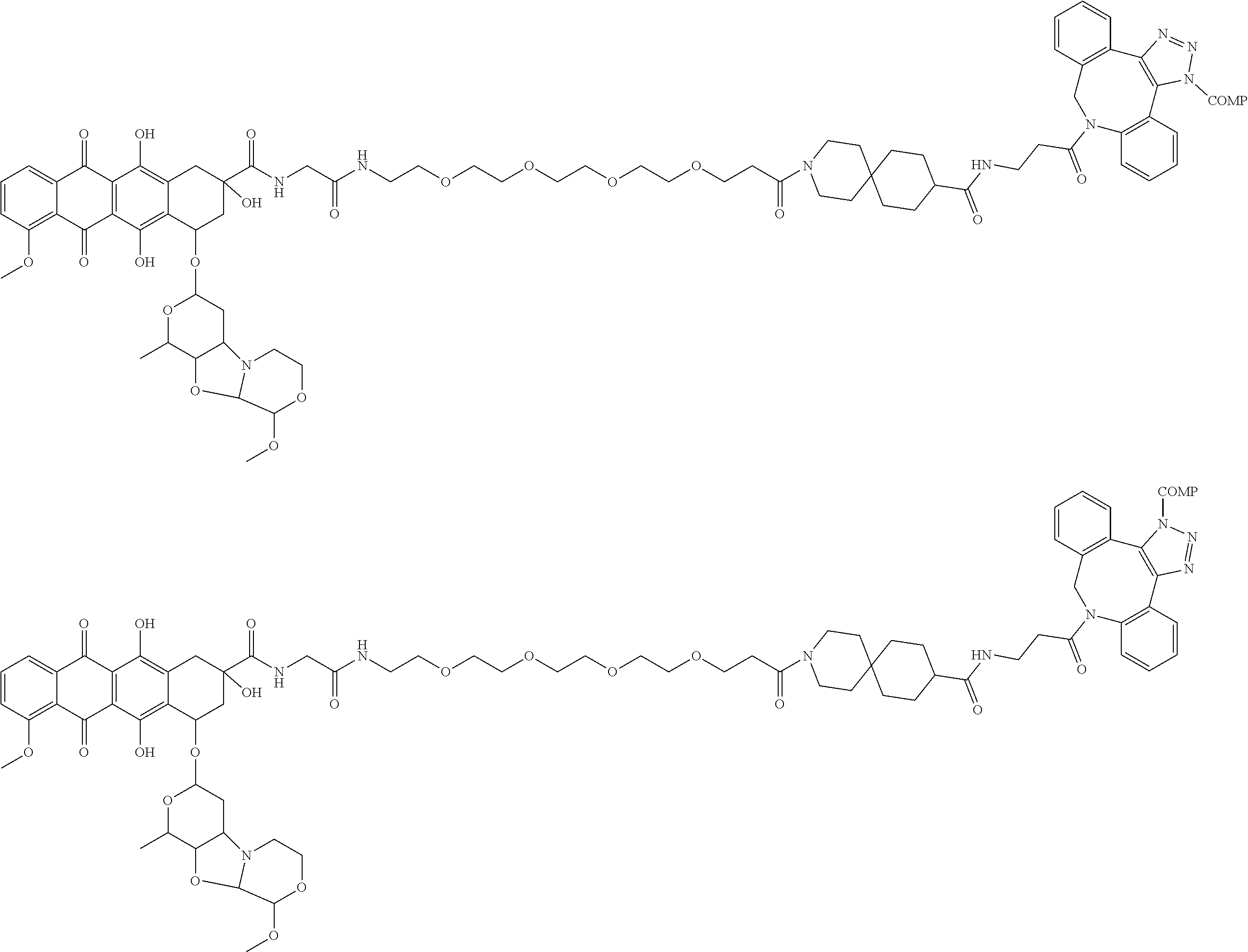

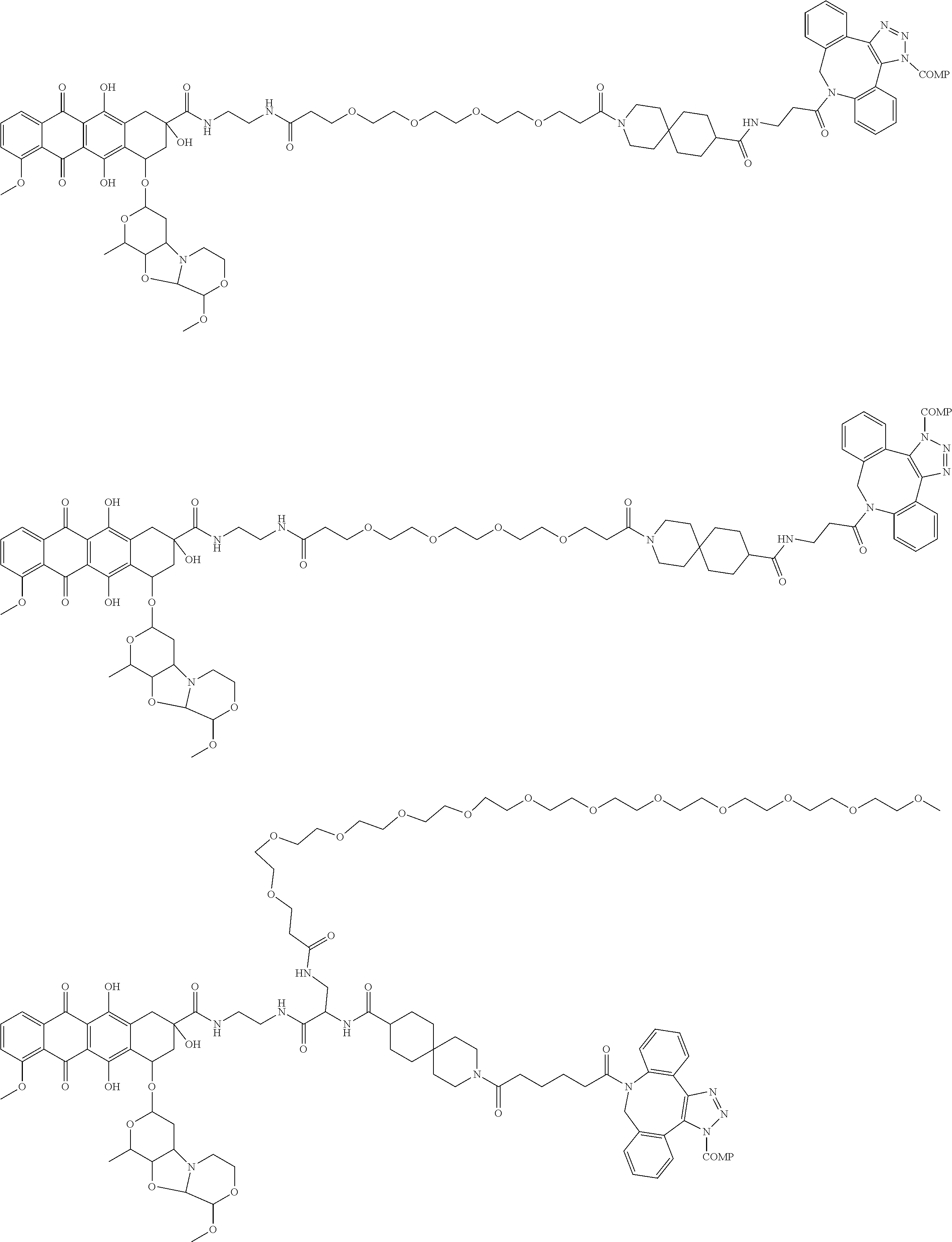

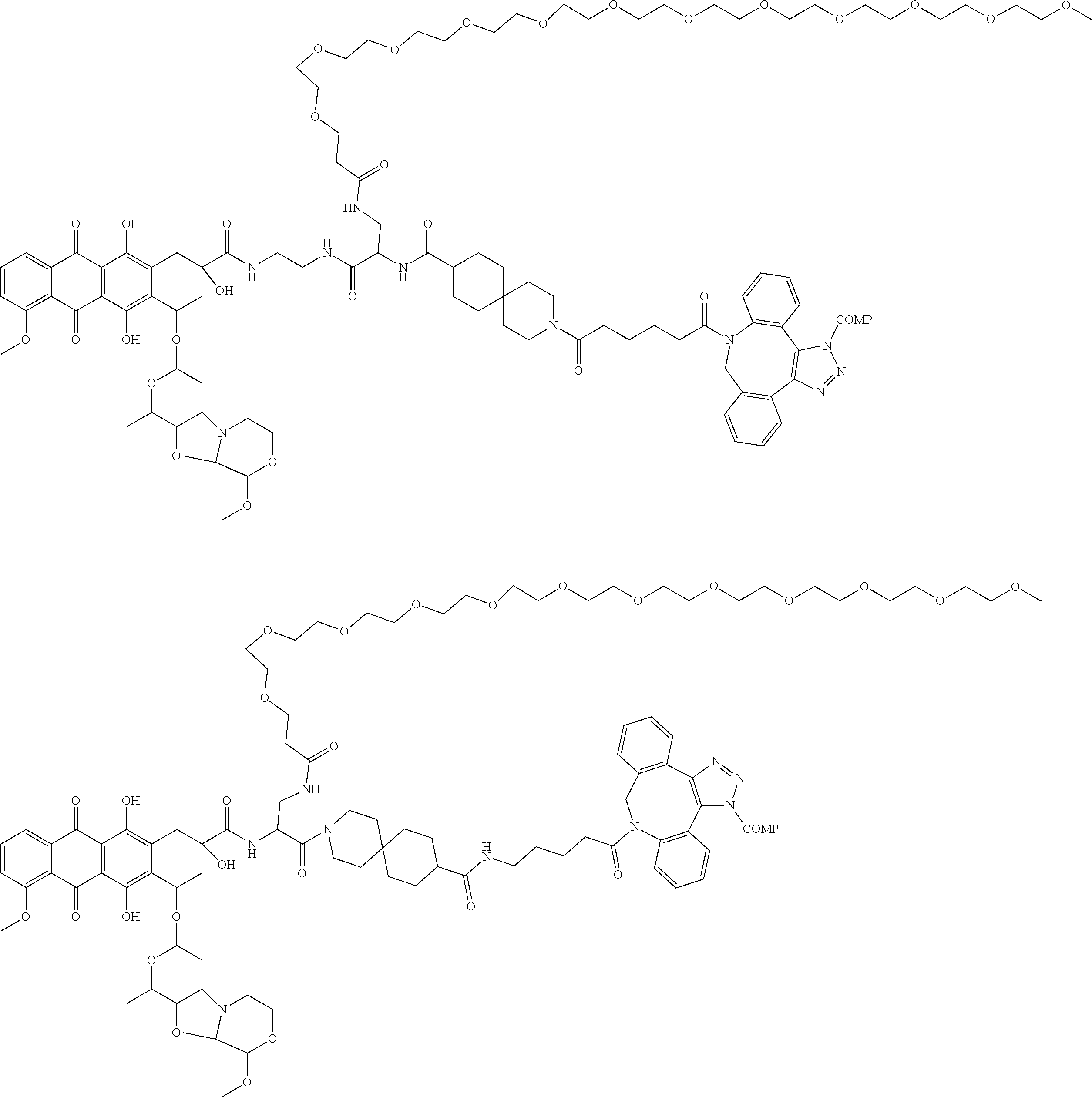

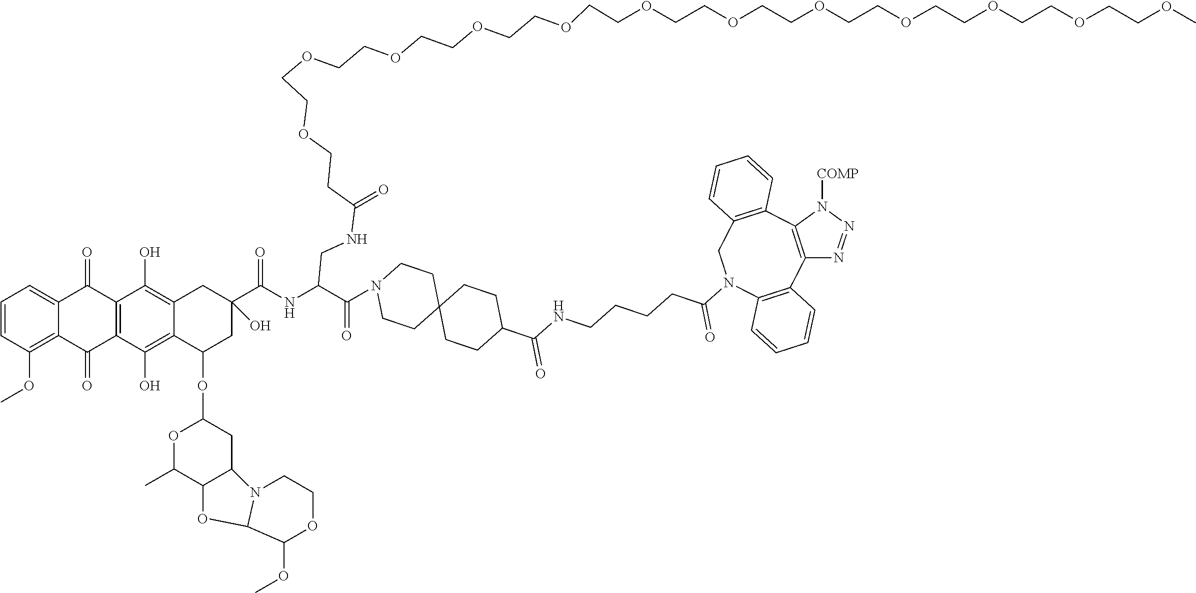

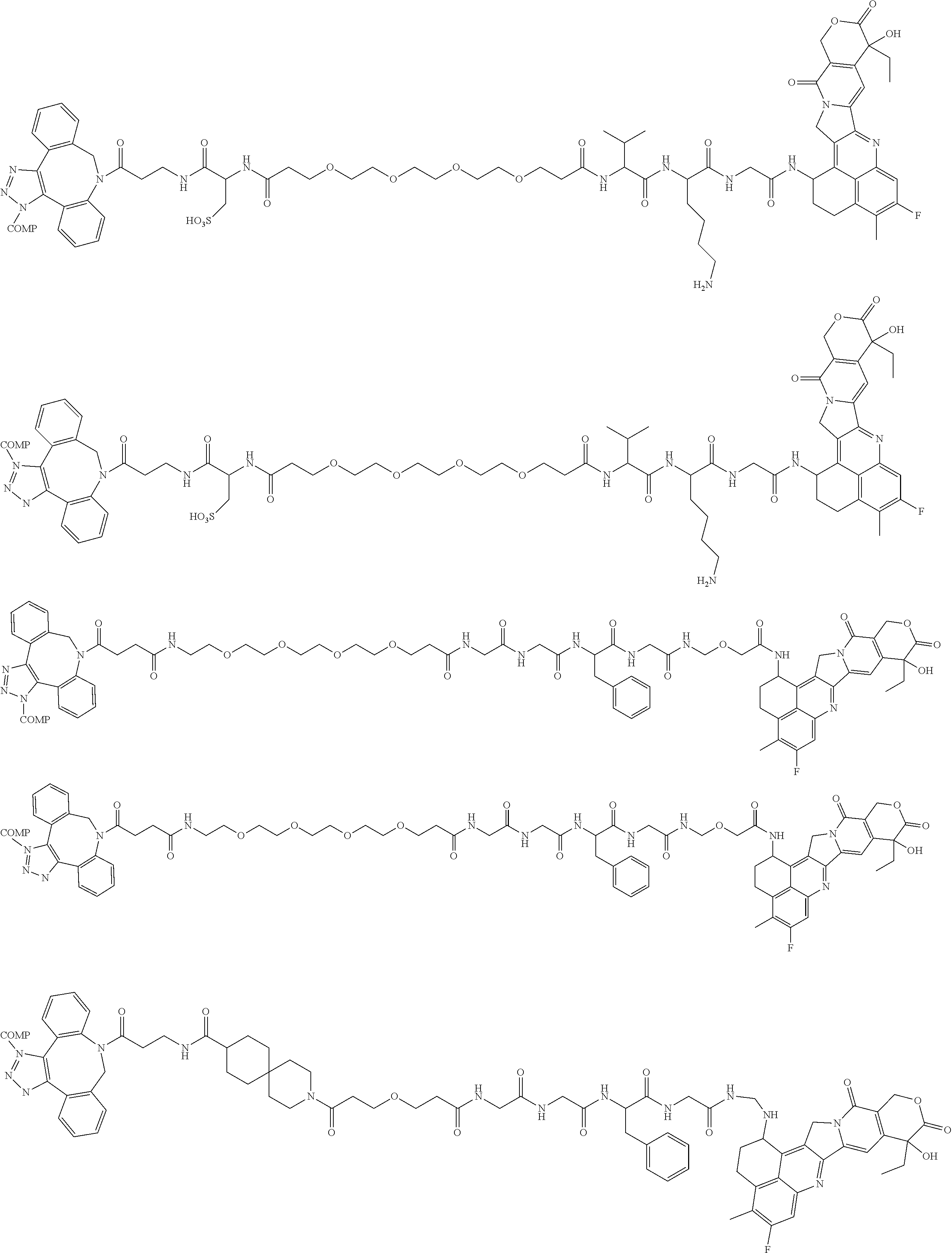

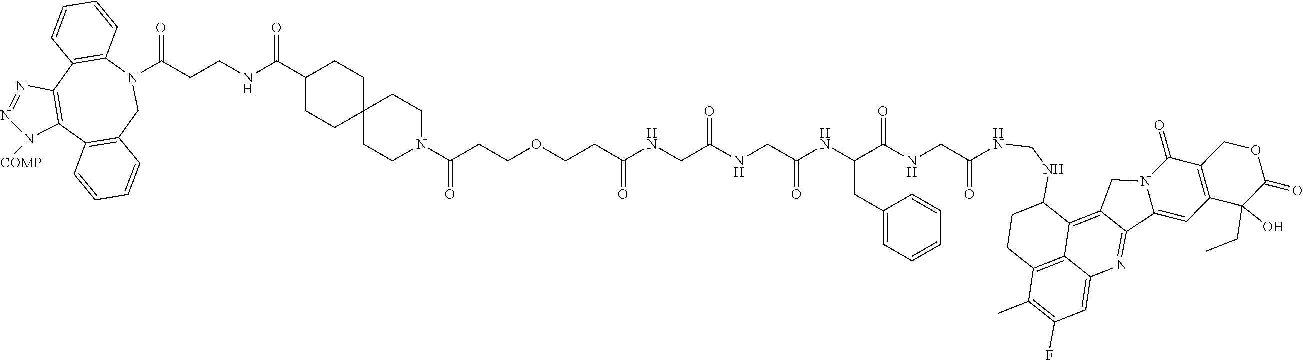

Representative conjugates of the present disclosure are shown in Table B.

TABLE B

| | |
|---|---|
| 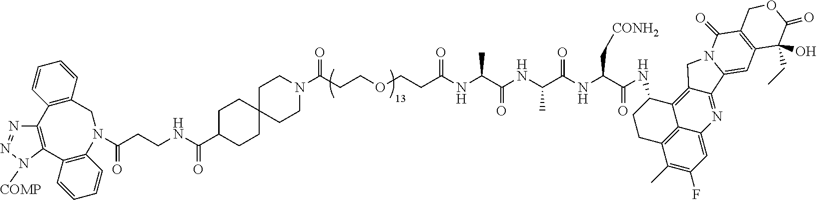 | 101A |
| 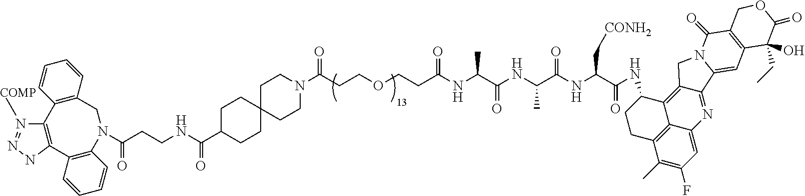 | 101B |
| 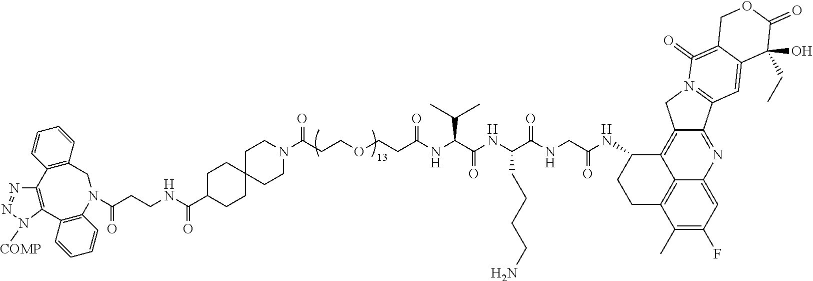 | 102A |
| 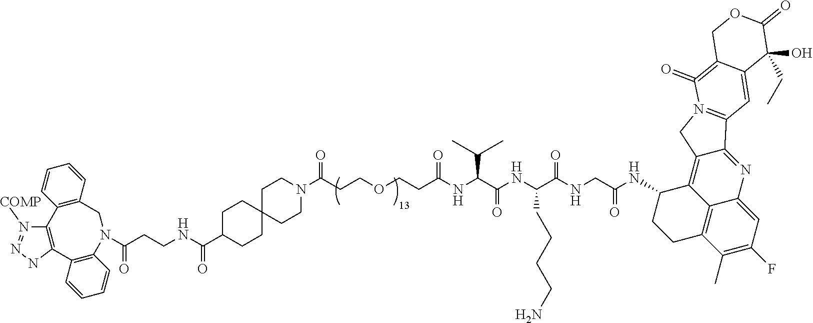 | 102B |
| 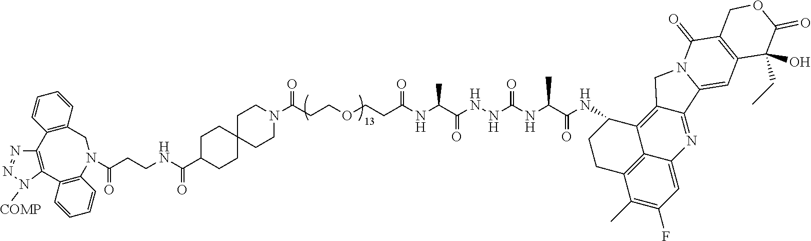 | 103A |
| 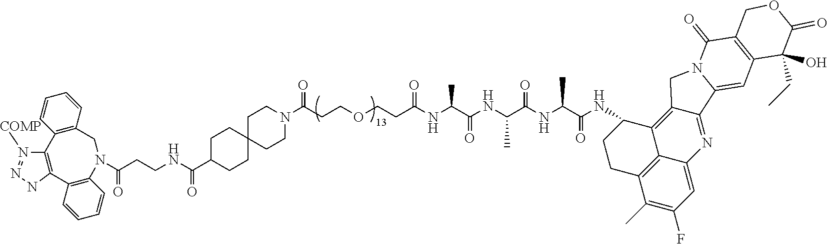 | 103B |
| 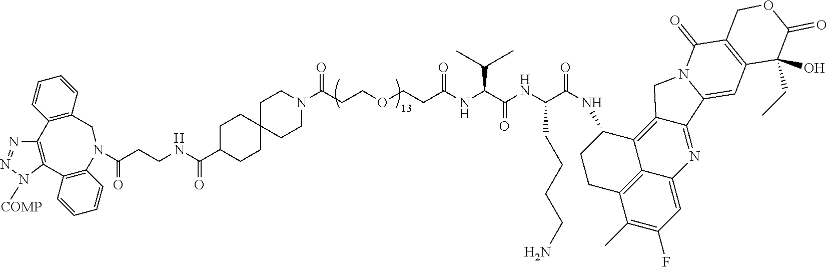 | 104A |
| 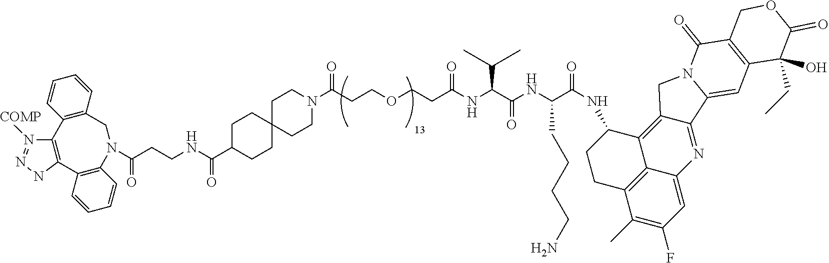 | 104B |
| 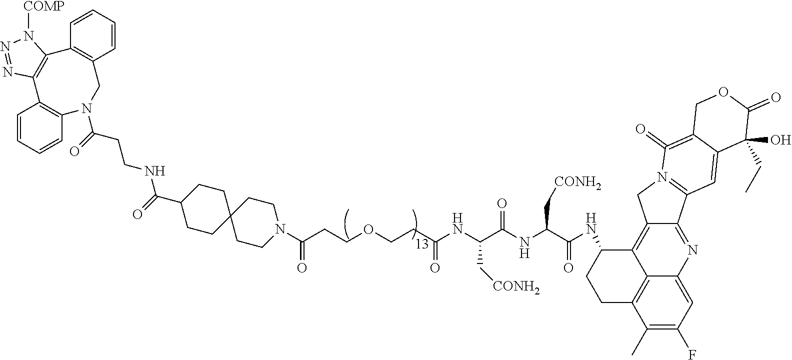 | 105A |
| 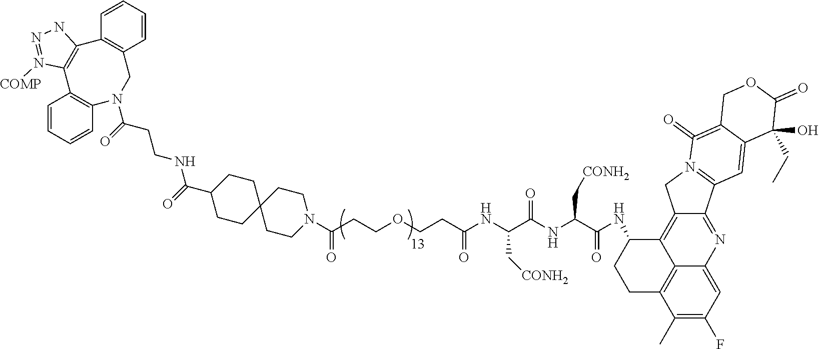 | 105B |
| 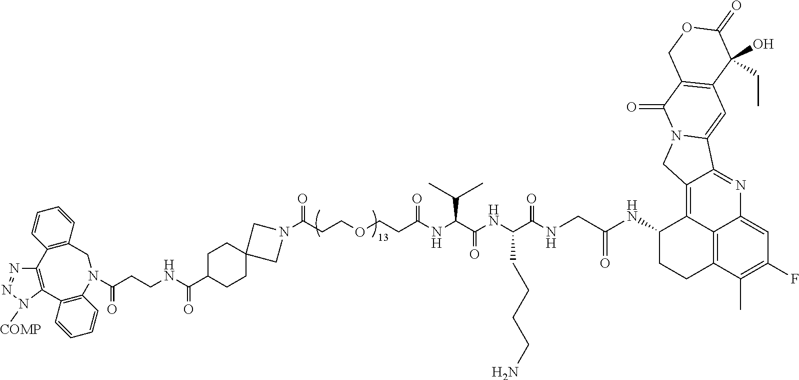 | 106A |
| 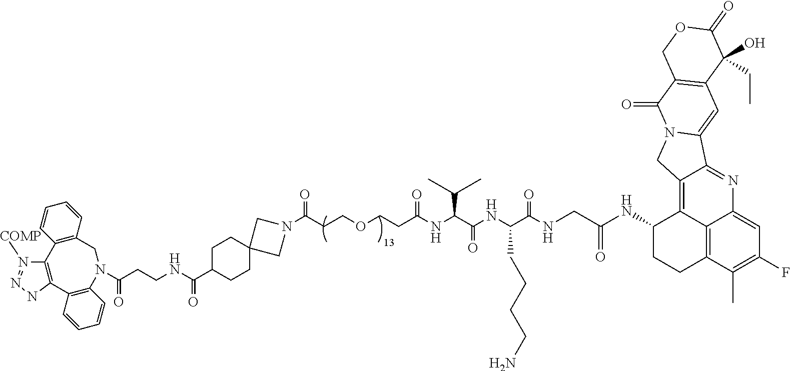 | 106B |
| 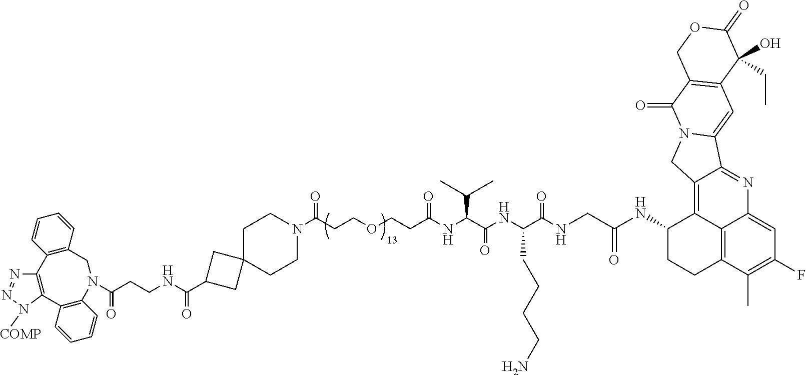 | 107A |
| 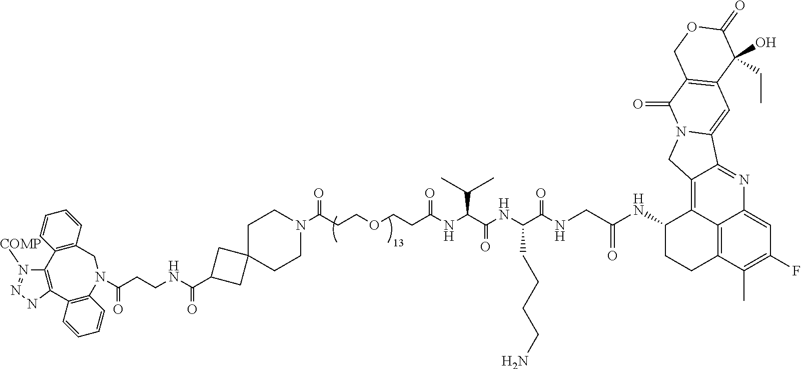 | 107B |
| 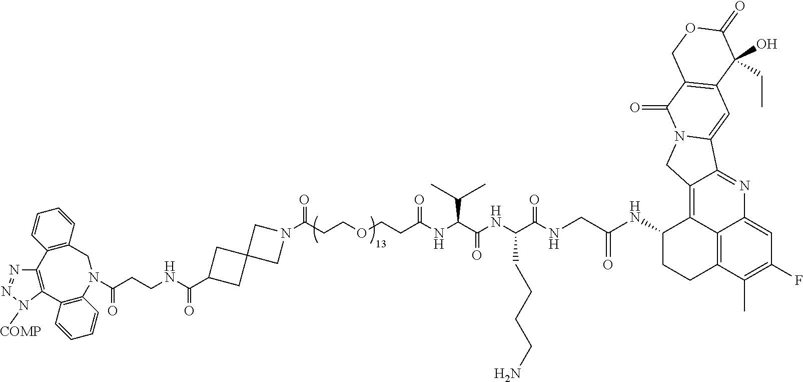 | 108A |
| 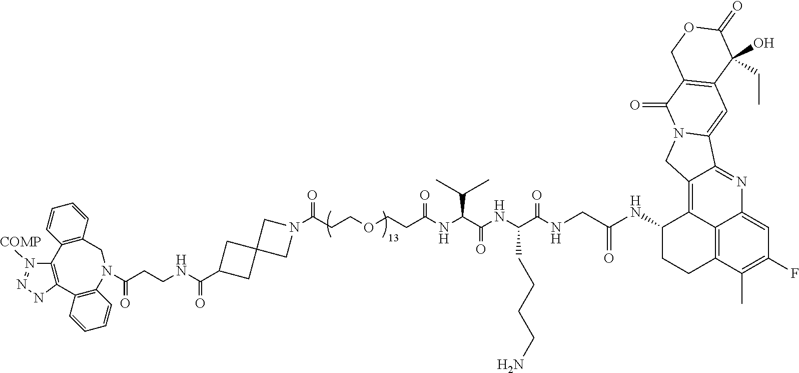 | 108B |
| 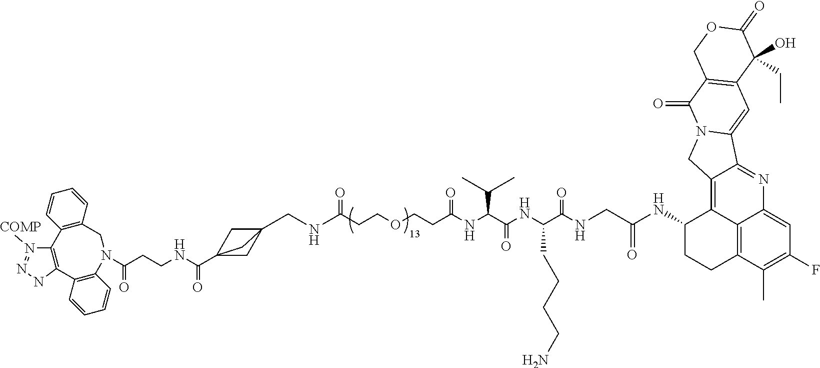 | 109A |
| 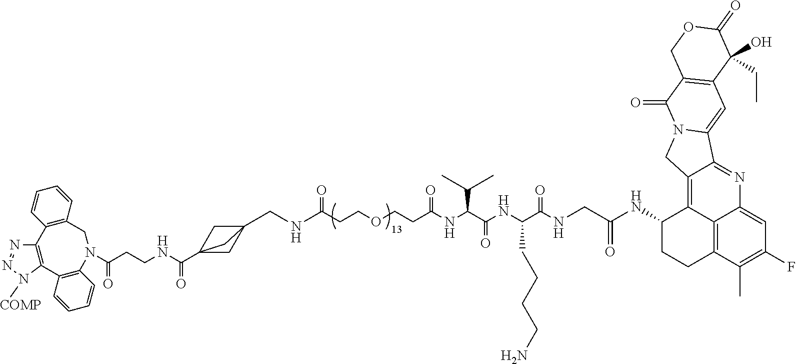 | 109B |
| 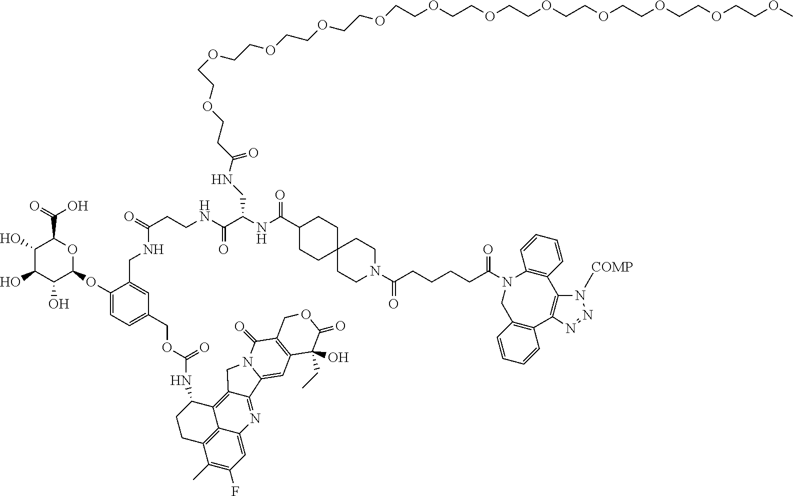 | 110A |
| 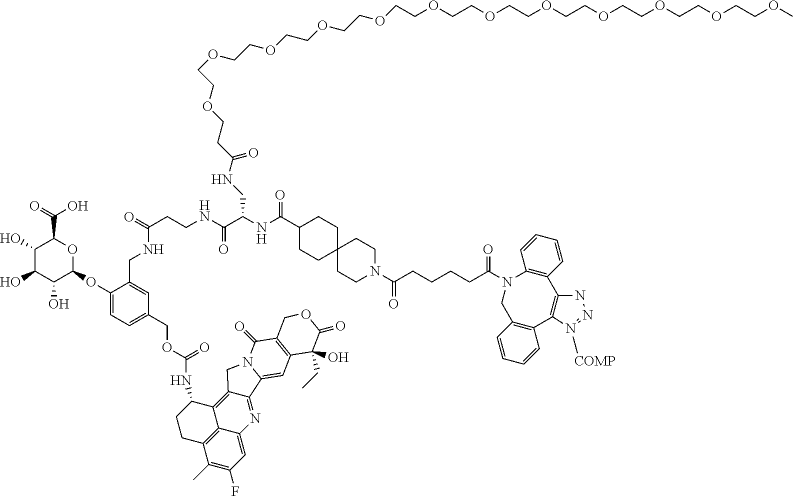 | 110B |
| 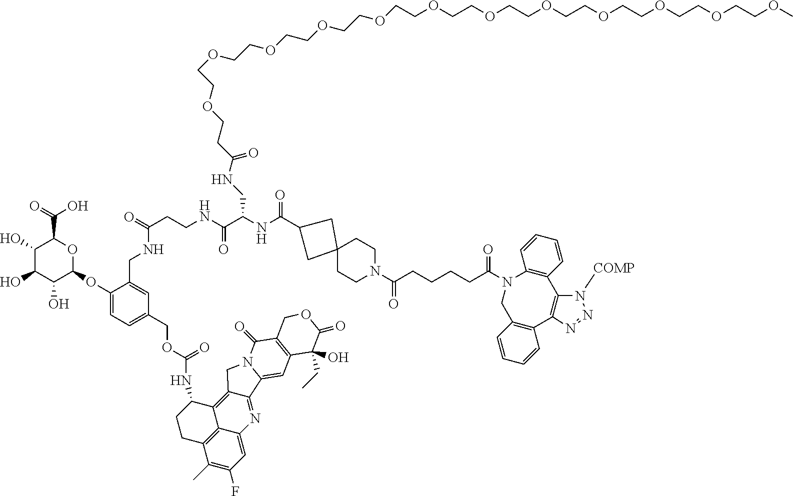 | 111A |
| 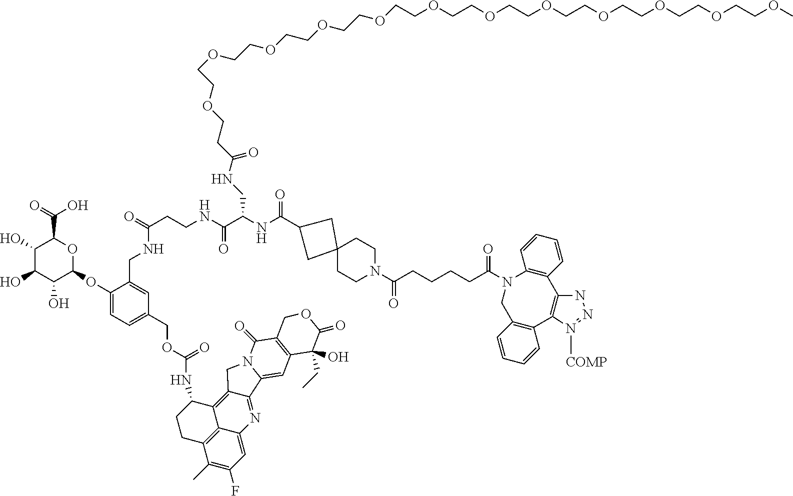 | 111B |
| 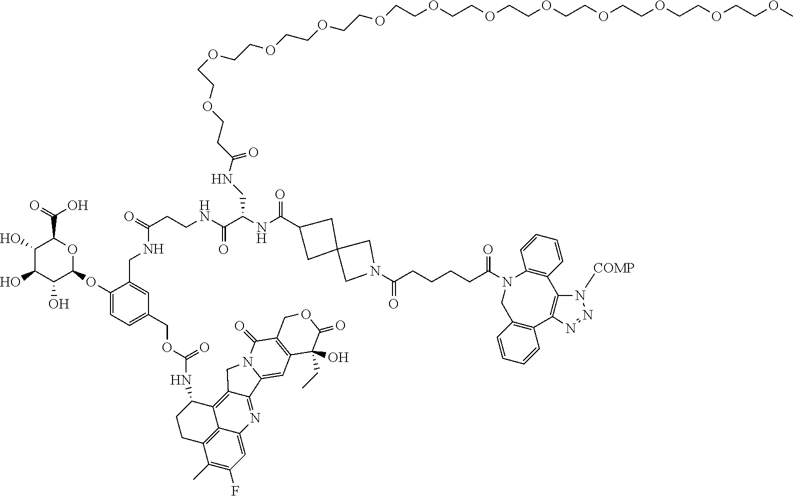 | 112A |
| 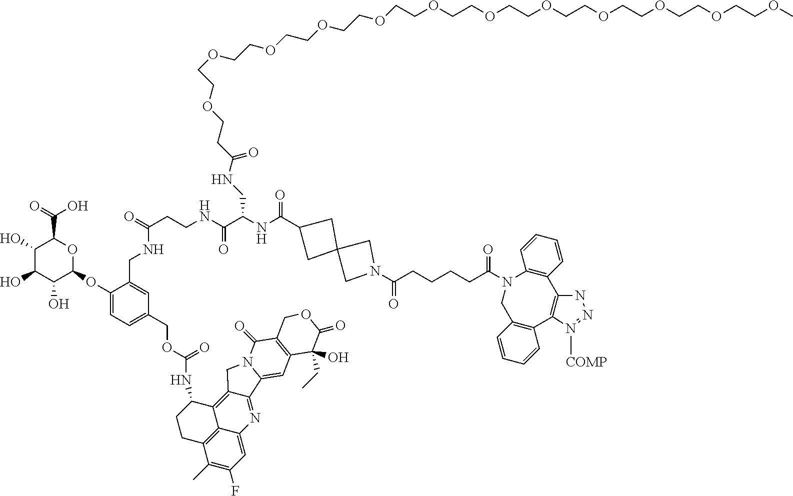 | 112B |
| 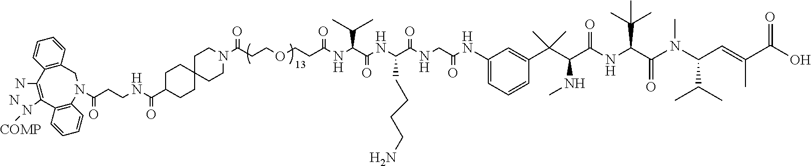 | 113A |
| 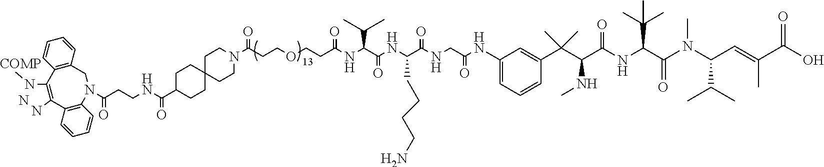 | 113B |
| 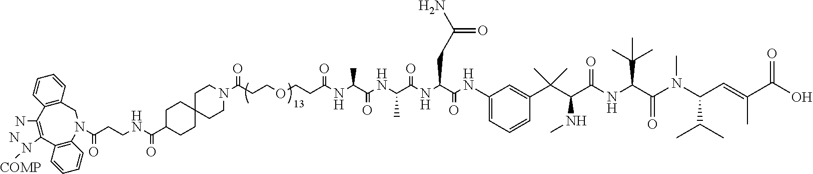 | 114A |
| 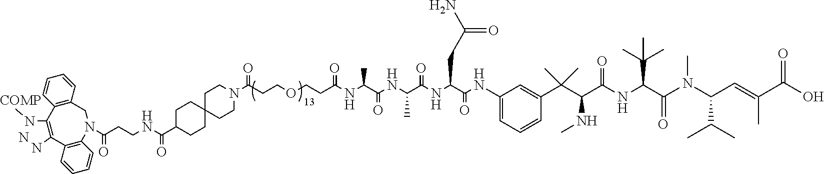 | 114B |
| 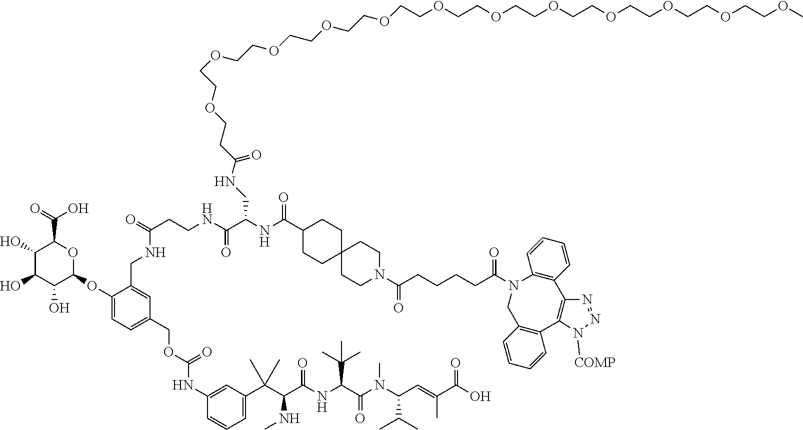 | 115A |
| 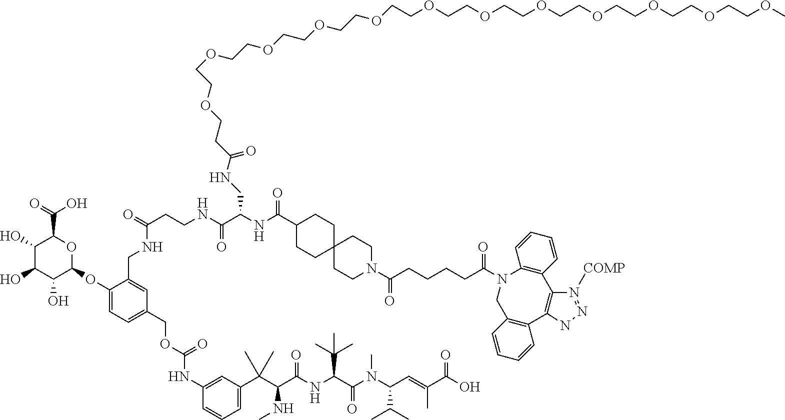 | 115B |
| 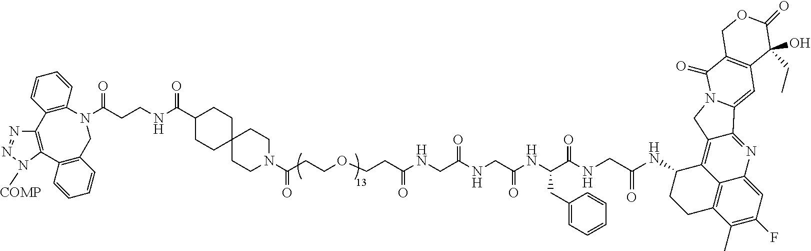 | 116A |
| 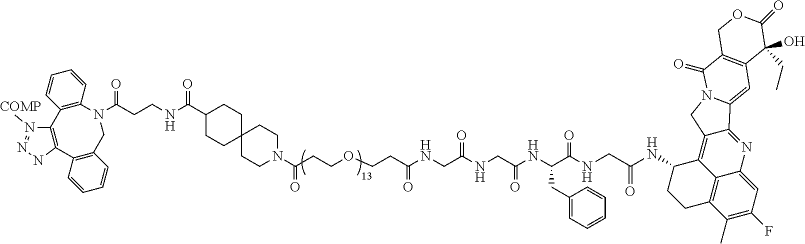 | 116B |
| 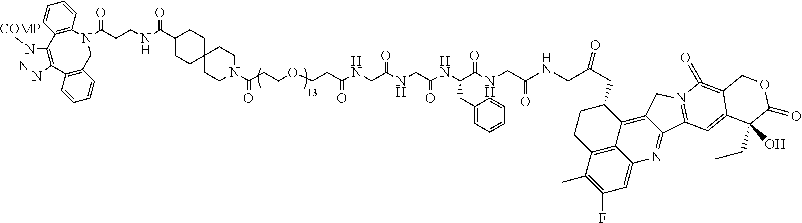 | 117A |
| 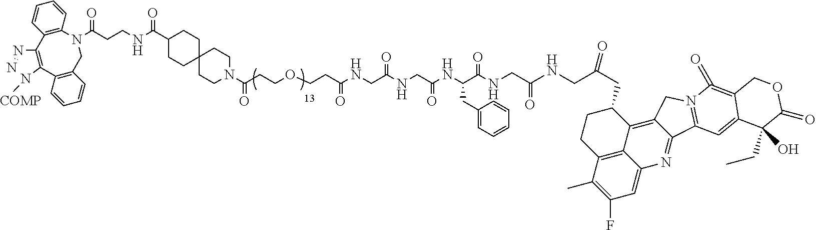 | 117B |
| 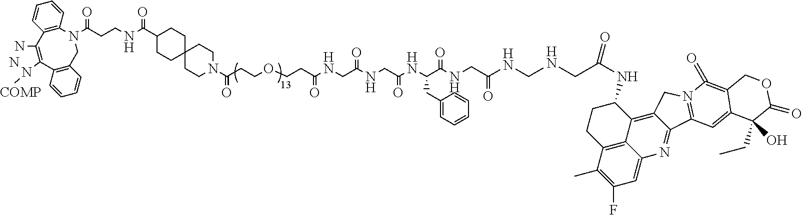 | 118A |
| 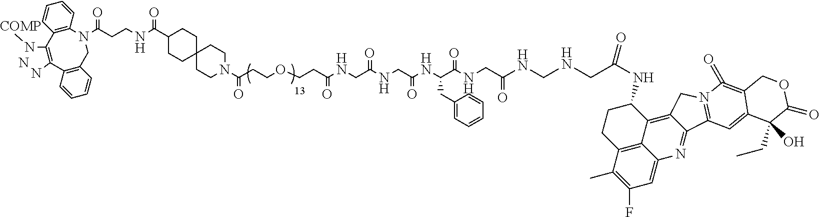 | 118B |
| 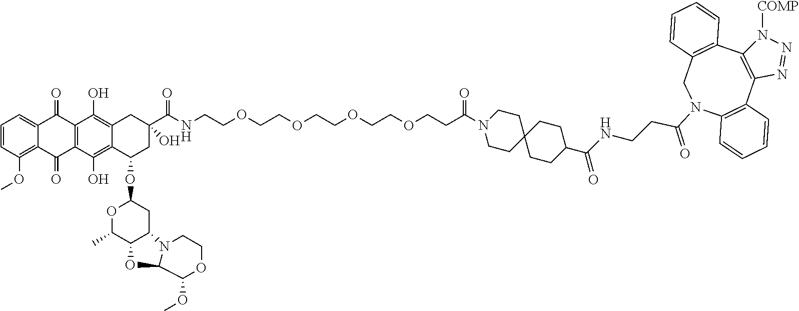 | 119A |
| 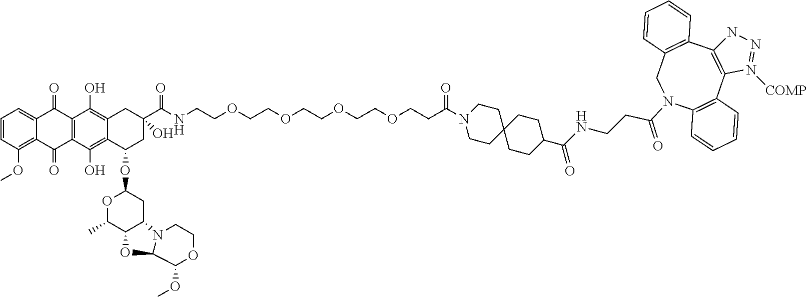 | 119B |
| 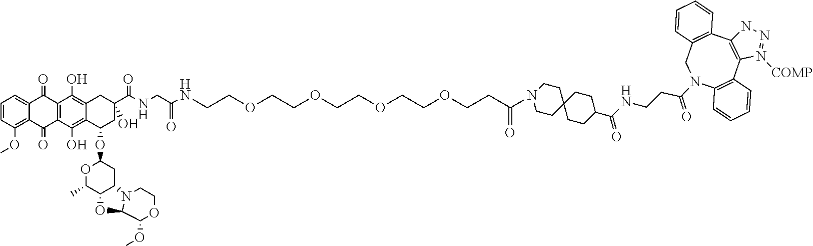 | 120A |
| 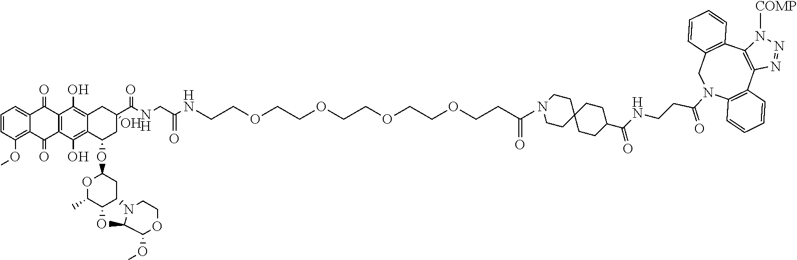 | 120B |
| 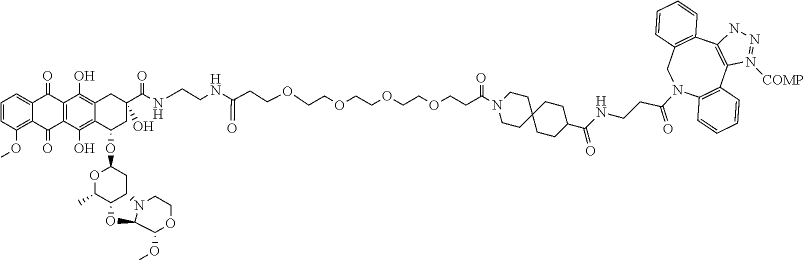 | 121A |
| 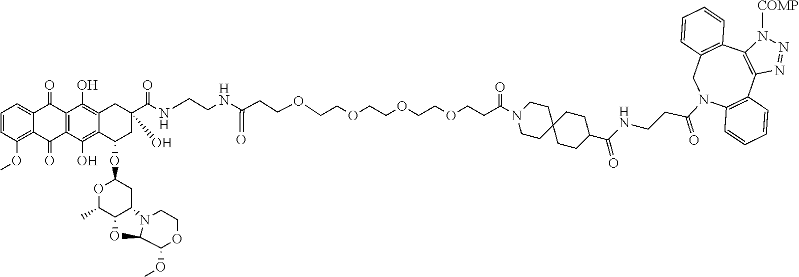 | 121B |
| 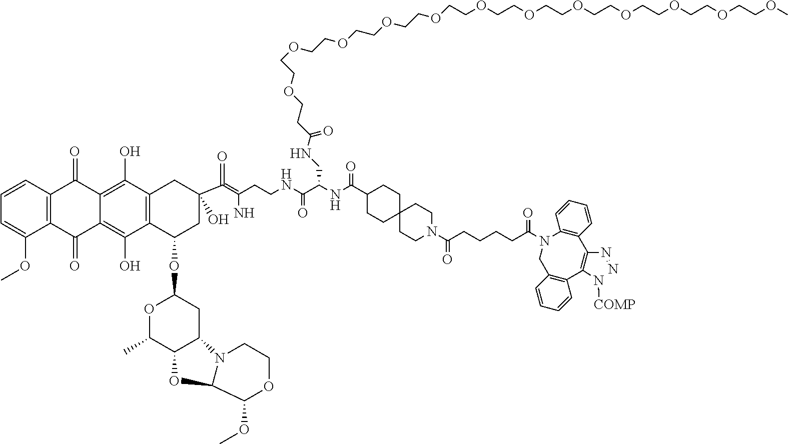 | 122A |
| 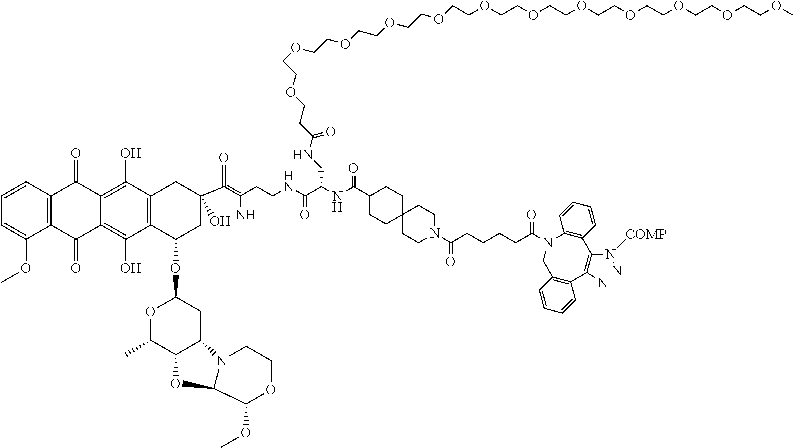 | 122B |
| 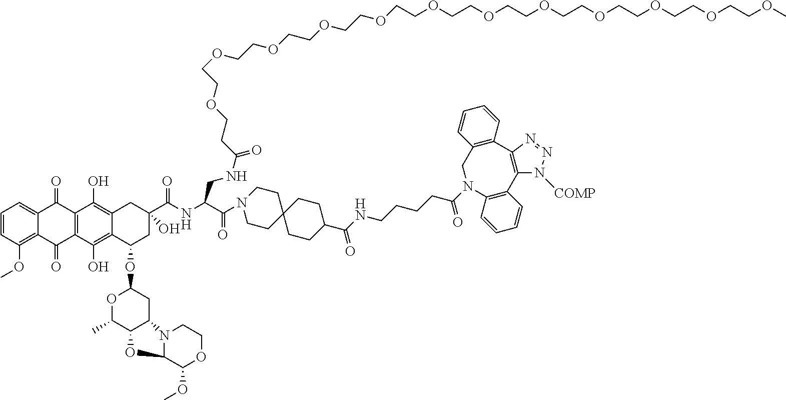 | 123A |
| 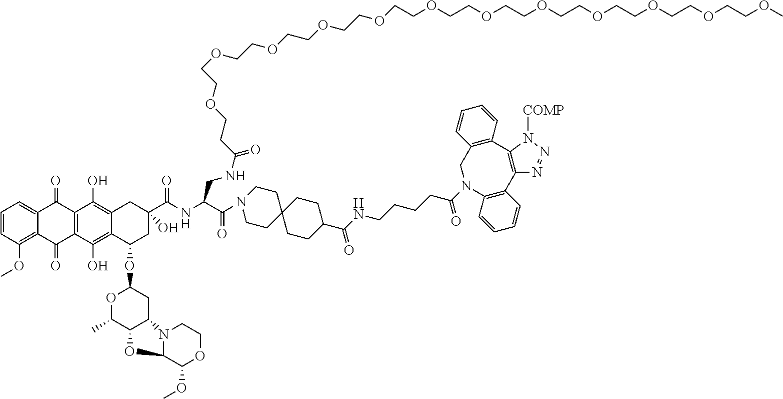 | 123B |
| 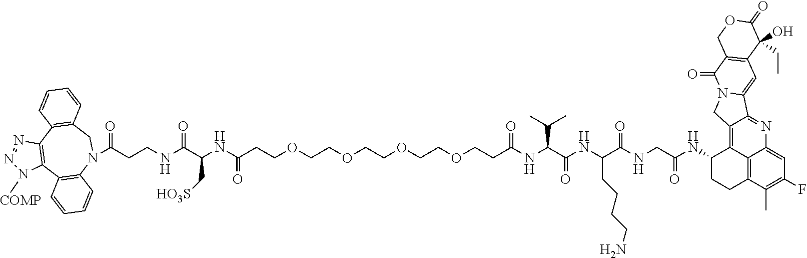 | 124A |
| 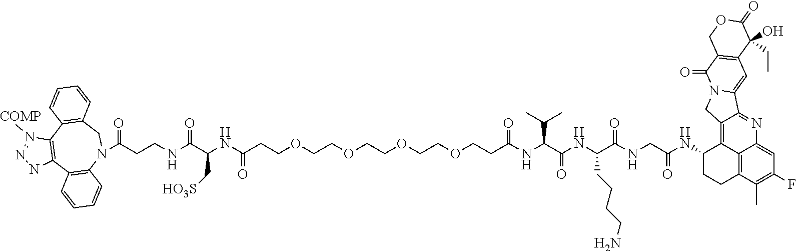 | 124B |
| 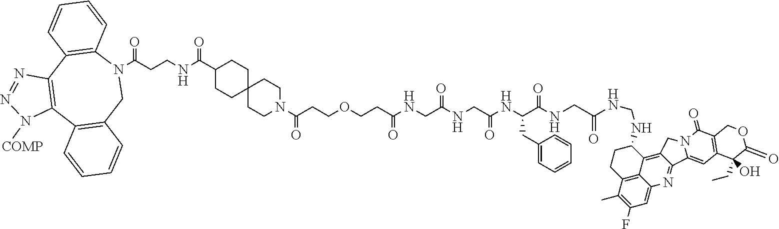 | 125A |
| 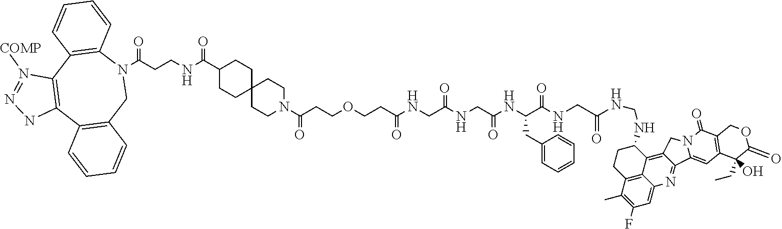 | 125B |

### Optically Active Compounds

In certain embodiments, compounds provided herein may have several chiral centers and may exist in and be isolated in optically active and racemic forms. In certain embodiments, some compounds may exhibit polymorphism. A person of skill in the art will appreciate that compounds provided herein can exist in any racemic, optically-active, diastereomeric, polymorphic, regioisomeric and/or stereoisomeric form, and/or mixtures thereof.

A person of skill in the art will also appreciate that such compounds described herein that possess the useful properties also described herein is within the scope of this disclosure. A person of skill in the art will further appreciate how to prepare optically active forms of the compounds described herein, for example, by resolution of racemic forms via recrystallization techniques, by synthesis from optically-active starting materials, by chiral synthesis, or by chromatographic separation using a chiral stationary phase. In addition, most amino acids are chiral (i.e., designated as L- or D-, wherein the L- enantiomer is the naturally occurring configuration) and can exist as separate enantiomers.

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist (i.e., the material is a conglomerate, and the crystals are visually distinct);
ii) simultaneous crystallization—a technique whereby the individual enantiomers are separately crystallized from a solution of the racemate, only if the latter is a conglomerate in the solid state;
iii) enzymatic resolutions—a technique wherein partial or complete separation of a racemate is accomplished by virtue of different rates of reaction of the enantiomers in the presence of an enzyme;
iv) enzymatic asymmetric synthesis—a synthetic technique wherein at least one step of the synthesis uses an enzymatic reaction to obtain an enantiomerically pure or enriched synthetic precursor of the desired enantiomer;
v) chemical asymmetric synthesis—a synthetic technique wherein the desired enantiomer is synthesized from an achiral precursor using chiral catalysts or chiral auxiliaries to produce asymmetry (i.e., chirality) in the product;
vi) diastereomer separations—a technique wherein a racemic compound is treated with an enantiomerically pure reagent (a chiral auxiliary) that converts the individual enantiomers to diastereomers. The resulting diastereomers are then separated by chromatography or crystallization by virtue of their now more distinct diastereomeric differences, and then the chiral auxiliary is removed to obtain each enantiomer;
vii) first- and second-order asymmetric transformations—a technique wherein diastereomers of the racemate equilibrate in solution to yield a preponderance of a diastereomer of the desired enantiomer, or where kinetic or thermodynamic crystallization of the diastereomer of the desired enantiomer perturbs the equilibrium such that eventually in principle all the material is converted to the crystalline diastereomer of the desired enantiomer. The desired enantiomer is then derived from the diastereomer;
viii) kinetic resolutions—this technique refers to the achievement of partial or complete resolution of a racemate (or of a further resolution of a partially resolved compound) by virtue of unequal reaction rates of the enantiomers with a chiral or non-racemic reagent or catalyst under kinetic conditions;
ix) enantiospecific synthesis from non-racemic precursors—a synthetic technique wherein the desired enantiomer is obtained from chiral starting materials and where the stereochemical integrity is not or is only minimally compromised over the course of the synthesis;
x) chiral liquid chromatography—a technique wherein the enantiomers of a racemate are separated in a liquid mobile phase by virtue of their different interactions with a stationary phase. The stationary phase can be made of chiral material or the mobile phase can contain an additional chiral material to provoke the different interactions;
xi) chiral gas chromatography—a technique wherein the racemate is volatilized and enantiomers are separated by virtue of their different interactions in the gaseous mobile phase with a column containing a fixed non-racemic adsorbent phase;
xii) extraction with chiral solvents—a technique wherein the enantiomers are separated by virtue of kinetic or thermodynamic dissolution of one enantiomer into a particular chiral solvent;
xiii) transport across chiral membranes—a technique wherein a racemate is placed in contact with a thin membrane barrier. The barrier typically separates two miscible fluids, one containing the racemate, and a driving force such as a concentration or pressure differential causes preferential transport across the membrane barrier. Separation occurs as a result of the non-racemic nature of the membrane which allows only one enantiomer of the racemate to pass through.

In some embodiments, provided herein are compositions of the compounds of the present disclosure, including compounds of Formula (I), (IA), (IB), (IC), (ID), (IE), (IF), (IG), (IH), (III), (IIIA), and (IIIB) and conjugates of Formula (II), (IIA), (IIB), (IIC), (IID), (IIE), (IIF), (IIG), (IIH), (IV), (IVA), and (IVB) that are substantially free of a designated stereoisomer of that compound or conjugate. In certain embodiments, in the methods, compounds, and conjugates of this disclosure, the compounds or conjugates are substantially free of other stereoisomers. In some embodiments, the composition includes a compound or conjugate that is at least 85%, 90%, 95%, 98%, or 99% to 100% by weight of the compound or conjugate, the remainder comprising other chemical species or enantiomers. In some embodiments, provided herein are compositions of compounds of Formula (I), (IA), (IB), (IC), (ID), (IE), (IF), (IG), (IH), (III), (IIIA), and (IIIB) and conjugates of Formula (II), (IIA), (IIB), (IIC), (IID), (IIE), (IIF), (IIG), (IIH), (IV), (IVA), and (IVB) that are substantially free of a designated enantiomer of that compound or conjugate. In certain embodiments, in the methods, compounds, and conjugates of this disclosure, the compounds or conjugates are substantially free of other enantiomers. In some embodiments, the composition includes a compound or conjugate that is at least 85%, 90%, 95%, 98%, or 99% to 100% by weight of the compound or conjugate, the remainder comprising other chemical species or enantiomers.

### Isotopically Enriched Compounds

Also provided herein are isotopically enriched compounds and conjugates including, but not limited to, isotopically enriched compounds of Formula (I), (IA), (IB), (IC), (ID), (IE), (IF), (IG), (IH), (III), (IIIA), and (IIIB) and conjugates of Formula (II), (IIA), (IIB), (IIC), (IID), (IIE), (IIF), (IIG), (IIH), (IV), (IVA), and (IVB).

Isotopic enrichment (for example, deuteration) of pharmaceuticals to improve pharmacokinetics (“PK”), pharmacodynamics (“PD”), and/or toxicity profiles, has been previously demonstrated within some classes of drugs. See, for example, Lijinsky et al., Food Cosmet. Toxicol., 20: 393 (1982); Lijinsky et al., J. Nat. Cancer Inst., 69: 1127 (1982); Mangold et al., Mutation Res. 308: 33 (1994); Gordon et al., Drug Metab. Dispos., 15: 589 (1987); Zello et al., Metabolism, 43: 487 (1994); Gately et al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites, if any are formed; and/or (6) decrease the production of deleterious metabolites in specific tissues. Isotopic enrichment of a drug can also be used to create a more effective and/or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a (heavier) isotope for that reactive hydrogen will cause a decrease in the reaction rate. The Deuterium Kinetic Isotope Effect (“DKIE”) is the most common form of KIE. (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen and the C-D bond is broken. The DKIE can range from about one (no isotope effect) to very large numbers, such as 50 or more, meaning that the reaction can be fifty, or more, times slower when deuterium has been substituted for hydrogen.

Substitution of tritium (“T”) for hydrogen results in yet a stronger bond than deuterium and gives numerically larger isotope effects. Similarly, substitution of isotopes for other elements including, but not limited to, ¹³C or ¹⁴C for carbon; ³³S, ³⁴S, or ³⁶S for sulfur; ¹⁵N for nitrogen; and ¹⁷O or ¹⁸O for oxygen may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C═C) pi-bond. The resultant metabolites may be stable or unstable under physiological conditions, and can have substantially different PK/PD, and acute and long-term toxicity profiles relative to the parent compounds. For many drugs, such oxidations are rapid. Therefore, these drugs often require the administration of multiple or high daily doses.

Therefore, isotopic enrichment at certain positions of a compound provided herein will produce a detectable KIE that will affect the pharmacologic, PK, PD, and/or toxicological profiles of a compound provided herein in comparison with a similar compound having a natural isotopic composition.

### Conjugates of Formula (II), (IA), (IIB), (IIC), (IID), (IIE), (IIF), (IIG), (IIH), (III), (IIIA), and (IIIB)

Provided herein are conjugates of macromolecules with one of the compounds of Formula (I)—(IH), (III), (IIIA), and (IIIB) as described herein. The conjugates are covalently linked directly or indirectly, via a linker. In certain embodiments, the conjugate comprises a macromolecule conjugated to one or more compounds of Formula (I)—(IH), (III), (IIIA), and (IIIB) as described herein. In certain embodiments, the conjugate comprises more than one macromolecule. In certain embodiments, the macromolecule is linked to one, two, three, four, five, six, seven, eight, or more compounds of Formula (I)—(IH), (III), (IIIA), and (IIIB).

The linker can be any linker capable of forming at least one bond to the macromolecule and at least one bond to a compound of Formula (I)—(IH), (III), (IIIA), and (IIIB). Useful linkers are described in the sections and examples herein and in particular, below.

The macromolecule can be any macromolecule deemed suitable by the person of skill in the art. In certain embodiments, the macromolecule is a second compound. In certain embodiments, COMP is a residue of the second compound. In certain embodiments, the macromolecule is a protein, peptide, antibody or antigen-binding fragment thereof, nucleic acid, carbohydrate, or other large molecule composed of polymerized monomers. In certain embodiments, the macromolecule is a peptide of two or more residues. In certain embodiments, the macromolecule is a peptide of ten or more residues. In certain embodiments, the macromolecule is at least 1000 Da in mass. In certain embodiments, the macromolecule comprises at least 1000 atoms. Useful macromolecules are described in the sections below.

### Macromolecules (COMP)

The macromolecule (COMP) can be any macromolecule deemed suitable by the person of skill in the art. In certain embodiments, the macromolecule is a protein, peptide, antibody or antigen binding fragment thereof, nucleic acid, carbohydrate, or other large molecule composed of polymerized monomers. In certain embodiments, the macromolecule is a protein. In certain embodiments, the macromolecule is an antibody, or an antigen binding fragment thereof. In some embodiments, COMP is a residue of a polypeptide. In some embodiments, COMP is a residue of an antibody. In some embodiments, COMP is a residue of an antibody chain.

In some embodiments, the macromolecule is an antibody or an antigen binding fragment thereof. In some embodiments, the macromolecule is a known antibody. Useful antibodies include, but are not limited to, rituximab (Rituxan®, IDEC/Genentech/Roche) (see, e.g., U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT Application No. PCT/US2003/040426), trastuzumab (Herceptin®, Genentech) (see, e.g., U.S. Pat. No. 5,677,171), a humanized anti-HER2/neu antibody approved to treat breast cancer, pertuzumab (rhuMab-2C4, Omnitarg®), currently being developed by Genentech; an anti-HER2 antibody (U.S. Pat. No. 4,753,894; cetuximab (Erbitux®, Imclone) (U.S. Pat. No. 4,943,533; PCT Publication No. WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. No. 7,247,301), currently being developed by Genmab; 425, EMD55900, EMD62000, and EMD72000 (Merck KGaA) (U.S. Pat. No. 5,558,864; Murthy, et al. (1987) Arch. Biochem. Biophys. 252(2): 549-60; Rodeck, et al. (1987) J. Cell. Biochem. 35(4): 315-20: Kettleborough, et al. (1991) Protein Eng. 4(7): 773-83); ICR62 (Institute of Cancer Research) (PCT Publication No. WO 95/20045: Modjtahedi, et al. (1993) J. Cell. Biophys. 22(I-3): 129-46; Modjtahedi, et al. (1993) Br. J. Cancer 67(2): 247-53; Modjtahedi, et al. (1996) Br. J. Cancer 73(2): 228-35; Modjtahedi, et al. (2003) Int. J. Cancer 105(2): 273-80); TheraCIM hR3 (YM Biosciences, Canada and Centro de Immunologia Molecular, Cuba (U.S. Pat. Nos. 5,891,996; 6,506,883; Mateo, et al. (1997) Immunotechnol. 3(1): 71-81); mAb-806 (Ludwig Institute for Cancer Research, Memorial Sloan-Kettering) (Jungbluth, et al. (2003) Proc. Natl. Acad. Sci. USA. 100(2): 639-44); KSB-102 (KS Biomedix); MR1-1 (IVAX, National Cancer Institute) (PCT Publication No. WO 01/62931A2); and SC100 (Scancell) (PCT Publication No. WO 01/88138); alemtuzumab (Campath®, Millenium), a humanized mAb currently approved for treatment of B-cell chronic lymphocytic leukemia: muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, Ienercept, an p55TNF receptor Fc fusion previously developed by Roche, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-β antibody being developed by Cambridge Antibody Technology, ABT 874 (J695), an anti-IL-12 p40 antibody being developed by Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-B® an anti-Blys antibody being developed by Cambridge Antibody Technology and Human Genome Sciences Inc., TRAIL-R1 mAb, an anti-TRAIL-R1 antibody being developed by Cambridge Antibody Technology and Human Genome Sciences, Inc., Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumab), an anti-IgE antibody being developed by Genentech, Raptiva® (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GlycoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD 23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (Iabetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-HER2 antibody being developed by Medarex and Immuno-Designed Molecules, HuMax®-CD4, an anti-CD4 antibody being developed by Medarex and Genmab, HuMax-IL15, an anti-IL 15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF®, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLN01, an anti-Beta2 integrin antibody being developed by Xoma.

In another embodiment, the therapeutics include KRN330 (Kirin); huA33 antibody (A33, Ludwig Institute for Cancer Research); CNTO 95 (alpha V integrins, Centocor); MEDI-522 (alpha Vβ3integrin, Medimmune); volociximab (alpha Vβ1 integrin, Biogen/PDL); Human mAb 216 (B cell glycosolated epitope, NCl); BiTE MT103 (bispecific CD19×CD3, Medimmune); 4G7×H22 (Bispecific Bcell×FcgammaR1, Medarex/Merck Kga); rM28 (Bispecific CD28×MAPG, EP Patent No. EP1444268); MDX447 (EMD 82633) (Bispecific CD64×EGFR, Medarex); Catunmaxomab (removab) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Bristol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCl); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD122 (CD40, Novartis); SGN-40 (CD40, Seattle Genentics); MabCampath (Alemtuzumab) (CD52, Genzyme); MDX-1411 (CD70, Medarex); hLL1 (EPB-1) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Bristol Myers Squibb); Tremelimumab (Ticilimumab, CP-675,2) (CTLA4, Pfizer); HGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab)(DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam, Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF1-R, Pfizer); IMC-A12 (IGF1-R, Imclone); BIIB022(IGF-1R, Biogen); Mik-beta-1 (IL-2Rb (CD122), Hoffman-La Roche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTβR, Biogen); HuHMFG1 (MUC1, Antisoma/NCl); RAV12 (N-linked carbohydrate epitope, Raven); CAL (parathyroid hormone-related protein (PTH-rP), University of California); CT-011 (PD1, CureTech); MDX-1106 (ono-4538) (PD1, Medarex/Ono); Mab CT-011 (PD1, Curetech); IMC-3G3 (PDGFRa, Imclone); bavituximab (phosphatidylserine, Peregrine): huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFa, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab, PCT Publication No. WO/2000/034337, University of Texas); IMC-18F1 (VEGFR1, Imclone); IMC-1121 (VEGFR2, Imclone).

Examples of useful bispecific antibodies include, but are not limited to, those with one antibody directed against a tumor cell antigen and the other antibody directed against a cytotoxic trigger molecule such as anti-FcγRI/anti-CD 15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; bispecific antibodies with one antibody which binds specifically to a tumor antigen and another antibody which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; bispecific antibodies for converting enzyme activated prodrugs such as anti-CD30/anti-alkaline phosphatase (which catalyzes conversion of mitomycin phosphate prodrug to mitomycin alcohol); bispecific antibodies which can be used as fibrinolytic agents such as anti-fibrin/anti-tissue plasminogen activator (tPA), anti-fibrin/anti-urokinase-type plasminogen activator (uPA); bispecific antibodies for targeting immune complexes to cell surface receptors such as anti-low density lipoprotein (LDL)/anti-Fc receptor (e.g., FcγRI, FcγRII or FcγRIII); bispecific antibodies for use in therapy of infectious diseases such as anti-CD3/anti-herpes simplex virus (HSV), anti-T-cell receptor:CD3 complex/anti-influenza, anti-FcγR/anti-HIV; bispecific antibodies for tumor detection in vitro or in vivo such as anti-CEA/anti-EOTUBE, anti-CEA/anti-DPTA, anti- anti-p185^{HER2}/anti-hapten; bispecific antibodies as vaccine adjuvants (see Fanger, M W et al., Crit Rev Immunol. 1992; 12(34):101-24, which is incorporated by reference herein); and bispecific antibodies as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase (see Nolan, O, and O'Kennedy, R., Biochim Biophys Acta. 1990 Aug. 1; 1040(1):1-11, which is incorporated by reference herein). Examples of trispecific antibodies include anti-CD3/anti-CD4/anti-CD37, anti-CD3/anti-CD5/anti-CD37 and anti-CD3/anti-CD8/anti-CD37.

### Conjugation

In certain embodiments, the conjugate can be formed from a macromolecule that comprises one or more reactive groups. In certain embodiments, the conjugate can be formed from a macromolecule comprising all naturally encoded amino acids. Those of skill in the art will recognize that several naturally encoded amino acids include reactive groups capable of conjugation to a compound of Formula (I), (IA), (IB), (IC), (ID), (IE), (IF), (IG), (IH), (III), (IIIA), and (IIIB) or to a linker. These reactive groups include cysteine side chains, lysine side chains, and amino-terminal groups. In these embodiments, the conjugate can comprise a compound of Formula (I), (IA), (IB), (IC), (ID), (IE), (IF), (IG), (IH), (III), (IIIA), and (IIIB) or linker linked to the residue of an antibody reactive group. In these embodiments, the compound of Formula (I), (IA), (IB), (IC), (ID), (IE), (IF), (IG), (IH), (III), (IIIA), and (IIIB) precursor or linker precursor comprises a reactive group capable of forming a bond with an antibody or antigen binding fragment thereof reactive group. Typical reactive groups include maleimide groups, activated carbonates (including, but not limited to, p-nitrophenyl ester), activated esters (including, but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Further reactive groups are described in the sections and examples below.

### Reactive Groups

Reactive groups facilitate conjugation of the compounds of Formula (I)—(IH), (III), (IIIA), or (IIIB) as described herein to a second compound, such as an macromolecule (i.e., COMP) described herein to form a conjugate of Formula (II)-(IIH), (III), (IIIA), or (IIIB) as described herein. In certain embodiments, the reactive group is designated RG herein. Reactive groups can react via any suitable reaction mechanism known to those of skill in the art. In certain embodiments, a reactive group (RG) reacts through a [3+2] alkyne-azide cycloaddition reaction, inverse-electron demand Diels-Alder ligation reaction, thiol-electrophile reaction, or carbonyl-oxyamine reaction, as described in detail herein. In certain embodiments, the reactive group (RG) comprises an alkyne, strained alkyne, tetrazine, thiol, para-acetyl-phenylalanine residue, oxyamine, maleimide, or azide. In certain embodiments, the reactive group is

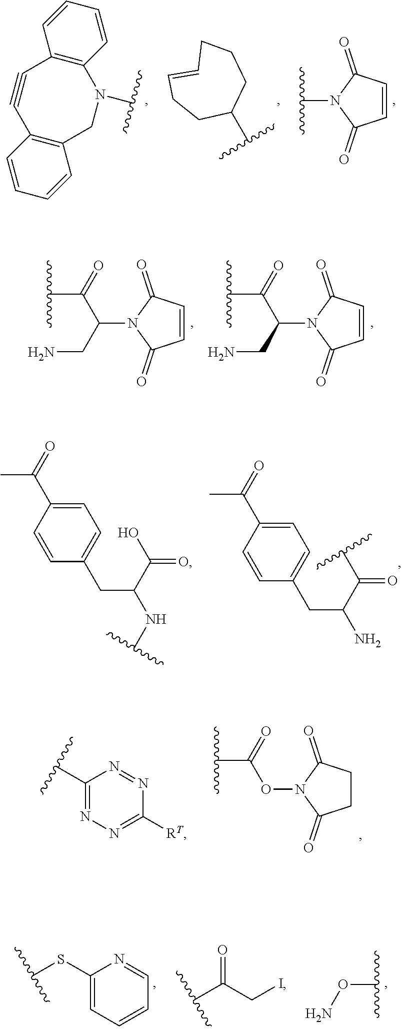

—N₃, or —SH; wherein R^{T} is lower alkyl. In certain embodiments, R^{T} is methyl, ethyl, or propyl. In some embodiments, R^{T} is methyl. In some embodiments, R^{T} is ethyl. In some embodiments, R^{T} is propyl. Additional reactive groups are described in, for example, U.S. Patent Application Publication No. 2014/0356385, U.S. Patent Application Publication No. 2013/0189287, U.S. Patent Application Publication No. 2013/0251783, U.S. Pat. Nos. 8,703,936, 9,145,361, 9,222,940, and 8,431,558.

After conjugation, a divalent residue of the reactive group (referred to as RL herein) is formed and is bonded to the residue of a second compound (e.g., COMP). The structure of the divalent residue is determined by the type of conjugation reaction employed to form the conjugate.

### [3+2] Alkyne-Azide Cycloaddition Reaction

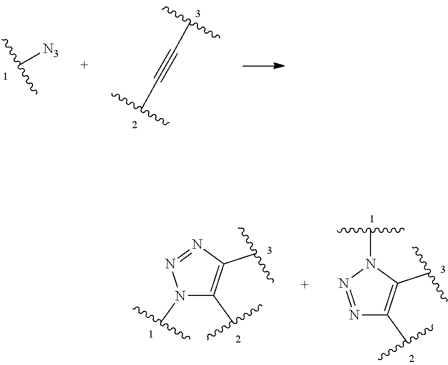

Advantageously, the compounds described herein comprising a conjugating alkyne group or an azide group facilitate selective and efficient reactions with a second compound comprising a complementary azide group or alkyne group. It is believed the azide and alkyne groups react in a 1,3-dipolar cycloaddition reaction to form a 1,2,3-triazolylene moiety which links the compounds described herein comprising an alkyne group or an azide group to the second compound. This reaction between an azide and alkyne to form a triazole is generally known to those in the art as a Huisgen cycloaddition reaction or a └3+2┘ alkyne-azide cycloaddition reaction.

The unique reactivity of azide and alkyne functional groups makes them useful for the selective modification of polypeptides and other biological molecules. Organic azides, particularly aliphatic azides, and alkynes are generally stable toward common reactive chemical conditions. In particular, both the azide and the alkyne functional groups are inert toward the side chains of the twenty common amino acids found in naturally-occurring polypeptides. It is believed that, when brought into close proximity, the “spring-loaded” nature of the azide and alkyne groups is revealed and azide and alkyne groups react selectively and efficiently via a [3+2] alkyne-azide cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the ┌3+2┐ alkyne-azide cycloaddition reaction involves a selective cycloaddition reaction [see, e.g., Padwa, A., in COMPREHENSIVE ORGANIC SYNTHESIS, Vol. 4, (ed. Trost, B. M., 1991), pp. 1069-1109; Huisgen, R. in 1,3-DIPOLAR CYCLOADDITION CHEMISTRY, (ed. Padwa, A., 1984), pp. 1-176] rather than a nucleophilic substitution, the incorporation of non-naturally encoded amino acids bearing azide and alkyne-containing side chains permits the resultant polypeptides to be modified selectively at the position of the non-naturally encoded amino acid. Cycloaddition reactions involving azide or alkyne-containing compounds can be carried out at room temperature under aqueous conditions by the addition of Cu(II) (including, but not limited to, catalytic amounts of CuSO₄) in the presence of a reducing agent for reducing Cu(II) to Cu(I), in situ, in catalytic amounts. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, but are not limited to, ascorbate, metallic copper, quinine, hydroquinone, vitamin K, glutathione, cysteine, Fe²⁺, Co²⁺, and an applied electric potential.

In certain embodiments when a conjugate is formed through a [3+2] alkyne-azide cycloaddition reaction, the divalent residue of the reactive group (e.g., RL) comprises a triazole ring or fused cyclic group comprising a triazole ring. In certain embodiments, when a conjugate is formed through a strain-promoted [3+2] alkyne-azide cycloaddition (SPAAC) reaction, the divalent residue of the reactive group (e.g., RL) is

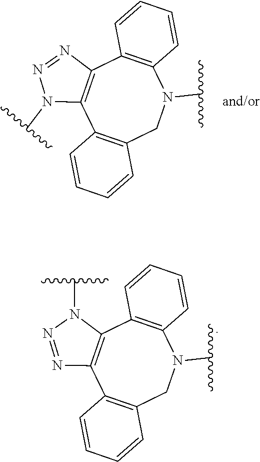 [SMILES: *N1Cc2ccccc2-c2c(nnn2*)-c2ccccc21 ; *N1Cc2ccccc2-c2nnn(*)c2-c2ccccc21]

If a conjugates of Formula (II)-(IIH), (IV), (IVA), or (IVB) is formed by a [3+2] alkyne-azide cycloaddition, the conjugate encompasses both regioisomers. In certain embodiments, a conjugate of Formula (II)-(IIH), (IV), (IVA), or (IVB) is a mixture of regioisomers formed from a [3+2] alkyne-azide cycloaddition.

### Inverse Electron Demand Ligation Reaction

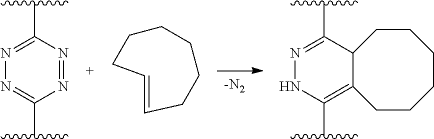

Advantageously, compounds comprising a terminal tetrazine or strained alkene group facilitate selective and efficient reactions with a second compound comprising a strained alkene or tetrazine group. It is believed that the tetrazine and strained alkene react in an inverse-demand Diels-Alder reaction followed by a retro-Diels-Alder reaction which links compounds comprising a terminal tetrazine or strained alkene group to the second compound. The reaction is believed to be specific, with little to no cross-reactivity with functional groups within biomolecules. The reaction may be carried out under mild conditions, for example, at room temperature and without a catalyst. This reaction between a tetrazine and a strained alkene is generally known to those in the art as a tetrazine ligation reaction.

In certain embodiments, when a conjugate is formed through a tetrazine inverse electron demand Diels-Alder ligation reaction, the divalent residue of the reactive group (e.g., RL) comprises a fused bicyclic ring having at least two adjacent nitrogen atoms in the ring. In certain embodiments, when a conjugate is formed through a tetrazine inverse electron demand Diels-Alder ligation reaction, the divalent residue of the reactive group (e.g., RL) is

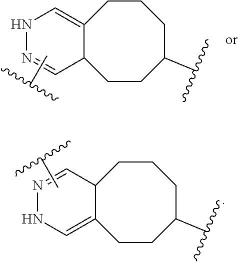

If a conjugates of Formula (II)-(IIH), (IV), (IVA), or (IVB) is formed by an inverse electron demand ligation reaction, the conjugate encompasses both regioisomers. In certain embodiments, a conjugate of Formula (II)-(IIH), (IV), (IVA), or (IVB) is a mixture of regioisomers formed from a inverse electron demand ligation reaction.

### Thiol Reactions

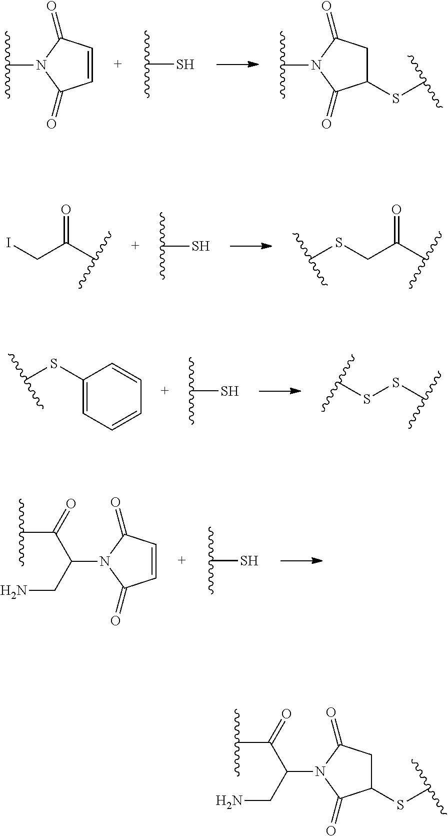

Advantageously, compounds comprising a terminal thiol group or suitable electrophilic or disulfide-forming group facilitate selective and efficient reactions with a second compound comprising a complementary electrophilic or disulfide-forming group or thiol group. These reactions are believed to be selective with little to no cross-reactivity with functional groups within biomolecules. In some embodiments, the thiol reaction does not include reaction of a maleimide group.

In certain embodiments, when a conjugate is formed through a thiol-maleimide reaction, the divalent residue of the reactive group comprises

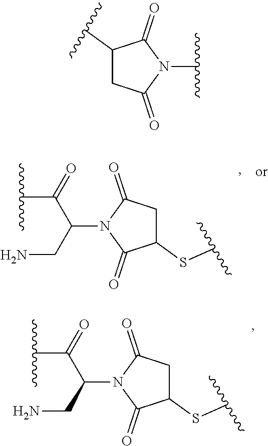

and a sulfur linkage. In certain embodiments, when a conjugate of Formula (II)-(IIH), (IV), (IVA), or (IVB) is formed through a thiol-maleimide reaction,

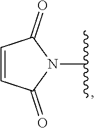 [SMILES: *N1C(=O)C=CC1=O]

the divalent residue of the reactive group (e.g., RL) is

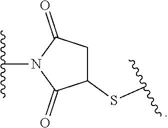 [SMILES: *SC1CC(=O)N(*)C1=O]

In other embodiments, when a conjugate of Formula (II)-(IIH), (IV), (IVA), or (IVB) is formed through a thiol-maleimide reaction,

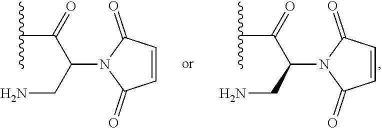 [SMILES: *C(=O)C(CN)N1C(=O)C=CC1=O ; *C(=O)[C@H](CN)N1C(=O)C=CC1=O]

the divalent residue of the reactive group (e.g., RL) is

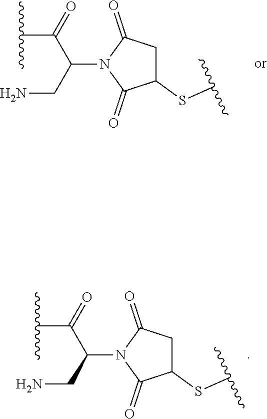 [SMILES: *SC1CC(=O)N(C(CN)C(*)=O)C1=O ; *SC1CC(=O)N([C@@H](CN)C(*)=O)C1=O]

### Carbonyl-Oxyamine Reaction

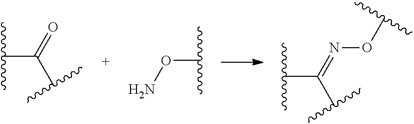

Advantageously, compounds comprising a terminal carbonyl or oxyamine group facilitate selective and efficient reactions with a second compound comprising an oxyamine or carbonyl group. It is believed that the carbonyl and oxyamine react to form an oxime linkage. The reaction is believed to be specific, with little to no cross-reactivity with functional groups within biomolecules.

In certain embodiments when a conjugate of Formula (II)-(IIH), (IV), (IVA), or (IVB) is formed through an oxime conjugation reaction, the divalent residue of the reactive group comprises a divalent residue of a non-natural amino acid. In certain embodiments when a conjugate of Formula (II)-(IIH), (IV), (IVA), or (IVB) is formed through an oxime conjugation reaction, the divalent residue of the reactive group (e.g., RL) is

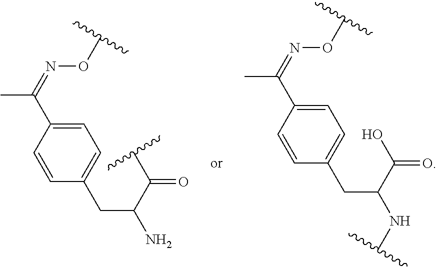 [SMILES: *O/N=C(/C)c1ccc(CC(N)C(*)=O)cc1 ; *NC(Cc1ccc(/C(C)=N\O*)cc1)C(=O)O]

In certain embodiments when a conjugate of Formula (II)-(IIH), (IV), (IVA), or (IVB) is formed through an oxime conjugation reaction, the divalent residue of the reactive group comprises an oxime linkage. In certain embodiments when a conjugate of Formula (II)-(IIH), (IV), (IVA), or (IVB) is formed through an oxime conjugation reaction, the divalent residue of the reactive group (e.g., RL) is

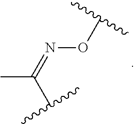 [SMILES: *O/N=C(\*)C]

### Thiol-N-Hydroxysuccinimide Reaction

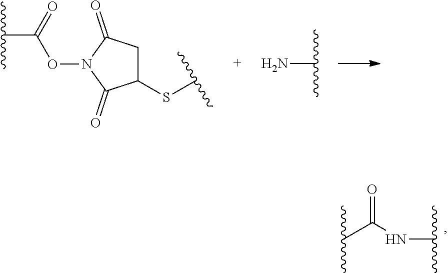

Advantageously, compounds comprising a terminal thiol-N-hydroxysuccinimide facilitate a reaction with a second compound comprising an amine group, for example a glutamine, to form an amide.

In certain embodiments when a conjugate of Formula (II)-(IIH), (IV), (IVA), or (IVB) is formed through a thiol-N-hydroxysuccinimide conjugation reaction, the divalent residue of the reactive group comprises a divalent residue of an amino acid, for example glutamine. In certain embodiments when a conjugate of Formula (II)-(IIH), (IV), (IVA), or (IVB) is formed through a thiol-N-hydroxysuccinimide conjugation reaction, the divalent residue of the reactive group (e.g., RL) is

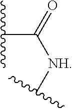 [SMILES: *NC(*)=O]

In certain embodiments when a conjugate of Formula (II)-(IIH), (IV), (IVA), or (IVB) is formed through a thiol-N-hydroxysuccinimide conjugation reaction, the divalent residue of the reactive group comprises an amide linkage.

### Other Reactions

Other suitable conjugation reactions are described in the literature. See, for example, Lang, K. and Chin, J. 2014, Bioorthogonal Reactions for Labeling Proteins, ACS Chem Biol 9, 16-20; Paterson, D. M. et al. 2014, Finding the Right (Bioorthogonal) Chemistry, ACS Chem Biol 9, 592-605; King, M. and Wagner, A. 2014, Developments in the Field of Bioorthogonal Bond Forming Reactions—Past and Present Trends, Bioconjugate Chem., 2014, 25 (5), pp 825-839; and Ramil, C. P. and Lin, Q., 2013, Bioorthogonal chemistry: strategies and recent developments, Chem Commun 49, 11007-11022.

### Releasing Reactions

Releasing Reactions are reactions that act to release a biologically active portion of a compound or conjugate described herein from the compound or conjugate in vivo and/or in vitro. In certain embodiments, the released biologically active portion is a compound described elsewhere herein (e.g., cytotoxic agents), or a pharmaceutically acceptable salt, solvate, stereoisomer, or tautomer thereof. One example of a releasing reaction is an intramolecular reaction between an eliminator group and a release trigger group of a compound or conjugate described herein to release a biologically active portion of a compound or conjugate described herein. The eliminator group may itself devolve into two reactive components, as exemplified in these reactions where X is a drug having a heteroatom nitrogen or oxygen for linkage. Exemplary Releasing Reactions are depicted in the scheme below:

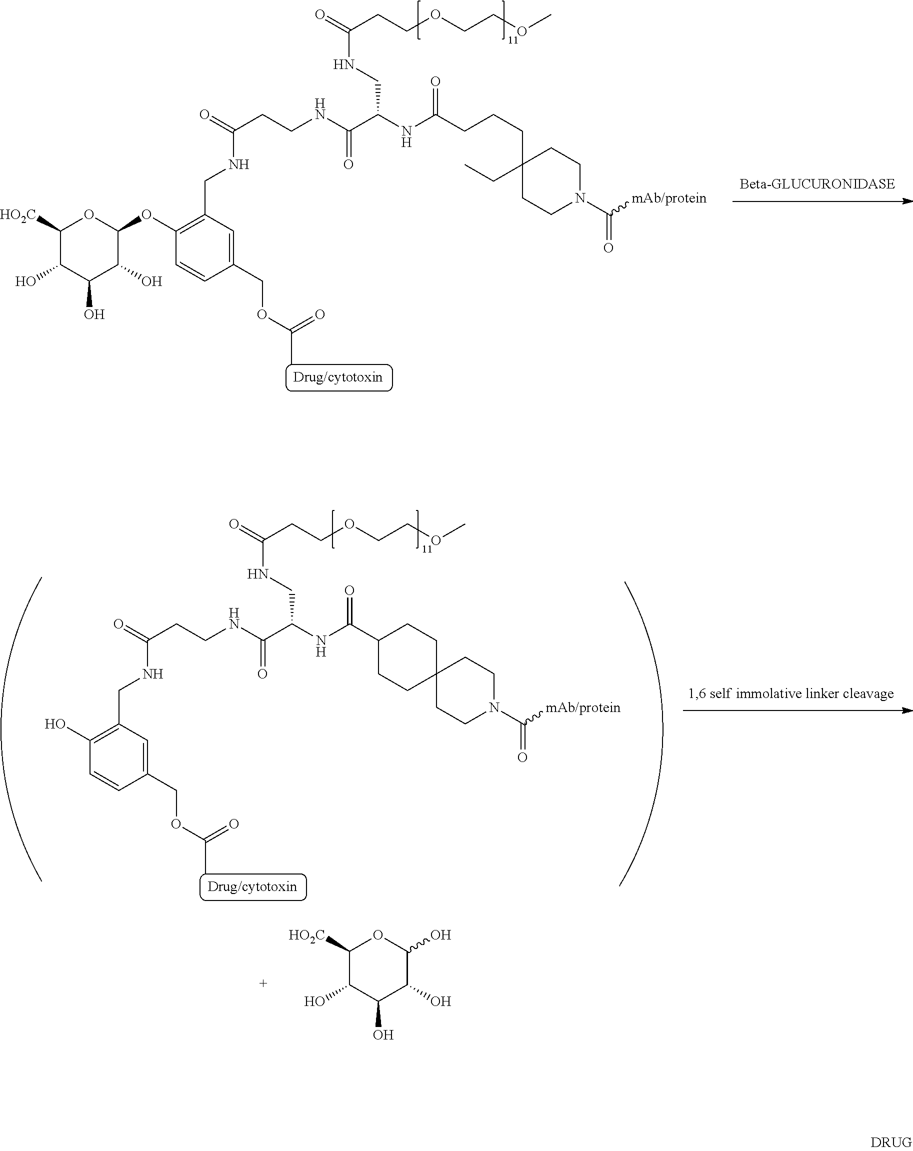

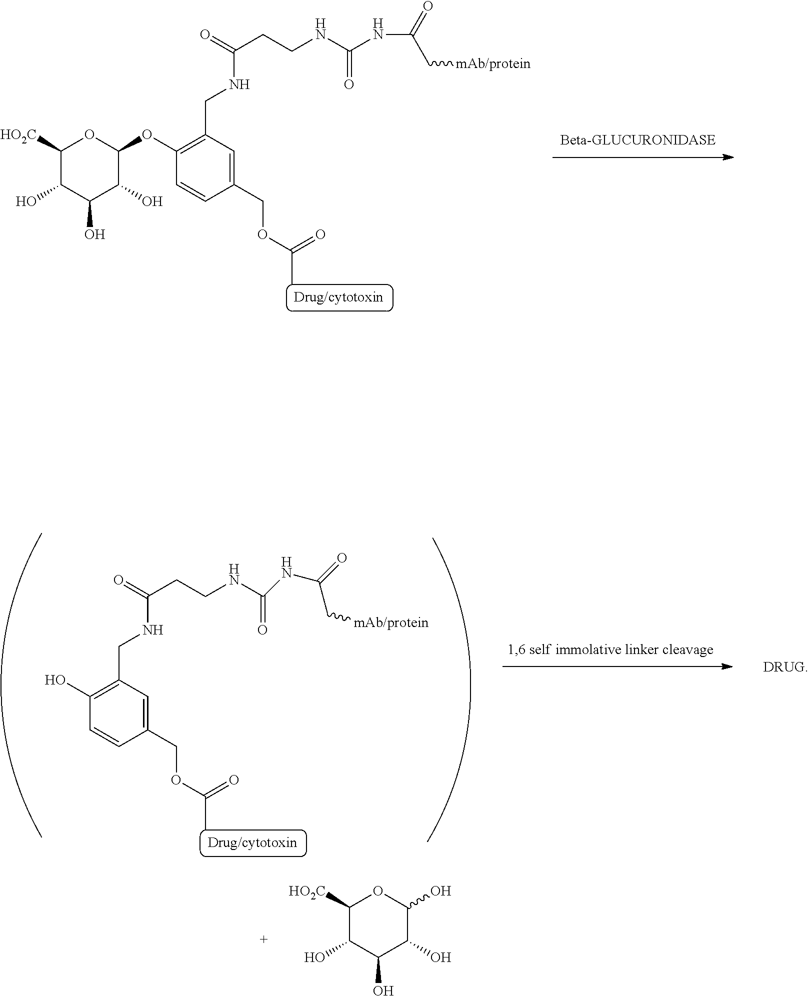

### Water Soluble Polymers

In certain embodiments, a compound or conjugate described herein comprises one or more water soluble polymers. A wide variety of macromolecular polymers and other molecules can be linked to the polypeptides described herein to modulate biological properties of the polypeptide, and/or provide new biological properties to the polypeptide. These macromolecular polymers can be linked to the polypeptide via a naturally encoded amino acid, via anon-naturally encoded amino acid, or any functional substituent of a natural or modified amino acid, or any substituent or functional group added to a natural or modified amino acid. The molecular weight of the polymer may include a wide range including, but not limited to, between about 100 Da and about 100,000 Da or more.

The polymer selected may be water soluble so that a protein to which it is attached does not precipitate in an aqueous environment, such as a physiological environment. The polymer may be branched or unbranched. In certain embodiments, for therapeutic use of the end-product preparation, the polymer will be pharmaceutically acceptable.

In certain embodiments, the proportion of polyethylene glycol molecules to polypeptide molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. Regarding molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer:protein ratio.

The water soluble polymer may be any structural form including, but not limited to, linear, forked, or branched. Typically, the water soluble polymer is a poly(alkylene glycol), such as poly(ethylene glycol) (PEG), but other water soluble polymers can also be employed. By way of example, PEG is used to describe certain embodiments.

PEG is a well-known, water soluble polymer that is commercially available or can be prepared by ring-opening polymerization of ethylene oxide according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of a PEG, and can be represented as linked to a polypeptide by the formula: X′O—(CH₂CH₂O)ₙ—CH₂CH₂—Y′ where n is an integer selected from 2 to 10,000, X′ is hydrogen or a terminal modification including, but not limited to, C₁₋₄ alkyl, and Y′ is the attachment point to the polypeptide.

In some cases, a PEG terminates on one end with hydroxy or methoxy, i.e., X′ is hydrogen or CH₃ (aka “methoxy PEG”). Alternatively, the PEG can terminate with a PEG reactive group, thereby forming a bifunctional polymer. Typical PEG reactive groups can include those reactive groups that are commonly used to react with the functional groups found in the twenty common amino acids (including, but not limited to, maleimide groups, activated carbonates (including, but not limited to, p-nitrophenyl ester), activated esters (including, but not limited to, N-hydroxysuccinimide, p-nitrophenyl ester, and aldehydes) as well as functional groups that are inert to the twenty common amino acids, but that react specifically with complementary functional groups present in non-naturally encoded amino acids (including, but not limited to, azide groups and/or alkyne groups). It is noted that the other end of the PEG, which is shown in the above formula by Y′, will attach either directly or indirectly to a polypeptide via a naturally-occurring or non-naturally encoded amino acid. For instance, Y′ may be an amide, carbamate, or urea linkage to an amine group (including, but not limited to, the epsilon amine of lysine or the N-terminus) of the polypeptide. Alternatively, Y′ may be a maleimide linkage to a thiol group (including, but not limited to, the thiol group of cysteine). Alternatively, Y′ may be a linkage to a residue not commonly accessible via the twenty common amino acids. For example, an azide group on the PEG can be reacted with an alkyne group on the polypeptide to form a Huisgen [3+2] cycloaddition product. Alternatively, an alkyne group on the PEG can be reacted with an azide group present in a non-naturally encoded amino acid, such as the modified amino acids described herein, to form a similar product. In some embodiments, a strong nucleophile (including, but not limited to, hydrazine, hydrazide, hydroxylamine, or semicarbazide) can be reacted with an aldehyde or ketone group present in a non-naturally encoded amino acid to form a hydrazone, oxime, or semicarbazone, as applicable, which in some cases can be further reduced by treatment with an appropriate reducing agent. Alternatively, the strong nucleophile can be incorporated into the polypeptide via a non-naturally encoded amino acid and used to react preferentially with a ketone or aldehyde group present in the water soluble polymer.

Any molecular mass for a PEG can be used as practically desired including, but not limited to, from about 100 Daltons (Da) to 100,000 Da or more as desired (including, but not limited to, in certain embodiments 0.1-50 kDa or 10-40 kDa). Branched chain PEGs including, but not limited to, PEG molecules with each chain having a molecular weight (MW) ranging from 1-100 kDa (including, but not limited to, 1-50 kDa or 5-20 kDa) can also be used. A wide range of PEG molecules are described in the Shearwater Polymers, Inc. catalog, and the Nektar Therapeutics catalog, each incorporated herein by reference.

Generally, at least one terminus of the PEG molecule is available for reaction with the remainder of the compound of Formula (I)—(IVB). For example, PEG derivatives bearing alkyne and azide moieties for reaction with amino acid side chains can be used to attach PEG to non-naturally encoded amino acids as described herein. If the non-naturally encoded amino acid comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the non-naturally encoded amino acid comprises an alkyne, then the PEG will typically contain an azide moiety to effect formation of the [3+2] Huisgen cycloaddition product. If the non-naturally encoded amino acid comprises a carbonyl group, the PEG will typically comprise a nucleophile (including, but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described herein can be used (i.e., an azide moiety in the non-naturally encoded amino acid can be reacted with a PEG derivative containing an alkyne).

In some embodiments, the polypeptide variant with a PEG derivative contains a chemical functionality that is reactive with the chemical functionality present on the side chain of the non-naturally encoded amino acid.

In certain embodiments, the water soluble polymer is an azide- or acetylene-containing polymer comprising a water soluble polymer backbone having an average molecular weight from about 800 Da to about 100,000 Da. The polymer backbone of the water-soluble polymer can be poly(ethylene glycol). However, it should be understood that a wide variety of water soluble polymers including, but not limited to, poly(ethylene)glycol and other related polymers, including poly(dextran) and poly(propylene glycol), are also suitable for use and that the use of the term “PEG” or “poly(ethylene glycol)” is intended to encompass and include all such molecules. The term “PEG” further includes, but is not limited to, poly(ethylene glycol) in any of its forms, including bifunctional PEG, multiarmed PEG, derivatized PEG, forked PEG, branched PEG, pendent PEG (i.e., PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol, and sorbitol. The central branch moiety can also be derived from several amino acids, such as lysine. The branched poly(ethylene glycol) can be represented in general form as R-(-PEG-OH)ₘ in which R is derived from a core moiety, such as glycerol, glycerol oligomers, or pentaerythritol, and m represents the number of arms. Multi-armed PEG molecules, such as those described in U.S. Pat. Nos. 5,932,462; 5,643,575; 5,229,490; and 4,289,872; U.S. Pat. Appl. No. 2003/0143596; and WO 96/21469 and WO 93/21259, each of which is incorporated by reference herein in its entirety, can also be used as the polymer backbone.

Branched PEG can also be in the form of a forked PEG represented by PEG(-Y″CHZ₂)ₙ, where Y″ is a linking group and Z is an activated terminal group linked to CH by a chain of atoms of defined length.

Yet another branched form, the pendant PEG, has PEG reactive groups, such as carboxyl, along the PEG backbone rather than at the end of PEG chains.

In addition to these forms of PEG, the polymer can also be prepared with weak or degradable linkages in the backbone. For example, PEG can be prepared with ester linkages in the polymer backbone that are subject to hydrolysis. As shown herein, this hydrolysis results in cleavage of the polymer into fragments of lower molecular weight: -PEG-CO₂-PEG-+H₂O→PEG-CO₂H+HO-PEG-. It is understood by those skilled in the art that the term “poly(ethylene glycol)” or “PEG” represents or includes all the forms known in the art including, but not limited to, those disclosed herein.

Many other polymers are also suitable for use. In some embodiments, polymer backbones that are water-soluble, with from two to about three hundred termini, are particularly suitable. Examples of suitable polymers include, but are not limited to, other poly(alkylene glycols), such as poly(propylene glycol) (“PPG”), copolymers thereof (including, but not limited to, copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

Those of ordinary skill in the art will recognize that the foregoing list for substantially water-soluble backbones is by no means exhaustive and is merely exemplary, and that all polymeric materials having the qualities described herein are contemplated as being suitable for use.

In some embodiments the polymer derivatives are “multi-functional,” meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

### Compositions and Uses

### Pharmaceutical Compositions and Methods of Administration

The conjugates provided herein can be formulated into pharmaceutical compositions using methods available in the art and those disclosed herein. Any of the conjugates provided herein can be provided in the appropriate pharmaceutical composition and be administered by a suitable route of administration.

The methods provided herein encompass administering pharmaceutical compositions comprising at least one conjugate provided herein and one or more compatible and pharmaceutically acceptable carriers. In this context, the term “pharmaceutically acceptable” means approved by a regulatory agency of the Federal or state government, or listed in the U.S. Pharmacopeia or other generally recognized pharmacopeia for use in animals, and in certain embodiments in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils including petroleum, animal, vegetable, or oils of synthetic origin, such as peanut oil, soybean oil, mineral oil, sesame oil, and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin. E. W., Remington's Pharmaceutical Sciences.

In clinical practice the pharmaceutical compositions or conjugates provided herein may be administered by any route known in the art. Exemplary routes of administration include, but are not limited to, oral, inhalation, intraarterial, intradermal, intramuscular, intraperitoneal, intravenous, nasal, parenteral, pulmonary, and subcutaneous routes. In some embodiments, a pharmaceutical composition or conjugate provided herein is administered orally. In some embodiments, a pharmaceutical composition or conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing, and stabilizing agents. Sterilization can be carried out in several ways, for example, using a bacteriological filter, via radiation, or via heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic conjugates.

The pharmaceutical composition may comprise one or more pharmaceutical excipients. Any suitable pharmaceutical excipient may be used, wherein a person of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. Whether a particular excipient is suitable for incorporation into a pharmaceutical composition or dosage form depends on a variety of factors well known in the art including, but not limited to, the way in which the dosage form will be administered to a subject and the specific conjugate in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6^{th} Ed. (2009), incorporated by reference herein in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long-chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6^{th} Ed. (2009), The Pharmaceutical Press, incorporated by reference herein in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising a conjugate, since, in some embodiments, water can facilitate the degradation of some antibodies or antigen binding fragments thereof.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmacopeia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which a conjugate will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to Water for Injection USP; aqueous vehicles such as, but not limited to, Sodium Chloride Injection, Ringer's Injection, Dextrose Injection, Dextrose and Sodium Chloride Injection, and Lactated Ringer's Injection, water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### Dosage and Unit Dosage Forms

In human therapeutics, the physician will determine the posology considered most appropriate according to a preventive or curative treatment and according to the age, weight, condition, and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies or antigen binding fragments thereof.

The amount of the conjugate or composition which will be effective in the prevention or treatment of a disorder or one or more symptoms thereof will vary with the nature and severity of the disease or condition, and the route by which the conjugate is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a conjugate or composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram).

The dose can be administered according to a suitable schedule. It may be necessary to use dosages of the antibody conjugate outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat, or ameliorate such disorders, but insufficient to cause, or sufficient to reduce, adverse effects associated with the antibodies or antigen binding fragments thereof provided herein are also encompassed by the described dosage amounts and dose frequency schedules herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of a conjugate or composition provided herein followed by one or more maintenance doses.

In certain embodiments, a dose of a conjugate or composition provided herein can be administered to achieve a steady-state concentration of the conjugate in blood or serum of the subject. The steady-state concentration can be determined by measurement according to techniques available to those of skill or can be based on the physical characteristics of the subject such as height, weight, and age.

### Therapeutic Applications

For therapeutic applications, the conjugates are administered to a mammal, in certain embodiments, a human, in a pharmaceutically acceptable dosage form such as those known in the art and those discussed herein. For example, the conjugates of this disclosure may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The conjugates also are suitably administered by peritumoral, intralesional, or perilesional routes, to exert local as well as systemic therapeutic effects. The intraperitoneal route may be particularly useful, for example, in the treatment of ovarian tumors.

The conjugates provided herein may be useful for the treatment of any disease or condition described herein (e.g., inflammatory and/or proliferative disease or condition). In some embodiments, the disease or condition is a disease or condition that can be diagnosed by overexpression of an antigen. In some embodiments, the disease or condition is a disease or condition that can benefit from treatment with a macromolecule. In some embodiments, the disease or condition is abnormal cellular proliferation.

In some embodiments, the disease or condition is a cancer. In certain embodiments, the cancer is small cell lung cancer, non-small cell lung cancer, ovarian cancer, platinum-resistant ovarian cancer, ovarian adenocarcinoma, endometrial cancer, breast cancer, breast cancer which overexpresses HER2, triple-negative breast cancer, a lymphoma, large cell lymphoma; diffuse mixed histiocytic and lymphocytic lymphoma; follicular B cell lymphoma, colon cancer, colon carcinoma, colon adenocarcinoma, colorectal adenocarcinoma, melanoma, prostate cancer, or multiple myeloma.

### Diagnostic Applications

In some embodiments, the conjugates provided herein are used in diagnostic applications. These assays may be useful, for example, in making a diagnosis and/or prognosis for a disease, such as a cancer.

In some diagnostic and prognostic applications or embodiments, the conjugate may be labeled with a detectable moiety. Suitable detectable moieties include, but are not limited to, radioisotopes, fluorescent labels, and enzyme-substrate labels. In another embodiment, the conjugate need not be labeled, and the presence of the conjugate can be detected using a labeled antibody or antigen binding fragment thereof which specifically binds to the conjugate.

### Kits

In some embodiments, a conjugate provided herein is provided in the form of a kit (i.e., a packaged combination of reagents in predetermined amounts with instructions for performing a procedure). In some embodiments, the procedure is a diagnostic assay. In certain embodiments, the procedure is a therapeutic procedure.

In some embodiments, the kit further comprises a solvent for the reconstitution of the conjugate. In some embodiments, the conjugate is provided in the form of a pharmaceutical composition.

In some embodiments, the kits can include a conjugate or composition provided herein, an optional second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a conjugate or a composition provided herein, or a second agent or composition, can include a dosage such that when administered to a subject, a therapeutically or prophylactically effective plasma level of the compound or composition can be maintained in the subject for at least one day. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, plastic-foil laminated envelopes, and the like. If e-beam sterilization techniques are employed, the packaging should have sufficiently low density to permit sterilization of the contents.

### Preparation and Synthetic Procedures

### Conjugation

The conjugates can be prepared by standard techniques. In certain embodiments, a macromolecule is contacted with a compound of Formula (I)—(IH), (III), (IIIA), or (IIIB) under conditions suitable for forming a bond from the macromolecule to the compound of Formula (I)—(IH), (III), (IIIA), or (IIIB) to form a conjugate, for example of Formula (II)-(IIH), (IV), (IVA), or (IVB). In certain embodiments, a macromolecule is contacted with a linker precursor under conditions suitable for forming a bond from the macromolecule to the linker. The resulting macromolecule-linker is contacted with a compound or drug moiety under conditions suitable for forming a bond from the macromolecule-linker to the compound or drug moiety to form a conjugate. In certain embodiments, a compound or drug moiety is contacted with a linker precursor under conditions suitable for forming a bond from the compound or drug moiety to the linker. The resulting compound-linker or drug moiety-linker is contacted with a macromolecule under conditions suitable for forming a bond from the compound-linker or drug moiety-linker to the macromolecule to form a conjugate. For example, in certain embodiments, the second compound comprises a tetrazine; and RG comprises a strained alkene. In some embodiments, RG is

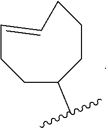 [SMILES: *C1CC/C=C/CCC1]

In certain embodiments, the second compound comprises an azide; and RG comprises an alkyne. In some embodiments, RG is

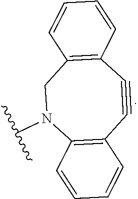 [SMILES: *N1Cc2ccccc2C#Cc2ccccc21]

In certain embodiments, the second compound comprises an alkyne; and RG comprises an azide. In certain embodiments, the second compound comprises a strained alkene; and RG comprises a tetrazine. In certain embodiments, the second compound comprises a thiol; and RG comprises a maleimide. In some embodiments, RG is

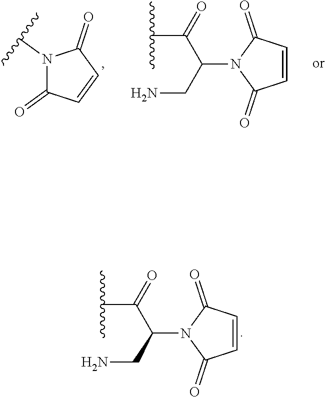 [SMILES: *N1C(=O)C=CC1=O ; *C(=O)C(CN)N1C(=O)C=CC1=O ; *C(=O)[C@H](CN)N1C(=O)C=CC1=O]

In certain embodiments, the second compound comprises a maleimide; and RG comprises a thiol. In some embodiments, the second compound comprises

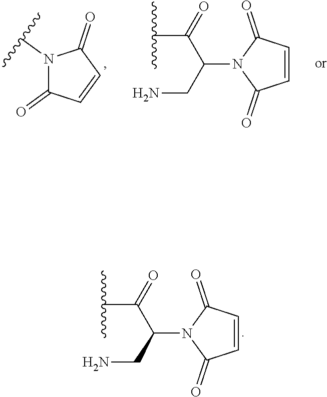 [SMILES: *N1C(=O)C=CC1=O ; *C(=O)C(CN)N1C(=O)C=CC1=O ; *C(=O)[C@H](CN)N1C(=O)C=CC1=O]

In certain embodiments, the second compound comprises a carbonyl; and RG comprises an oxyamine. In some embodiments, RG is

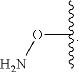 [SMILES: *ON]

In some embodiments, second compound comprises

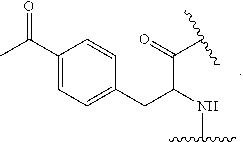 [SMILES: *NC(Cc1ccc(C(C)=O)cc1)C(*)=O]

In certain embodiments, the second compound comprises an oxyamine; and RG comprises a carbonyl. In certain embodiments, RG is

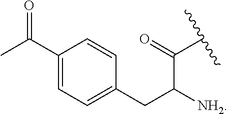 [SMILES: *C(=O)C(N)Cc1ccc(C(C)=O)cc1]

In some embodiments, RG is

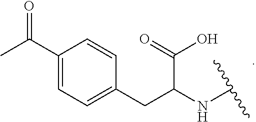 [SMILES: *NC(Cc1ccc(C(C)=O)cc1)C(=O)O]

In some embodiments, RG is

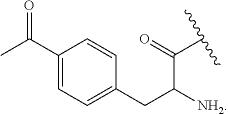 [SMILES: *C(=O)C(N)Cc1ccc(C(C)=O)cc1]

In certain embodiments, second compound comprises

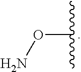 [SMILES: *ON]

In certain embodiments, the second compound is a polypeptide. In certain embodiments, the second compound is an antibody. In certain embodiments, the second compound is an antibody chain. Suitable linkers for preparing the conjugates are disclosed herein, and exemplary conditions for conjugation are described in the Examples below.

### EXAMPLES

The compounds provided herein can be prepared, isolated, or obtained by any method apparent to those of skill in the art. Compounds provided herein can be prepared according to the exemplary preparation schemes provided below. Reaction conditions, steps, and reactants not provided in the exemplary preparation schemes would be apparent to, and known by, those skilled in the art. As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); h, hr, or hrs (hours); min (minutes); MS (mass spectrometry); ESI (electrospray ionization); LCMS (Liquid chromatography-mass spectrometry) TLC (thin layer chromatography); HPLC (high performance liquid chromatography); rt (room temperature); atm (atmospheres); calcd (calculated); equiv (equivalents); CDCl₃ (deuterated chloroform); DBCO (dibenzocyclooctyne-amine); DCE (dichloroethane); DCM (dichloromethane); DIPEA (diisopropylethylamine); DMSO (dimethylsulfoxide); DMSO-d₆ (deuterated dimethylsulfoxide); ; EtOAc (ethyl acetate); EtOH (ethanol); MeCN (acetonitrile); MeOH (methanol); RB (round-bottomed flask); TFA (trifluoroacetic acid); THF (tetrahydrofuran); DMF (dimethylformamide); and BOC (t-butyloxycarbonyl).

For all of the following examples, standard workup and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

Unless otherwise indicated, all anhydrous solvents were commercially obtained and stored in Sure-Seal bottles under nitrogen. All other reagents and solvents were purchased as the highest grade available and used without further purification. NMR spectra were recorded on Avance II HD (500 MHz) spectrometer equipped with 5 mm Prodiogy H/F—BBO cryoprobe, BCU—I temperature controller. Chemical shifts (δ) are reported in parts per million (ppm) referenced to tetramethylsilane at δ 0.00 and coupling constants (J) are reported in Hz. Low resolution mass spectral data were acquired on a Agilent G6125B spectrometer interfaced with an Agilent 1260 high performance liquid chromatography instrument for LC-MS. Products were purified by RP-HPLC method, System: Shimadzu LC with CTC IFC, Phenomenex Gemini NX 5μ, C18, 110 Å, 150×50 mm reverse phase column using a linear gradient of mobile phase B (CH₃CN) in A (water with 0.1% TFA) at 50 mL/min. Analytical HPLC was conducted on a Waters 2695 instrument. For analytical HPLC the stationary phase used was a Phenomenex Gemini NX 5μ, C18, 110 Å, 150×4.6 mm RP column. Products were eluted on either acidic linear gradients (designated gradient A) of mobile phase B (CH₃CN with 0.05% TFA; 5% to 95% over 20 min) in A (0.05% aqueous TFA) at a flow rate of 1.0 mL/min. Preparative HPLC purifications were performed on Shimadzu LC with CTC IFC. All other preparative normal phase purifications were done by standard flash silica gel chromatography using an ISCO flash system.

### Example 1. Synthesis of DBCO-nnAA-PEG13-AAN-Exatecan (Compound 1)

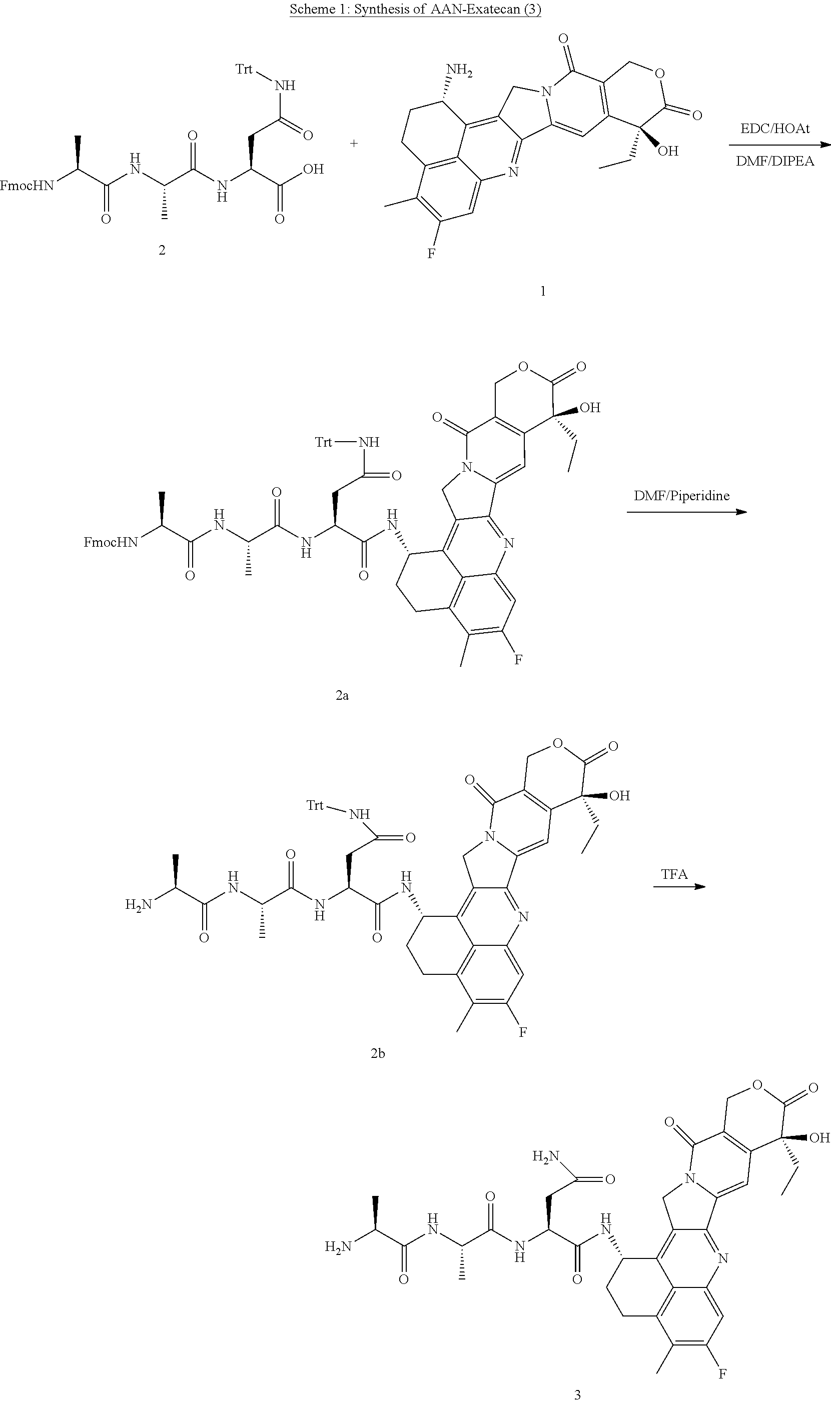

To a suspension of exatecan mesylate 1 (MsOH salt, 150 mg, 0.28 mmol) in anhydrous DMF (3 mL) at room temperature was added Fmoc-AAN(Trt)-OH 2 (250 mg, 0.33 mmol), EDC (65 mg, 0.34 mmol), HOAt (46 mg, 0.34 mmol), and DIPEA (53 uL). The reaction was stirred at RT for 1 h, LCMS showed the desired product, then 0.3 mL of piperidine was added. The mixture was stirred for 5 min, and then added to a 1/1 mixture of hexane/diethyl ether (45 mL). The precipitate was collected by centrifugation and the solvent was removed by decantation to give the compound 2b. The residue 2b was dissolved in 3 mL of TFA and the mixture was stirred for 10 min at RT. Then the TFA was removed under reduced pressure, the crude mixture was purified by reverse phase HPLC. Pure Fractions were lyophilized to give compound 3 as a TFA salt (63 mg); LCMS m/z (ESI⁺): calculated for C₃₄H₃₈FN₇O₈, 691.28. found 692.4 (M+H).

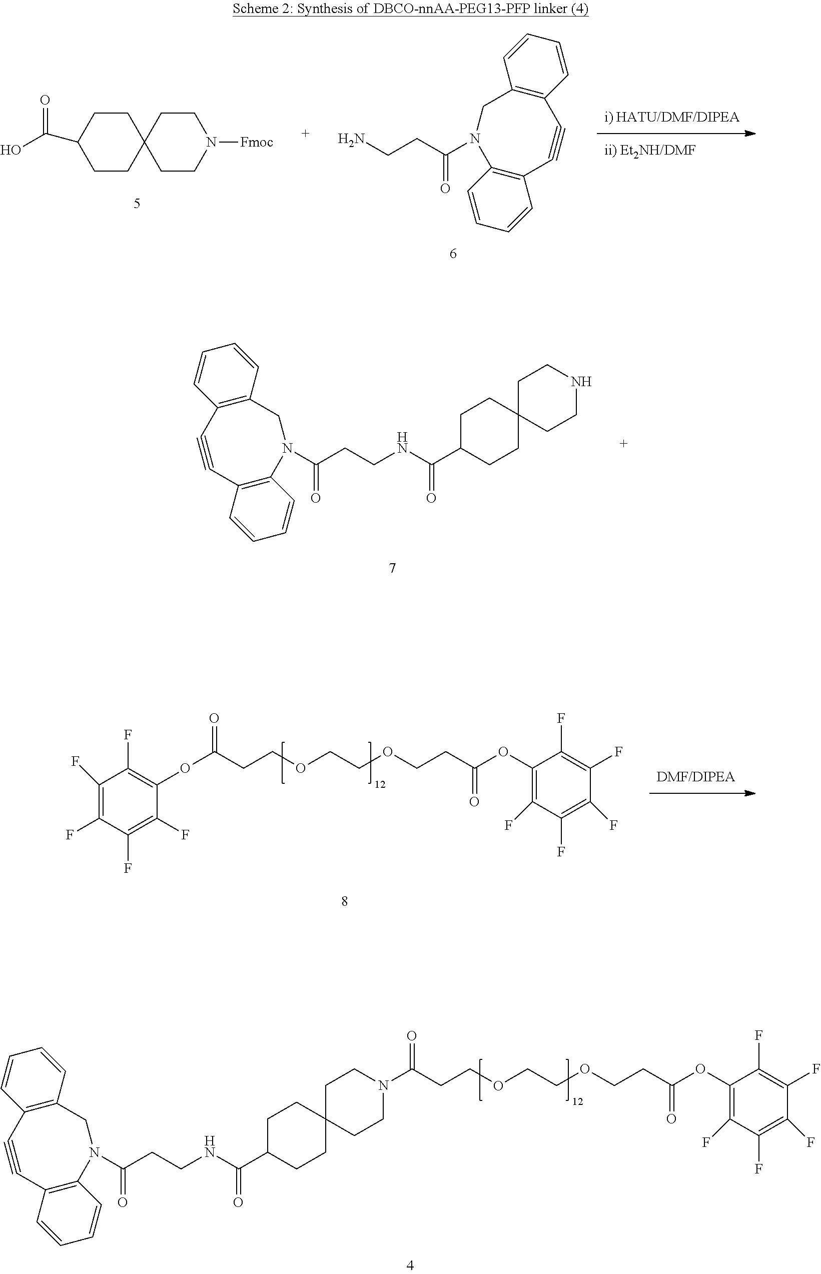

Synthesis of compound 7: Compound 5 (1.4 g, 3.34 mmol) was dissolved in DMF (10 mL). To the clear solution was added HATU (1.2 g, 3.34 mmol) and DIPEA (861 mg, 6.68 mmol). The solution was stirred at room temperature for about 30 sec. followed by the addition of DBCO-amine 7 (922 mg, 3.34 mmol) in DMF (2 mL). After the mixture was stirred at room temperature for 20 minutes, diethyl amine (2 mL) was added into and allowed to stir for another 30 minutes, LCMS showed completion of the reaction. The reaction solution was concentrated under reduced pressure and purified by reverse phase HPLC to afford compound 7 (790 mg); LCMS m/z (ESI⁺): calculated for C₂₉H₃₃N₃O₂, 455.26. found 456.4 (M+H).

Synthesis of compound 4: A solution of compound 8 (3 g, 2.9 mmol), compound 7 (780 mg, 1.37 mmol) and DIPEA (353 mg, 2.74 mmol) in DMF (20 mL) was stirred for 20 minutes, LCMS showed completion of the reaction. The reaction mixture was directly purified by reverse phase HPLC to give compound 4 (1.52 g); LCMS m/z (ESI⁺): calculated for C₆₅H₈₈F₅N₃O₁₈, 1293.60. found 1293.7 (M+H).

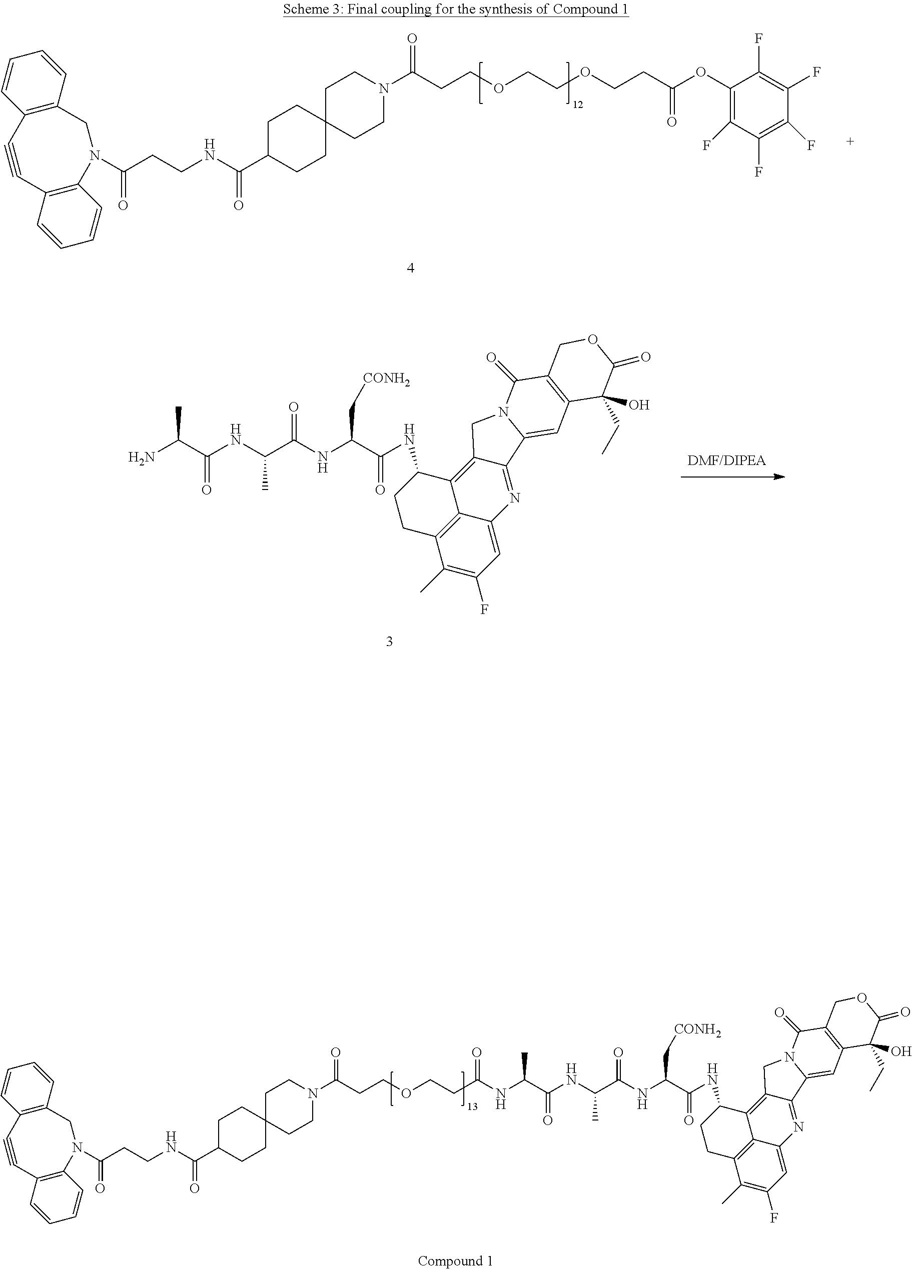

To a solution of compound 4 (770 mg, 0.6 mmol) in anhydrous DMF (3 mL) was added compound 3 (TFA salt, 478 mg, 0.6 mmol) and DIPEA (206 μL). The mixture was stirred at room temperature for 10 min, LCMS showed completion of the reaction. Then the mixture was purified directly by reverse phase HPLC to give Compound 1 as a light yellowish solid (710 mg); ¹H NMR (500 MHz, DMSO-d6) δ 11.41 (s, 1H), 8.25 (d, J=8.5 Hz, 1H), 8.07-7.91 (m, 3H), 7.77 (d, J=10.9 Hz, 1H), 7.68-7.54 (m, 2H), 7.47 (dddd, J=13.4, 7.6, 4.9, 2.8 Hz, 4H), 7.43-7.22 (m, 5H), 6.98-6.83 (m. 1H), 5.51 (dt, J=8.8, 4.5 Hz, 1H), 5.43 (s, 2H), 5.22 (s, 2H), 5.04 (d, J=14.0 Hz, 1H), 4.47 (q, J=6.8 Hz, 1H), 4.09 (dp, J=18.0, 7.1 Hz, 3H), 3.73 (s, 10H), 3.67-3.27 (m, 61H), 3.22-3.01 (m, 3H), 2.92 (dq, J=13.3, 6.7 Hz, 1H), 2.68-2.45 (m, 8H), 2.45-2.28 (m, 6H), 2.19 (dq, J=9.3, 4.8 Hz, 1H), 1.87 (qd, J=13.8, 7.1 Hz, 4H), 1.61 (d, J=13.1 Hz, 2H), 1.49-1.21 (m, 7H), 1.13 (dd, J=26.8, 7.1 Hz, 7H), 0.97 (ddd, J=16.1, 11.6, 6.4 Hz, 2H), 0.88 (t, J=7.3 Hz, 3H); LCMS m/z (ESI⁺): calculated for C₉₃H₁₂₅FN₁₀O₂₅, 1801.9. found 1802.9 [M+H]⁺.

### Example 2: Synthesis of DBCO-nnAA-PEG13-VKG-Exatecan (Compound 2)

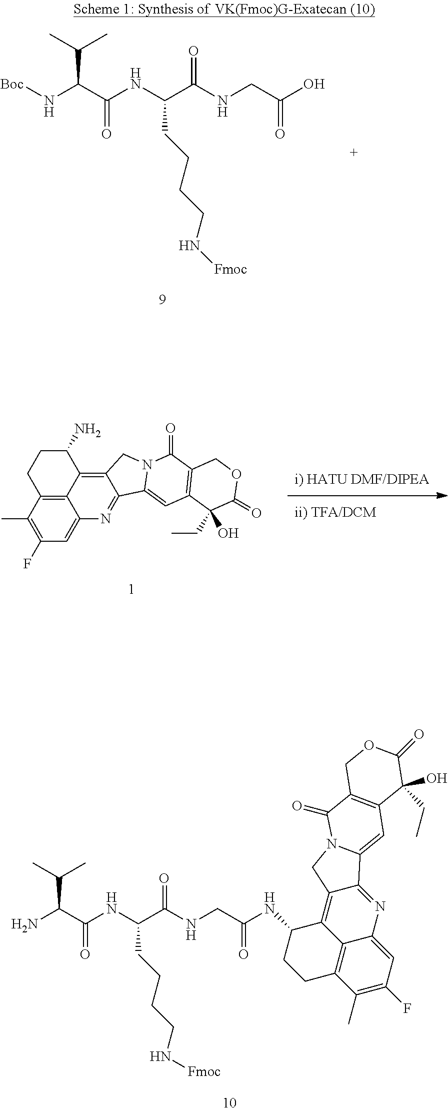

To a solution of compound Boc-VK(Fmoc)-G-OH 9 (562 mg, 0.9 mmol) in anhydrous DMF (10 mL) was added Exatecan mesylate (1) (478 mg, 0.9 mmol) and DIPEA (470 μL). After all components dissolved, HATU (342 mg, 0.9 mmol) was added, and the mixture was stirred at room temperature for 10 min. The reaction was then diluted with water (80 mL) and extracted with EtOAc (150 mL). The organic layer was washed with hydrochloric acid (0.2 M, 50 mL) and brine (50 mL), dried over Na₂SO₄ and evaporated to dryness under reduced pressure. The residue obtained was treated with TFA/DCM (1/4, 20 mL) at room temperature for 30 min and the reaction was evaporated to dryness under reduced pressure. The crude mixture was dissolved in 5 mL of DMF and purified by reverse phase HPLC to give compound 10 as a yellowish solid (771 mg). MS calculated for C₅₂H₅₆FN₇O₉, 941.4. found 942.9 [M+H]⁺

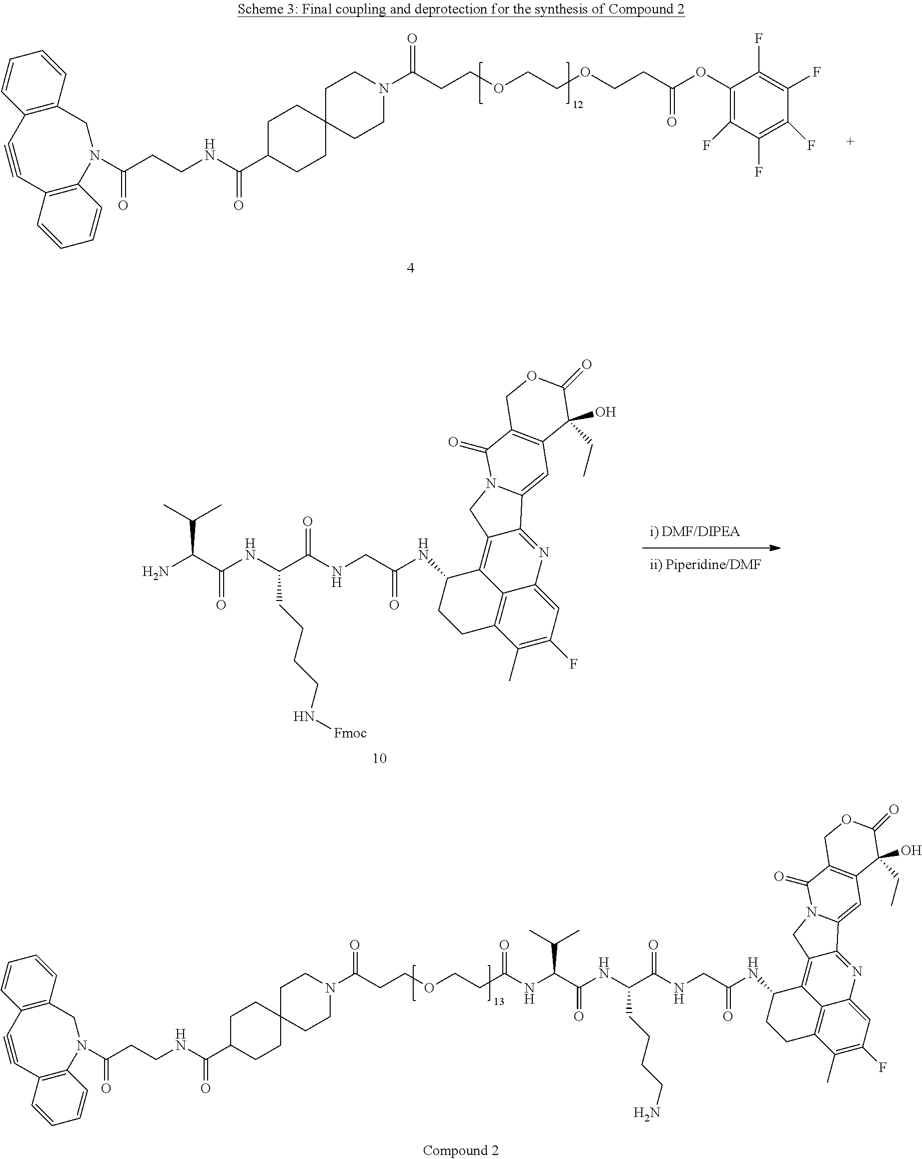

To a solution of compound 4 (426 mg, 0.33 mmol) in anhydrous DMF (3 mL) was added compound 10 (TFA salt, 347 mg, 0.33 mmol) and DIPEA (130 μL). The mixture was stirred at room temperature for 15 min, the LCMS showed the desired product formation. Then DBU (366 μL) was added dropwise, and the mixture was stirred at room temperature for additional 10 min. LCMS showed completion of the reaction. Then the mixture was purified directly by reverse phase HPLC to give Compound 2 as a yellowish solid (350 mg); HRMS m/z (ESI⁺): calculated for C₉₉H₁₃₃FN₁₀O₂₄, 1828.94. found 1829.95 [M+H]⁺; ¹H NMR (500 MHz, DMSO) δ 8.43 (d, J=8.5 Hz, 11H), 8.14 (t, J=5.6 Hz, 1H), 7.98 (d, J=7.4 Hz, 1H), 7.81 (d, J=10.9 Hz, 1H), 7.77-7.53 (m, 7H), 7.54-7.23 (m, 10H), 5.57 (dt, J=8.7, 4.4 Hz, 1H), 5.47-5.38 (m, 2H), 5.25 (d, J=3.3 Hz, 2H), 5.04 (d, J=14.1 Hz, 2H), 4.16 (td, J=8.1, 5.9 Hz, 3H), 4.08 (dd, J=8.5, 6.8 Hz, 3H), 3.88-3.68 (m, 13H), 3.50 (d, J=3.6 Hz, 71H), 3.25-3.13 (m, 4H), 3.13-3.03 (m, 2H), 2.92 (dq, J=13.4, 6.8 Hz, 2H), 2.78 (q, J=6.8 Hz, 3H), 2.60-2.26 (m, 18H), 2.26-2.03 (m, 3H), 1.98-1.74 (m, 6H), 1.74-1.46 (m, 8H), 1.46-1.09 (m, 12H), 0.97 (s, 2H), 0.88 (t, J=7.3 Hz, 3H), 0.78 (dd, J=14.1, 6.7 Hz, 7H).

### Example 3: Synthesis of DBCO-nnAA-PEG13-AAA-Exatecan (Compound 3)

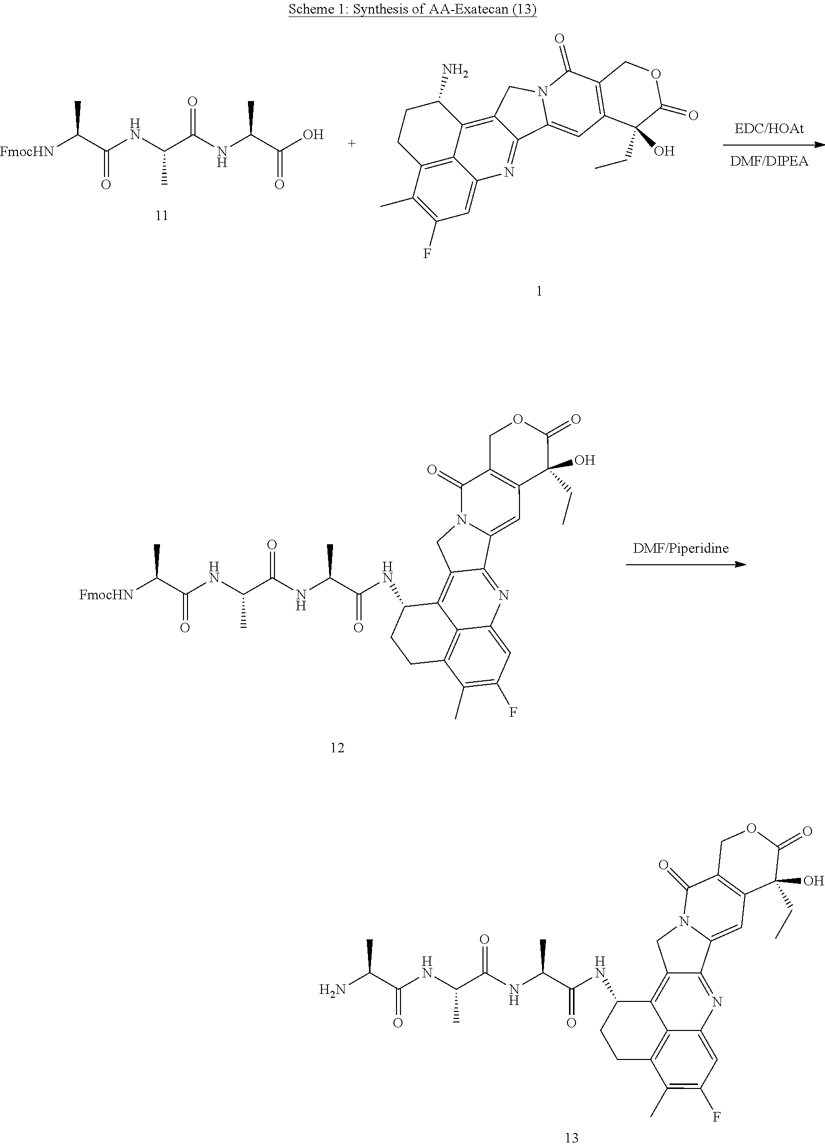

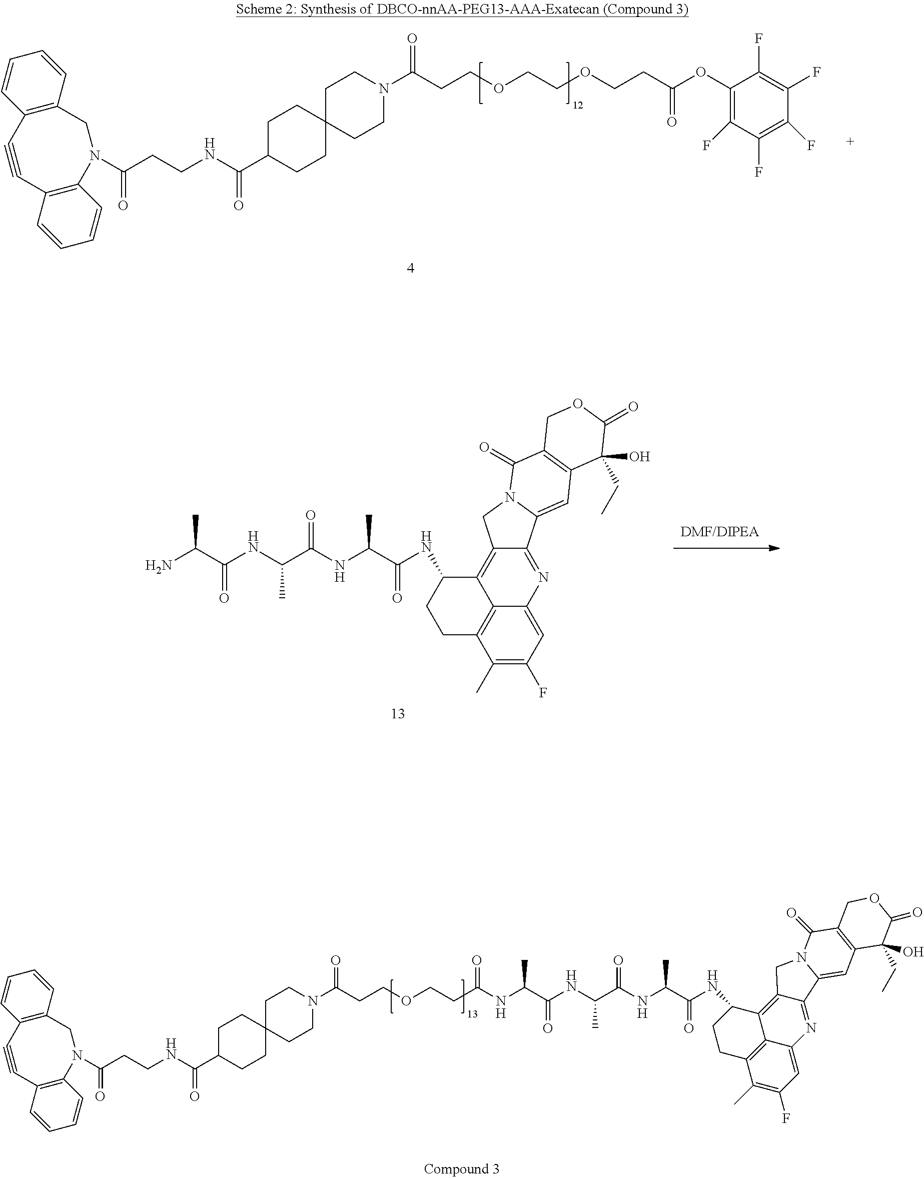

Compound 3 was synthesized in an analogous fashion using the same methods as described above. LCMS m/z (ESI⁺): calculated for C₉₂H₁₂₄FN₉O₂₄, 1757.87; found 1759.1 [M+H]⁺.

### Example 4: Synthesis of DBCO-nnAA-PEG13-VK-Exatecan (Compound 4)

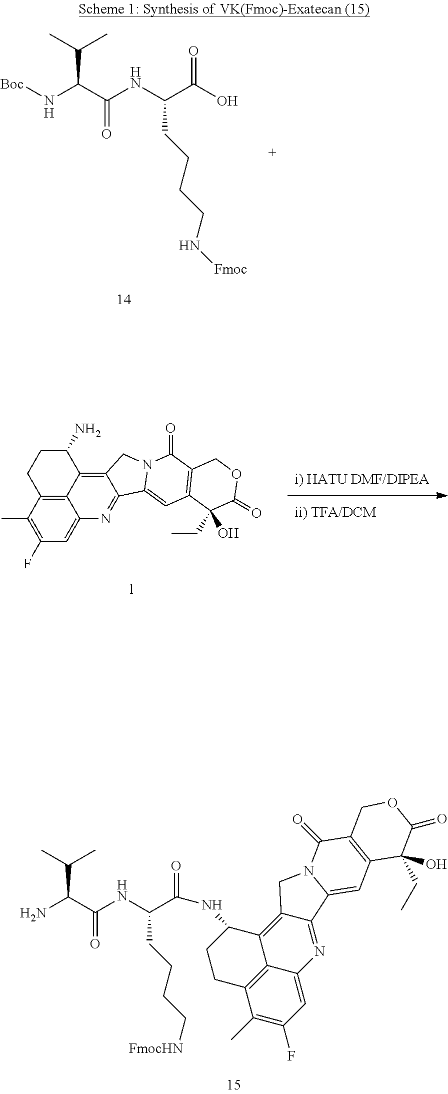

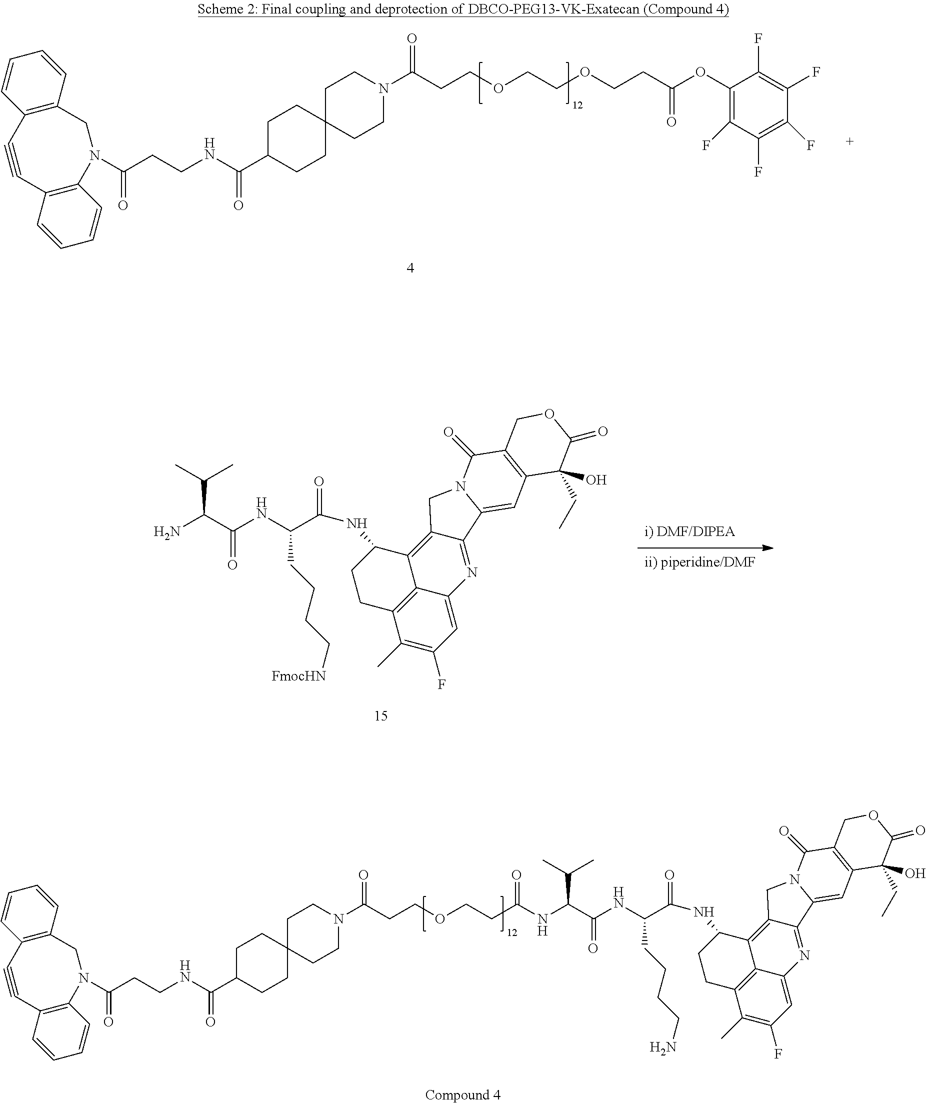

Compound 4 was synthesized in an analogous fashion using the same methods as described above. LCMS m/z (ESI⁺): calculated for C₉₄H₁₃₀FN₉O₂₃, 1771.93; found 1773.1 [M+H]⁺.

### Example 5: Synthesis of DBCO-nnAA-PEG13-NN-Exatecan (Compound 5)

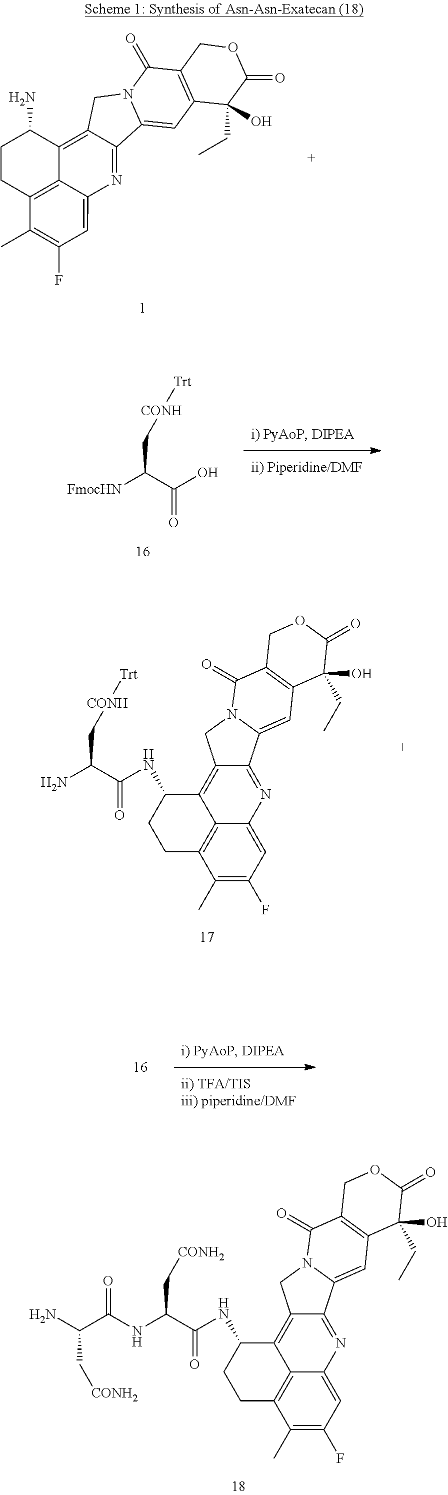

To a solution of Fmoc-Asn(Trt)-OH 16 (120 mg, 0.2 mmol) and exatecan mesylate 1 (106 mg, 0.2 mmol) in anhydrous DMF (4 mL) was added PyAOP (110 mg), followed by DIPEA (0.14 mL). The reaction was stirred at room temperature for 1 h, LCMS showed the desired product formation. hydrochloric acid (0.5 M, 30 mL) was added to the reaction. The mixture was extracted with EtOAc (60 mL) and the organic layer was dried over Na₂SO₄ and evaporated to dryness under reduced pressure. The residue obtained was dissolved in DMF (3 mL) and piperidine (0.4 mL) was added. The mixture was stirred at room temperature. After 20 min, the mixture was concentrated under reduced pressure to about 2 mL and the residue was triturated with diethyl ether/hexanes (1/1, 50 mL) to obtain compound 17. The compound 17 was diluted with DMF (5 mL), to this mixture, Fmoc-Asn(Trt)-OH 16 (360 mg, 0.6 mmol) was added, followed by PyAOP (330 mg) and DIPEA (0.21 mL). The mixture was stirred at room temperature for 10 min and diluted with EtOAc (100 mL). The mixture was washed with 0.5 M hydrochloric acid (50 mL) and water (50 mL). The organic layer was dried over Na₂SO₄ and evaporated to dryness under reduced pressure. The gum obtained was treated with TFA/TIS (9/1, 5 mL) for 20 min at room temperature. 100 mL of diethyl ether was added and the precipitated solid was collected by filtration to give the crude product, which was treated with piperidine (20% in DMF, 3 mL) at room temperature for 10 min LCMS showed completion of the reaction. The mixture was purified by RP-HPLC to give compound 18 (120 mg) as a pale-yellow solid after lyophilization; LCMS m/z (ESI⁺): calculated for C₃₂H₃₄FN₇O₈, 663.25. found 664.3 [M+H]⁺

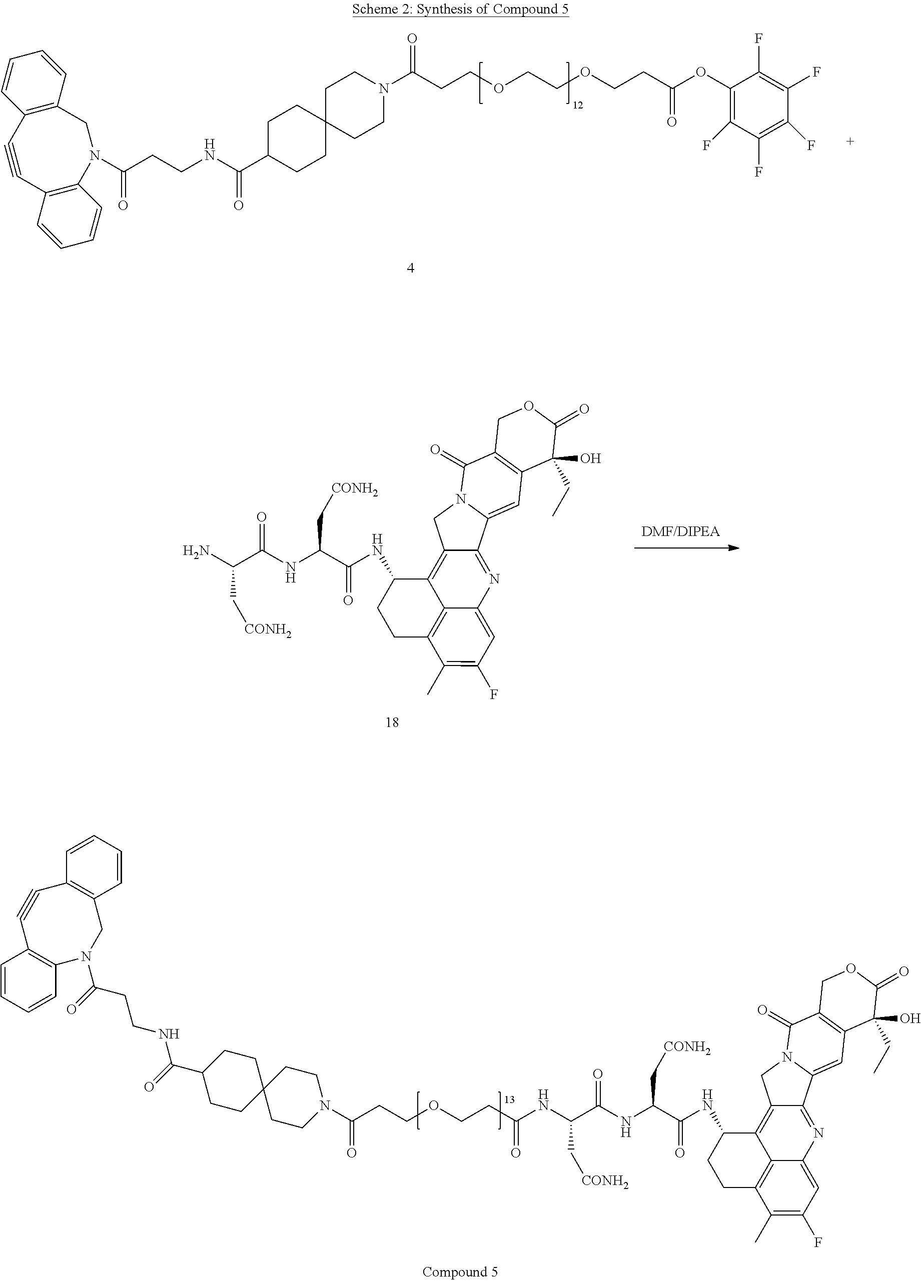

Compound 4 and compound 18 was coupled using the same methods as described above to obtain Compound 5 as yellow solid. LCMS m/z (ESI⁺): calculated for C₉₁H₁₂₁FN₁₀O₂₅, 1772.85. found 1773.91 [M+H]⁺.

### Example 6. Synthesis of Compound 6, Compound 7, Compound 8, and Compound 9

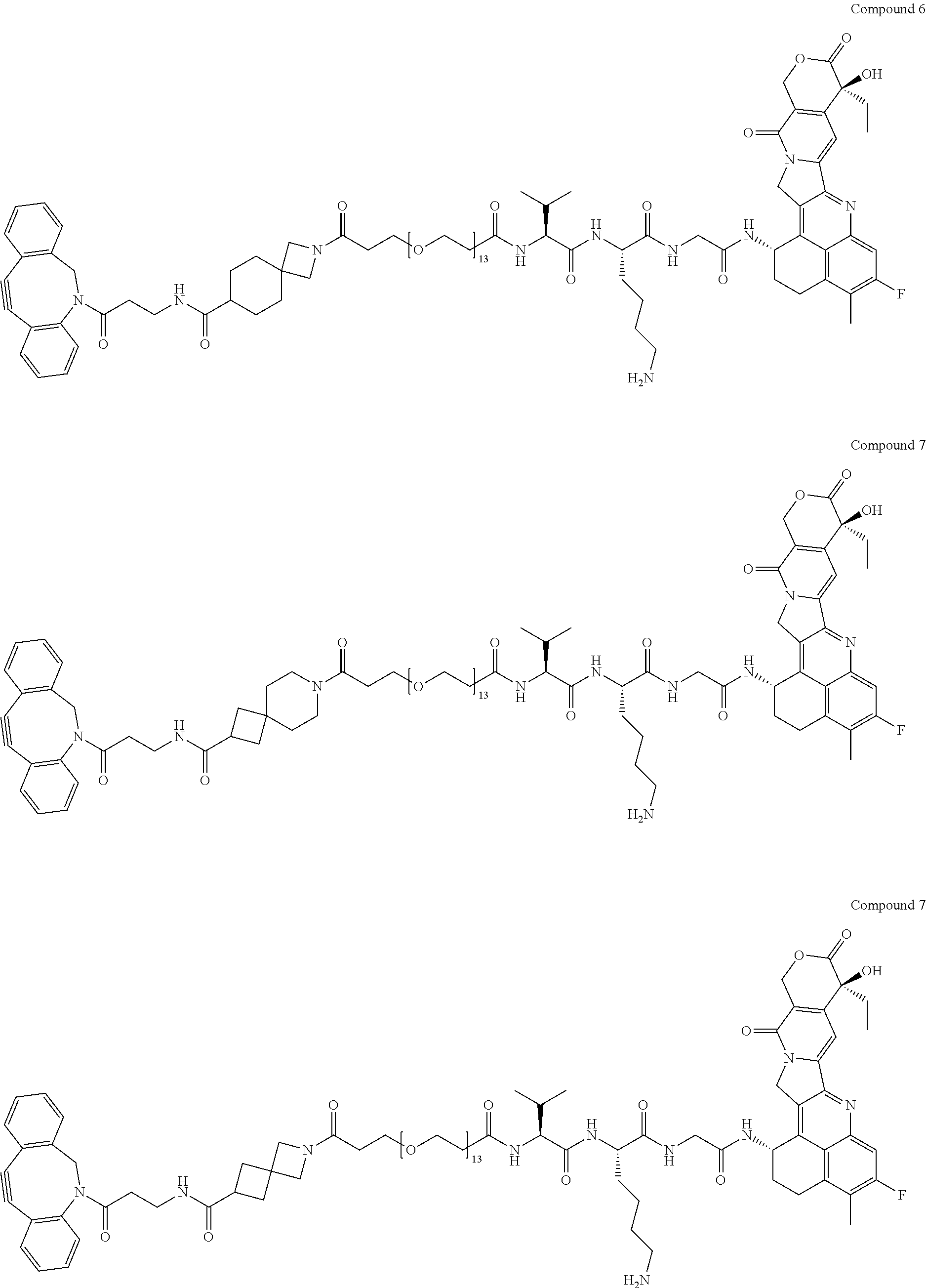

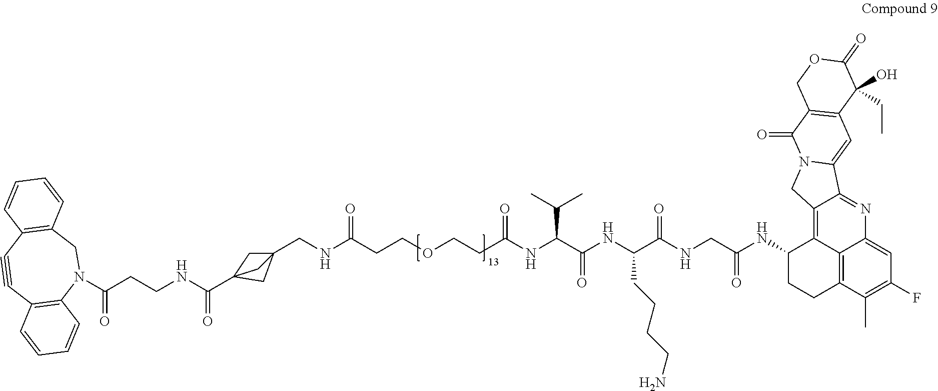

Compound 6, Compound 7, Compound 8, and Compound 9 are synthesized, purified, and characterized in an analogous fashion using the same methods as described above from the common intermediate compound 10.

### Example 7. Synthesis of Compound 10

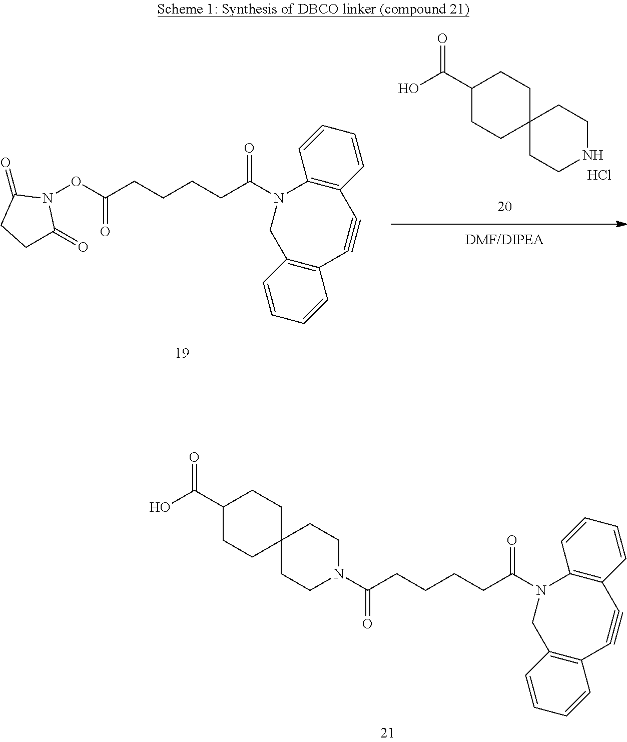 [SMILES: O=C(CCCCC(=O)N1Cc2ccccc2C#Cc2ccccc21)ON1C(=O)CCC1=O ; O=C(O)C1CCC2(CCNCC2)CC1 ; Cl ; O=C(O)C1CCC2(CC1)CCN(C(=O)CCCCC(=O)N1Cc3ccccc3C#Cc3ccccc31)CC2]

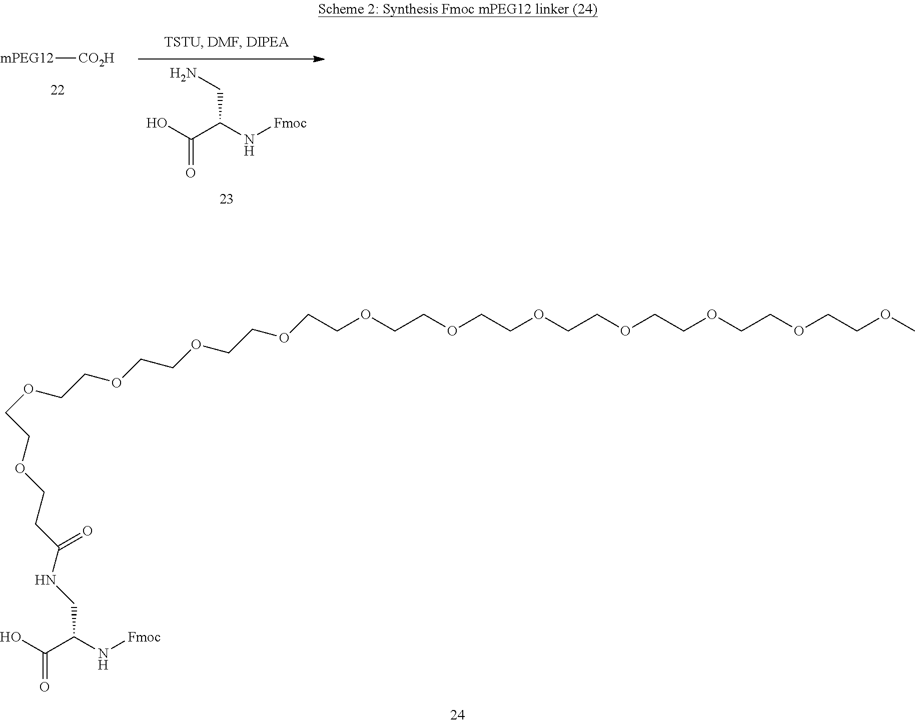

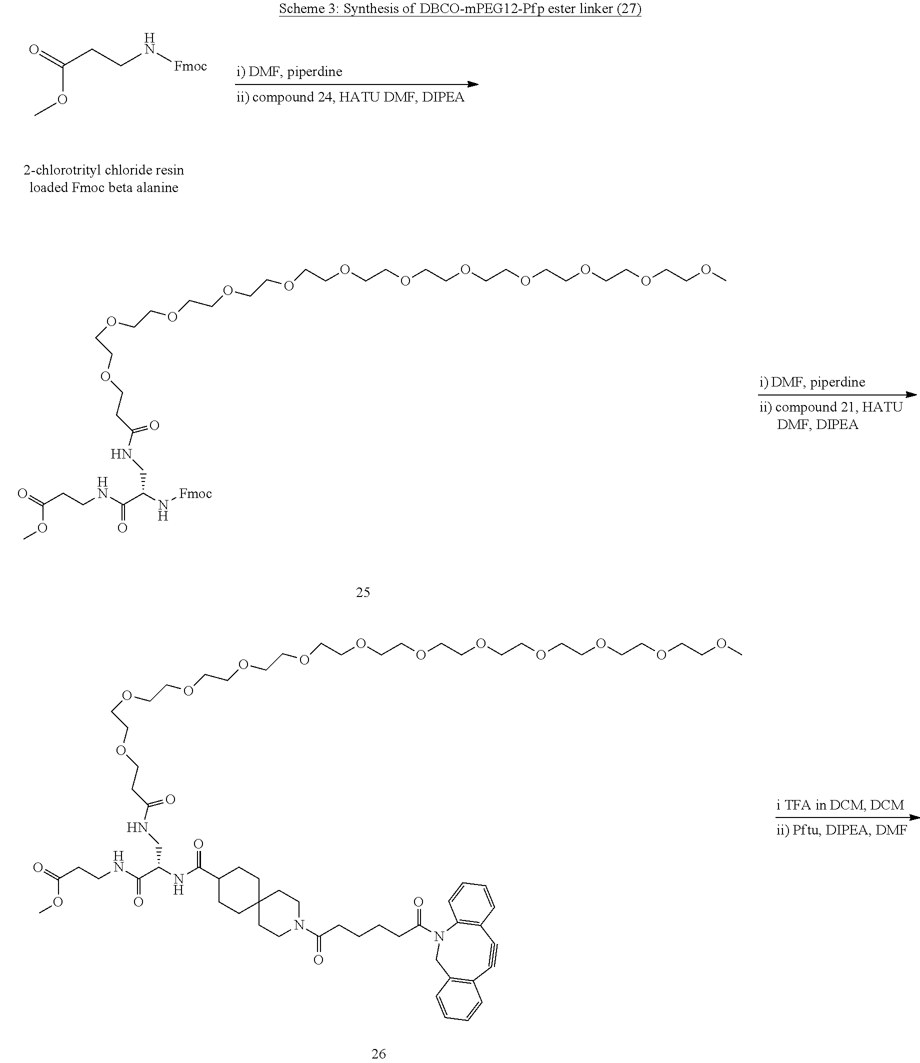

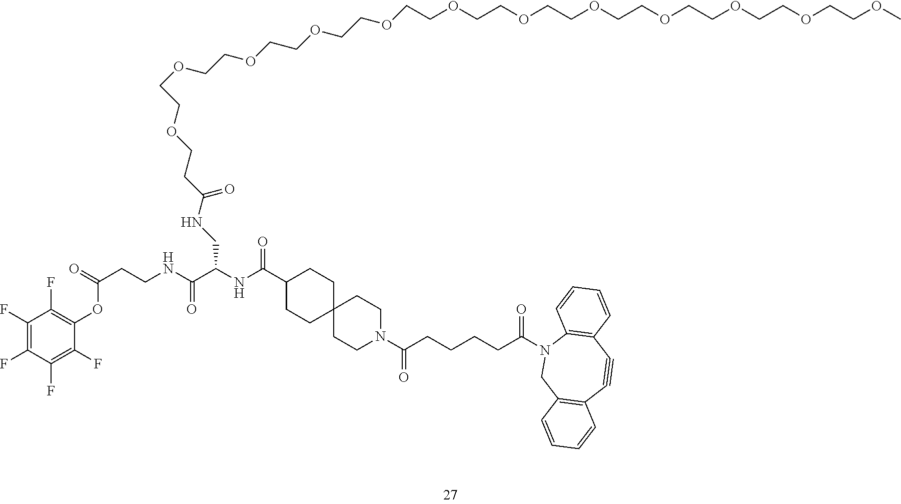 [SMILES: COCCOCCOCCOCCOCCOCCOCCOCCOCCOCCOCCOCCC(=O)NC[C@H](NC(=O)C1CCC2(CC1)CCN(C(=O)CCCCC(=O)N1Cc3ccccc3C#Cc3ccccc31)CC2)C(=O)NCCC(=O)Oc1c(F)c(F)c(F)c(F)c1F]

Compound 27 was synthesized on 2-chlorotrityl chloride resin solid support using the general methods 2-chlorotrityl chloride resin loading, Fmoc removal with DMF/piperidine, chain elongation and amide coupling was done with HATU, DMF, DIPEA and the final resin was cleaved by treating 2 mL of TFA/DCM (4%). The crude product carboxylic acid was dissolved in DMF (4 mL), and Pftu (86 mg, 0.2 mmol) was added, followed by DIEA (70 μL). The mixture was stirred at room temperature for 10 min and purified directly by reverse phase HPLC to give compound 27 (185 mg) as a viscous syrup after lyophilization.

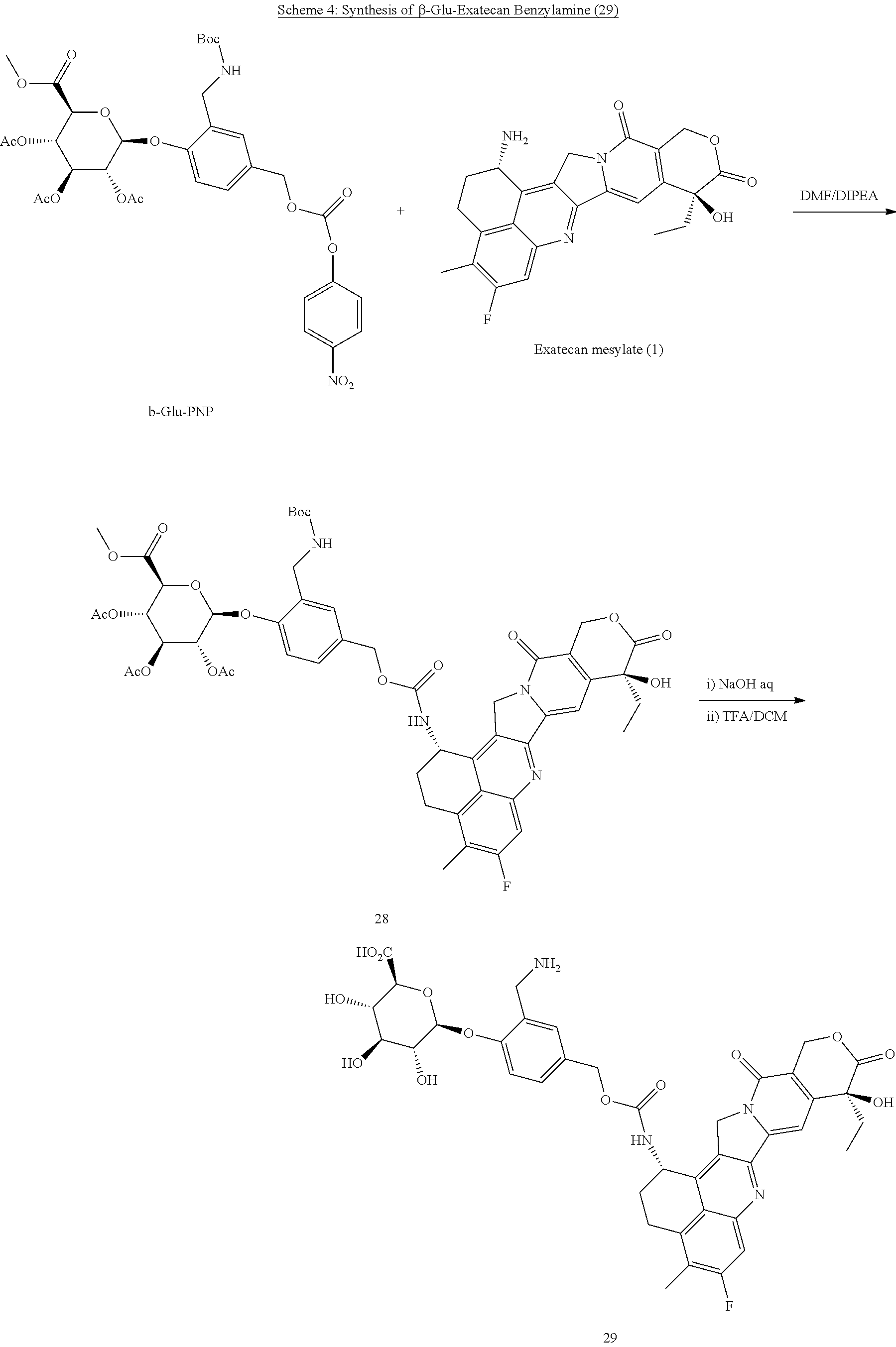

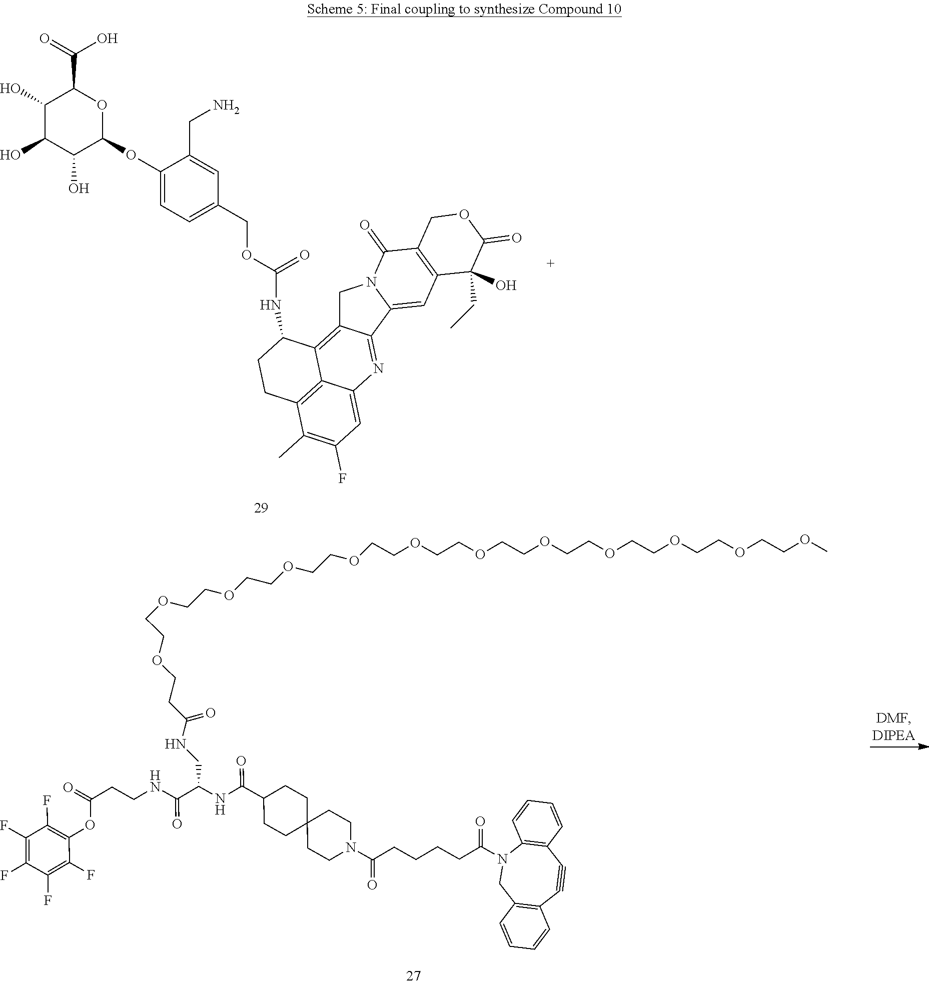 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CN)c1)CC3 ; COCCOCCOCCOCCOCCOCCOCCOCCOCCOCCOCCOCCC(=O)NC[C@H](NC(=O)C1CCC2(CC1)CCN(C(=O)CCCCC(=O)N1Cc3ccccc3C#Cc3ccccc31)CC2)C(=O)NCCC(=O)Oc1c(F)c(F)c(F)c(F)c1F]

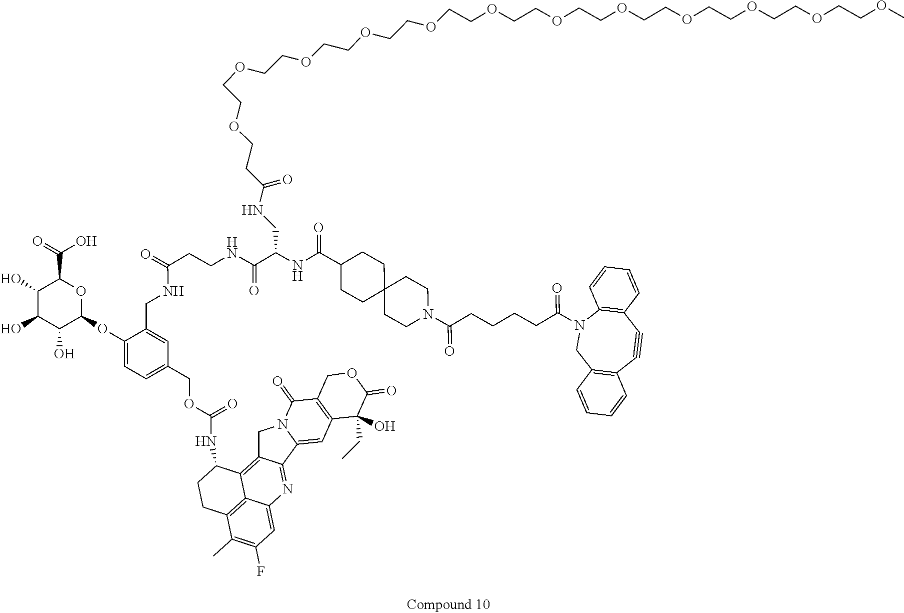 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CNC(=O)CCOCCOCCOCCOCCOCCOCCOCCOCCOCCOCCOCCOC)NC(=O)C2CCC4(CC2)CCN(C(=O)CCCCC(=O)N2Cc5ccccc5C#Cc5ccccc52)CC4)c1)CC3]

To a solution compound 29 (TFA salt, 40 mg, 44 μmol) and compound 27 (55 mg, 40 μmol) in 2 mL of anhydrous DMF was added DIPEA (38 μL) and the mixture was stirring at RT for 30 min, LCMS showed completion of the reaction. Then the mixture was purified by reverse phase HPLC to give Compound 10 (52.9 mg) as a yellow solid. LCMS m/z (ESI⁺): calculated for C₁₀₃H₁₃₄FN₉O₃₁, 2011.92: found 2013.1 [M+H]⁺.

### Example 8. Synthesis of Compound 11 and Compound 12

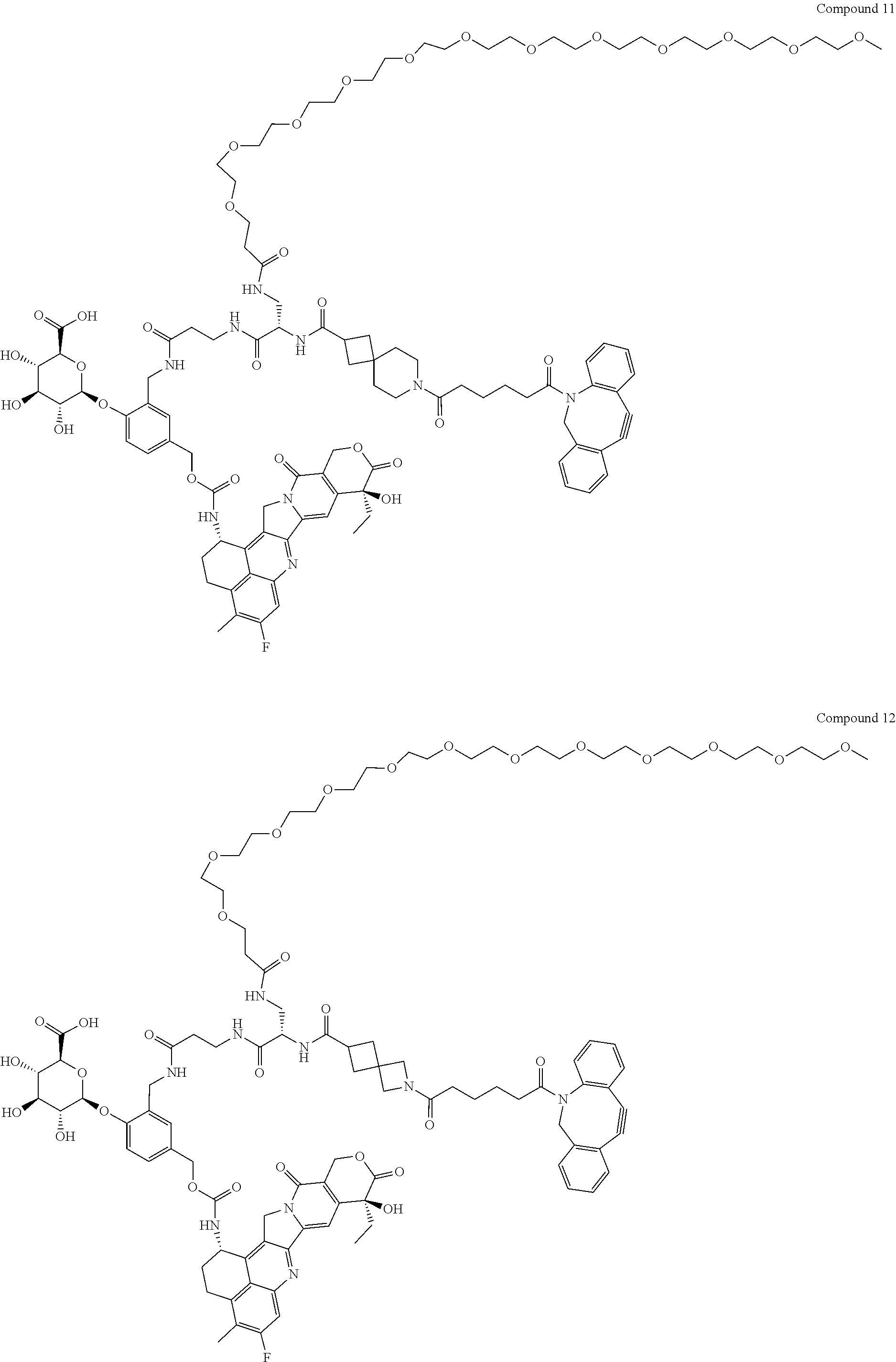

Compound 11 and Compound 12 are synthesized, purified, and characterized in an analogous fashion using the same methods as above example Compound 10 from the common intermediate compound 29.

### Example 9. Synthesis of Compound 13

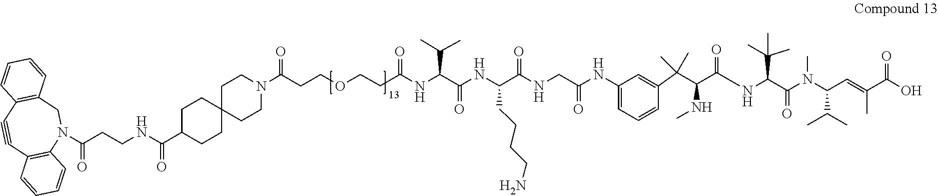

Compound 13 is synthesized in an analogous fashion using the same methods as Compound 2 as described in Example 2.

### Example 10. Synthesis of Compound 14

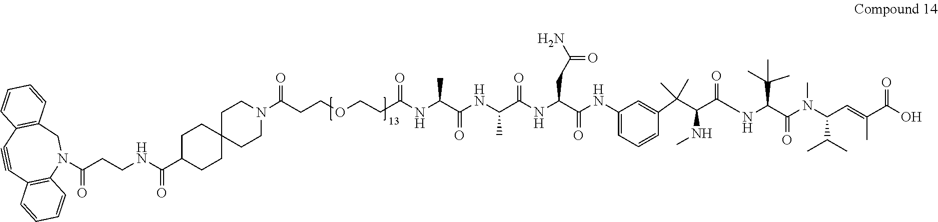

Compound 14 is synthesized in an analogous fashion using the same methods as Compound 1 as described in Example 1.

### Example 11. Synthesis of Compound 15

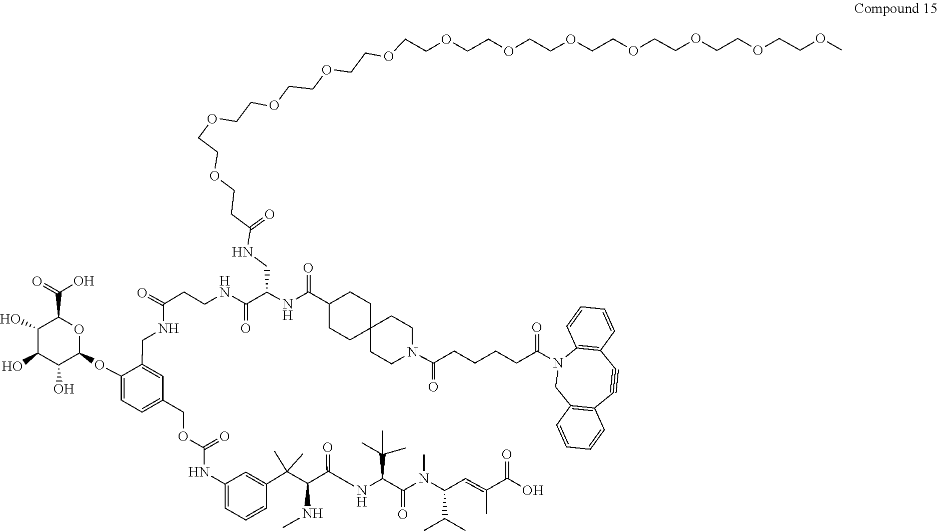 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(O[C@@H]3O[C@H](C(=O)O)[C@@H](O)[C@H](O)[C@H]3O)c(CNC(=O)CCNC(=O)[C@H](CNC(=O)CCOCCOCCOCCOCCOCCOCCOCCOCCOCCOCCOCCOC)NC(=O)C3CCC4(CC3)CCN(C(=O)CCCCC(=O)N3Cc5ccccc5C#Cc5ccccc53)CC4)c2)c1]

Compound 15 is synthesized in an analogous fashion using the same methods as Compound 10 as described in Example 7.

### Example 12: Synthesis of Compound 16

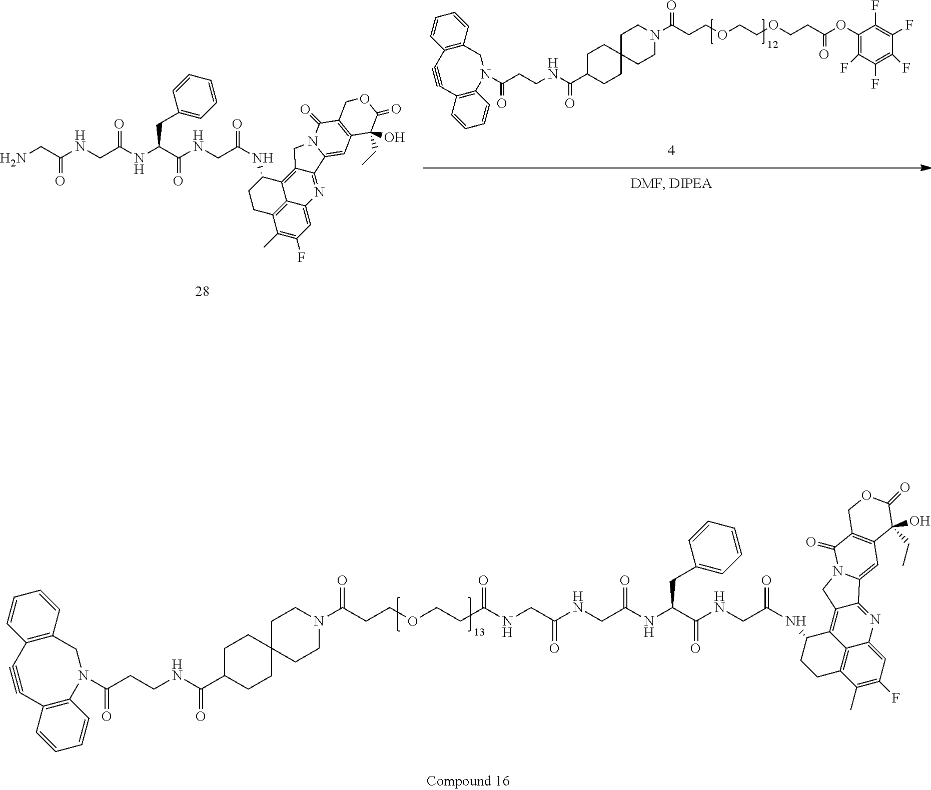

To a solution of compound 28 (44 mg, 51 umol) in anhydrous DMF (2 mL) was added compound 4 (66 mg, 51 umol) and DIPEA (18 uL, 156 umol). The mixture was stirred at room temperature for 20 min, LCMS showed completion of the reaction. Then the mixture was purified directly by reverse phase HPLC to give Compound 16 as a yellow solid (61 mg). LCMS mi (ESI⁺): calculated for C₉₈H₁₂₇FN₁₀O₂₅, 1862.90. found 1864.1 [M+H]⁺.

### Example 13: Synthesis of Compound 17

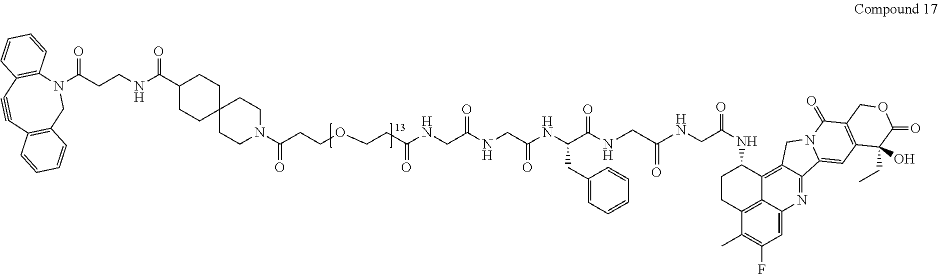

Compound 17 is synthesized in an analogous fashion using the same methods as Compound 16 as described in Example 12.

### Example 14: Synthesis of Compound 18

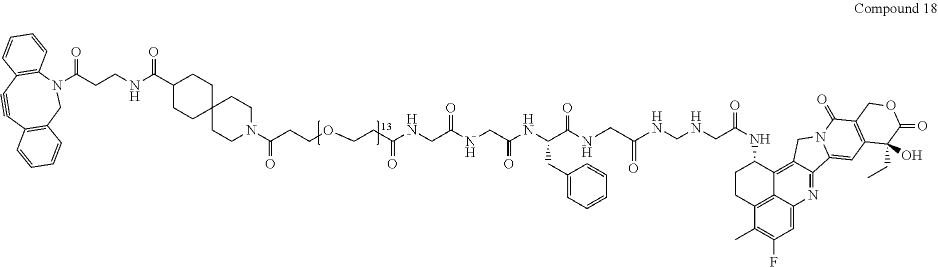

Compound 18 is synthesized in an analogous fashion using the same methods as Compound 16 as described in Example 12.

### Example 15. Synthesis of Compound 19, Compound 20, Compound 21, Compound 22, and Compound 23

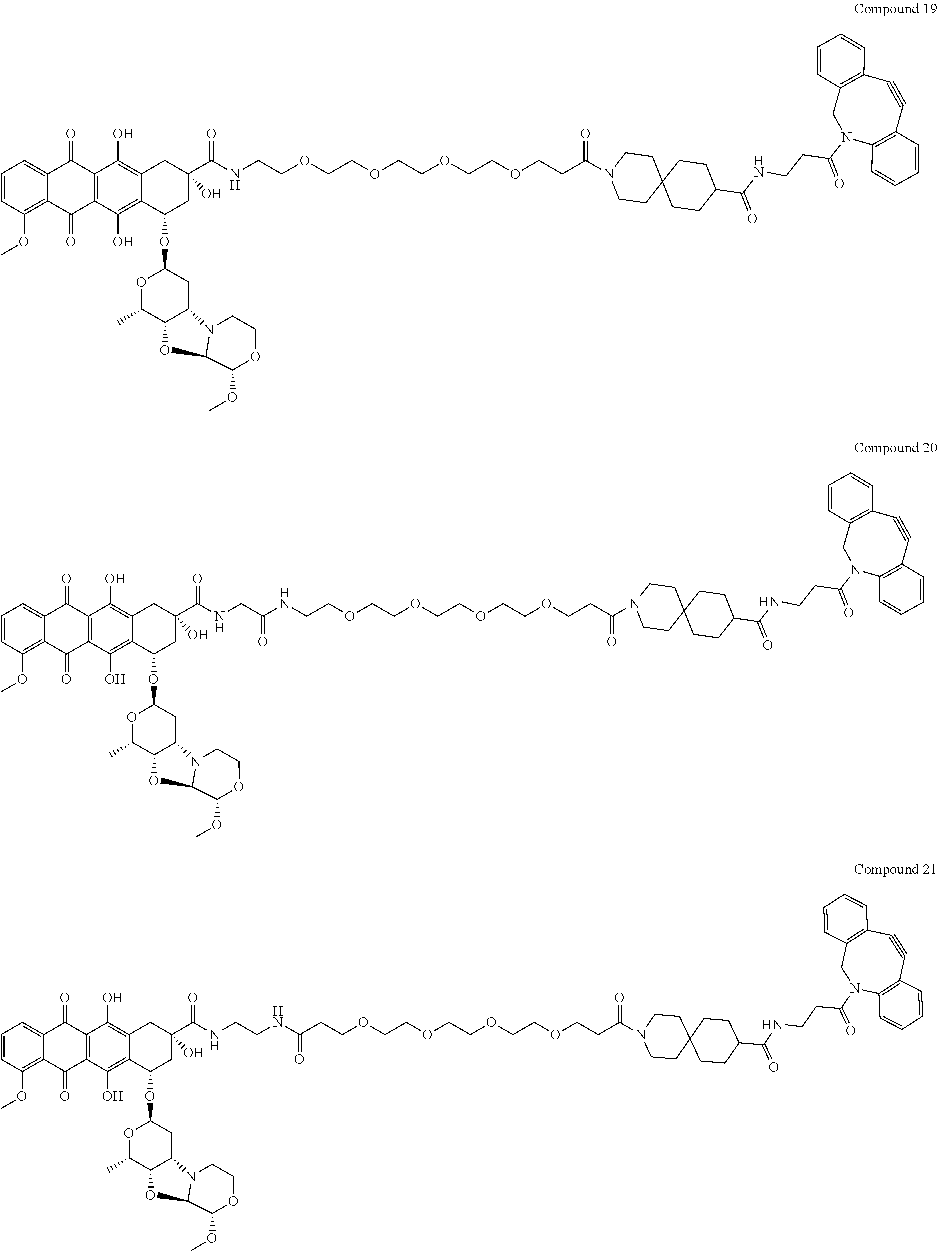

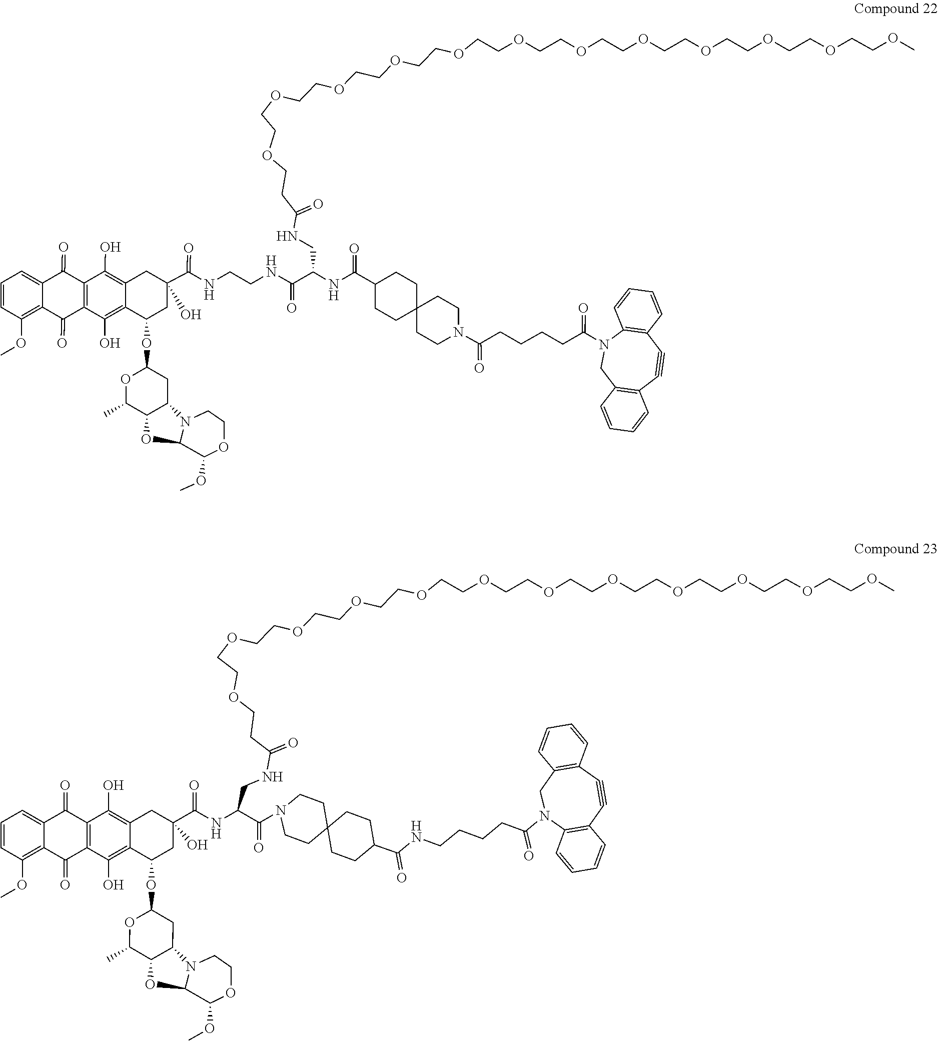 [SMILES: COCCOCCOCCOCCOCCOCCOCCOCCOCCOCCOCCOCCC(=O)NC[C@H](NC(=O)C1CCC2(CC1)CCN(C(=O)CCCCC(=O)N1Cc3ccccc3C#Cc3ccccc31)CC2)C(=O)NCCNC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O ; COCCOCCOCCOCCOCCOCCOCCOCCOCCOCCOCCOCCC(=O)NC[C@H](NC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O)C(=O)N1CCC2(CCC(C(=O)NCCCCC(=O)N3Cc4ccccc4C#Cc4ccccc43)CC2)CC1]

Compound 19, Compound 20, Compound 21, Compound 22, and Compound 23 were synthesized using the same methods described above.

### Example 16. Conjugation of Linker-Payload Compounds

Small molecule drug linker was dissolved in DMSO to a final concentration of 5 mM. The conjugation was carried out in 1×PBS at antibody concentration of 1 mg/mL, drug linker to pAMF ratio of 3, and with 15% of DMSO. The reaction mixture was incubated at 30° C. for overnight. The conjugation efficiency was measured by MALDI. Unconjugated drug linker was removed by desalting. Purity of the conjugate was measured by Sepax SEC-300. The conjugate was formulated in 1×PBS.

Linker drugs were conjugated to aFolR mAb with 4 pAMF sites and 8 pAMF sites incorporated at heavy chain Y180F404 sites and light chain K42E161 sites. Following the conjugation condition described above, over 94% conjugation efficiency was achieved for all the linker payloads. The result of analytical SEC showed that all the conjugates exhibited high purity with >99% monomer (Table 1).

TABLE 1
Results of Conjugation of Select Linker-Payloads

| Conjugate | | Compd | | monomer |
|---|---|---|---|---|
| # | mAb | # | DAR | % |
| 102-1 | 1848-H01 HC-Y180F404/ | 2 | 7.9 | 98 |
| | LC-K42E161 | | | |
| 101-1 | 1848-H01 HC-Y180F404/ | 1 | 7.2 | 100 |
| | LC-K42E161 | | | |
| 103-1 | 1848-H01 HC-Y180F404/ | 3 | 7.1 | 89 |
| | LC-K42E161 | | | |
| 106-1 | 1848-H01 HC-Y180F404/ | 6 | 7.9 | 100 |
| | LC-K42E161 | | | |
| 107-1 | 1848-H01 HC-Y180F404/ | 7 | 7.9 | 100 |
| | LC-K42E161 | | | |
| 108-1 | 1848-H01 HC-Y180F404/ | 8 | 7.8 | 100 |
| | LC-K42E161 | | | |
| 109-1 | 1848-H01 HC-Y180F404/ | 9 | 7.9 | 100 |
| | LC-K42E161 | | | |
| 113-1 | 1848-H01 Y180F404 | 13 | 3.98 | 100 |
| 114-1 | 1848-H01 Y180F404 | 14 | 3.99 | 100 |
| 115-1 | 1848-H01 Y180F404 | 15 | 3.99 | 100 |
| 124-1 | 1848-H01 HC-Y180F404/ | 24 | 7.6 | 100 |
| | LC-K42E161 | | | |
| 126-1 | 1848-H01 HC-Y180F404/ | 26 | 7.1 | 100 |
| | LC-K42E161 | | | |

### Example 17: Free Drug Cell Killing Activity

The in vitro cell killing activity of exatecan, gly-exatecan, and hemiasterlin were evaluated in a panel of breast cancer and lung cancer cell lines.

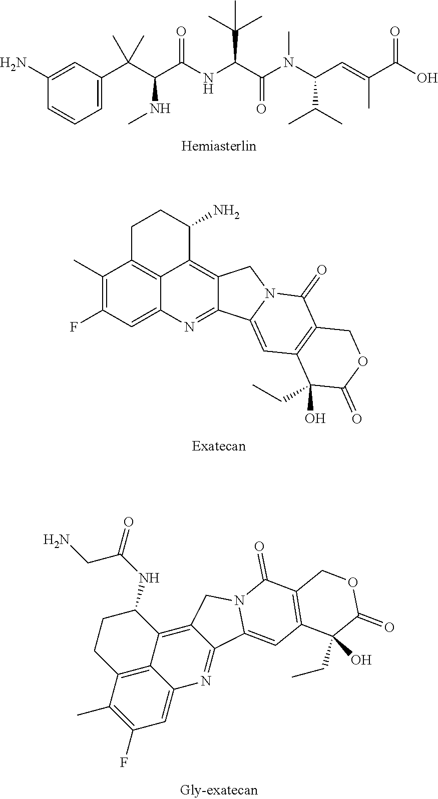 [SMILES: CN[C@H](C(=O)N[C@H](C(=O)N(C)[C@H](/C=C(\C)C(=O)O)C(C)C)C(C)(C)C)C(C)(C)c1cccc(N)c1 ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CN)CC3]

MDA-MB-361, SKBR3, BT20, HCC38, HCC1143, HCC1937, JIMT1, MCF-7, A549, NCI-H441, NCI-H520, NCI-H1703, NCI-H1975, NCI-H2110, NCI-H226, NCI-H292, NCI-H358 cells were purchased from ATCC (American Type Culture Collection, Manassas, VA, USA). All the cell lines were maintained in DMEM/F12 (1:1), high glucose (Corning, Corning, NY) supplemented with 10% heat-inactivated fetal bovine serum (Thermo Scientific, Grand Island, NY), 2 mM glutamax (Thermo Scientific, Grand Island, NY), and 1× Penicillin/Streptomycin (Corning, Corning, NY). Cytotoxicity effect of the Exatecan warhead was measured with a cell proliferation assay. Cells were seeded in a 384-well flat bottom white polystyrene plate at 625 cells/25 uL the day before the actual assay starts. Exatecan was formulated at 2× starting concentration in cell culture medium, serial diluted (1:3) under sterile conditions and added onto cells in triplicates. Plates were cultured at 37° C. in a CO₂ incubator for 120 hours. For cell viability measurement, 30 microliter of Cell Titer-Glo® reagent (Promega Corp, Madison, WI) was added into each well, and plates processed as per product instructions. Relative luminescence was measured on an ENVISION® plate reader (Perkin-Elmer; Waltham, MA). Relative luminescence readings were converted to % viability using untreated cells as controls. Data was fitted with non-linear regression analysis, using log (inhibitor) vs. response, variable slope, 4-parameter fit equation using GraphPad Prism.

Exatecan free warhead exhibited potent cell killing on all the cell lines tested, with EC₅₀ values ranging from 0.32 nM to 4.2 nM (Table 2), similar to hemiasterlin. Gly-exatecan showed slightly less cell killing activity with EC₅₀ values ranging from 3.8 nM to 44 nM (Table 2).

TABLE 2
Free Drug Cell killing activity

| | Hemiasterlin | | Exatecan | | Gly-exatecan | |
|---|---|---|---|---|---|---|
| | EC₅₀ | Span | EC₅₀ | Span | EC₅₀ | Span |
| Cell Line | (nM) | (%) | (nM) | (%) | (nM) | (%) |
| MDA-MB- | 0.51 | 81 | 0.59 | 97 | 5.3 | 91 |
| 468 | | | | | | |
| MDA-MB- | 2.7 | 70 | 0.84 | 64 | 3.8 | 60 |
| 231 | | | | | | |
| MDA-MB- | 0.8 | 94 | 1.2 | 96 | 4.5 | 98 |
| 361 | | | | | | |
| SKBR3 | 0.4 | 91 | 0.39 | 96 | 4.2 | 95 |
| BT20 | 0.8 | 87 | 0.91 | 96 | 21 | 92 |
| HCC38 | 1.39 | 73 | 0.38 | 96 | 6.1 | 93 |
| HCC1143 | 0.21 | 70 | 0.85 | 76 | 14 | 76 |
| HCC1937 | 1.2 | 55 | 3.5 | 94 | 35 | 87 |
| JIMT1 | 1.2 | 95 | 1.6 | 98 | 15 | 70 |
| MCF-7 | 0.96 | 77 | 1.2 | 83 | 8.8 | 75 |
| NCI-H441 | 0.89 | 61 | 0.32 | 93 | 5.1 | 90 |
| NCI-H520 | 0.68 | 96 | 0.6 | 96 | 11 | 80 |
| A549 | 4.2 | 81 | 1.2 | 96 | 44 | 90 |
| NCI-H1703 | 0.3 | 98 | 1.1 | 93 | 25 | 91 |
| NCI-H1975 | 2.4 | 92 | 0.87 | 90 | 43 | 84 |
| NCI-H2110 | 0.66 | 91 | 0.61 | 91 | 9.2 | 89 |
| NCI-H2343 | 1.18 | 26 | 12 | 70 | 30 | 55 |
| NCI-H226 | 2.4 | 92 | 1.08 | 98 | 23 | 99 |
| NCI-H292 | 0.99 | 94 | 0.36 | 98 | 4 | 94 |
| NCI-H358 | 1.3 | 88 | 0.53 | 96 | 7.5 | 95 |

### Example 18: In Vitro Cell Killing Activity of Exatecan ADCs

Anti-FolRα ADCs were generated by conjugating anti-FolRa antibody 1848-H01 with exatecan linker warheads at DAR=4 and DAR=8. Anti-FolRa antibody 1848-H01 conjugated to Compound 27 and Compound 26 at DAR=4 and DAR=8 were used as positive controls.

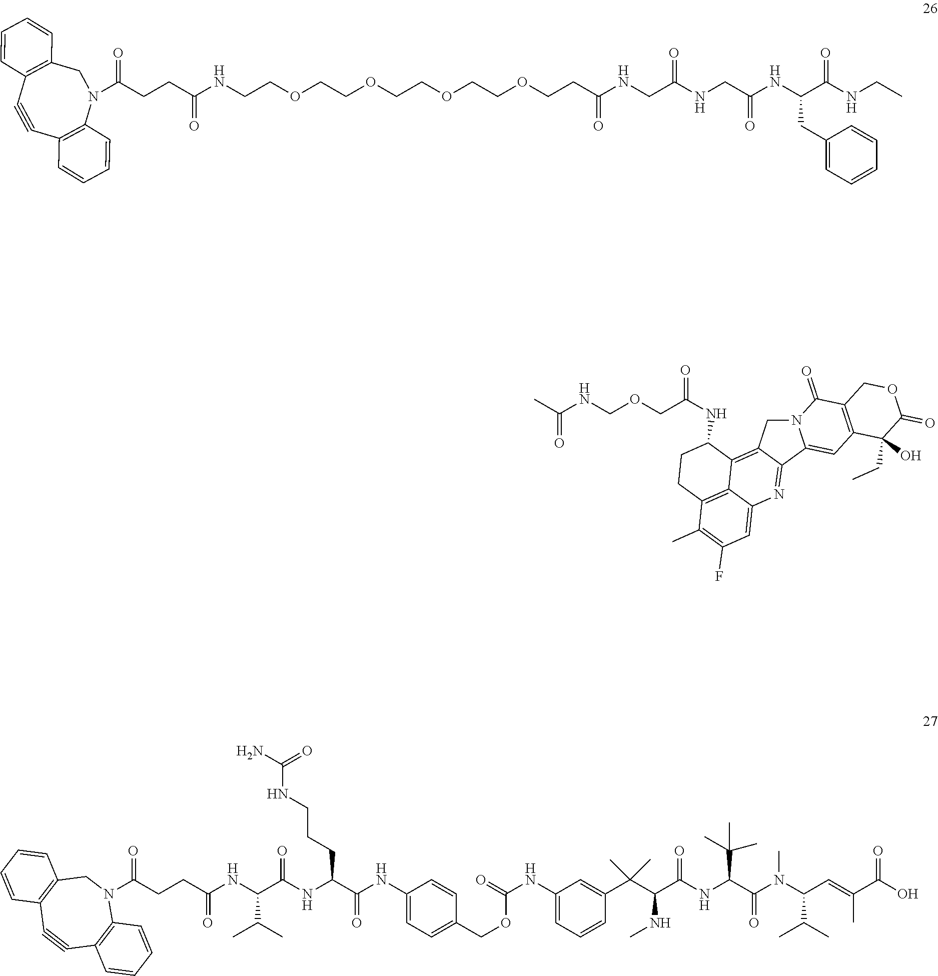

FolRα positive Igrov1 cells were licensed from NCI (National Cancer Institute at Frederick, Maryland). FolRa negative A549 were purchased from ATCC (American Type Culture Collection). Both cell lines were maintained in DMEM/F12 (1:1), high glucose (Corning) supplemented with 10% heat-inactivated fetal bovine serum (Thermo Scientific), 2 mM glutamax (Thermo Scientific), and 1× Penicillin/Streptomycin (Corning). Cytotoxicity effects of the ADCs were measured with a cell proliferation assay. Igrov1 and A549 cells at a concentration of 625 cells/25 ul were seeded in a 384-well flat bottom white polystyrene plate the day before the assay. ADCs were formulated at 2× starting concentration in cell culture medium and sterile filtered through SpinX 0.22 um filtered centritube (Corning Costar). Filter sterilized samples were serial diluted (1:3) under sterile conditions and added onto cells in triplicates. Plates were cultured at 37° C. in a CO2 incubator for 120 hours. For cell viability measurement, 30 microliter of Cell Titer-Glo® reagent (Promega Corp, Madison, WI) was added into each well, and plates processed as per product instructions. Relative luminescence was measured on an ENVISIONS plate reader (Perkin-Elmer; Waltham, MA). Relative luminescence readings were converted to % viability using untreated cells as controls. Data was fitted with non-linear regression analysis, using log (inhibitor) vs. response, variable slope, 4-parameter fit equation using GraphPad Prism.

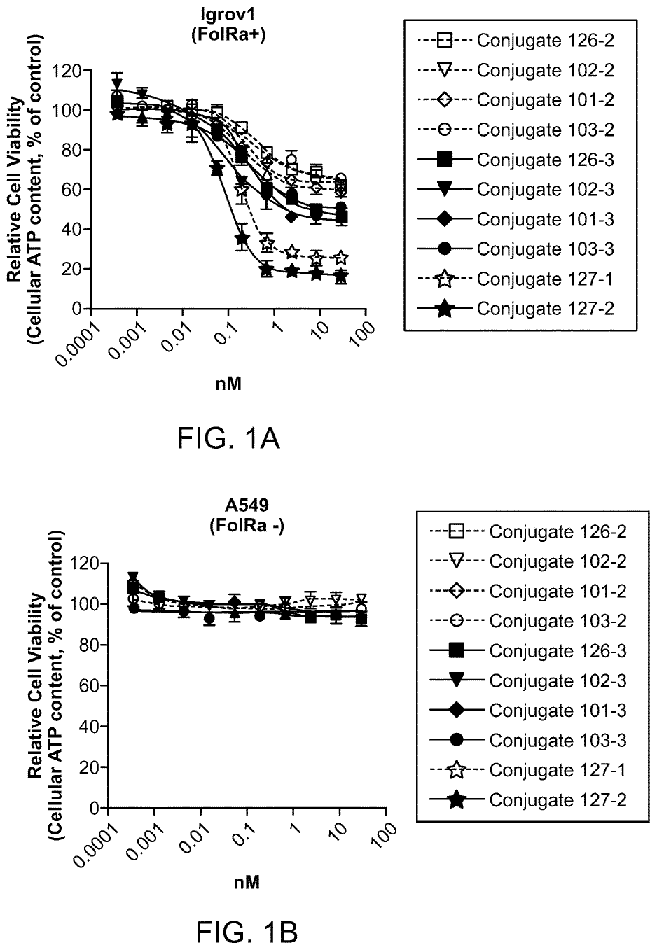

As shown in FIG. 1A and FIG. 1B, anti-FolRα ADCs conjugated to Compound 1, Compound 2, and Compound 3 at DAR=4 and DAR8 showed potent cell killing on FolRa positive Igrov1 cells, while no cell killing was observed on FolRa negative A549 cells. This is an indication that there was no non-specific release of the free warheads to kill the target negative cells, which means that all the linkers tested were stable in the cell culture medium for 5 days. Compared to ADCs at DAR4, ADCs at DAR8 showed more potent cell killing with lower EC₅₀ and bigger killing span.

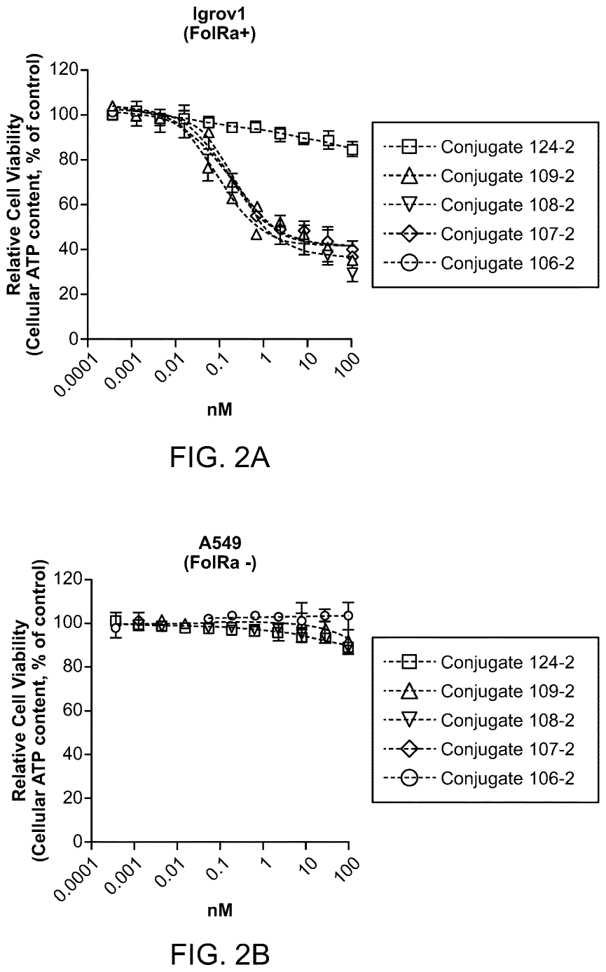

As shown in FIG. 2A and FIG. 2B, anti-FolRα ADCs conjugated Compound 6, Compound 7, Compound 8, Compound 9, at DAR=8 showed potent cell killing on FolRa positive Igrov1 cells, while no cell killing was observed on hFolRα negative A549 cells. Anti-FolRα ADCs conjugated to Compound 24 showed no cell killing on FolRα positive Igrov1 cells, nor on FolRa negative A549 cells.

The cell killing activity of Exatecan ADCs is summarized in Table 3 and Table 4.

TABLE 3
Cell killing activity of Exatecan ADCs

| Con- | | | Igrov1 | | A549 | |
|---|---|---|---|---|---|---|
| jugate | | | EC₅₀ | Span | EC₅₀ | Span |
| # | Sample | DAR | (nM) | (%) | (nM) | (%) |
| 126-2 | 1848-H01 Y180F404 + 26 | 3.7 | 1.3 | 51 | >10 | <10 |
| 102-2 | 1848-H01 Y180F404 + 2 | 3.9 | 0.31 | 40 | >10 | <10 |
| 101-2 | 1848-H01 Y180F404 + 1 | 3.8 | 0.25 | 44 | >10 | <10 |
| 103-2 | 1848-H01 Y180F404 + 3 | 3.7 | 0.26 | 40 | >10 | <10 |
| 126-3 | 1848-H01 | 7.1 | 0.31 | 64 | >10 | <10 |
| | Y180F404/K42/E161 + 26 | | | | | |
| 102-3 | 1848-H01Y180F404/ | 7.6 | 0.075 | 68 | >10 | <10 |
| | K42/E161 + 2 | | | | | |
| 101-3 | 1848-H01 | 7.3 | 0.31 | 57 | >10 | <10 |
| | Y180F404/K42/E161 + 1 | | | | | |
| 103-3 | 1848-H01 | 7.1 | 0.38 | 55 | >10 | <10 |
| | Y180F404/K42/E161 + 3 | | | | | |
| 127-1 | 1848-H01 Y180F404 + 27 | 3.7 | 0.18 | 75 | >10 | <10 |
| 127-2 | 1848-H01 | 7.4 | 0.083 | 84 | >10 | <10 |
| | Y180F404/K42/E161 + 27 | | | | | |

TABLE 4
Cell killing activity of Exatecan ADCs

| Con- | | | Igrov1 | | A549 | |
|---|---|---|---|---|---|---|
| jugate | | | EC₅₀ | Span | EC₅₀ | Span |
| # | Name | DAR | (nM) | (%) | (nM) | (%) |
| 124-2 | 1848-H01 Y180F404/ | 7.6 | >10 | <10 | >10 | <10 |
| | K42/E161 + 24 | | | | | |
| 109-2 | 1848-H01 | 7.9 | 0.086 | 62 | >10 | <10 |
| | Y180F404/K42/E161 + 9 | | | | | |
| 108-2 | 1848-H01Y180F404/ | 7.8 | 0.198 | 65 | >10 | <10 |
| | K42/E161 + 8 | | | | | |
| 107-2 | 1848-H01 | 7.9 | 0.174 | 60 | >10 | <10 |
| | Y180F404/K42/E161 + 7 | | | | | |
| 106-2 | 1848-H01 | 7.9 | 0.212 | 59 | >10 | <10 |
| | Y180F404/K42/E161 + 6 | | | | | |

### Example 19: In Vitro Cell Killing Activity of Hemiasterlin ADCs

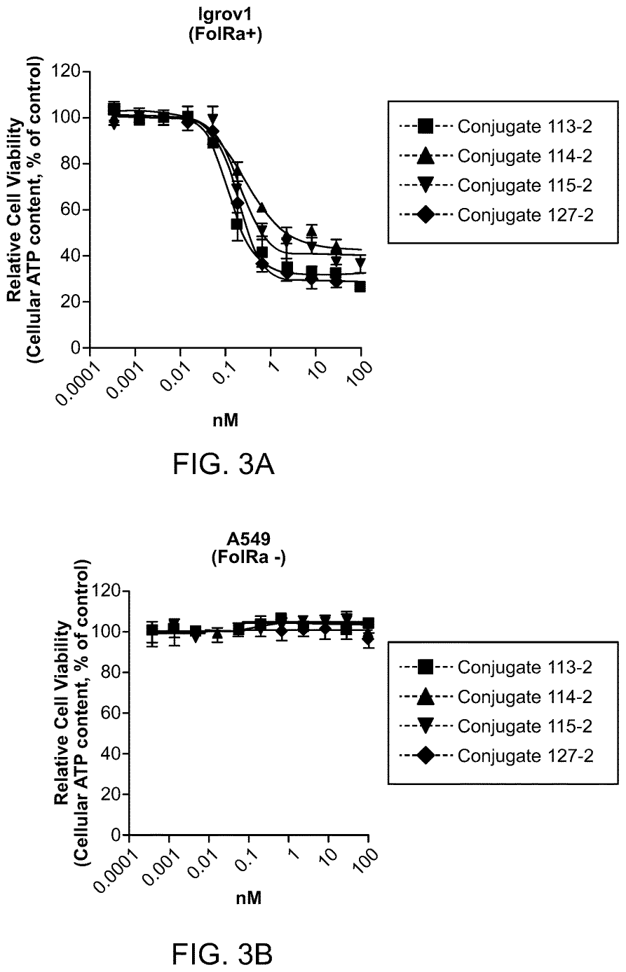

Anti-FolRα ADCs conjugated to hemiasterlin with different cathepsin cleavable linkers were also generated at DAR=4 and cell killing activities were evaluated on FolRα positive Igrov1 cells and FolRa negative A549 cells. As shown in FIG. 3A, FIG. 3B, and Table 5, similar to anti-FolRα ADC Conjugate 127-3, anti-FolRα ADCs conjugated to Compound 13, Compound 14, and Compound 15 at DAR4 showed potent cell killing on FolRα positive Igrov1 cells, while no cell killing was observed on FolRa negative A549 cells, which indicated that all the linkers tested were stable in the cell culture medium for 5 days.

TABLE 5
Cell killing activity of Hemiasterlin ADCs

| Con- | | | Igrov1 | | A549 | |
|---|---|---|---|---|---|---|
| jugate | | | EC₅₀ | Span | EC₅₀ | Span |
| # | Name | DAR | (nM) | (%) | (nM) | (%) |
| 113-2 | 1848-H01 Y180F404 + 13 | 4 | 0.126 | 70 | >10 | <10 |
| 114-2 | 1848-H01 Y180F404 + 14 | 4 | 0.303 | 58 | >10 | <10 |
| 115-2 | 1848-H01 Y180F404 + 15 | 4 | 0.212 | 60 | >10 | <10 |
| 127-3 | 1848-H01 Y180F404 + 27 | 3.7 | 0.189 | 70 | >10 | <10 |

The disclosure set forth above may encompass multiple distinct embodiments with independent utility. Although each of these embodiments has been disclosed, the specific embodiments thereof as disclosed and illustrated herein are not to be considered in a limiting sense, because numerous variations are possible. The subject matter of the embodiments includes all novel and nonobvious combinations and subcombinations of the various elements, features, functions, and/or properties disclosed herein. The following claims particularly point out certain combinations and subcombinations regarded as novel and nonobvious. Alternative embodiments as in other combinations and subcombinations of features, functions, elements, and/or properties may be claimed in this application, in applications claiming priority from this application, or in related applications. Such claims, whether directed to a different embodiment or to the same embodiment, and whether broader, narrower, equal, or different in scope in comparison to the original claims, also are regarded as included within the subject matter of this disclosure.

One or more features from any embodiments described herein or in the figures may be combined with one or more features of any other embodiments described herein or in the figures without departing from the scope of this disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A compound of Formula (I): 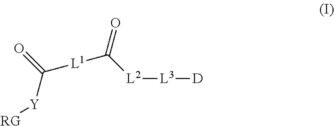
or a pharmaceutically acceptable salt and/or regioisomer thereof;
wherein
L¹ is selected from 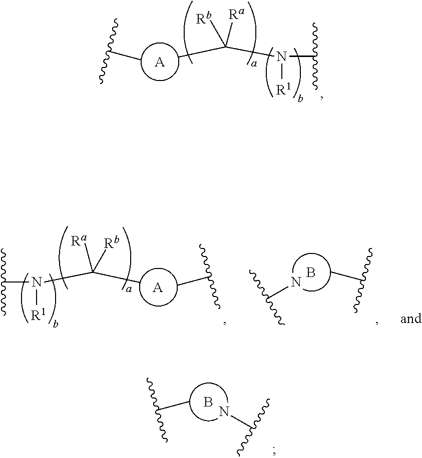
Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b in an integer selected from 0 and 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RG;
c is an integer selected from 1, 2, 3, 4, 5, and 6;
RG is a reactive group;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is 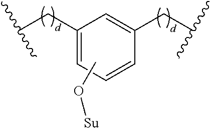
wherein Su is a hexose form of a monosaccharide;
d is an integer independently selected from 1, 2, and 3;
D is a cytotoxic payload; and [symbol]
represents attachment to the remainder of the compound.

2. The compound of claim 1, wherein L¹ is

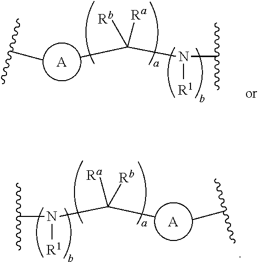

3. The compound of claim 2, wherein L¹ is

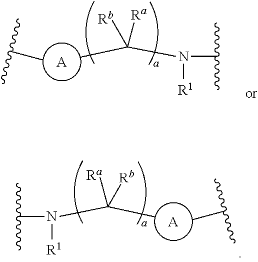

4. The compound of claim 1, wherein L¹ is

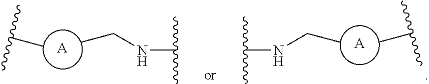

5. The compound of any one of claims 1-4, wherein Ring A of L¹ is an optionally substituted bridged, fused, or spirocyclic bicyclic carbocycle, wherein the carbocycle of Ring A is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl.

6. The compound of claim 5, wherein Ring A of L is an optionally substituted C₄₋₁₂ bridged, fused, or spirocyclic bicyclic carbocycle.

7. The compound of claim 5, wherein Ring A of L¹ is an optionally substituted C₄₋₁₂ bridged bicyclic carbocycle.

8. The compound of claim 5, wherein Ring A of L¹ is an optionally substituted C₄₋₈ bridged bicyclic carbocycle.

9. The compound of claim 1 or 4, wherein L¹ is

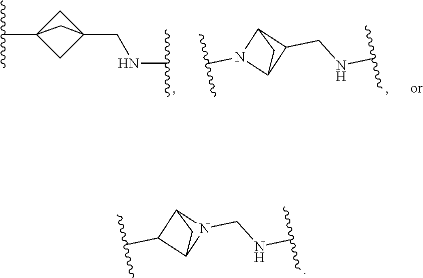

10. The compound of claim 9, wherein L¹ is

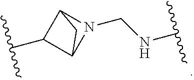 [SMILES: *NCN1C2CC1C2*]

11. The compound of claim 9, wherein L¹ is

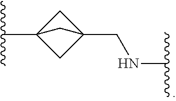 [SMILES: *NCC12CC(*)(C1)C2]

12. The compound of claim 1, wherein L¹ is

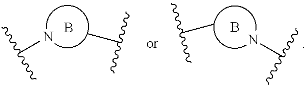

13. The compound of claim 1 or 12, wherein Ring B of L¹ is an optionally substituted 5- to 12-membered N-linked bridged, fused, or spirocyclic bicyclic heterocycle containing 1, 2, or 3 heteroatoms independently selected from N, O, and S including the N to which the ring is attached, wherein the heterocycle of Ring B is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl.

14. The compound of claim 13, wherein Ring B of L¹ is an optionally substituted 5- to 12-membered N-linked spirocyclic bicyclic heterocycle containing 1, 2, or 3 heteroatoms independently selected from N, O, and S including the N to which the ring is attached.

15. The compound of claim 1 or 12, wherein L¹ is selected from 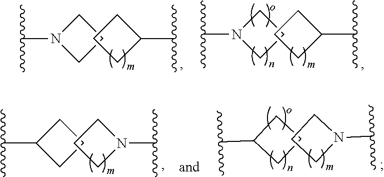
wherein m is an integer selected from 1, 2, 3, 4, and 5; and
each of n and o is an integer independently selected from 1, 2, and 3.

16. The compound of claim 1 or 12, wherein L¹ is selected from 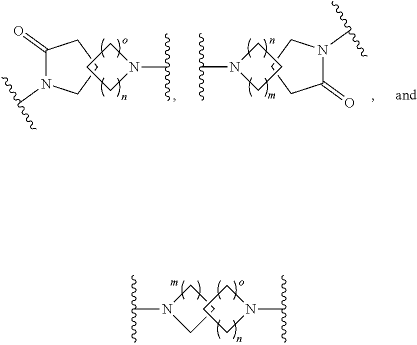
wherein m is an integer selected from 1, 2, 3, 4, and 5; and
each of n and o is an integer independently selected from 1, 2, and 3.

17. The compound of claim 1 or 12, wherein Ring B of L¹ is selected from 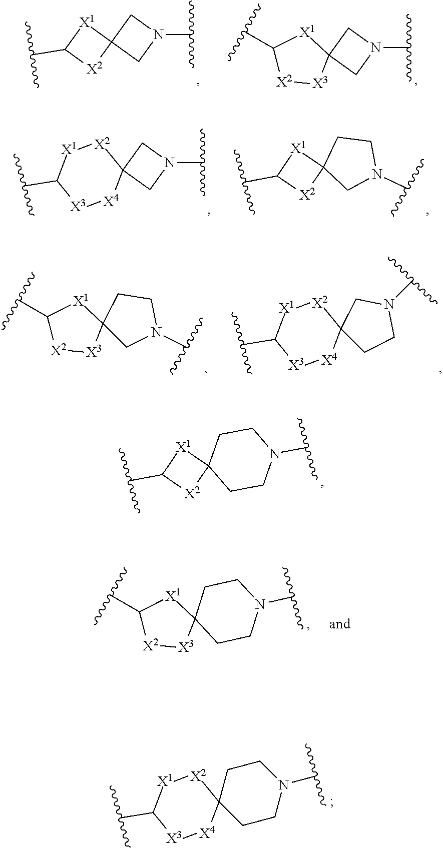
wherein X¹, X², X³, and X⁴ are independently selected from —C(R⁴)₂—, —NH—, —O—, and —S—, wherein when X¹, X², and X³ are present, at least one of X¹-X³ is —C(R⁴)₂— and when X¹, X², X³, and X⁴ are present, at least two of X¹-X⁴ are —C(R⁴)₂—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

18. The compound of claim 17, wherein Ring B is selected from

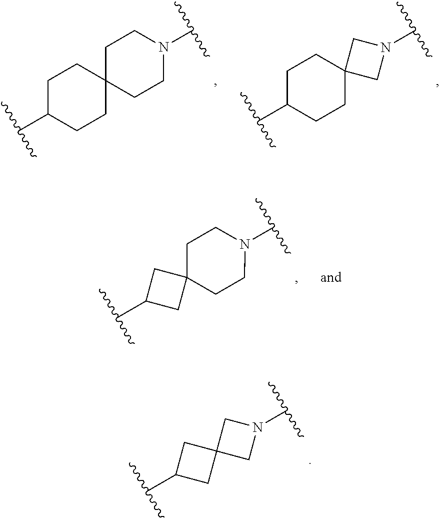 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

19. The compound of claim 1 or 12, where Ring B is selected from 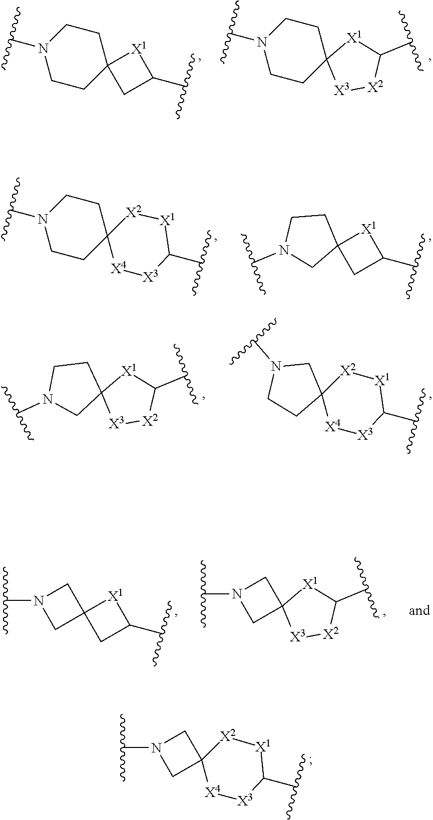
wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

20. The compound of claim 19, wherein Ring B is selected from

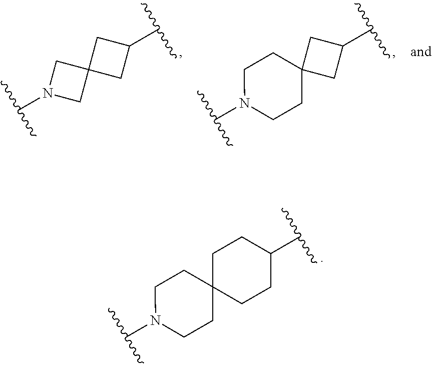 [SMILES: *C1CC2(C1)CN(*)C2 ; *C1CC2(CCN(*)CC2)C1 ; *C1CCC2(CC1)CCN(*)CC2]

21. The compound of any one of claims 1-4, wherein Ring A is selected from 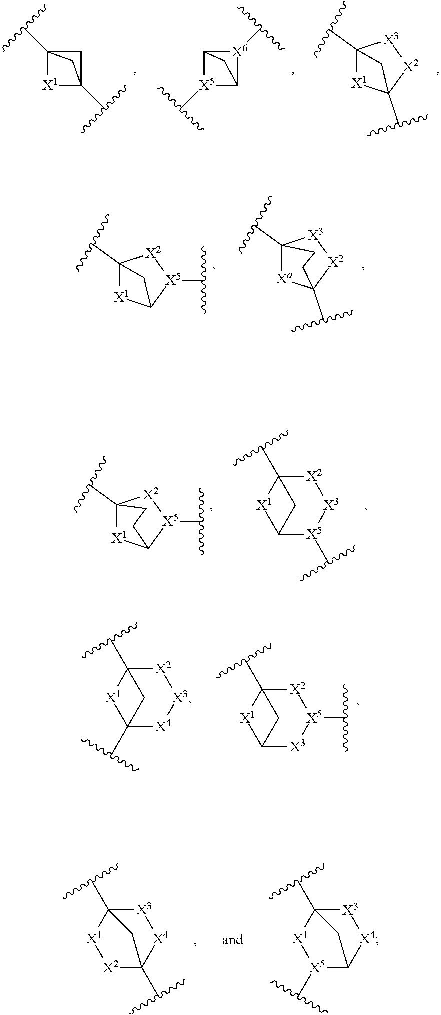
wherein X¹, X², X³, and X⁴ are independently selected from —C(R)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—;
X⁵ is —CR⁴— or —N—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

22. A compound of Formula (III): 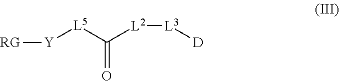
or a pharmaceutically acceptable salt and/or regioisomer thereof;
wherein
L⁵ is a linker comprising an unnatural amino acid;
Y is *—C(O)—(CR^{a}R^{b})—NH— or *—C(O)—(CR^{a}R^{b})_{c}—, wherein * represents where Y is bound to RG;
c is an integer selected from 1, 2, 3, 4, 5, and 6;
RG is a reactive group;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
Z is selected from —NR²— and —O—;
AA is an amino acid residue or a peptide residue;
L⁴ is 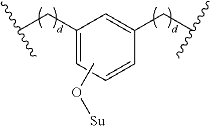
wherein Su is a hexose form of a monosaccharide;
d is an integer independently selected from 1, 2, and 3;
D is a cytotoxic payload;
and [symbol] represents attachment to the remainder of the compound.

23. The compound of claim 22, wherein L⁵ is a linker that comprises at least one amino acid selected from sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone.

24. The compound of claim 22, wherein L⁵ is a linker that comprises

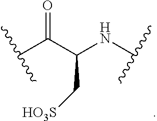 [SMILES: *N[C@@H](CS(=O)(=O)O)C(*)=O]

25. The compound of claim 22, wherein L⁵ is

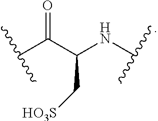 [SMILES: *N[C@@H](CS(=O)(=O)O)C(*)=O]

26. The compound of any one of claims 1-25, wherein L² is selected from —(CR^{a}R^{b})ₐ-POLY¹-, -POLY¹-, —(CR^{a}R^{b})ₐ-POLY¹-(CR^{a}R^{b})ₐ—, 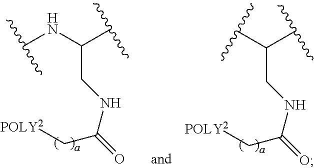
wherein POLY¹ is a divalent hydrophilic polymer residue; POLY² is a residue of a hydrophilic polymer; and [symbol] represents attachment to the remainder of the compound.

27. The compound of any one of claims 1-26, wherein Y is —C(O)—(CR^{a}R^{b})_{c}—NH—.

28. The compound of claim 27, wherein Y is —C(O)—CH₂CH₂—NH—.

29. The compound of any one of claims 1-26, wherein Y is —C(O)—(CR^{a}R^{b})_{c}—.

30. The compound of claim 29, wherein Y is —C(O)—(CH₂)₄—.

31. The compound of any one of claims 1-30, wherein RG comprises an alkyne, cyclooctyne, a strained alkene, a tetrazine, methylcyclopropene (Mecyp), a thiol, a para-acetyl-phenylalanine residue, an oxyamine, a maleimide, or an azide.

32. The compound of claim 31, wherein RG is selected from 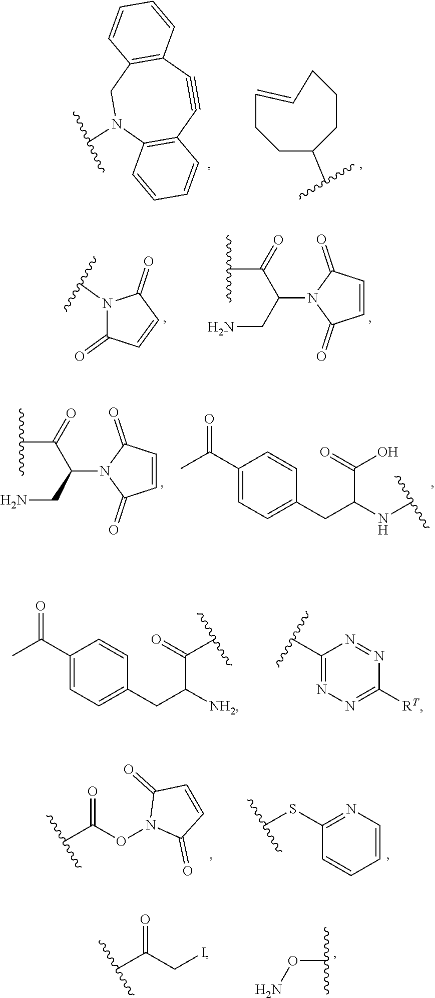
—N₃, —NH₂ and —SH; wherein R^{T} is C₁₋₆ alkyl; and [symbol] represents attachment to the remainder of the compound.

33. The compound of claim 32, wherein RG is selected from

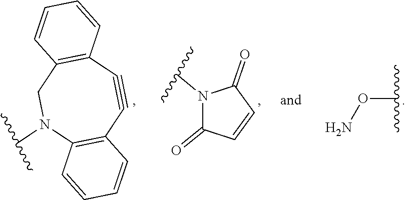

34. The compound of claim 32, wherein RG is

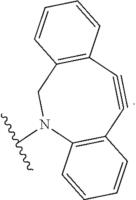 [SMILES: *N1Cc2ccccc2C#Cc2ccccc21]

35. The compound of any one of claims 1-25 and 31-34, wherein L²-(CR^{a}R^{b})ₐ-POLY¹-.

36. The compound of claim 35, wherein -POLY¹- is a divalent residue of polyethylene glycol (PEG), poly(propylene glycol) (PPG), or a copolymer of ethylene glycol and propylene glycol.

37. The compound of claim 36, wherein the divalent hydrophilic polymer residue has the formula 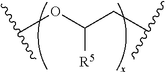
wherein R⁵ is hydrogen or methyl and x is an integer between 1 and 100, inclusive.

38. The compound of claim 37, wherein

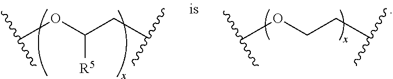

39. The compound of claim 36 or 37, wherein x is an integer between 1-25, inclusive.

40. The compound of claim 36 or 37, wherein x is an integer between 1-15, inclusive.

41. The compound of claim 36 or 37, wherein x is 13.

42. The compound of any one of claims 1-25, wherein L² is

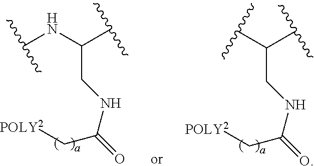

43. The compound of claim 42, wherein POLY² is residue of polyethylene glycol (PEG), methoxypolyethylene glycol (mPEG), poly(propylene glycol) (PPG), a copolymer of ethylene glycol and propylene glycol, or polysarcosine.

44. The compound of claim 43, wherein POLY² is 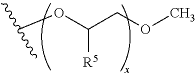
wherein R⁵ is hydrogen or methyl and x is an integer between 1 and 100, inclusive.

45. The compound of claim 44, wherein

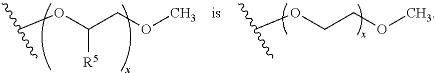

46. The compound of claim 43 or 44, wherein x is an integer between 1-25, inclusive.

47. The compound of claim 43 or 44, wherein x is an integer between 1-15, inclusive.

48. The compound of claim 43 or 44, wherein x is 11.

49. The compound of any one of claims 1-48, wherein L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, or —C(O)—.

50. The compound of any one of claims 1-48, wherein L³ is —C(O)-AA-.

51. The compound of any one of claims 1-48, wherein L³ is —C(O)-AA-Z—(CR^{a}R^{b})ₐ—Z—(CR^{a}R^{b})ₐ—C(O)—, —Z-AA-, or -AA-.

52. The compound of claim 51, wherein Z is —NH—.

53. The compound of any one of claims 1-48, wherein L³ is -AA-.

54. The compound of claim 53, wherein -AA- is

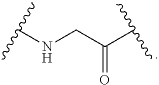 [SMILES: *NCC(*)=O]

55. The compound of any one of claims 50-53, wherein AA is a dipeptide residue, a tripeptide residue, a tetrapeptide residue, or a pentapeptide residue.

56. The compound of claim 55, wherein AA is a tripeptide residue.

57. The compound of any one of claims 50-53 and 55-56, wherein AA comprises at least one amino acid residue selected from alanine, glycine, valine, and asparagine.

58. The compound of any one of claims 50-53 and 55-56, wherein AA comprises at least one amino acid residue selected from alanine and glycine.

59. The compound of any one of claims 50-53, wherein AA is selected from the group consisting of

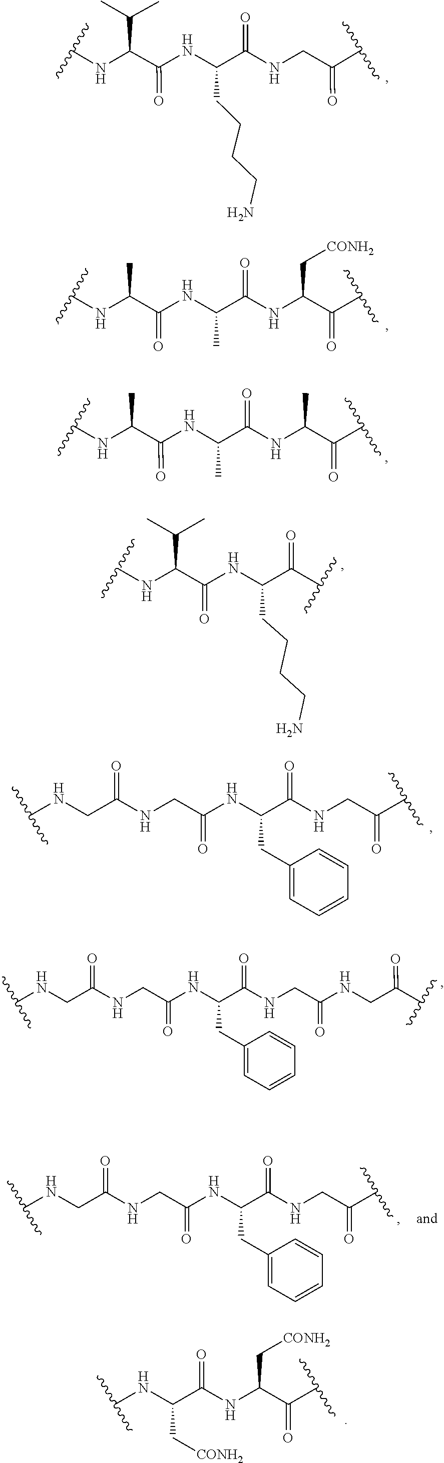

60. The compound of any one of claims 1-48, wherein
L³ is —C(O)—Z—(CR^{a}R^{b})ₐ—C(O)—Z-L⁴-OC(O)—;
L⁴ is 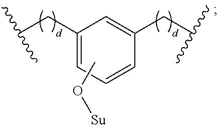
and
Su is a hexose form of a monosaccharide.

61. The compound of claim 60, wherein Su is

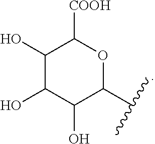 [SMILES: *C1OC(C(=O)O)C(O)C(O)C1O]

62. The compound of claim 61, wherein Su is

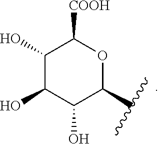 [SMILES: *[C@@H]1O[C@H](C(=O)O)[C@@H](O)[C@H](O)[C@H]1O]

63. The compound of any one of claims 1-62, wherein the D is a cytotoxic payload selected from a group consisting of a tubulin inhibitor, DNA damaging agent, a DNA topoisomerase I inhibitor, a DNA topoisomerase II inhibitor, and RNA polymerase II inhibitor.

64. The compound of claim 63, wherein D is a cytotoxic payload selected from the group consisting of a hemiasterlin or derivative thereof, a camptothecin or derivative thereof, an anthracycline or derivative thereof, a PNU-159682 or derivative thereof, a PBD or derivative thereof, and a duocarmycin or derivative thereof.

65. The compound of claim 63, wherein D is selected from: 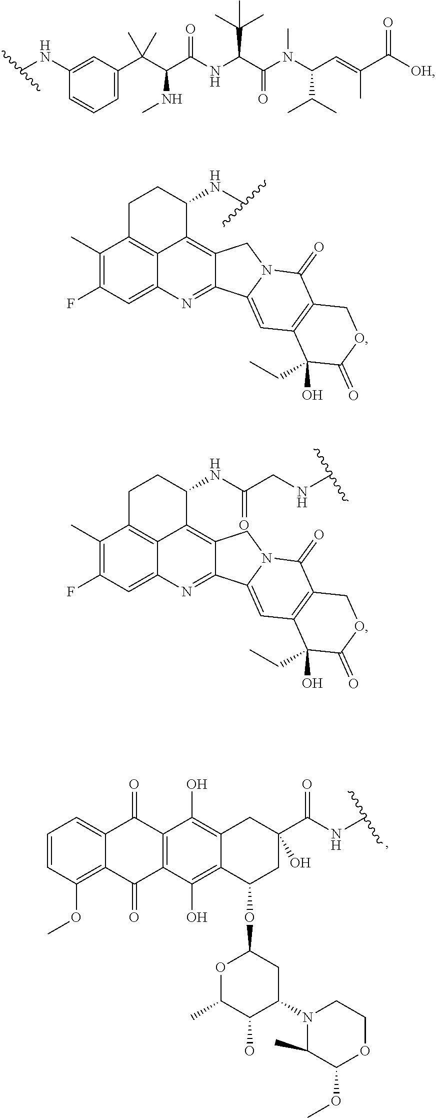 [SMILES: *Nc1cccc(C(C)(C)[C@H](NC)C(=O)N[C@H](C(=O)N(C)[C@H](/C=C(\C)C(=O)O)C(C)C)C(C)(C)C)c1 ; *N[C@H]1CCc2c(C)c(F)cc3nc4c(c1c23)Cn1c-4cc2c(c1=O)COC(=O)[C@]2(O)CC ; *NCC(=O)N[C@H]1CCc2c(C)c(F)cc3nc4c(c1c23)Cn1c-4cc2c(c1=O)COC(=O)[C@]2(O)CC ; *NC(=O)[C@]1(O)Cc2c(O)c3c(c(O)c2[C@@H](O[C@H]2C[C@H](N4CCO[C@H](OC)[C@H]4C)[C@H](O)[C@H](C)O2)C1)C(=O)c1c(OC)cccc1C3=O] 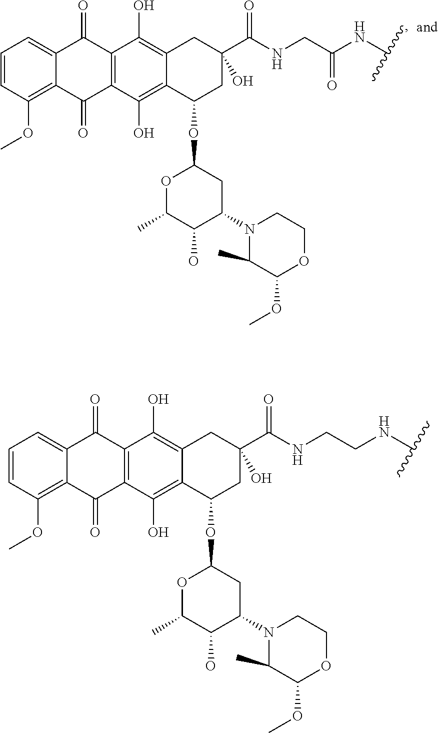 [SMILES: *NC(=O)CNC(=O)[C@]1(O)Cc2c(O)c3c(c(O)c2[C@@H](O[C@H]2C[C@H](N4CCO[C@H](OC)[C@H]4C)[C@H](O)[C@H](C)O2)C1)C(=O)c1c(OC)cccc1C3=O ; *NCCNC(=O)[C@]1(O)Cc2c(O)c3c(c(O)c2[C@@H](O[C@H]2C[C@H](N4CCO[C@H](OC)[C@H]4C)[C@H](O)[C@H](C)O2)C1)C(=O)c1c(OC)cccc1C3=O]
or a derivative thereof.

66. The compound of claim 63, wherein D is an exatecan payload.

67. The compound of claim 1, wherein the compound is represented by: 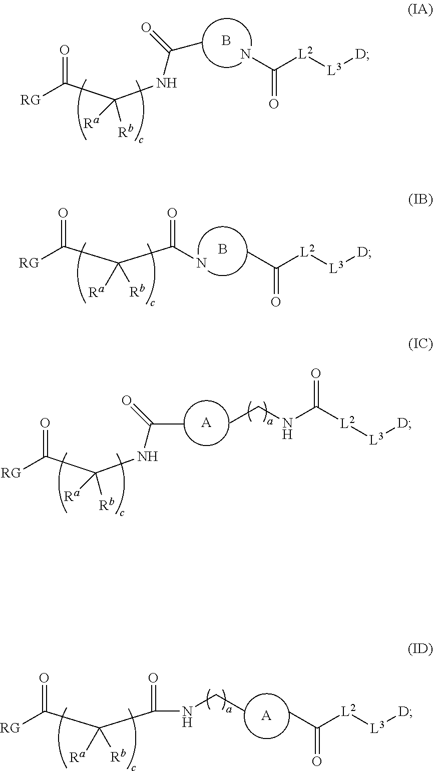
or a pharmaceutically acceptable salt thereof.

68. The compound of claim 67, wherein the compound is represented by: 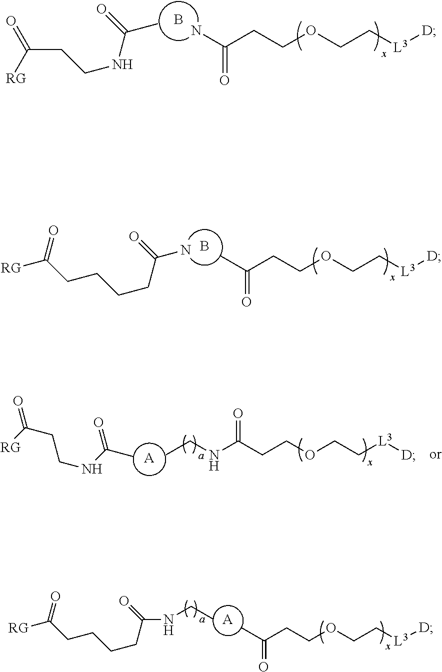
or a pharmaceutically acceptable salt thereof.

69. The compound of claim 67, wherein the compound is represented by: 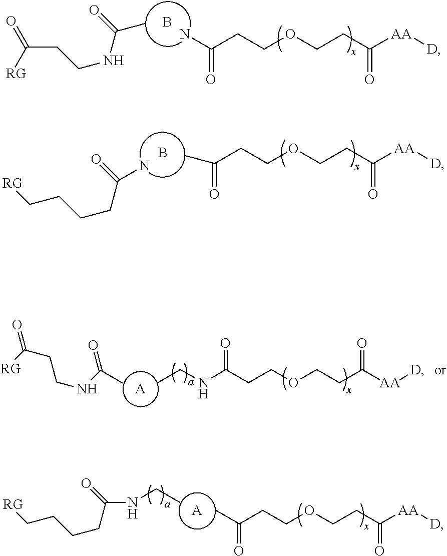
or a pharmaceutically acceptable salt thereof.

70. The compound of claim 1, wherein the compound is represented by: 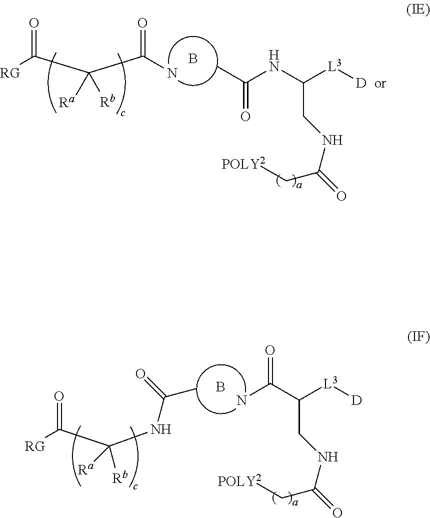
or a pharmaceutically acceptable salt thereof.

71. The compound of claim 70, wherein the compound is represented by: 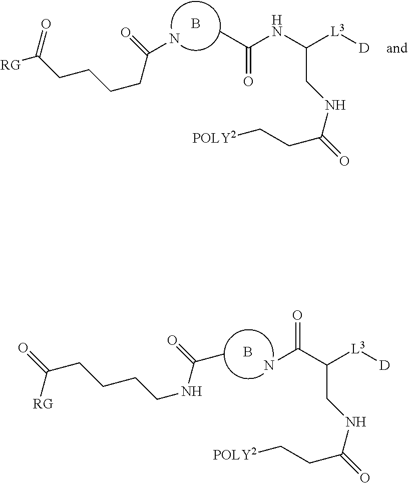
or a pharmaceutically acceptable salt thereof.

72. The compound of claim 71, wherein the compound is represented by: 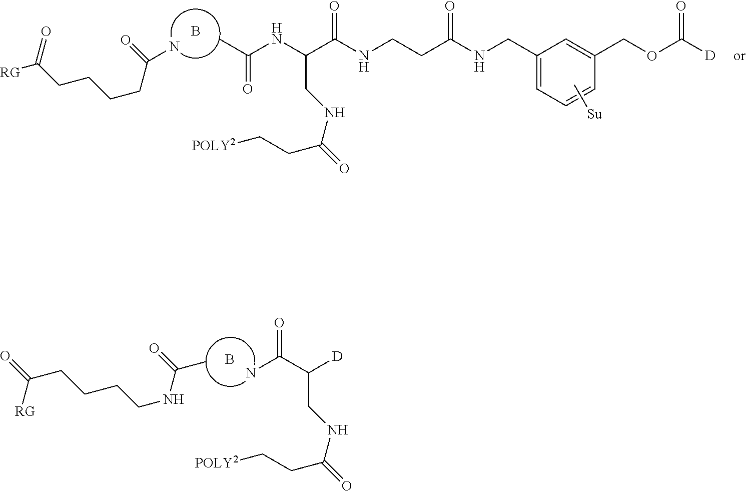
or a pharmaceutically acceptable salt thereof.

73. The compound of claim 1, wherein the compound is selected from: 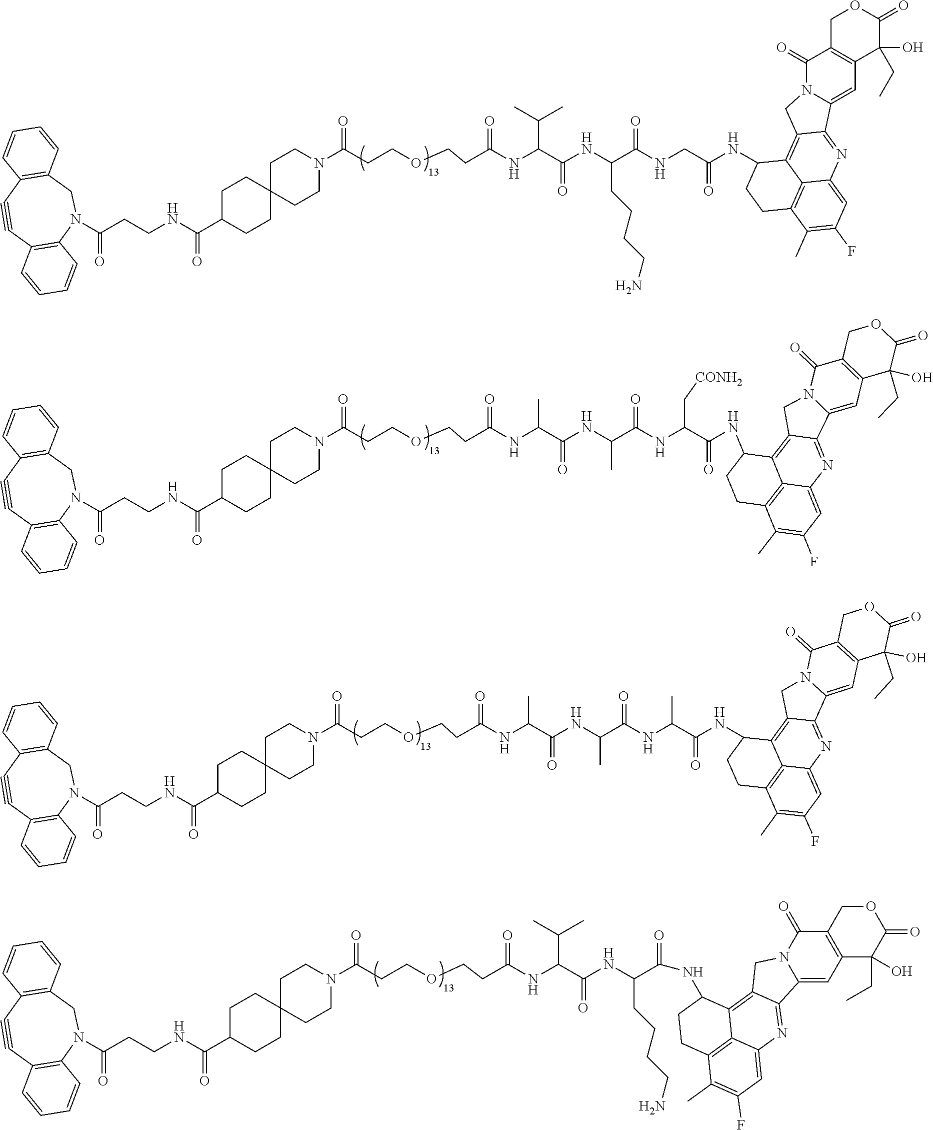 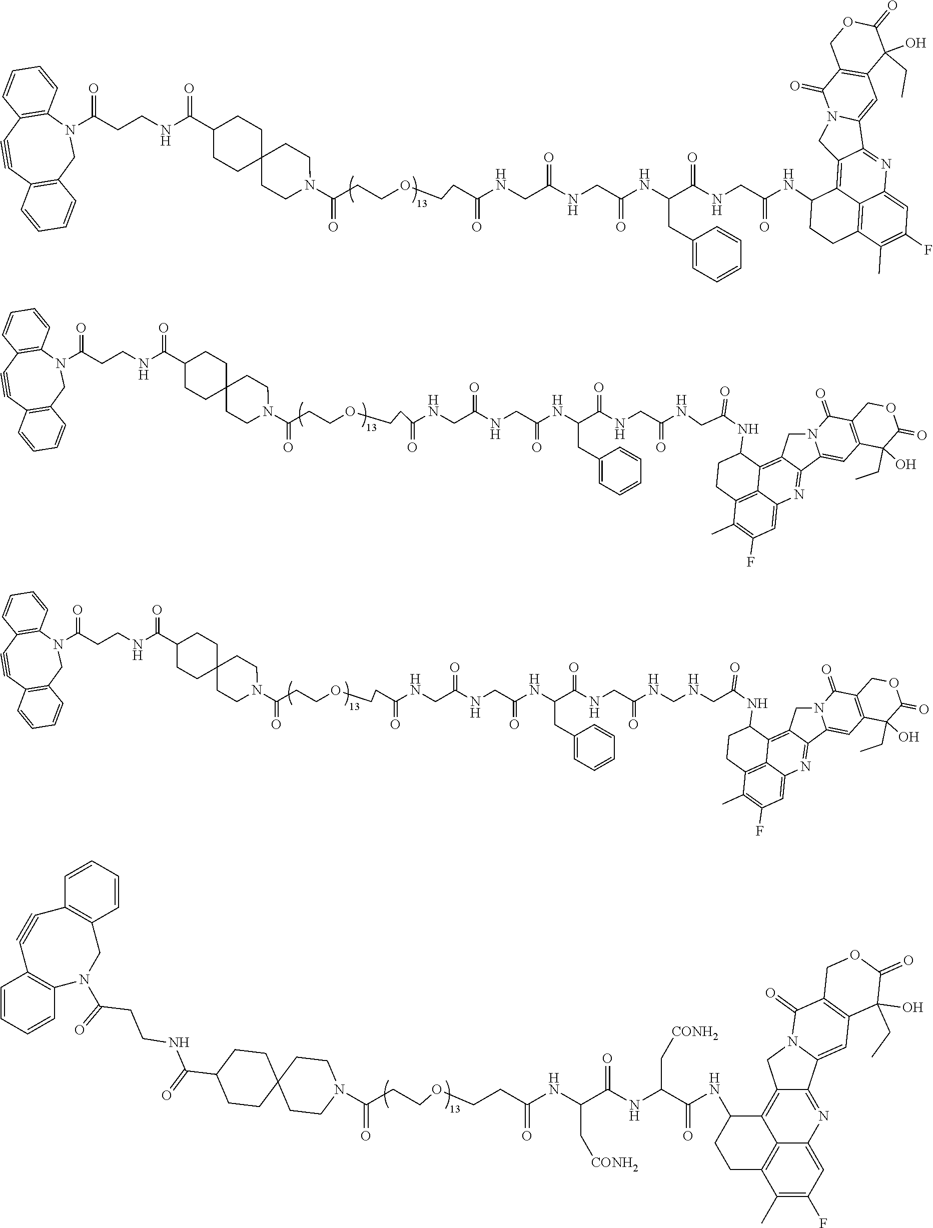 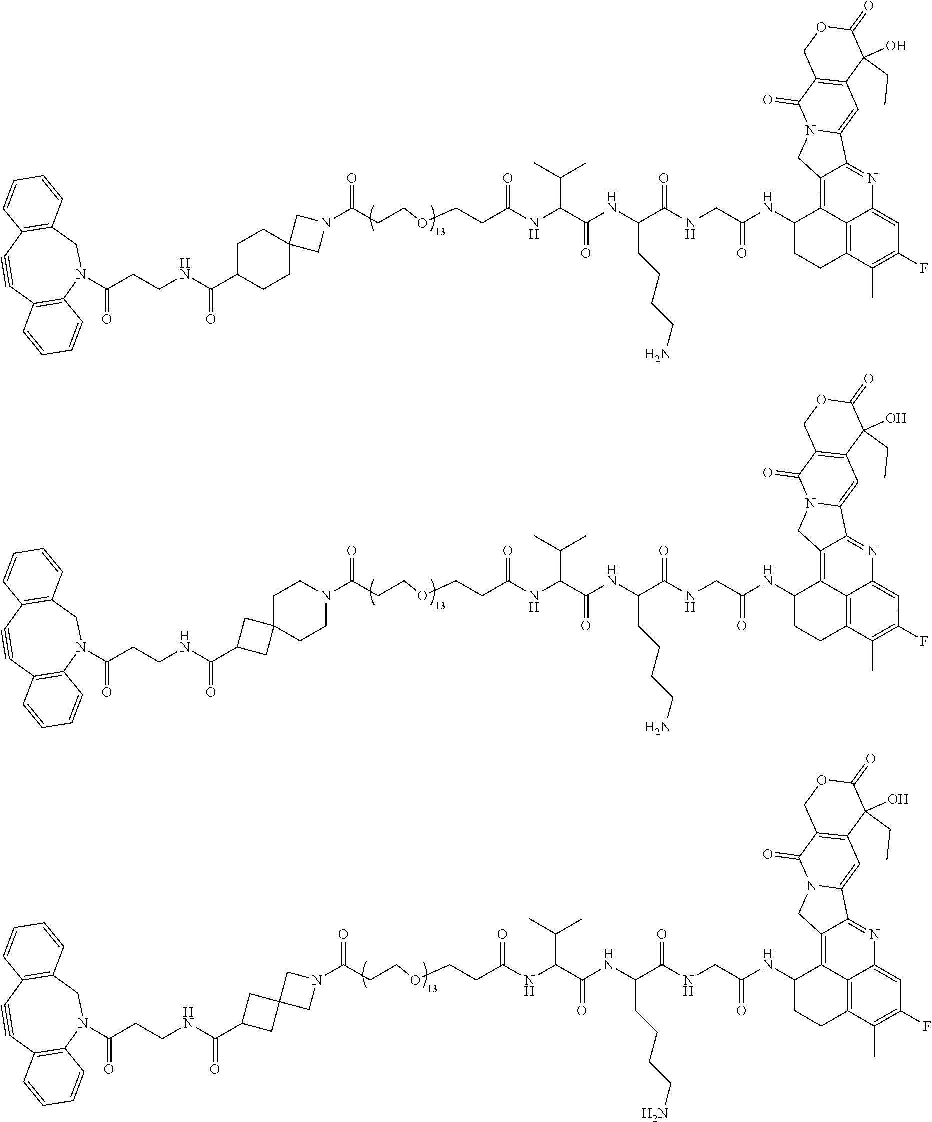 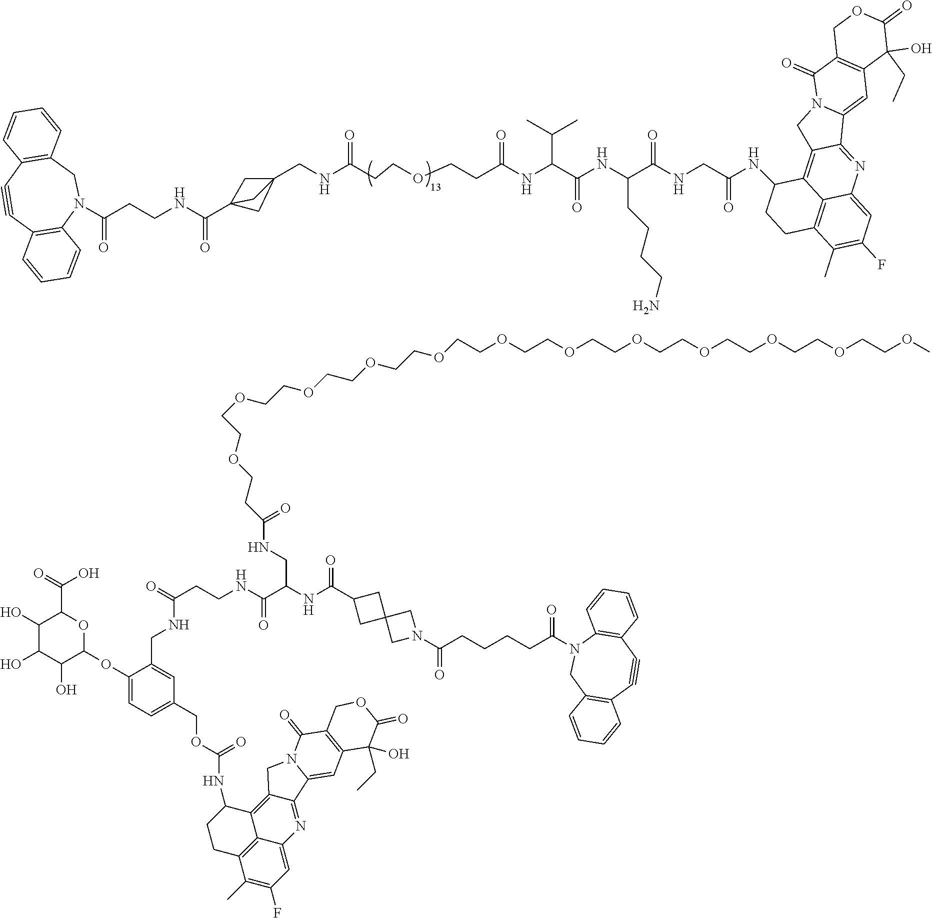 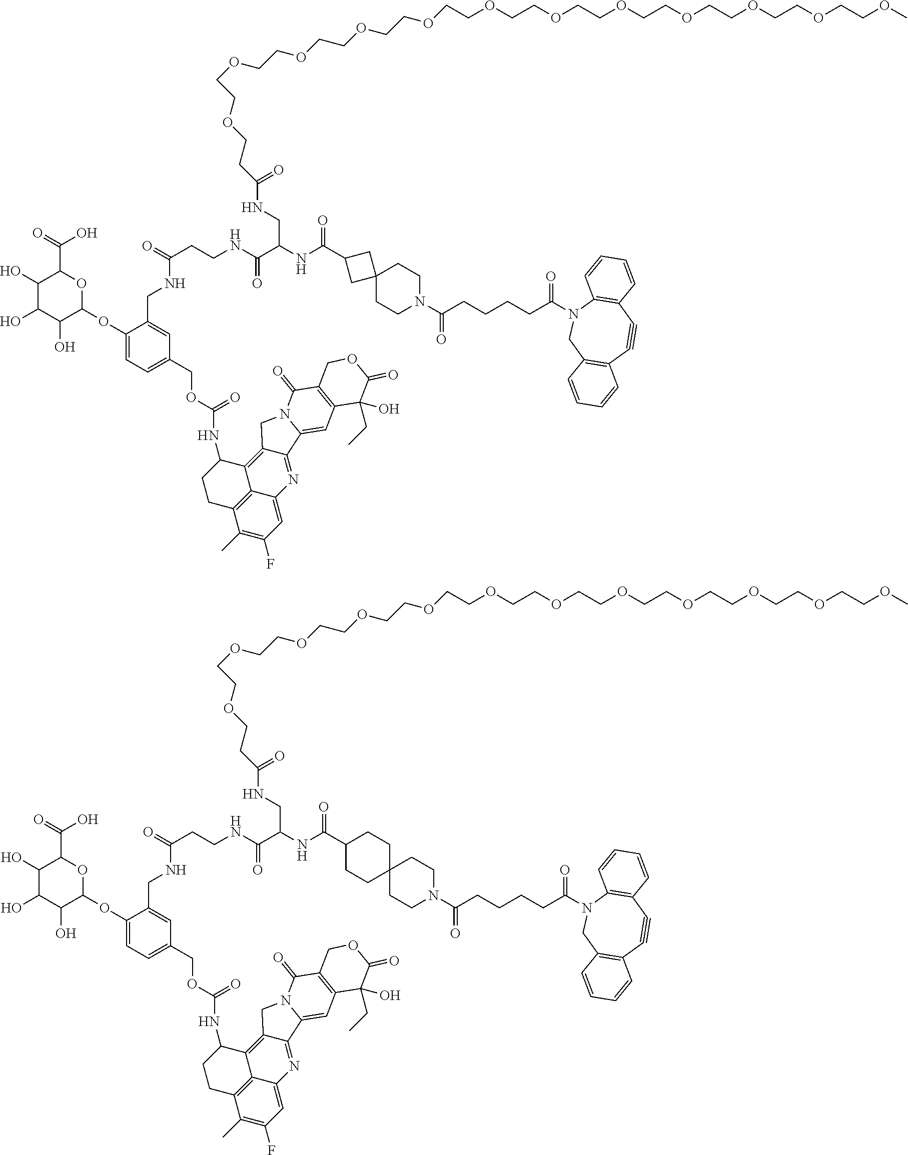 [SMILES: CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CC4(CCN(C(=O)CCCCC(=O)N5Cc6ccccc6C#Cc6ccccc65)CC4)C2)c1)CC3 ; CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CCC4(CC2)CCN(C(=O)CCCCC(=O)N2Cc5ccccc5C#Cc5ccccc52)CC4)c1)CC3] 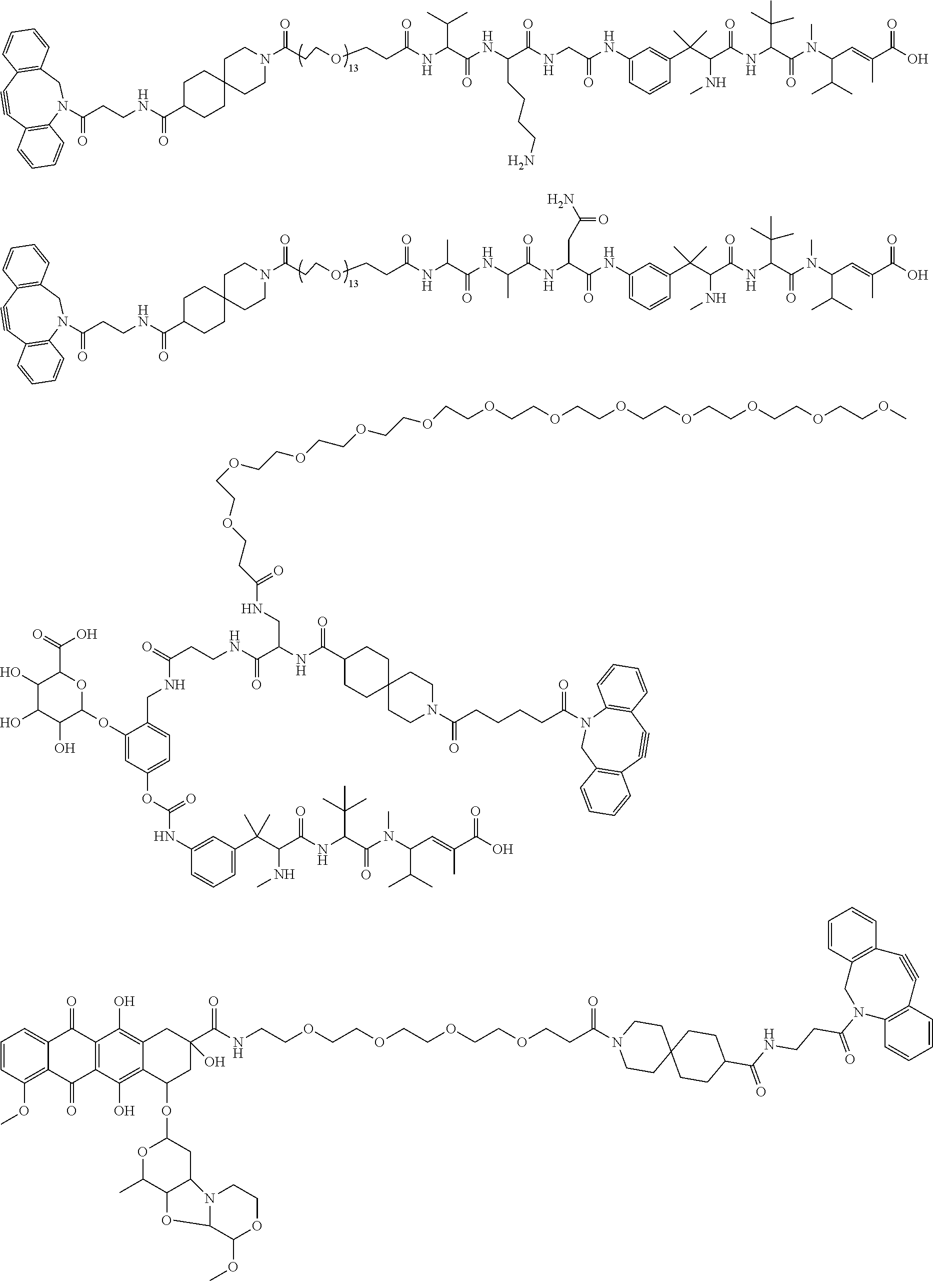 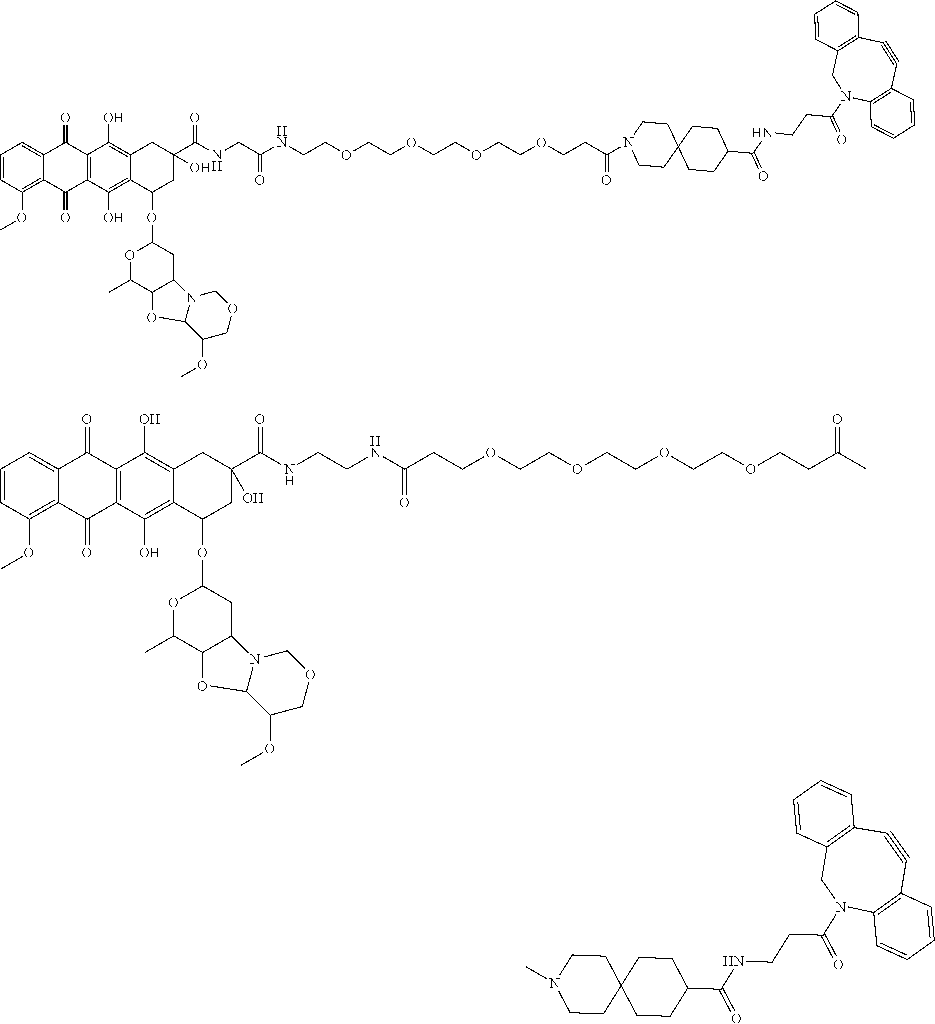 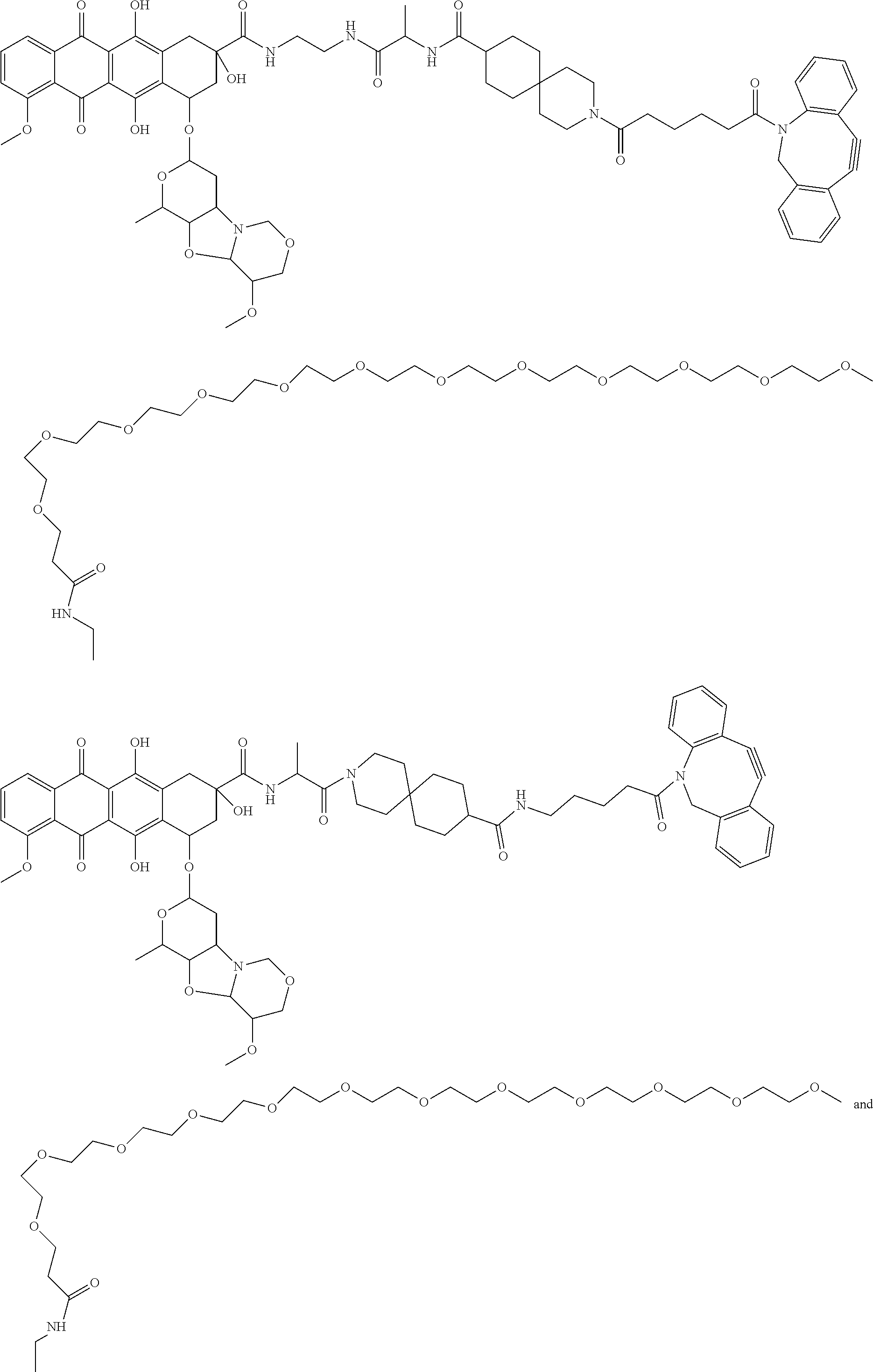 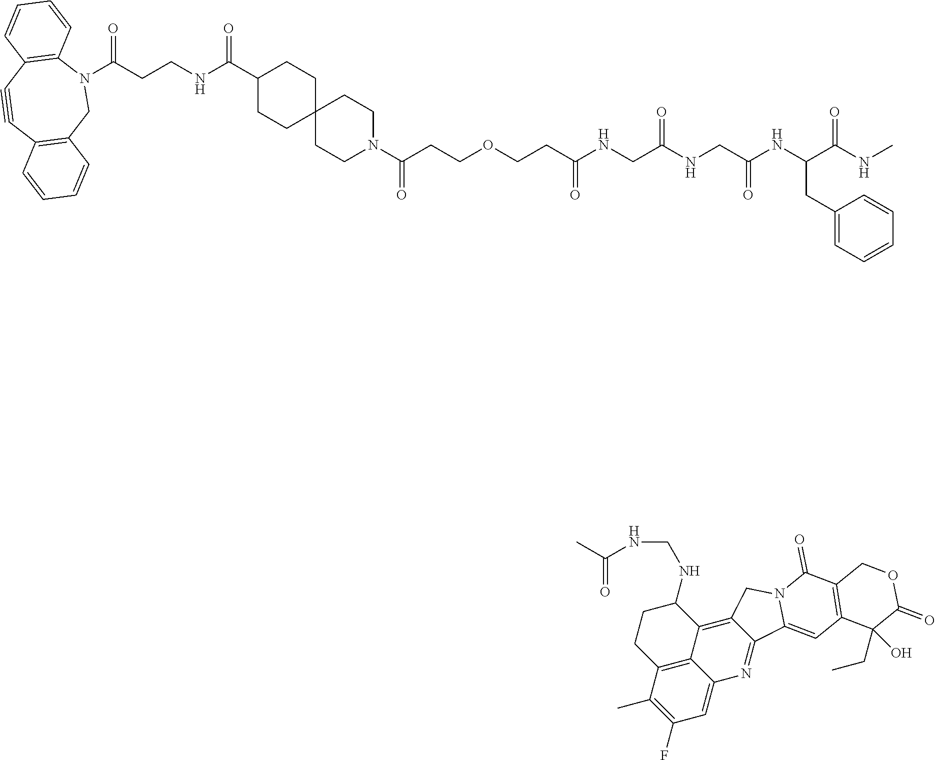 [SMILES: CNC(=O)C(Cc1ccccc1)NC(=O)CNC(=O)CNC(=O)CCOCCC(=O)N1CCC2(CCC(C(=O)NCCC(=O)N3Cc4ccccc4C#Cc4ccccc43)CC2)CC1 ; CCC1(O)C(=O)OCc2c1cc1n(c2=O)Cc2c-1nc1cc(F)c(C)c3c1c2C(NCNC(C)=O)CC3]
or a pharmaceutically acceptable salt thereof.

74. The compound of claim 73, wherein the compound is selected from: 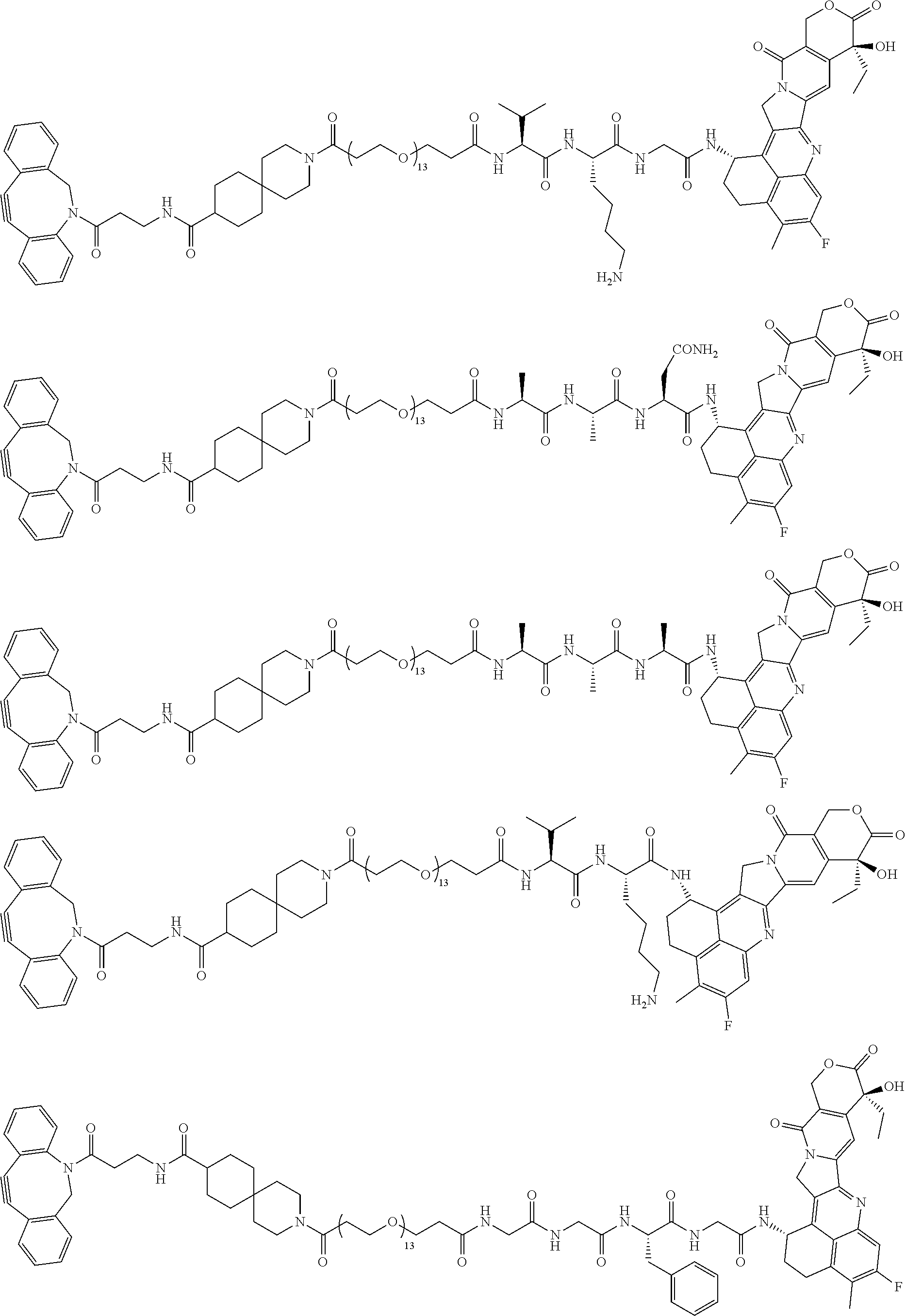 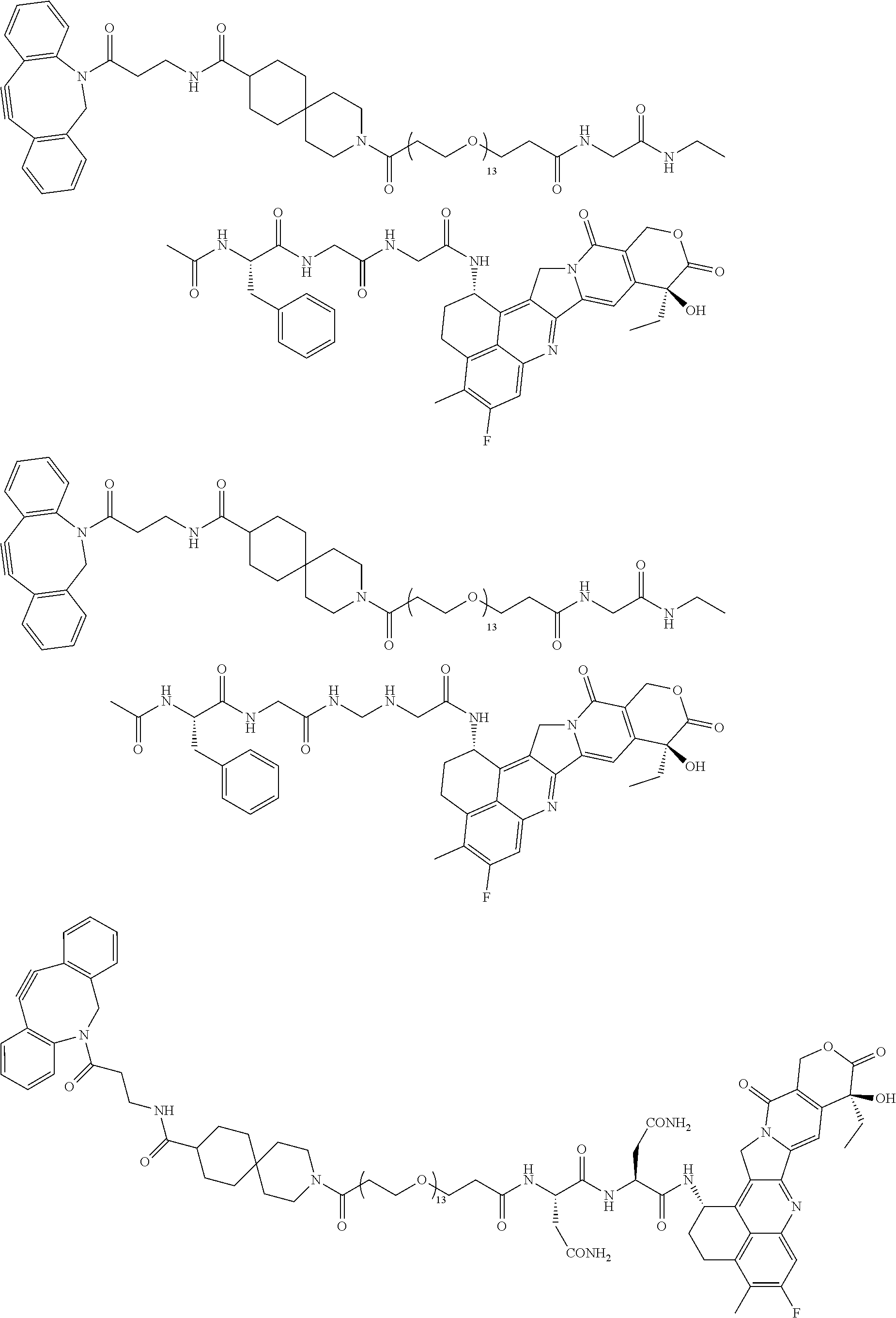 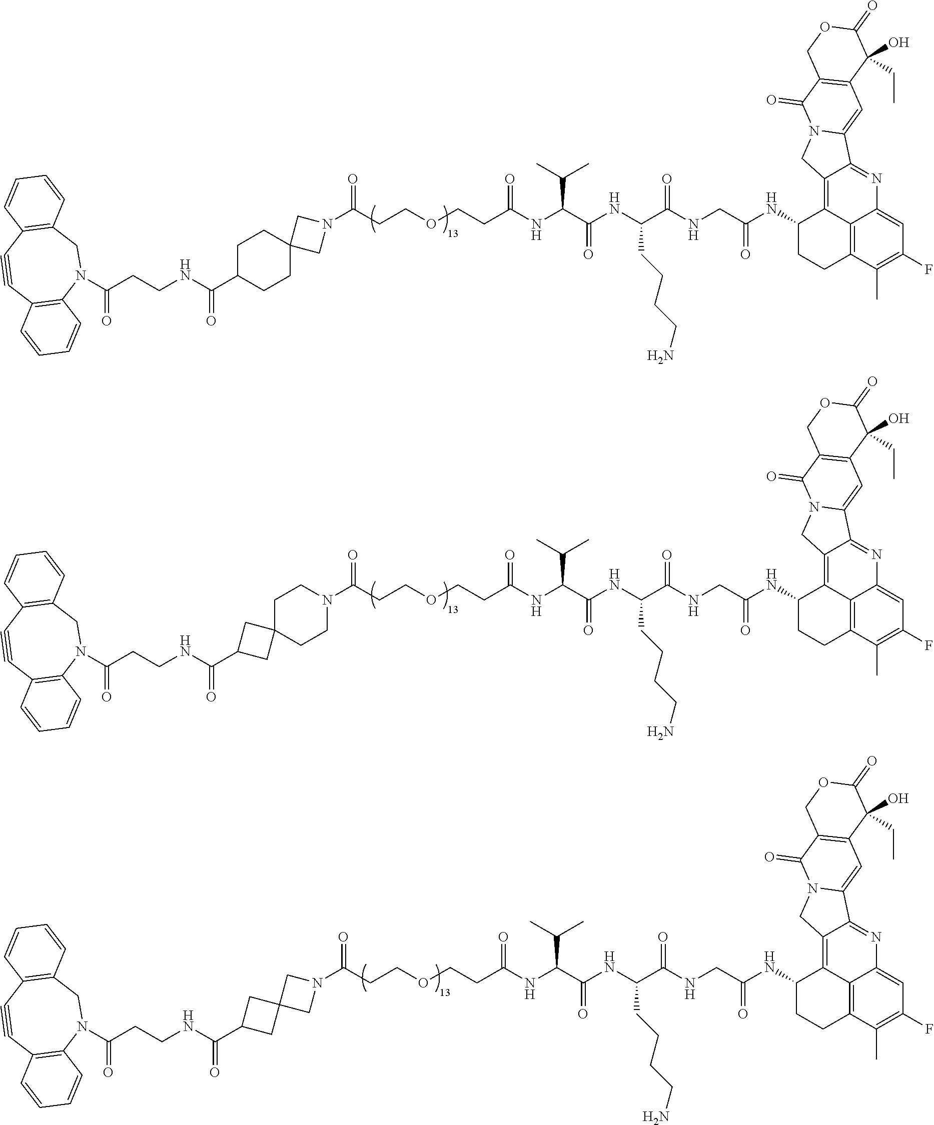 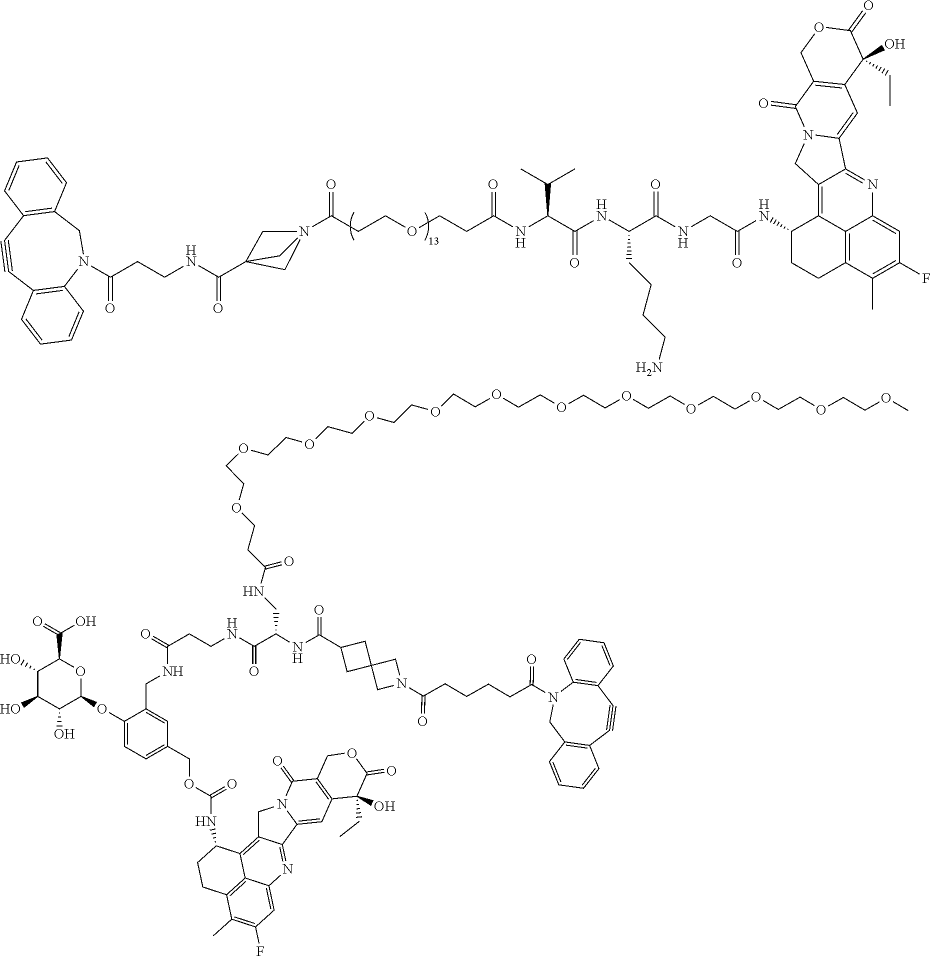 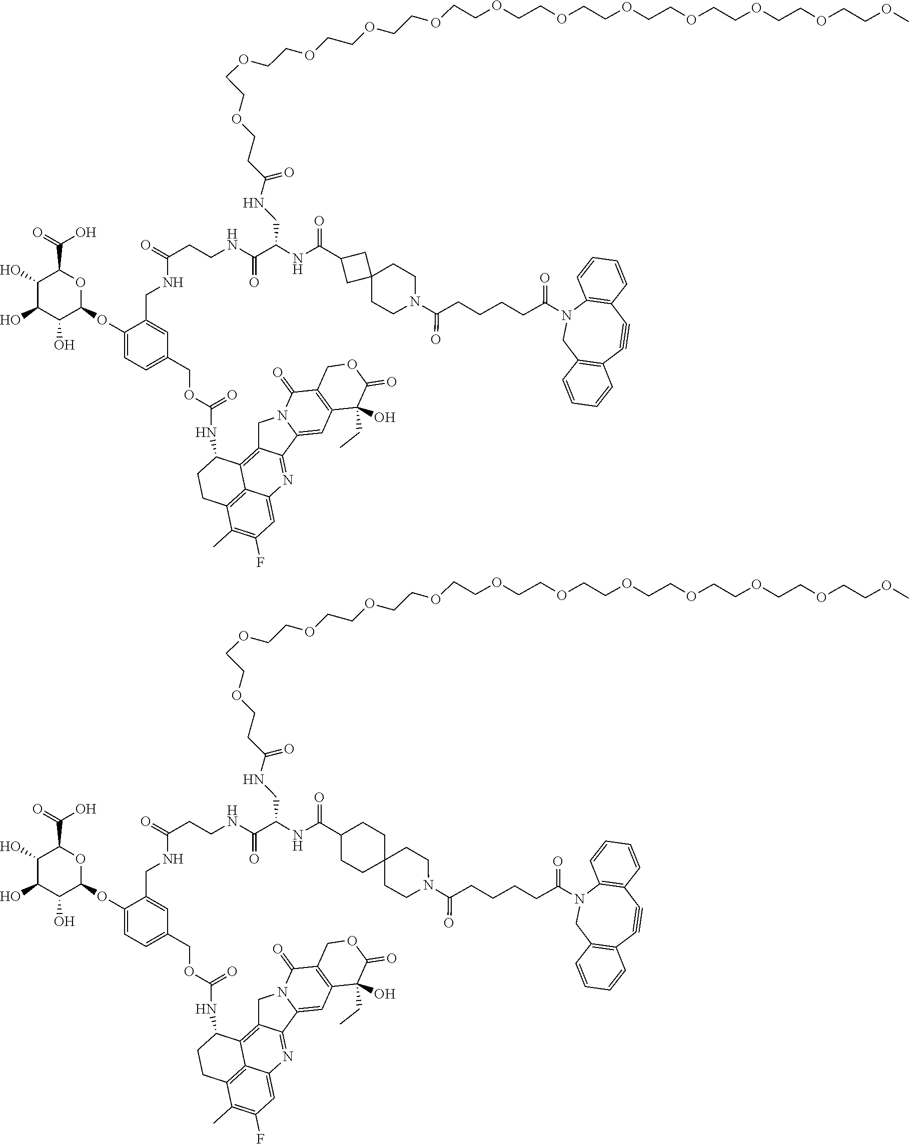 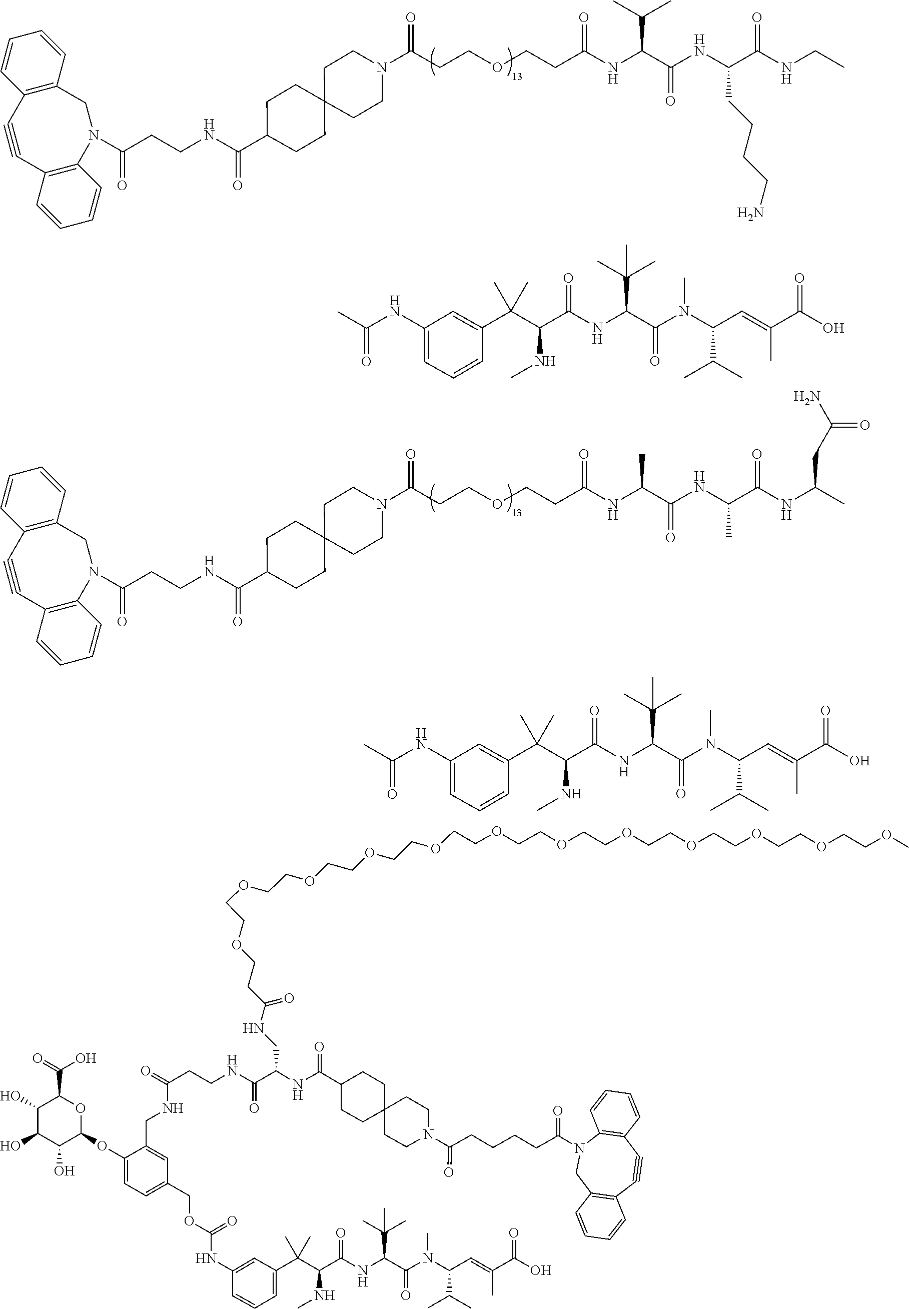 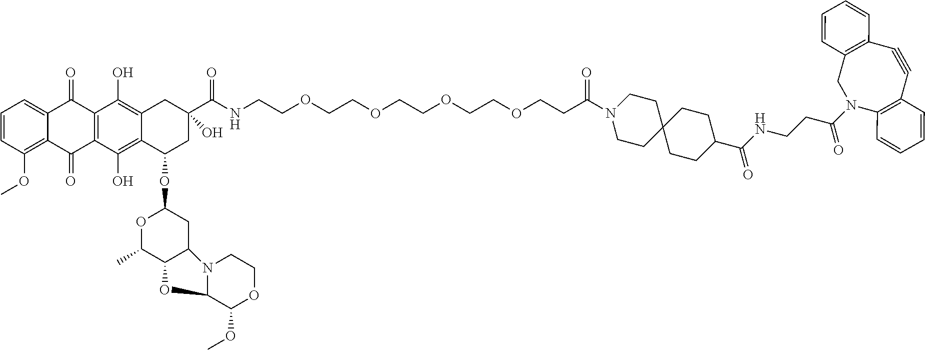 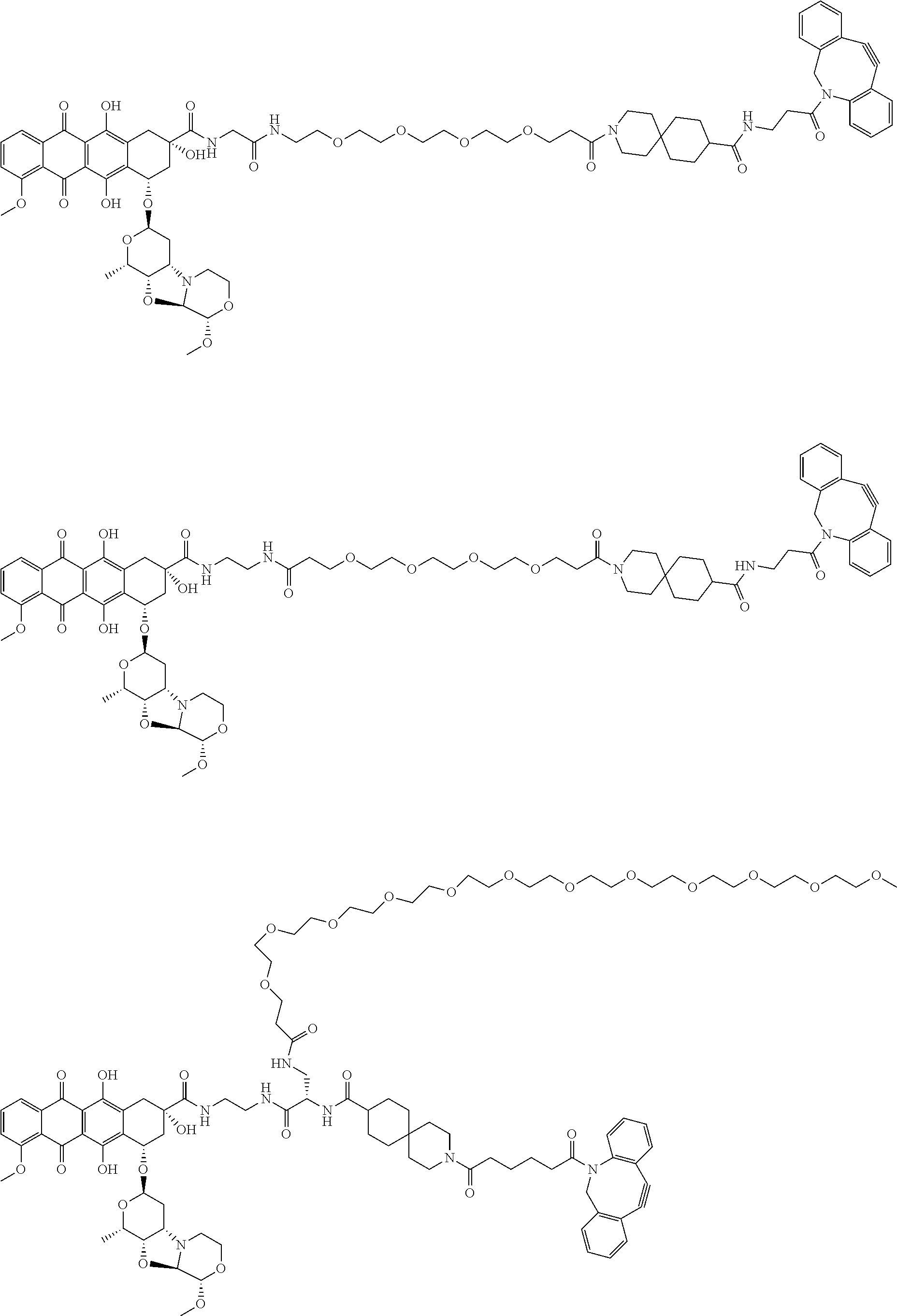 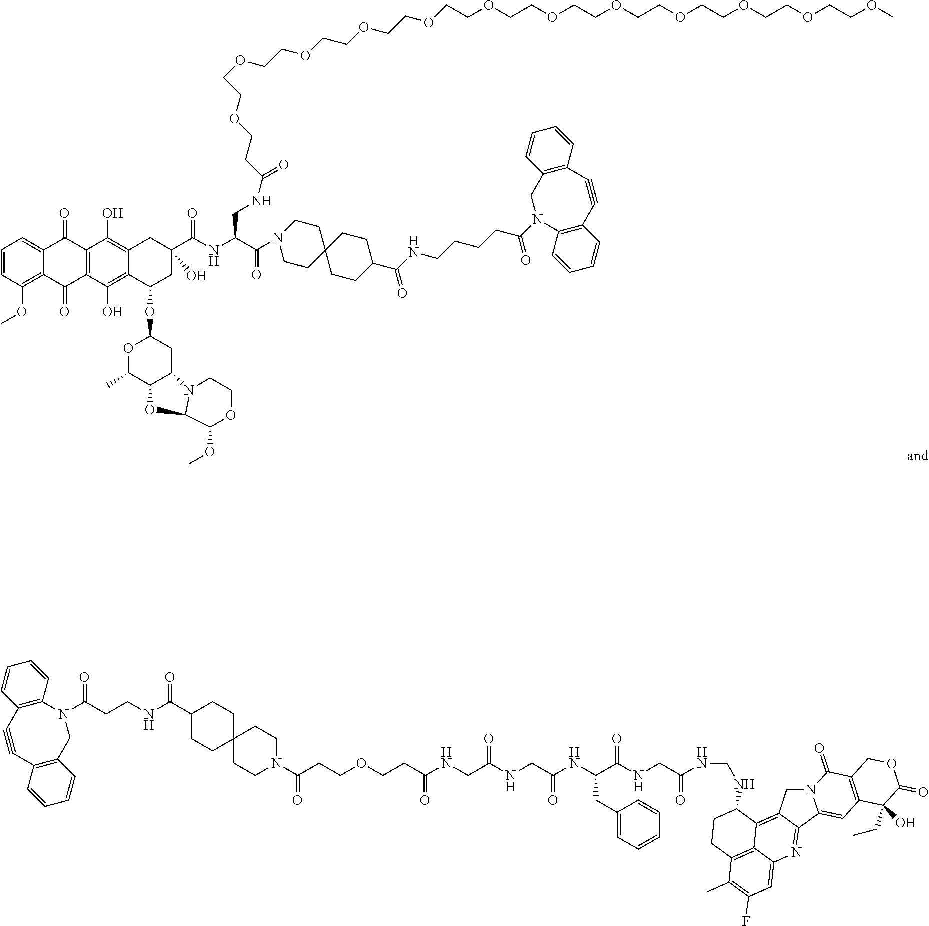 [SMILES: COCCOCCOCCOCCOCCOCCOCCOCCOCCOCCOCCOCCC(=O)NC[C@H](NC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O)C(=O)N1CCC2(CCC(C(=O)NCCCCC(=O)N3Cc4ccccc4C#Cc4ccccc43)CC2)CC1 ; CC[C@@]1(O)C(=O)OCc2c1cc1n(c2=O)Cc2c-1nc1cc(F)c(C)c3c1c2[C@@H](NCNC(=O)CNC(=O)[C@H](Cc1ccccc1)NC(=O)CNC(=O)CNC(=O)CCOCCC(=O)N1CCC2(CCC(C(=O)NCCC(=O)N4Cc5ccccc5C#Cc5ccccc54)CC2)CC1)CC3]
or a pharmaceutically acceptable salt thereof.

75. The compound of claim 72, wherein the compound is selected from: 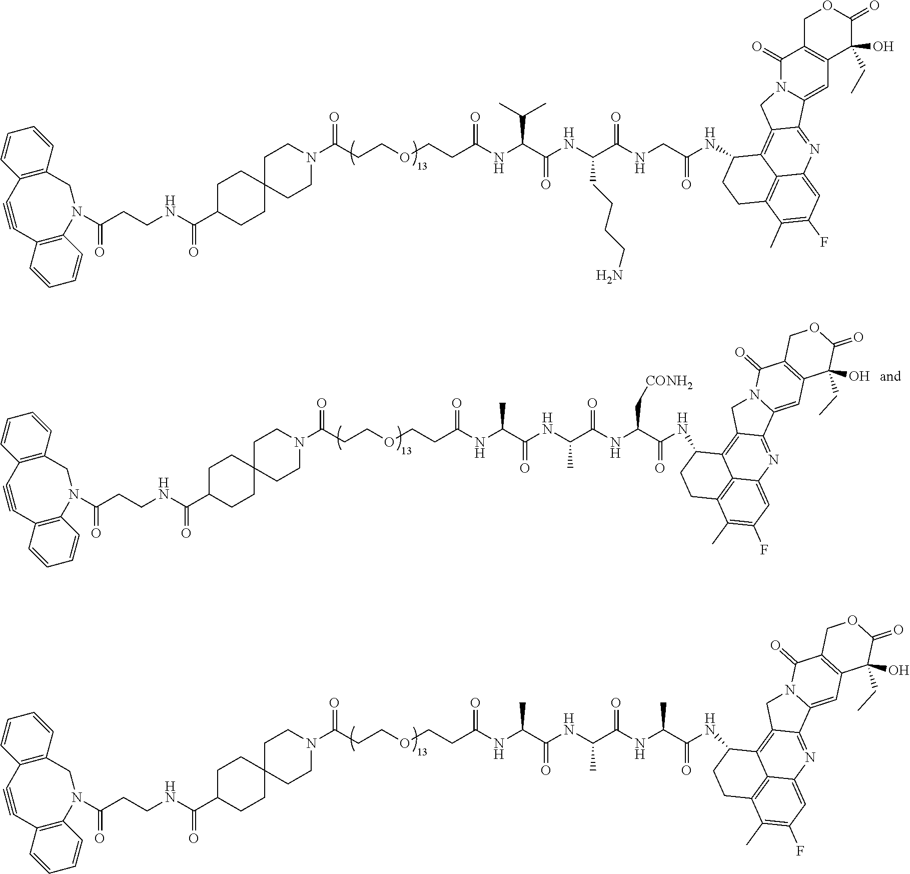
or a pharmaceutically acceptable salt thereof.

76. The compound of claim 22, of the formula 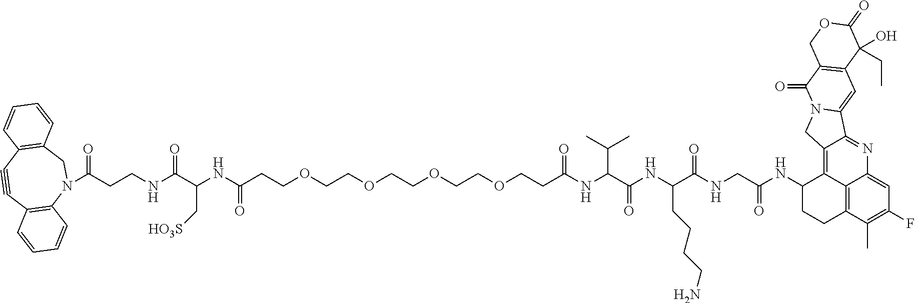 [SMILES: CCC1(O)C(=O)OCc2c1cc1n(c2=O)Cc2c-1nc1cc(F)c(C)c3c1c2C(NC(=O)CNC(=O)C(CCCCN)NC(=O)C(NC(=O)CCOCCOCCOCCOCCC(=O)NC(CS(=O)(=O)O)C(=O)NCCC(=O)N1Cc2ccccc2C#Cc2ccccc21)C(C)C)CC3]
or a pharmaceutically acceptable salt thereof.

77. The compound of claim 76, of the formula: 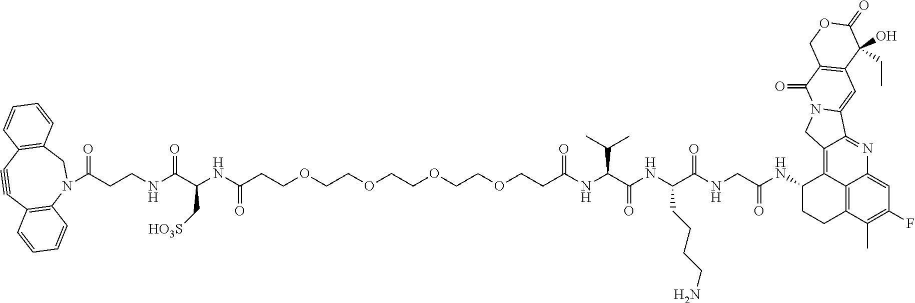 [SMILES: CC[C@@]1(O)C(=O)OCc2c1cc1n(c2=O)Cc2c-1nc1cc(F)c(C)c3c1c2[C@@H](NC(=O)CNC(=O)[C@H](CCCCN)NC(=O)[C@@H](NC(=O)CCOCCOCCOCCOCCC(=O)N[C@@H](CS(=O)(=O)O)C(=O)NCCC(=O)N1Cc2ccccc2C#Cc2ccccc21)C(C)C)CC3]
or a pharmaceutically acceptable salt thereof.

78. A conjugate comprising the compound of any one of claims 1-77, or a pharmaceutically acceptable salt thereof, linked to a second compound.

79. The conjugate of claim 78, of Formula II: 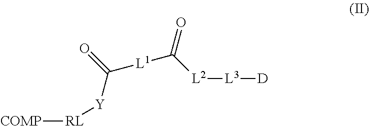
or a pharmaceutically acceptable salt thereof;
wherein
L¹ is selected from 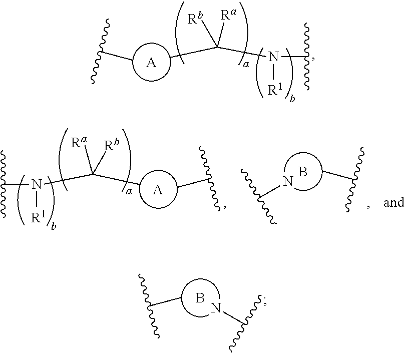
Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b is an integer selected from 0 and 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y is *—C(O)—(CR^{a}R^{b})_{c}—NH— or *—C(O)—(CR^{a}R^{b})_{c}—, wherein * represents where Y is bound to RL;
c is an integer selected from 1, 2, 3, 4, 5, and 6;
RL is a reactive residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is 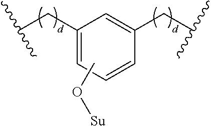
wherein Su is a hexose form of a monosaccharide;
d is an integer independently selected from 1, 2, and 3;
D is a cytotoxic payload;
COMP is a residue of a second compound, and [symbol]
represents attachment to the remainder of the compound.

80. The conjugate of claim 78, of Formula (IV): 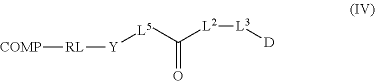
or a pharmaceutically acceptable salt and/or regioisomer thereof;
wherein
L⁵ is a linker comprising an unnatural amino acid;
Y is *—C(O)—(CR^{a}R^{b})_{c}—NH— or *—C(O)—(CR^{a}R^{b})_{c}—, wherein * represents where Y is bound to RL;
c is an integer selected from 1, 2, 3, 4, 5, and 6;
RL is a reactive residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})ₐ—Z-AA-C(O)—, —C(O)O-L⁴-Z—C(O)—(CR^{a}R^{b})ₐ—Z—C(O)—, -AA-Z—, or (CR^{a}R^{b})ₐ—Z-AA-C(O)—;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
Z is selected from —NR²— and —O—;
AA is an amino acid residue or a peptide residue;
L⁴ is 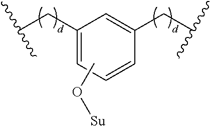
wherein Su is a hexose form of a monosaccharide;
d is an integer independently selected from 1, 2, and 3;
D is a cytotoxic payload;
COMP is a residue of a second compound; and [symbol]
represents attachment to the remainder of the compound.

81. The conjugate of claim 79 or 90, wherein COMP is a residue of a polypeptide.

82. The conjugate of claim 79 or 80, wherein COMP is a residue of an antibody.

83. The conjugate of claim 79 or 80, wherein COMP is a residue of an antibody chain.

84. The conjugate of any one of claims 79 and 81-83, wherein L¹ is

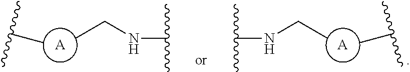

85. The conjugate of any one of claims 79 and 81-83, wherein Ring A of L is an optionally substituted bridged, fused, or spirocyclic bicyclic carbocycle, wherein the carbocycle of Ring A is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl.

86. The conjugate of claim 85, wherein Ring A of L¹ is an optionally substituted C₄₋₁₂ bridged, fused, or spirocyclic bicyclic carbocycle.

87. The conjugate of claim 86, wherein Ring A of L¹ is an optionally substituted C₄₋₁₂ bridged bicyclic carbocycle.

88. The conjugate of claim 87, wherein Ring A of L¹ is an optionally substituted C₄₋₈ bridged bicyclic carbocycle.

89. The conjugate of any one of claims 79 and 8-183, wherein L¹ is

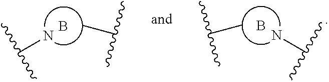

90. The conjugate of any one of claims 79 and 81-83, wherein Ring B of L¹ is an optionally substituted 5- to 12-membered N-linked bridged, fused, or spirocyclic bicyclic heterocycle containing 1, 2, or 3 heteroatoms independently selected from N, O, and S including the N to which the ring is attached, wherein the heterocycle of Ring B is optionally substituted with one or more substituents selected from C₁₋₁₂alkyl, C₂₋₁₂alkenyl, C₂₋₁₂alkynyl, C₃₋₁₂ cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl.

91. The conjugate of any one of claims 79 and 81-83, wherein Ring B of L¹ is an optionally substituted 5- to 12-membered N-linked spirocyclic bicyclic heterocycle containing 1, 2, or 3 heteroatoms independently selected from N, O, and S including the N to which the ring is attached.

92. The conjugate of any one of claims 79 and 81-83, wherein L¹ is selected from 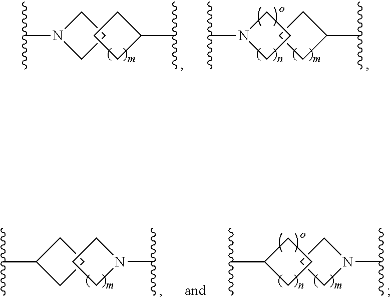
wherein m is an integer selected from 1, 2, 3, 4, and 5; and
each of n and o is an integer independently selected from 1, 2, and 3.

93. The conjugate of an one of claims 79 and 81-83 wherein L¹ is selected from 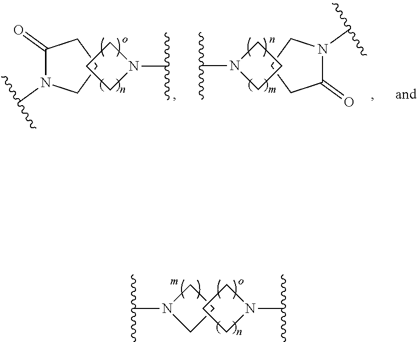
wherein m is an integer selected from 1, 2, 3, 4, and 5; and
each of n and o is an integer independently selected from 1, 2, and 3.

94. The conjugate of any one of claims 79 and 81-83, where Ring B is selected from 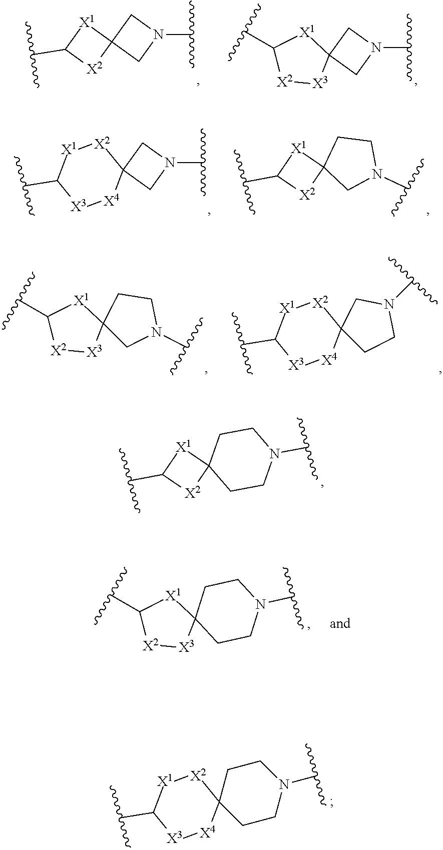
wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

95. The conjugate of claim 94, wherein Ring B is selected from

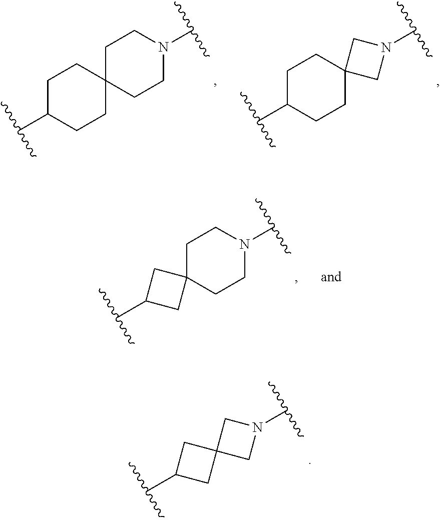 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

96. The conjugate of any one of claims 78 and 81-83, where Ring B is selected from 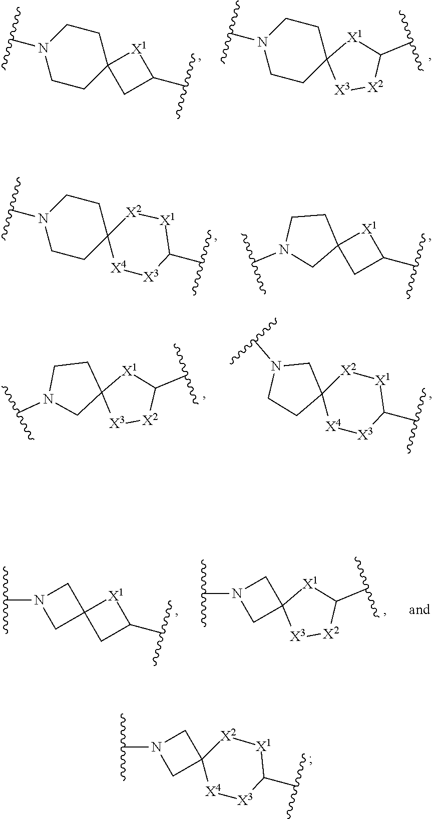
wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

97. The conjugate of claim 96, wherein Ring B is selected from

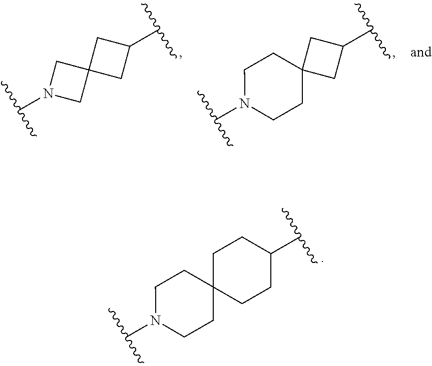 [SMILES: *C1CC2(C1)CN(*)C2 ; *C1CC2(CCN(*)CC2)C1 ; *C1CCC2(CC1)CCN(*)CC2]

98. The conjugate of any of claims 79 and 81-83, wherein ring A is selected from 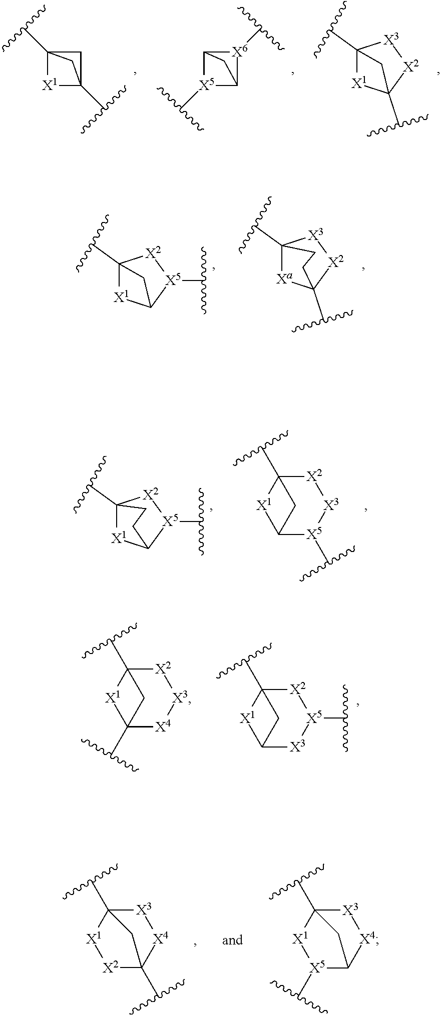
wherein X¹, X², X³, and X⁴ are independently selected from —C(R⁴)₂—, —NH—, —O—, and —S— wherein when X¹, X², and X³ are present, at least one of X¹-X³ is —C(R⁴)₂— and when X¹, X², X³, and X⁴ are present, at least two of X¹-X⁴ are —C(R⁴)₂;
X⁵ is N or CR⁴; and
R⁴ is independently selected from hydrogen, C₂₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

99. The conjugate of any one of claims 79 and 81-83, wherein L is

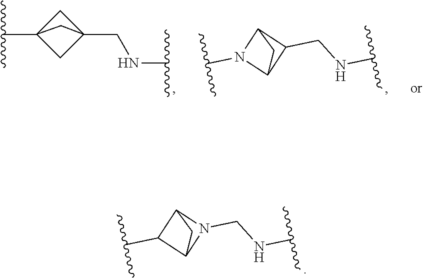

100. The conjugate of claim 99, wherein L¹ is

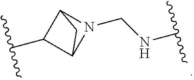 [SMILES: *NCN1C2CC1C2*]

101. The conjugate of claim 80, wherein L⁵ is a linker that comprises at least one amino acid selected from sulfoalanine, hydroxyproline (Hyp), beta-alanine, citrulline (Cit), omithine (Orn), norleucine (Nle), 3-nitrotyrosine, nitroarginine, pyroglutamic acid (Pyr), napthylalanine (Nal), 2,4-diaminobutyric acid (DAB), methionine sulfoxide, and methionine sulfone.

102. The conjugate of claim 101, wherein L⁵ is a linker that comprises

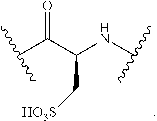 [SMILES: *N[C@@H](CS(=O)(=O)O)C(*)=O]

103. The conjugate of claim 102, wherein L⁵ is

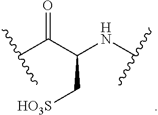 [SMILES: *N[C@@H](CS(=O)(=O)O)C(*)=O]

104. The conjugate of any one of claims 79-103, wherein L² is selected from —(CR^{a}R^{b})ₐ-POLY¹-, -POLY¹-, —(CR^{a}R^{b})ₐ-POLY¹-(CR^{a}R^{b})ₐ—, 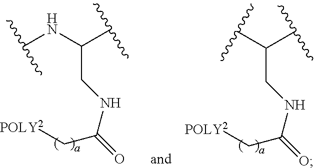
wherein POLY¹ is a divalent hydrophilic polymer residue, POLY² is a residue of a hydrophilic polymer; and [symbol] represents attachment to the remainder of the compound.

105. The conjugate of any one of claims 79-104, wherein Y is —C(O)—(CR^{a}R^{b})_{c}—NH—.

106. The conjugate of claim 105, wherein Y is —C(O)—CH₂CH₂—NH—.

107. The conjugate of any one of claims 79-104, wherein Y is —C(O)—(CR^{a}R^{b})_{c}—.

108. The conjugate of claim 107, wherein Y is —C(O)—(CH₂)₄—.

109. The conjugate of any one of claims 79-108, wherein RL comprises a triazole.

110. The conjugate of any one of claims 79-108, wherein RL is selected from the group consisting of

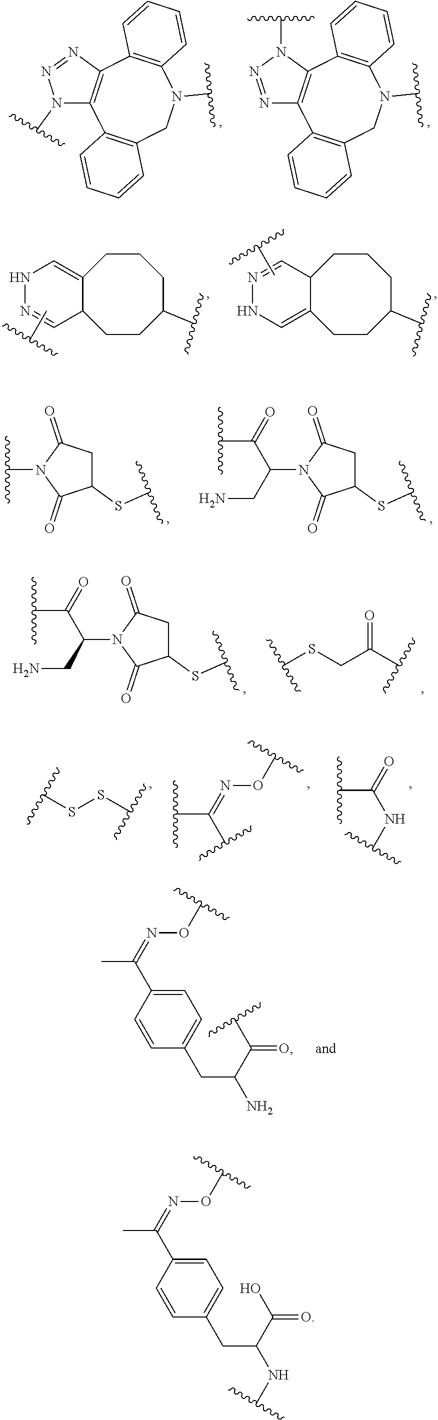

111. The conjugate of claim 110, wherein RL is 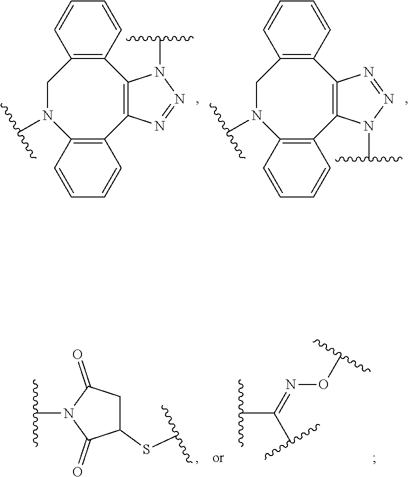 [SMILES: *N1Cc2ccccc2-c2c(nnn2*)-c2ccccc21 ; *N1Cc2ccccc2-c2nnn(*)c2-c2ccccc21 ; *SC1CC(=O)N(*)C1=O ; *ON=C(*)*]
and [symbol] represents attachment to the remainder of the compound.

112. The conjugate of any one of claims 79-103 and 109-111, wherein L²-(CR^{a}R^{b})ₐ-POLY¹-.

113. The conjugate of claim 112, wherein POLY¹- is a divalent residue of polyethylene glycol (PEG), poly(propylene glycol) (PPG), or a copolymer of ethylene glycol and propylene glycol.

114. The conjugate of claim 113, wherein the divalent hydrophilic polymer residue has the formula 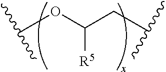
wherein R⁵ is hydrogen or methyl and x is an integer between 1 and 100, inclusive.

115. The conjugate of claim 114, wherein x is 1-25, inclusive.

116. The conjugate of claim 115, wherein x is 13.

117. The conjugate of any one of claims 79-103 and 109-111, wherein L² is 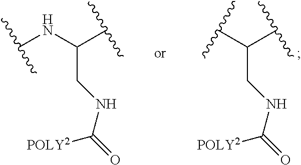
wherein [symbol] represents attachment to the remainder of the compound.

118. The conjugate of claim 117, wherein POLY² is residue of polyethylene glycol (PEG), methoxypolyethylene glycol (mPEG), poly(propylene glycol) (PPG), or a copolymer of ethylene glycol and propylene glycol.

119. The conjugate of claim 118, wherein POLY² is 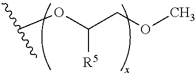
wherein R⁵ is hydrogen or methyl and x is an integer between 1 and 100, inclusive.

120. The conjugate of claim 119, wherein x is 1 to 25, inclusive.

121. The conjugate of claim 120, wherein x is 11.

122. The conjugate of any one of claims 79-121, wherein L³ is —C(O)-AA-.

123. The conjugate of any one of claims 79-121, wherein L³ is —C(O)-AA-Z—(CR^{a}R^{b})ₐ—Z—(CR^{a}R^{b})ₐ—C(O)—, —Z-AA-, or -AA-.

124. The conjugate of any one of claims 79-121, wherein L³ is -AA-.

125. The conjugate of claim 124, wherein -AA- is

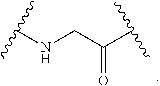 [SMILES: *NCC(*)=O]

126. The conjugate of any one of claims 122-124, wherein AA is a dipeptide residue, a tripeptide residue, a tetrapeptide residue, or a pentapeptide residue.

127. The conjugate of claim 126, wherein AA is a tripeptide residue.

128. The conjugate of any one of claims 122-124 and 126-127, where AA comprises at least one amino acid residue selected from alanine, glycine, valine, and asparagine.

129. The conjugate of any one of claims 122-124 and 126-127, where AA comprises at least one amino acid residue selected from alanine and glycine.

130. The conjugate of any one of claims 122-124 and 126-127, wherein AA is selected from the group consisting of

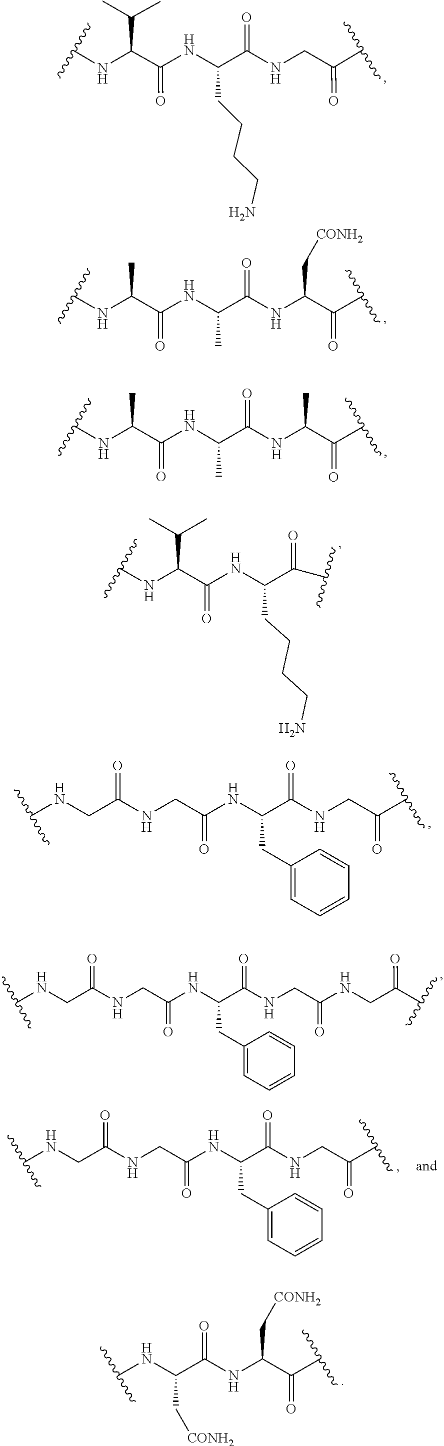

131. The conjugate of any of claim 79-121, wherein
L³ is —C(O)—Z—(CR^{a}R^{b})ₐ—C(O)—Z-L⁴-OC(O)—;
L⁴ is 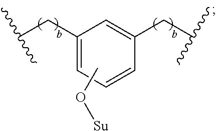
and
Su is a hexose form of a monosaccharide

132. The conjugate of claim 131, wherein Su is

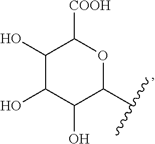 [SMILES: *C1OC(C(=O)O)C(O)C(O)C1O]

133. The conjugate of claim 132, wherein Su is

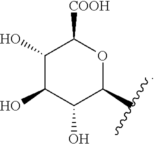 [SMILES: *[C@@H]1O[C@H](C(=O)O)[C@@H](O)[C@H](O)[C@H]1O]

134. The conjugate of any one of claims 79-133, wherein the D is a cytotoxic payload selected from a tubulin inhibitor, DNA damaging agents, a DNA topoisomerase I inhibitor, or a DNA topoisomerase II inhibitor, RNA polymerase II inhibitor.

135. The conjugate of claim 134, wherein D is a cytotoxic payload selected from the group consisting of a hemiasterlin or derivative thereof, a camptothecin or derivative thereof, camptothecin, an anthracycline or derivative thereof, PNU-159682 or derivative thereof, a PBD or derivative thereof, and a duocarmycin or derivative thereof.

136. The conjugate of claim 134, wherein D is selected from 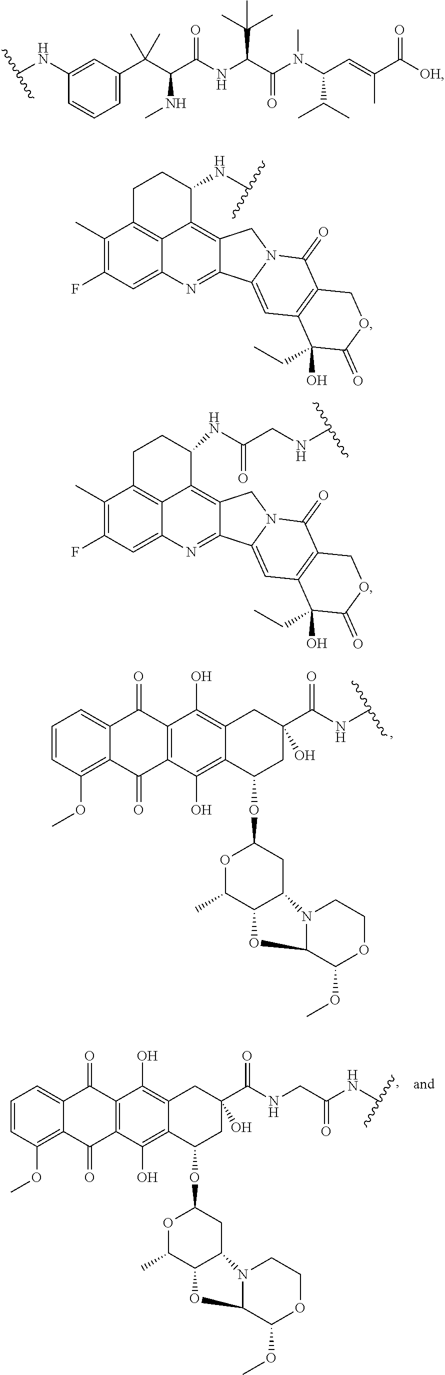 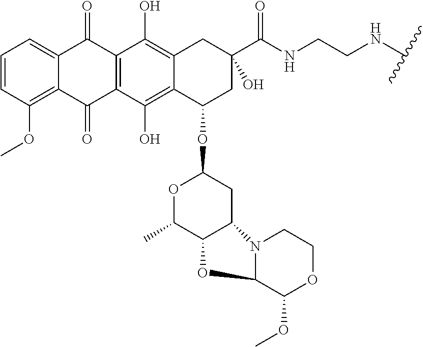
wherein [symbol] represents attachment to the remainder of the compound.

137. The conjugate of claim 134, wherein D is an exatecan payload.

138. The conjugate of claim 79, selected from the group consisting of: 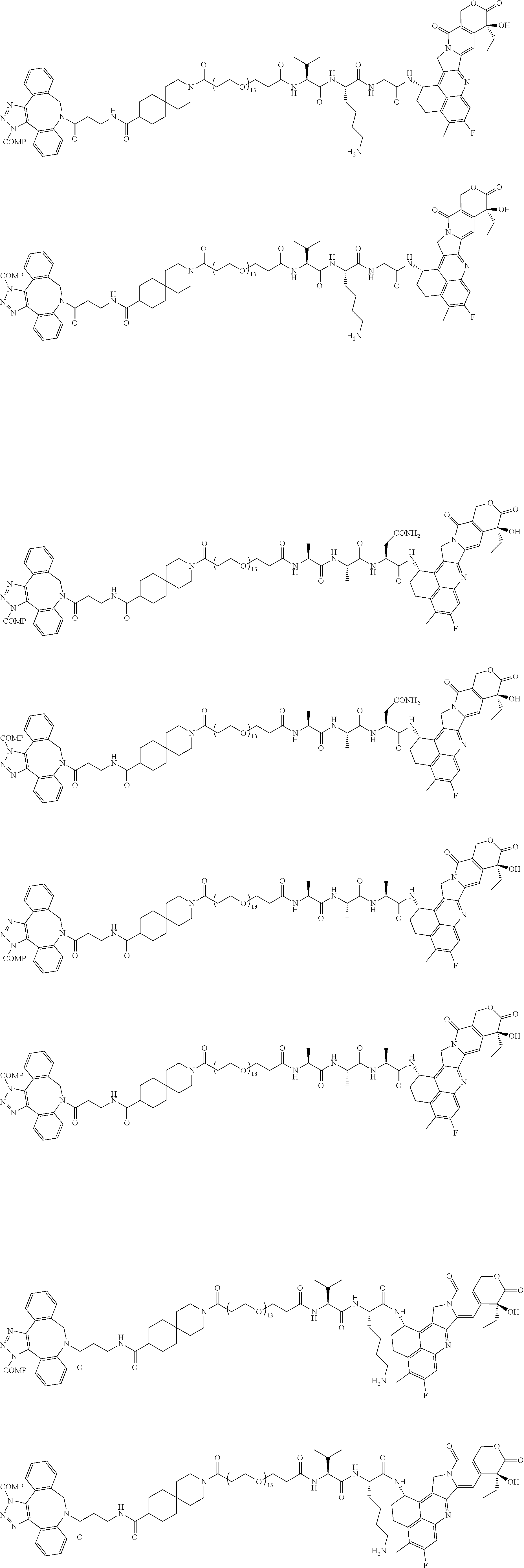 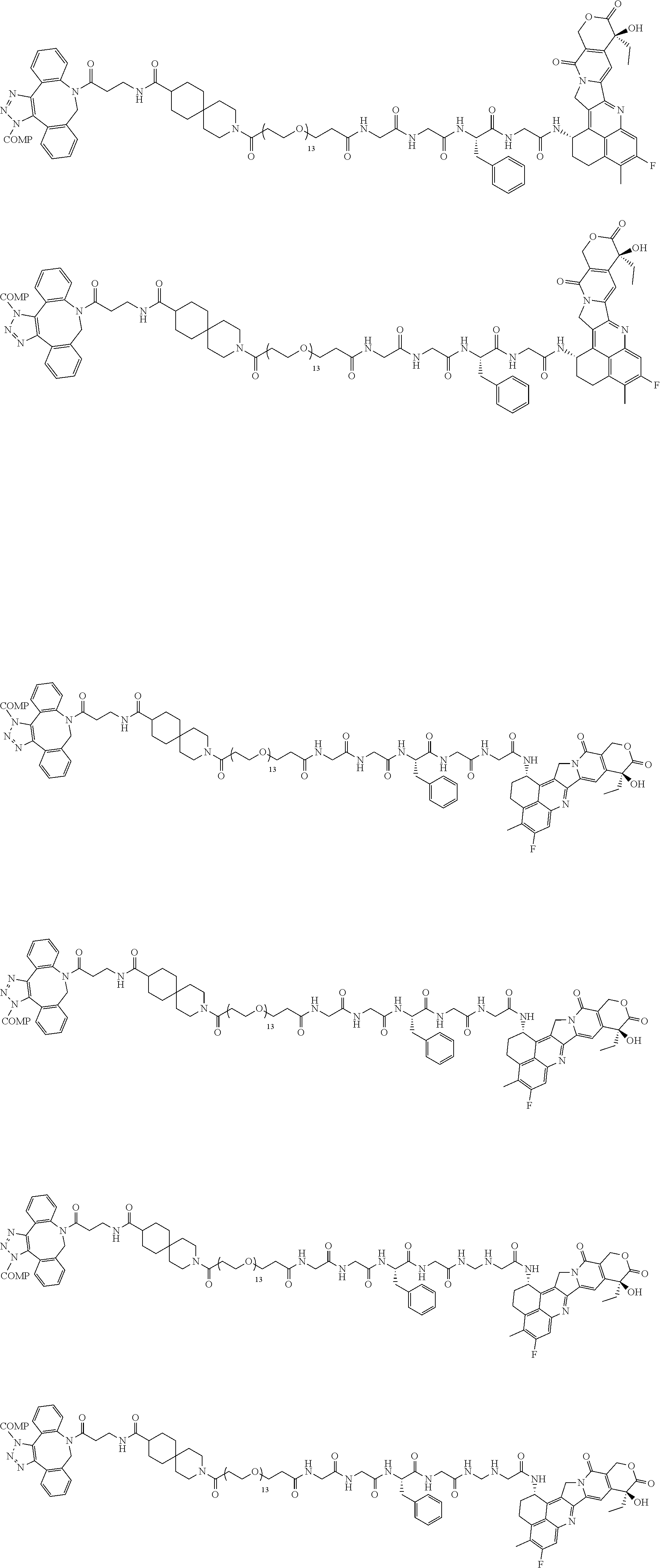 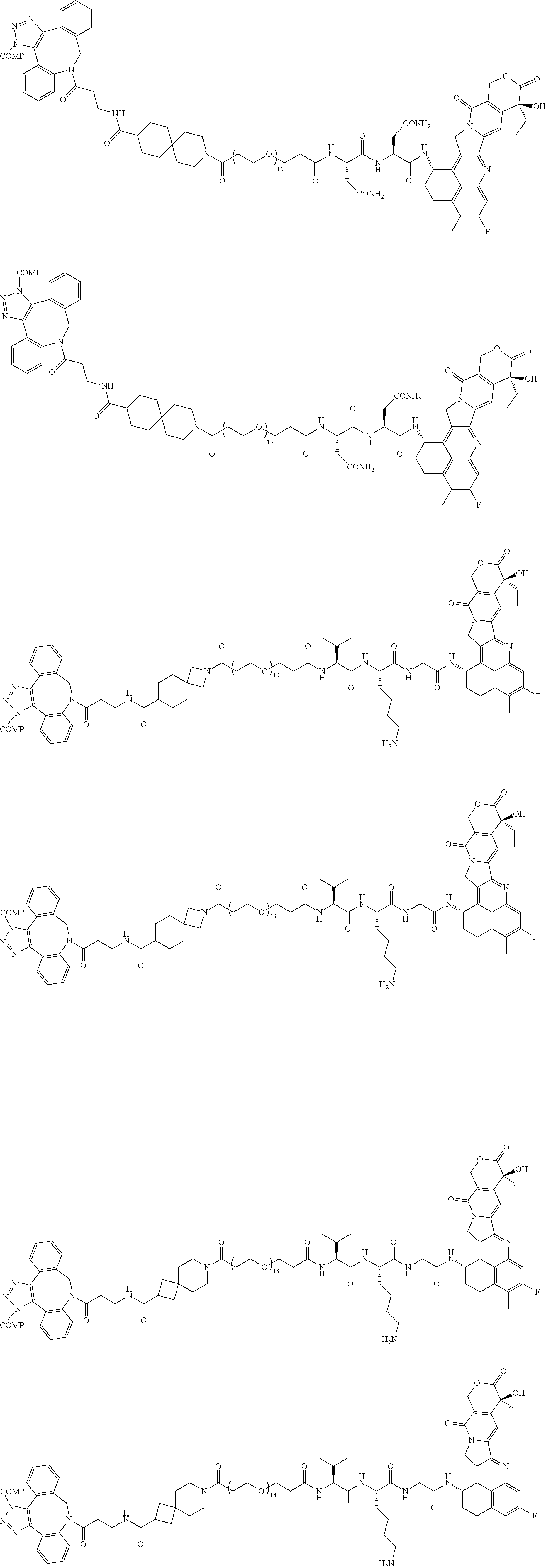 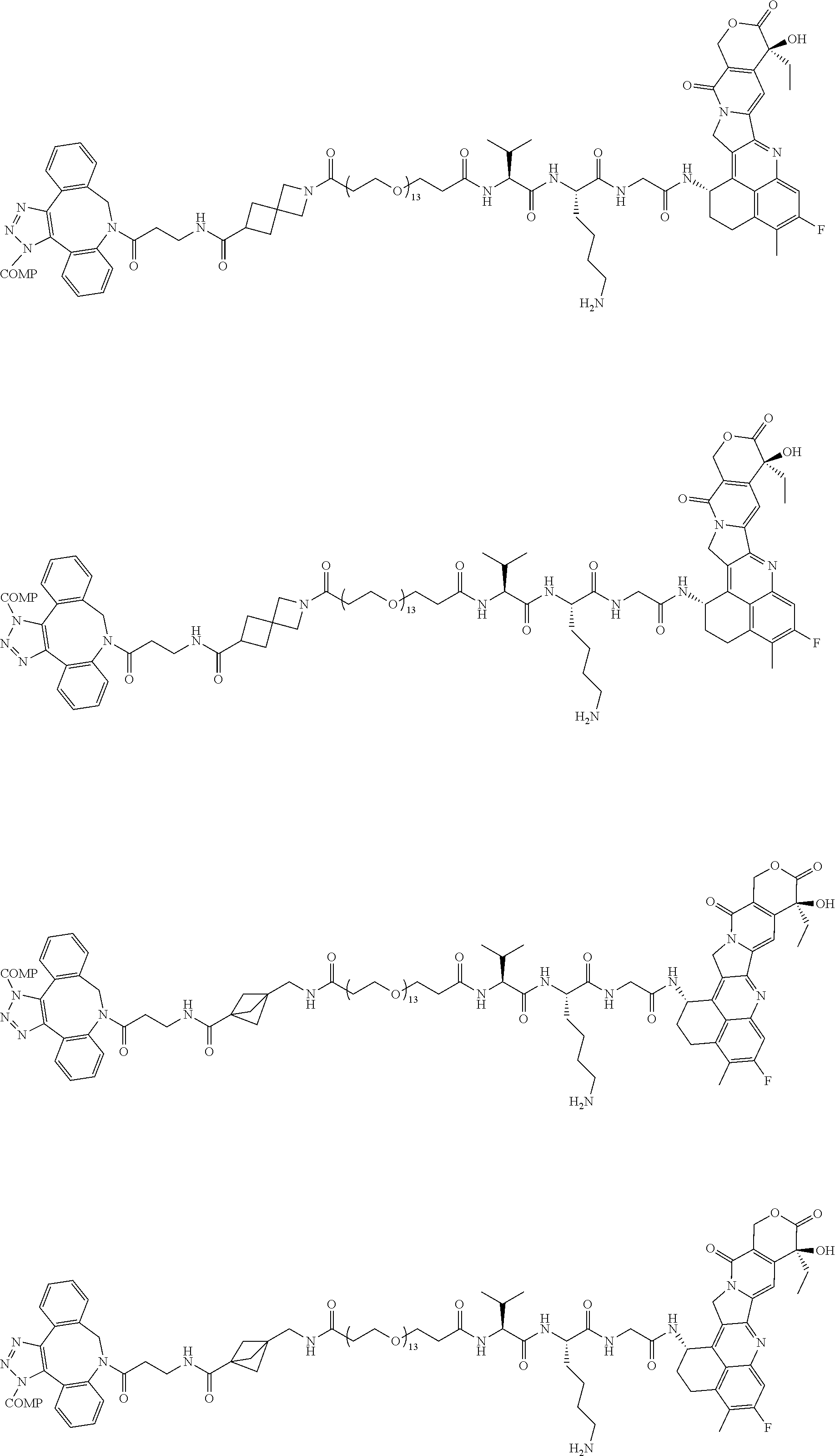 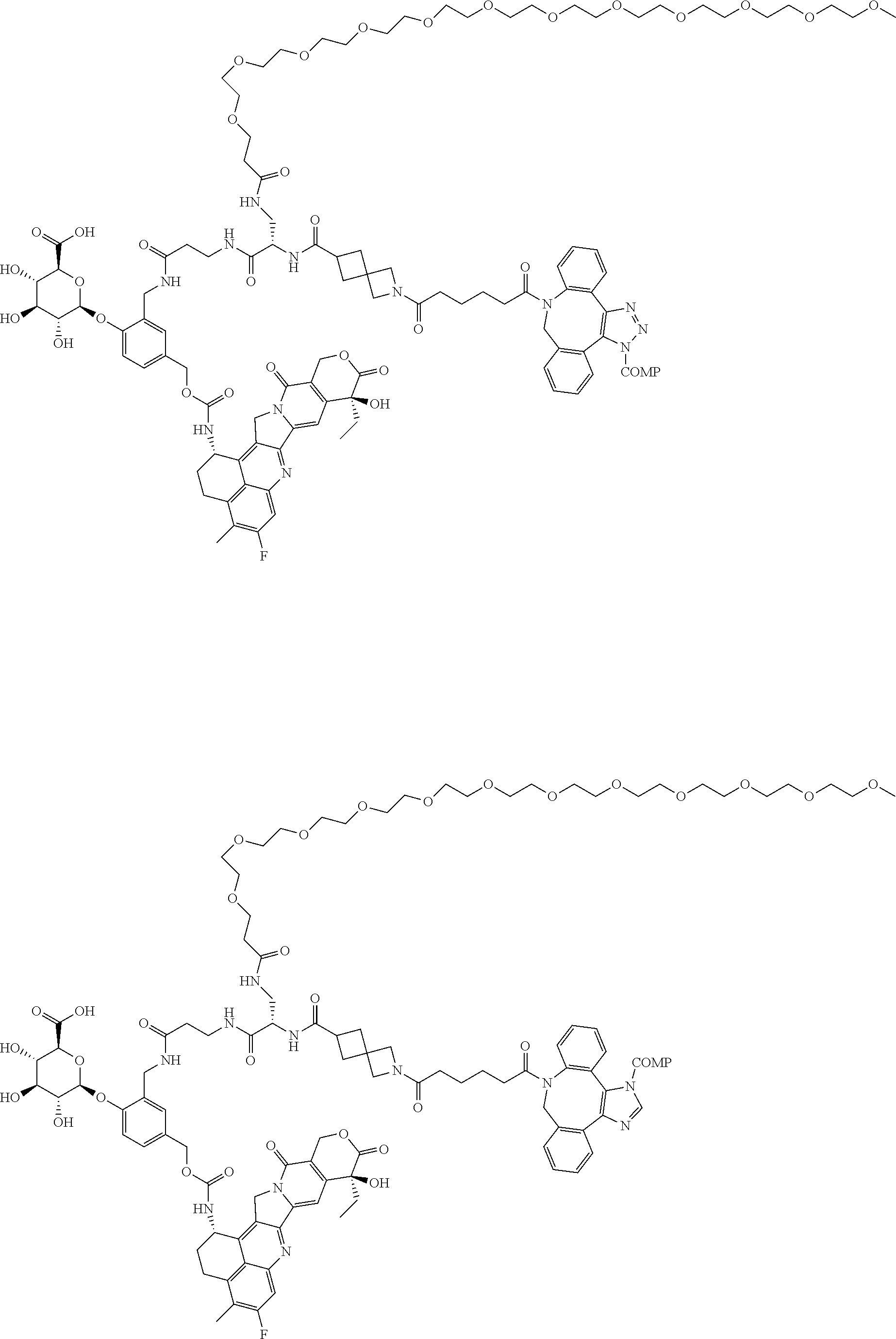 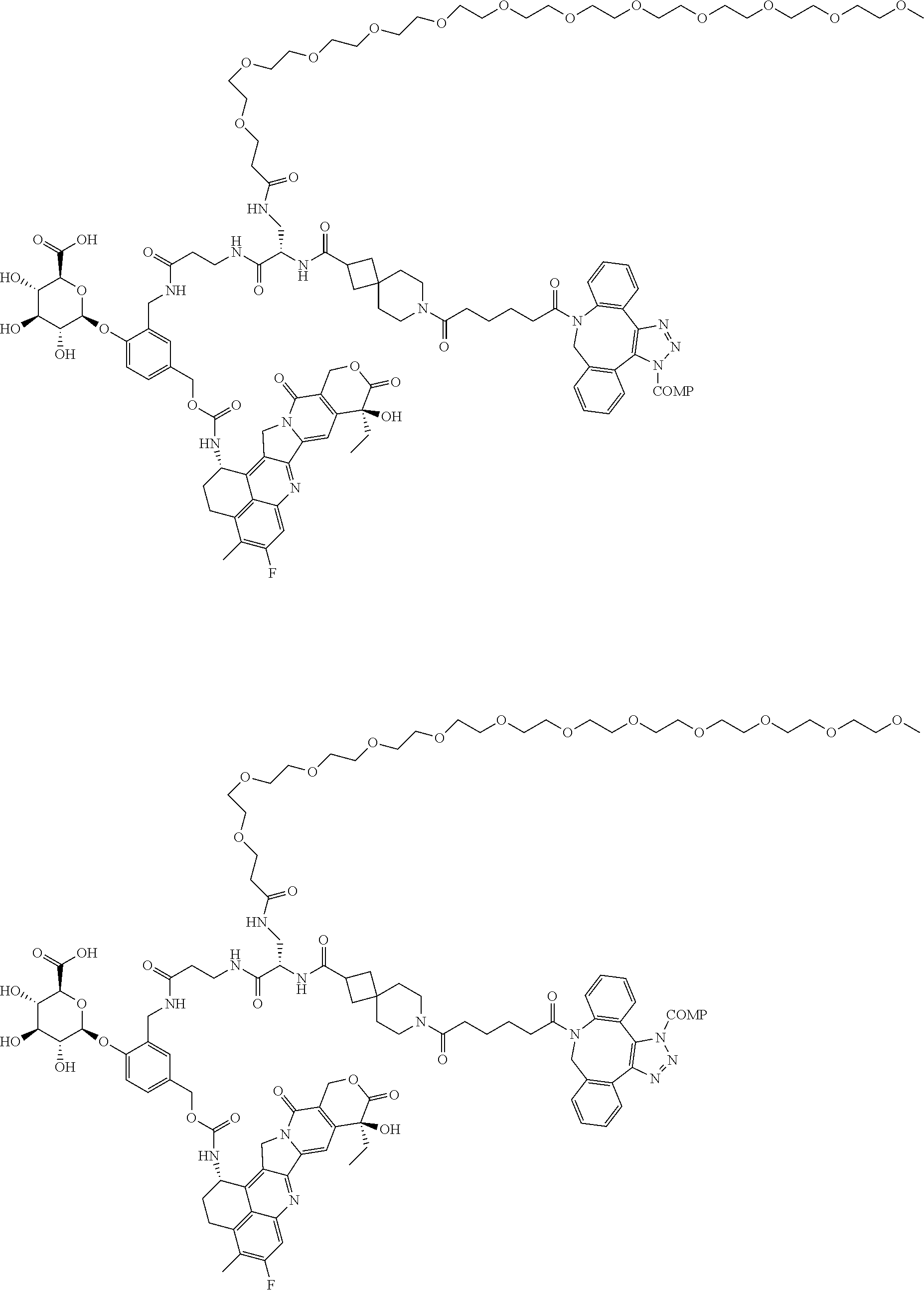 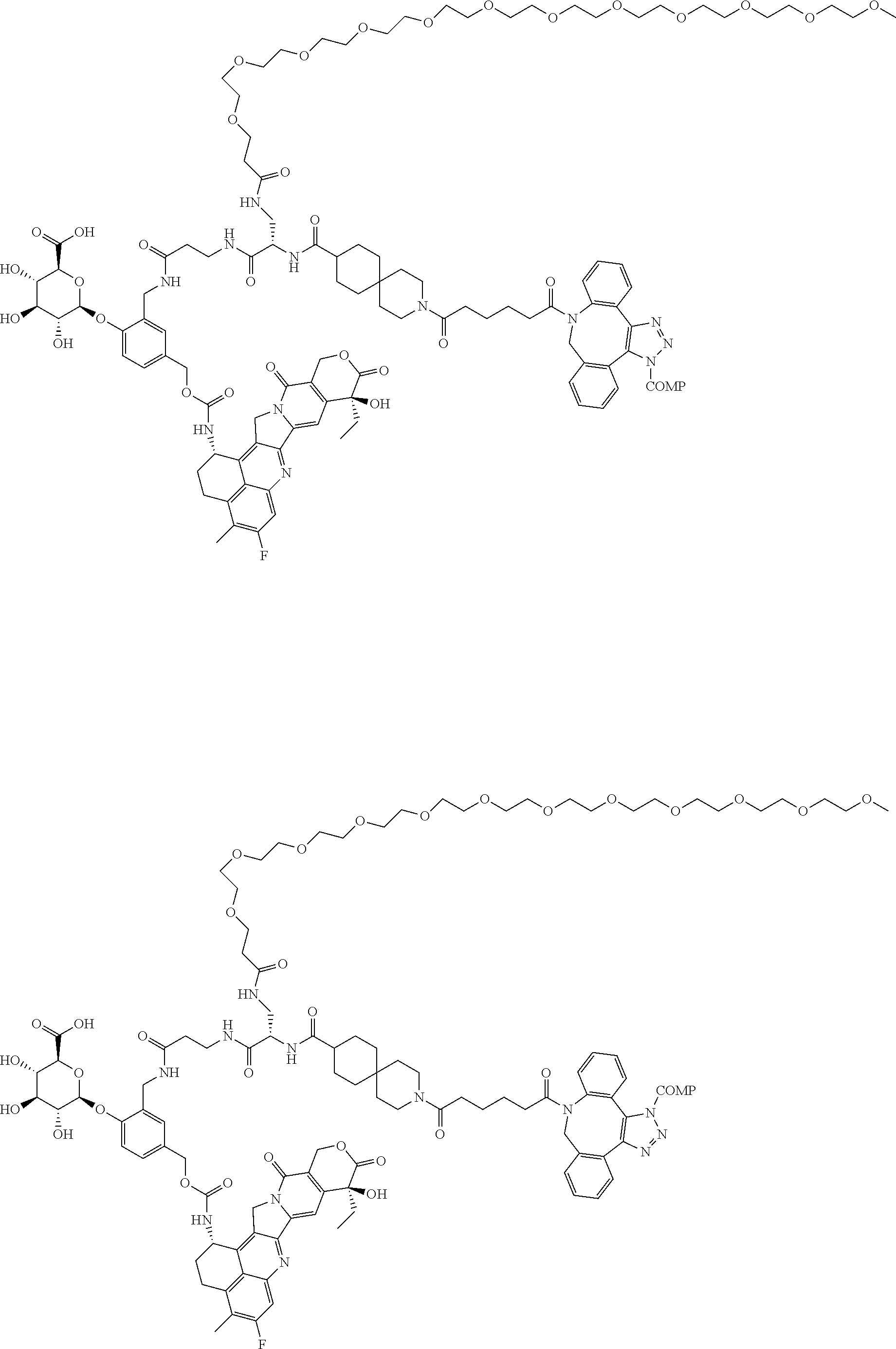 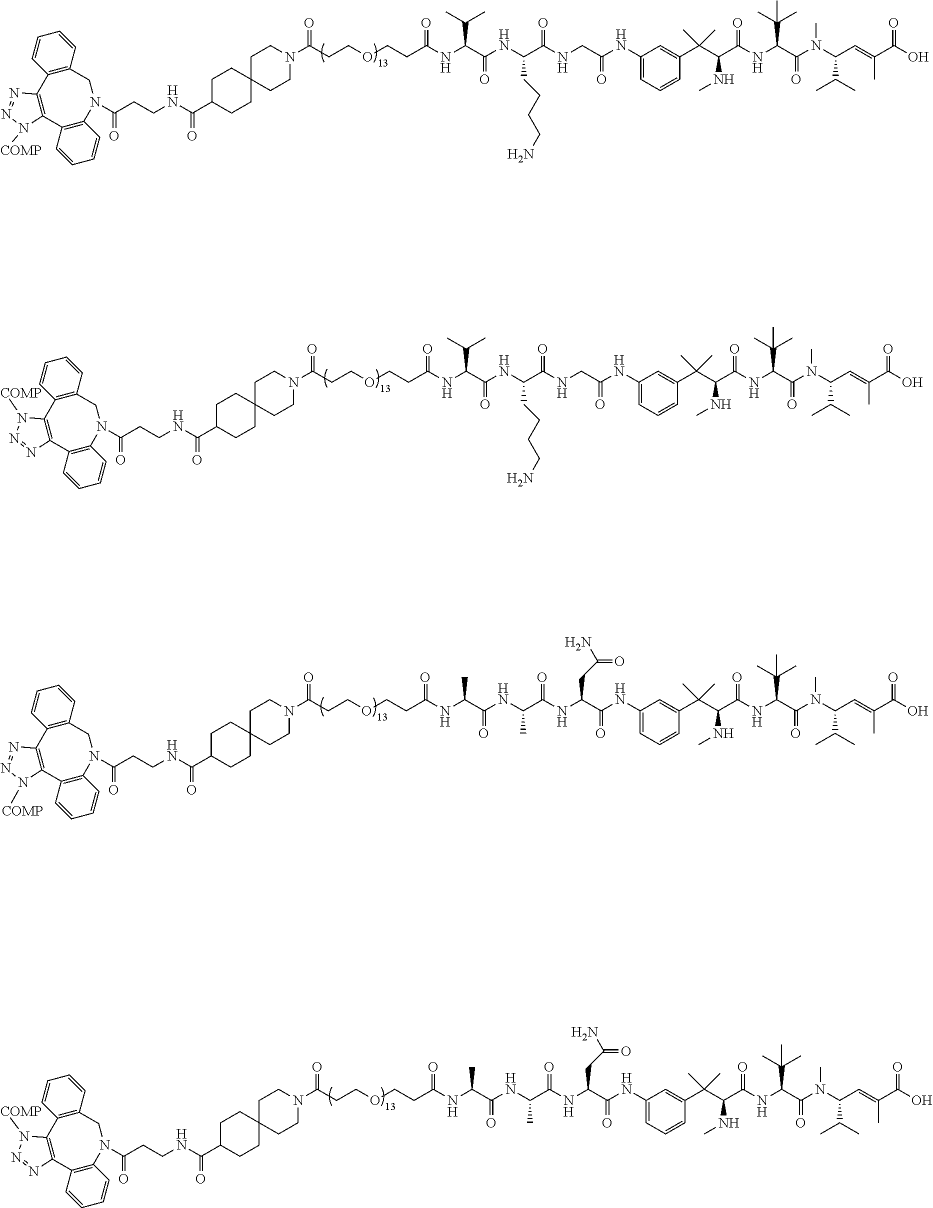 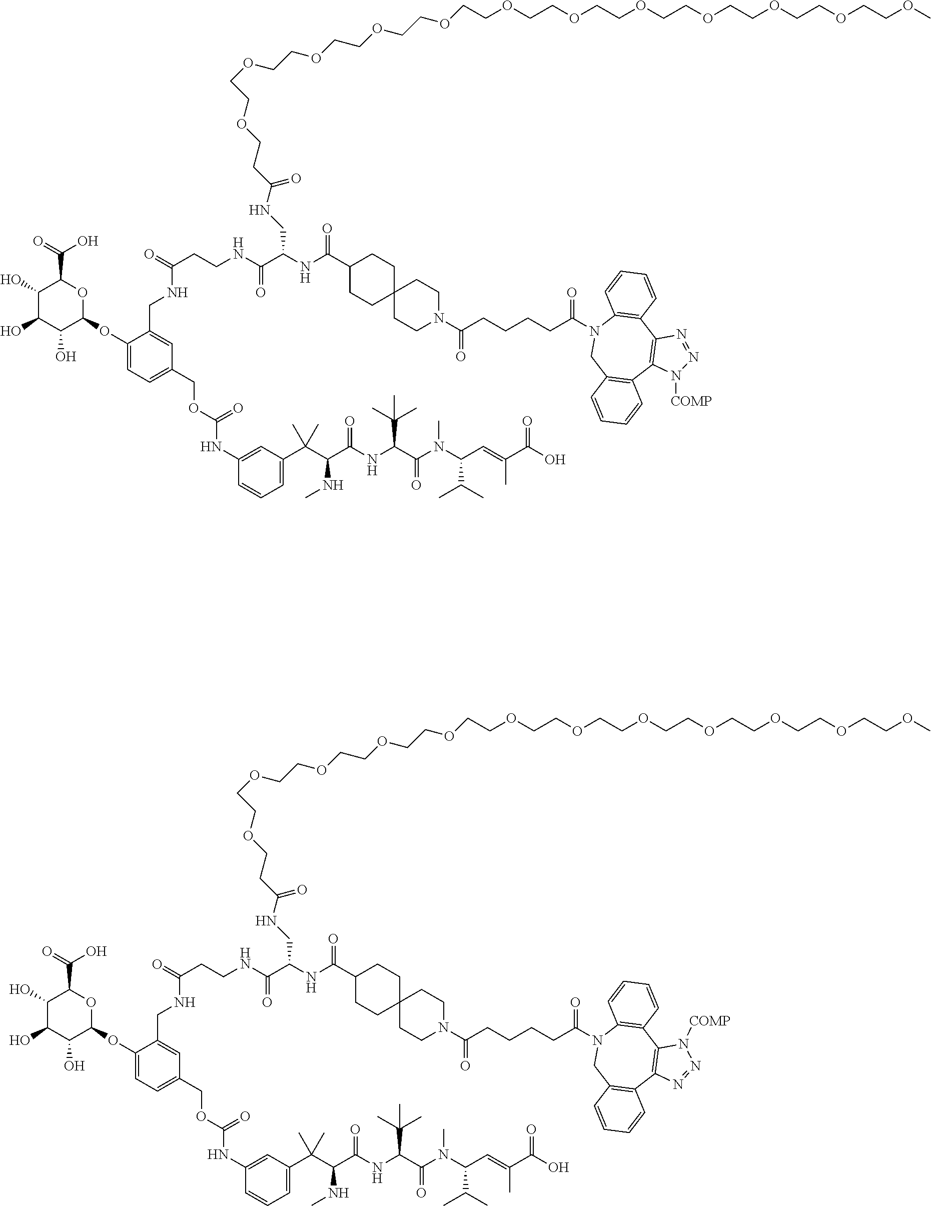 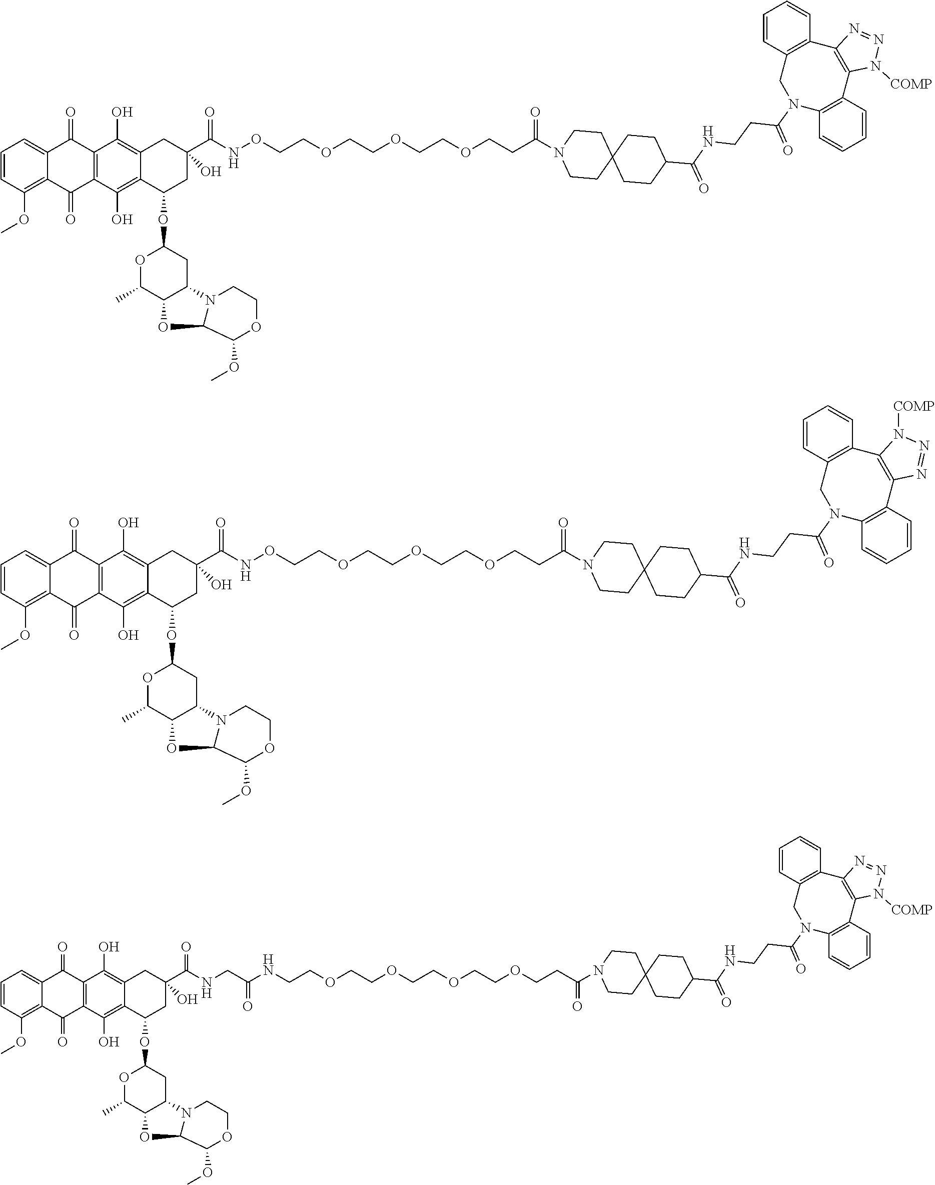 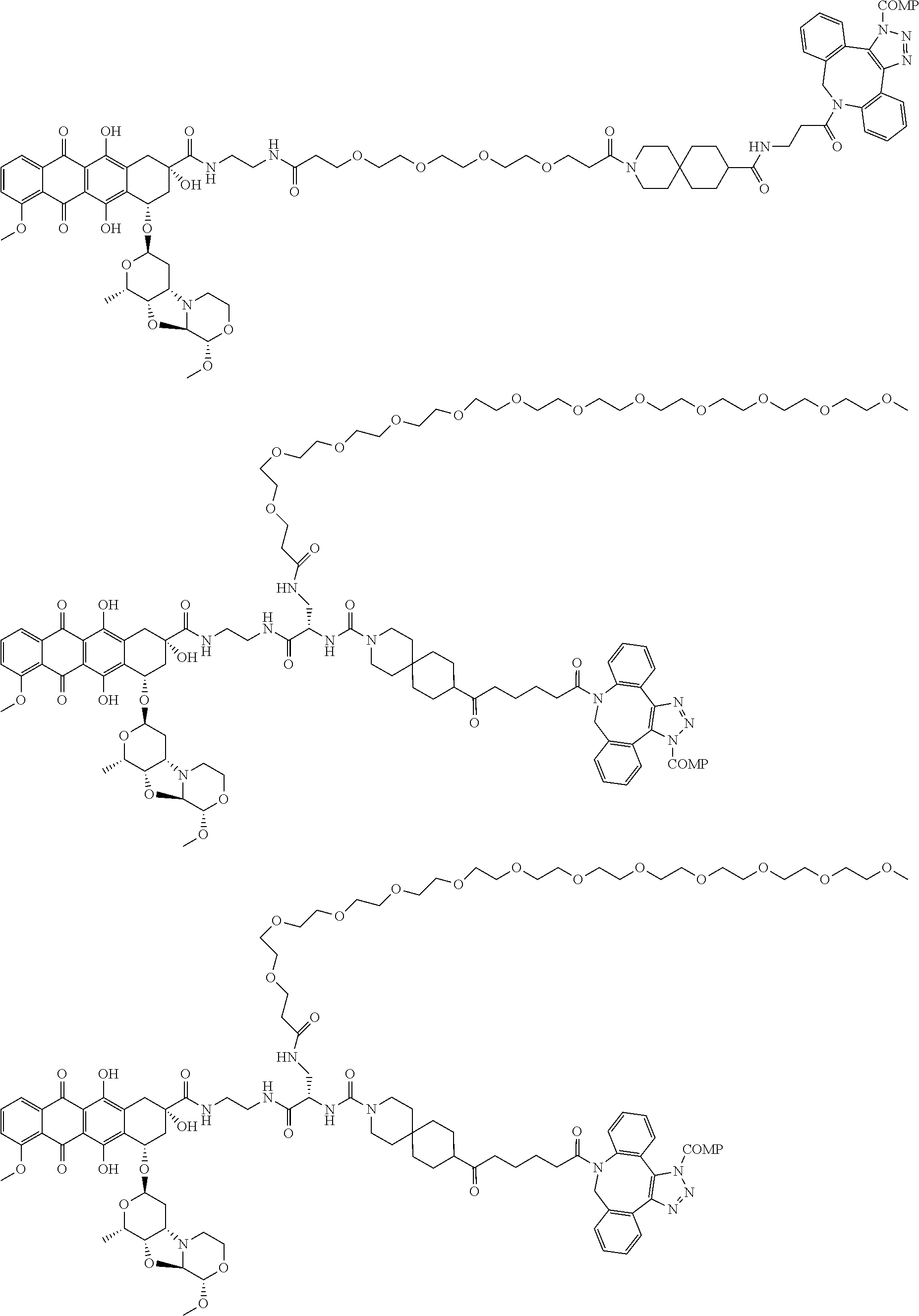 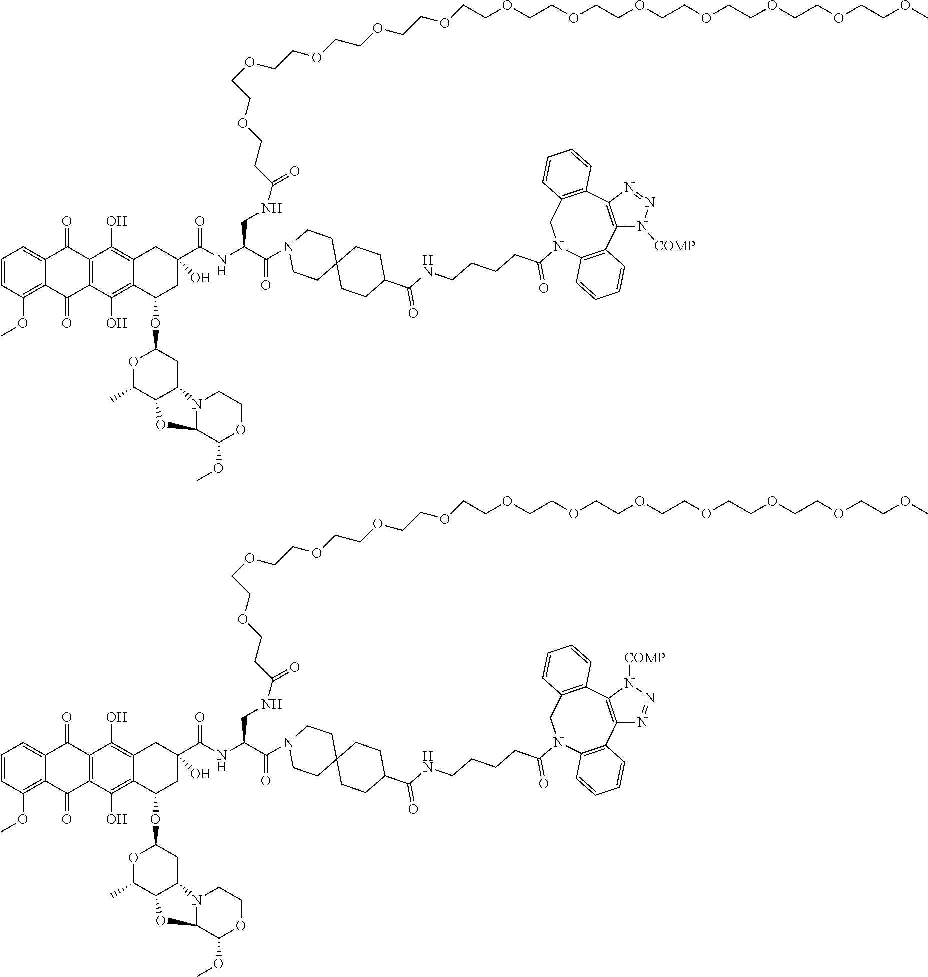 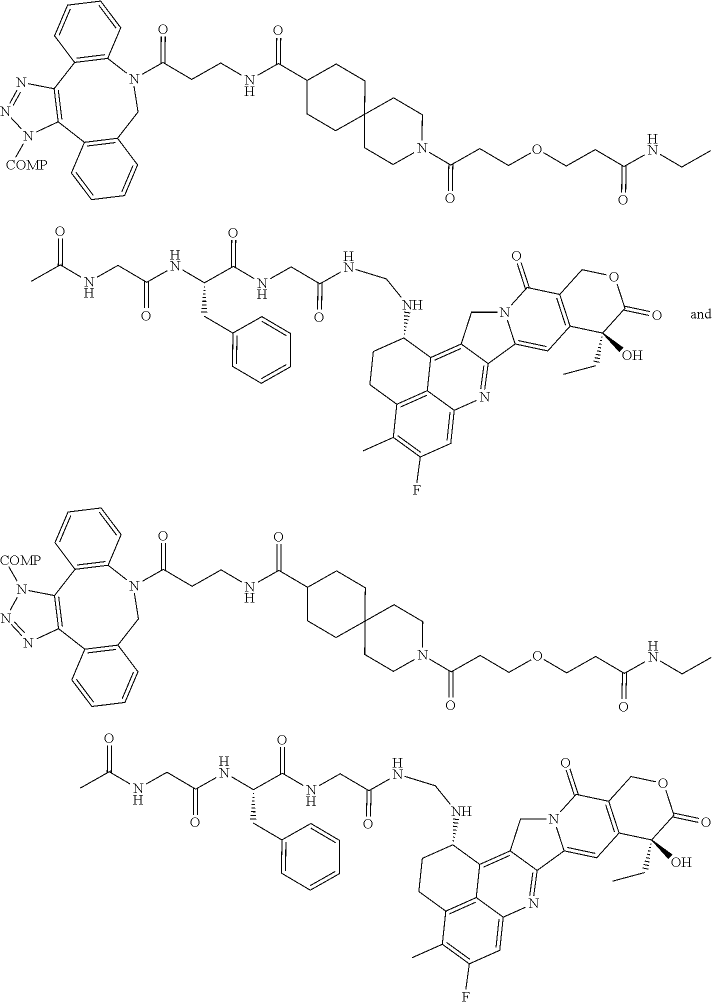
or a pharmaceutically acceptable salt thereof.

139. The conjugate of claim 138 of the formula: 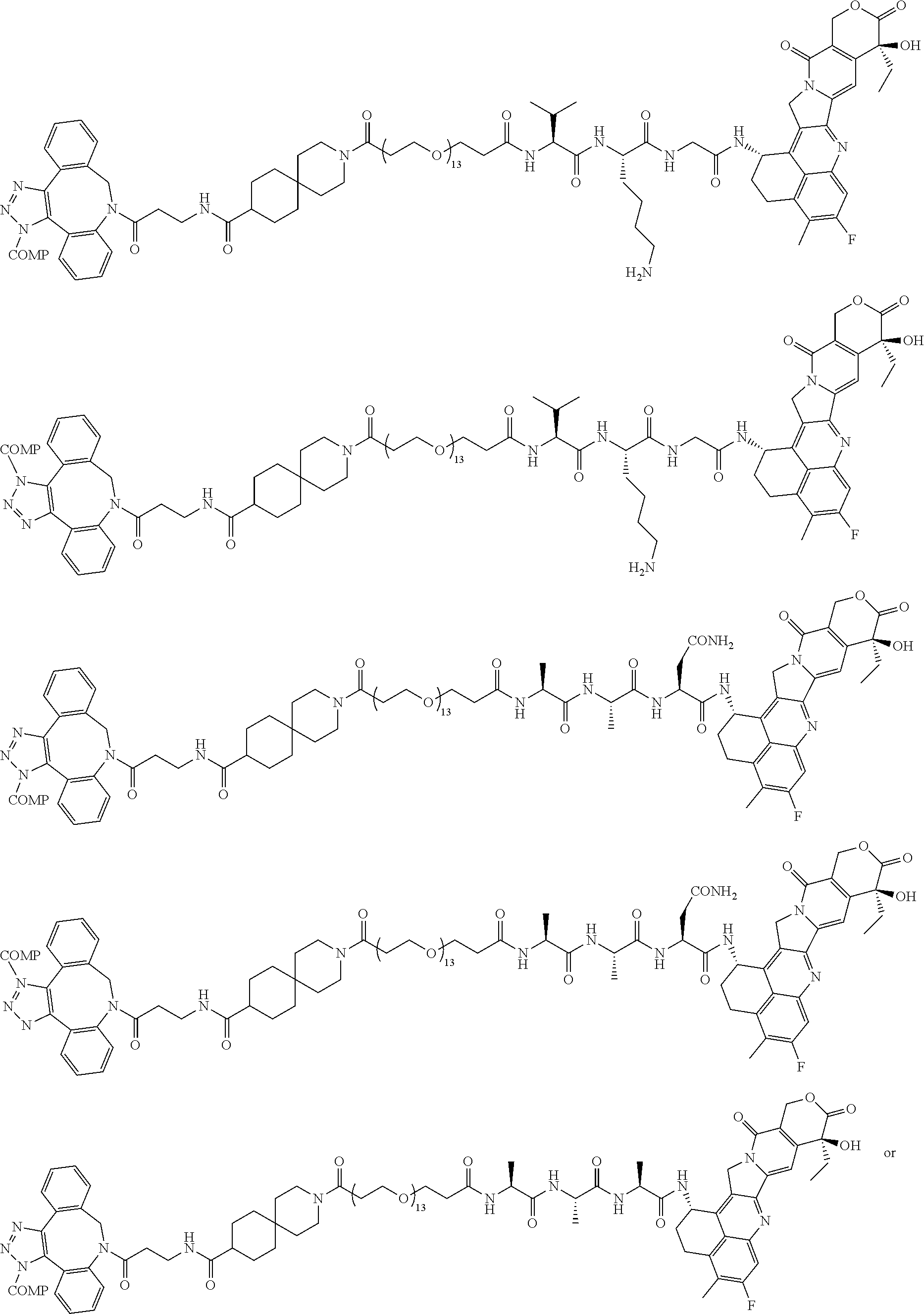 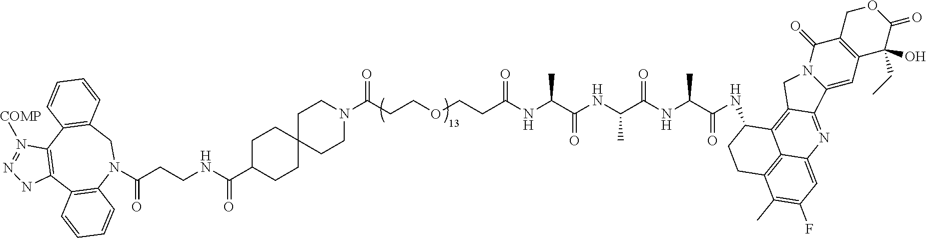
or a pharmaceutically acceptable salt thereof.

140. The conjugate of claim 80, of the formula: 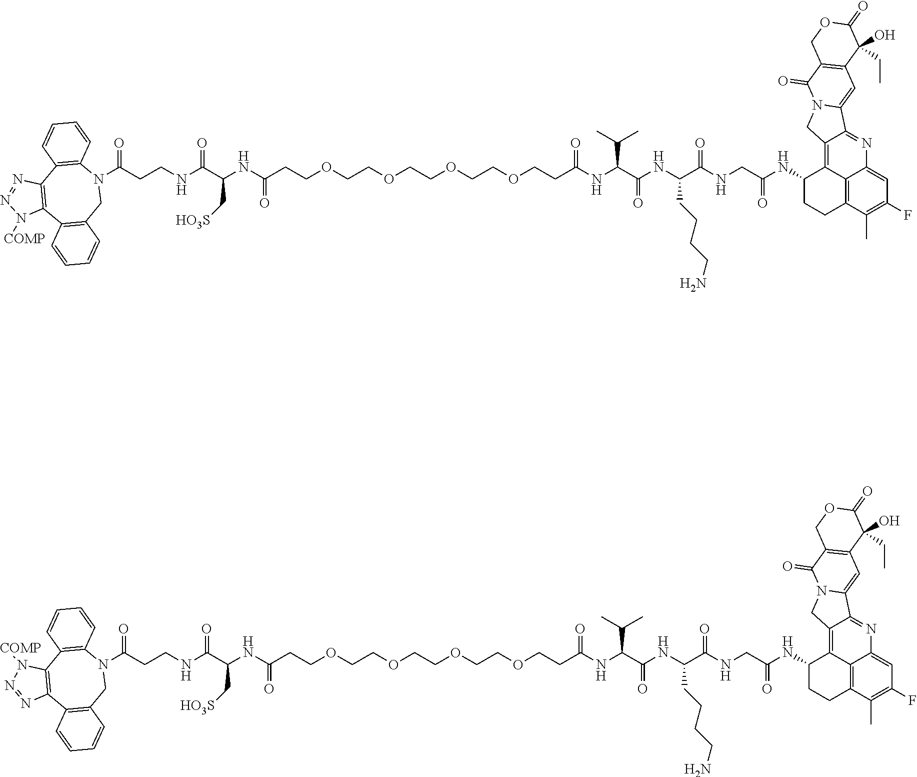
or a pharmaceutically acceptable salt thereof.

141. A pharmaceutical composition comprising the compound of any one of claims 1-77 or a pharmaceutically acceptable salt thereof or the conjugate of any one of claims 78-140 or a pharmaceutically acceptable salt thereof; and a pharmaceutically acceptable excipient, carrier, or diluent.

142. A method of treating a disease or disorder in a subject in need thereof comprising administering a compound of any one of claims 1-77 or a pharmaceutically acceptable salt thereof, a conjugate of any one of claims 78-140 or a pharmaceutically acceptable salt thereof, or the pharmaceutical composition of claim 141.

143. A method of inhibiting tubulin polymerization in a subject in need thereof comprising administering a compound of any one of claims 1-77 or a pharmaceutically acceptable salt thereof, a conjugate ofany one of claims 78-140 or a pharmaceutically acceptable salt thereof, or the pharmaceutical composition of claim 141.

144. The method of claim 142, wherein the disease or disorder is abnormal cellular proliferation.

145. The method of claim 144, wherein the abnormal cellular proliferation is cancer.

146. The method of claim 145, where the cancer is small cell lung cancer, non-small cell lung cancer, ovarian cancer, platinum-resistant ovarian cancer, ovarian adenocarcinoma, endometrial cancer, breast cancer, breast cancer which overexpresses HER2, triple-negative breast cancer, a lymphoma, large cell lymphoma, diffuse mixed histiocytic and lymphocytic lymphoma, follicular B cell lymphoma, colon cancer, colon carcinoma, colon adenocarcinoma, colorectal adenocarcinoma, melanoma, prostate cancer, or multiple myeloma.

147. Use of a compound of any one of claims 1-77 or a pharmaceutically acceptable salt thereof, a conjugate of any one of claims 78-140 or a pharmaceutically acceptable salt thereof, or the pharmaceutical composition of claim 141 for the treatment of a disease or disorder in a subject in need thereof.

148. Use of a compound of any one of claims 1-77 or a pharmaceutically acceptable salt thereof, a conjugate of any one of claims 78-140 or a pharmaceutically acceptable salt thereof, or the pharmaceutical composition of claim 141 for the preparation of a medicament for the treatment of a disease or disorder in a subject in need thereof.

149. The use of claim 147 or 148, wherein the treatment is the inhibition of tubulin polymerization. Use of a compound of any one of claims 1-77 or a pharmaceutically acceptable salt thereof, a conjugate of any one of claims 78-140 or a pharmaceutically acceptable salt thereof, or the pharmaceutical composition of claim 141 for the preparation of a medicament for inhibiting tubulin polymerization in a subject in need thereof.

150. The use of claim 147 or 148, wherein the disease or disorder is abnormal cellular proliferation or cancer.

151. A method of reducing cell proliferation in a subject in need thereof comprising administering a compound of any one of claims 1-77 or a pharmaceutically acceptable salt thereof, a conjugate of any one of claims 78-140 or a pharmaceutically acceptable salt thereof, or the pharmaceutical composition of claim 141.

152. A method of producing a conjugate, comprising contacting the compound of any of claims 1-77 with a second compound under conditions suitable for conjugating the compound of any of claims 1-77 with the second compound; wherein the second compound comprises an alkyne, cyclooctyne strained alkene, tetrazine, methylcyclopropene, thiol, maleimide, carbonyl, amine, oxyamine, or azide.
